(19) United States
(12) Patent Application Publication
(10) Pub. No.: US 2025/0180555 A1
(43) Pub. Date: Jun. 5, 2025

(54) SINGLE-MOLECULE ANALYSIS OF NUCLEIC ACID BINDING PROTEINS

(71) Applicant: UNIVERSITY OF PITTSBURGH - OF THE COMMONWEALTH SYSTEM OF HIGHER EDUCATION, Pittsburgh, PA (US)

(72) Inventors: Bennett Van Houten, Pittsburgh, PA (US); Brittani L. Schnable, Pittsburgh, PA (US); Matthew Schaich, Pittsburgh, PA (US); Namrata Kumar, Philadelphia, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/048,578

(22) Filed: Feb. 7, 2025

Related U.S. Application Data
(63) Continuation of application No. PCT/US2023/029754, filed on Aug. 8, 2023.
(60) Provisional application No. 63/396,089, filed on Aug. 8, 2022.

(51) Int. Cl. G01N 33/557; G01N 33/58

(52) U.S. Cl. CPC G01N 33/557; G01N 33/582

(57) ABSTRACT

The present disclosed subject matter relates to assays, methods, and kits for determining protein-nucleic acid association and dissociation kinetics.

### CROSS-REFERENCE TO RELATED APPLICATION

This application is a continuation of International Application No. PCT/US23/29754, filed on Aug. 8, 2023, which claims priority to U.S. Provisional Patent Application No. 63/396,089, filed on Aug. 8, 2022, the contents of each of which are incorporated in their entireties, and to each of which priority is claimed.

### GRANT INFORMATION

This invention was made with government support under Grant No. R35 ES031638-01 awarded by the National Institute of Health. The government has certain rights in the invention.

### SEQUENCE LISTING

The instant application contains a Sequence Listing, which has been submitted in XML format via EFS-Web and is hereby incorporated by reference in its entirety. Said XML copy, created on Feb. 3, 2025, is named 0723961063_ST26.xml and is 12,196 bytes in size.

### 1. FIELD

The present disclosed subject matter relates to assays, methods and kits for determining protein-nucleic acid association and dissociation kinetics.

### 2. BACKGROUND

Observing DNA-binding proteins interact with DNA substrates in real-time at the single-molecule level illuminates how proteins detect and bind to their targets at extraordinary detail. Key information regarding binding stoichiometry, order of assembly and disassembly, and how proteins diffuse to find their DNA targets are gained through single molecule analysis. Various imaging techniques and optical platforms have been employed to resolve fluorescent proteins to the single-molecule level, but most of these techniques cluster into two broad categories: studies performed with purified proteins with defined conditions or studies performed in living cells.

In single-molecule fluorescence studies of DNA-binding proteins, the molecules of interest must first be purified and then be labeled with a fluorescent tag, ranging in size from small chemical dyes to fluorescent proteins to large quantum dots (Qdots). These techniques hold the distinct advantage of knowing precisely what proteins are binding to the DNA substrates of interest held in a static location. However, overexpressing, purifying, and labeling some proteins can prove difficult due to loss of activity. In addition, even using Qdots conjugation with antibodies, labeling is less than 100%. Furthermore, other protein factors that may contribute to stabilizing or destabilizing ligand binding and/or catalytic activity are lost during purification. The resulting studies of purified DNA-binding proteins may therefore not accurately represent how these proteins work in the context of the complex cellular milieu of the nucleus.

Conversely, single-molecule studies of DNA-binding proteins have also been performed within living cells. These techniques were developed for prokaryotes initially, but recent work has allowed for this imaging even in mammalian cells. While these approaches are the most biologically relevant, watching DNA-binding proteins sort through the complex genome to find their specific binding sites has proven challenging, but technically possible. However, these approaches rely on having low enough fluorescence signal to resolve individual proteins, and therefore there are often many unlabeled proteins of interest competing and altering binding lifetimes. Furthermore, proteins diffusion along DNA cannot be studied when DNA strand orientation is unknown.

Accordingly, there is a need in the art for new techniques to analyze interactions between DNA-binding proteins and DNA.

### 3. SUMMARY

The present disclosed subject matter provides assays, methods and kits for determining protein-nucleic acid association and dissociation kinetics.

In a first aspect, the present disclosure provides assays for determining the binding kinetics of one or more proteins with a nucleic acid substrate, e.g., a DNA substrate or an RNA substrate. In certain embodiments, the assay includes expressing one or more recombinant proteins in a host cell, preparing a nuclear extract from the host cell expressing the one or more recombinant proteins, contacting the nuclear extract with a nucleic acid substrate, e.g., a DNA substrate, visualizing the one or more recombinant proteins binding to the nucleic acid substrate, e.g., the DNA substrate, and determining protein-nucleic acid, e.g., protein-DNA, association and dissociation kinetics.

In certain embodiments, the one or more recombinant proteins is a natural protein, synthetic protein, modified protein, or other protein analogue. In certain embodiments, the one or more recombinant proteins is a variant, homolog, derivative, mutant or a functional fragment thereof of a wild type protein. In certain embodiments, the one or more recombinant proteins is post-translationally modified. In certain embodiments, the post-translational modification comprises a proteolytic cleavage, glycosylation, or the addition of modifying group, such as acetyl, phosphoryl, glycosyl or methyl, to one or more amino acids of the protein.

In certain embodiments, the one or more recombinant proteins is labeled. In certain embodiments, the one or more recombinant proteins is fluorescently labeled. In certain embodiments, the fluorescent label is a dye, fluorophore or fluorescent protein.

In certain embodiments, the one or more recombinant proteins is selected from the group consisting of nucleic acid-binding proteins, e.g., DNA-binding proteins or RNA-binding proteins, nucleic acid repair proteins, e.g., DNA repair proteins, DNA modifying proteins, DNA damage response proteins, transcription factors, nucleases, chromatin remodeling factors, methylated DNA binding proteins, methylases, demethylases, acetylases, deacetylases, glycosylases, kinases, phosphatases, integrases, recombinases, ligases, topoisomerases, gyrases, polymerases (e.g., DNA polymerases or RNA polymerases), proteases, helicases or a combination thereof. In certain embodiments, the one or more recombinant proteins is selected from a group consisting of poly(ADP-ribose) polymerase 1 (PARP1), heterodimeric ultraviolet-damaged DNA-binding protein 1 and 2 (UV-DDB), xeroderma pigmentosum complementation group C protein (XPC), 8-oxoguanine glycosylase 1 (OGG1), apurinic/apyrimidinic endonuclease 1 (APE1), DNA polymerase beta (Polbeta), Thymine DNA glycosylase (TDG), X-ray repair cross complementing 1 (XRCC1), DNA ligase 3 (Lig3α), poly(ADP-ribose) polymerase 2 (PARP2), alkyladenine glycosylase (AAG), or a combination thereof. In certain embodiments, the host cell is a mammalian cell. In certain non-limiting embodiments, the mammalian cell is selected from a group consisting of a human cell, hamster cell, mouse cell, rat cell, sheep cell, goat cell, monkey cell, dog cell, cat cell, horse cell, cow cell, pig cell or a combination thereof. In certain non-limiting embodiments, the host cell is selected from a group consisting of a U2OS cell, Sf9 cell, CHO cell, COS-7 cell, HEK293 cell, BHK cell, TM4 cell, CV1 cell, VERO-76 cell, HELA cell, MDCK cell, BRL cell, W138 cell, Hep G2 cell, MMT cell, TRI cell, MRC 5 cell, FS4 cell, RPE cell, hTERT-RPE cell, hTERT-BJ fibroblast or a combination thereof.

In certain embodiments, the assay further comprises analyzing the expression level of the one or more recombinant proteins in the nuclear extract, e.g., by Western Blot.

In certain embodiments, the nucleic acid substrate, e.g., DNA substrate, is between about 10 and 100 kb in length, e.g., about 10 to about 70 kb in length. In certain embodiments, the nucleic acid substrate, e.g., DNA substrate, is damaged. In certain embodiments, the damage is a physical or a chemical change. In certain embodiments, the damage is induced by UV exposure, enzymatic digestion, or oxidative damage. In certain embodiments, the nucleic acid substrate comprises one or more nucleic acid analogues. In certain embodiments, the nucleic acid analogues are incorporated into the nucleic acid DNA by nick translation. In certain embodiments, nucleic acid analogue is selected from a group consisting of 5-formyl-dCTP (5fC), 5-hm-dUTP, 6-thio-dGTP, 5-fluoro-dUTP, ara-CTP, Cy3-dUTP, dITP or a combination thereof. In certain embodiments, the nucleic acid substrate (e.g., DNA substrate) can include one or more nucleosomes.

In certain embodiments, the nucleic acid substrate, e.g., DNA substrate, is positioned within a microfluidic cell system, and the nuclear extract is flowed through the microfluidic cell system to contact the nucleic acid substrate, e.g., DNA substrate. In certain embodiments, the microfluidic system further includes optical tweezers. In certain embodiments, the microfluidic system comprises a microfluidic cell having at least 4 channels separated by laminar flow. In certain embodiments, the channel 1 contains beads; channel 2 contains the nucleic acid substrate, e.g., DNA substrate; channel 3 contains the flow buffer; and/or channel 4 contains the cell extract. In certain embodiments, the beads are trapped in channel 1. In certain embodiments, the nucleic acid substrate, e.g., DNA substrate, is suspended between the beads in channel 2. In certain embodiments, a buffer solution is flowed through channel 3. In certain embodiments, the nuclear extract containing the one or more proteins contacts the nucleic acid substrate, e.g., DNA substrate, in channel 4. In certain embodiments, the flow rate is kept constant. In certain embodiments, the flow rate is pulsed. In certain embodiments, the flow is between 0.05 and 0.1 bar. In certain embodiments, the protein-nucleic acid interactions were observed without flow.

In certain embodiments, the beads have a diameter between about 1 and 10 μM. In certain embodiments, the beads are polystyrene. In certain embodiments, the beads are coated with a functional group to facilitate nucleic acid substrate, e.g., DNA substrate, attachment, e.g., streptavidin. In certain embodiments, the nucleic acid substrate, e.g., DNA substrate, contains a functional group to facilitate bead attachment, e.g., biotin. In certain embodiments, the nucleic acid substrate, e.g., DNA substrate, contains a functional group to facilitate bead attachment, e.g., poly-lysine. In certain embodiments, the nucleic acid substrate, e.g., DNA substrate, is tethered to the beads by a biotin-streptavidin interaction. In certain embodiments, the DNA substrate is held at a tension of about 5 to 40 pN.

In certain embodiments, the microfluidic cell system further includes fluorescence microscopy. In certain embodiments, the one or more recombinant proteins is detected by fluorescence microscopy. In certain embodiments, the fluorescence microscopy can resolve an individual one or more proteins binding to a specific location along the nucleic acid substrate, e.g., DNA substrate. In certain embodiments, the fluorescence microscopy comprises single-molecule-FRET imaging. In certain embodiments, the fluorescence microscopy comprises confocal imaging.

In certain embodiments, the association and dissociation kinetics of the one or more recombinant protein comprise: a binding event duration (k_{off}); number of binding events per second (kₒₙ); a binding position; and/or a movement on DNA or RNA (MSD/velocity).

In another aspect, the present disclosure provides a method for determining nucleic acid-binding kinetics of one or more proteins using an assay described herein. In certain embodiments, the present disclosure provides a method for determining nucleic acid, e.g., DNA, damage recognition of one or more proteins using an assay described herein. In certain embodiments, the present disclosure provides a method for determining DNA repair mechanisms using an assay described herein. In certain embodiments, the present disclosure provides a method for determining single molecule analysis of DNA-binding proteins from nuclear extract using an assay described herein.

The present disclosure further provides kits for performing the assays or methods described herein. In certain embodiments, the kit includes a microfluid cell; a buffer fluid; a set of beads; and/or a nucleic acid substrate, e.g., DNA substrate. In certain embodiments, the present disclosure the kit further includes instructions for performing single molecule analysis of nucleic acid binding proteins, e.g., DNA-binding proteins, from nuclear extracts; tracer dyes; and/or reagents for conjugating functional groups.

### 4. BRIEF DESCRIPTION OF THE DRAWINGS

FIGS. 1A-1D depict the workflow and experimental outcomes of single-molecule analysis of DNA-binding proteins from nuclear extracts (SMADNE). FIG. 1A depicts the SMADNE workflow. FIG. 1B depicts a diagram of the imaging techniques using four channels separated by laminar flow. FIG. 1C depicts a schematic of a DNA substrate for SMADNE suspended between two polystyrene beads and tagged proteins (yellow spheres) bound to sites of DNA damage. This substrate (nicked DNA) is shown as a 2D scan (one YFP-PARP1 binding event numbered and circled) and in kymograph mode (numbered spot marked). Event one dissociated before the kymograph started and then another event appeared at the same position later (asterisks). Binding events appear as lines in the kymograph because time is indicated on the X axis and position on the Y axis. FIG. 1D depicts the four major outcomes obtained from SMADNE characterization.

FIGS. 2A-2F depict DNA tension influenced DNA nick detection by poly(ADP-ribose) polymerase (PARP1). FIG. 2A depicts a structural model of PARP1 bound to nicked DNA with YFP tag (PDB codes 3ED8 and 4KLO) generated. FIG. 2B depicts a schematic of the DNA suspended between streptavidin beads containing 10 discrete nicks from the nickase Nt.BspQI. FIG. 2C depicts an example kymograph of PARP1 binding DNA at oscillating tensions from 5 pN to 30 pN. Binding events shown in yellow and tension measurements shown below in blue. FIG. 2D depicts the number of events per second at various DNA tensions held constant. Error bars represent the SEM of three experiments. Gray circle represents undamaged DNA. FIG. 2E depicts an example kymograph of PARP1 binding DNA at constant tension (30 pN). Positional analysis shown to the right showed biding at the expected sites, but also several sites that were bound multiple times that did not contain the recognition sequence by Nt.BspQI. FIG. 2F depicts undamaged DNA exhibited reduced YFP-PARP1 binding, even at 30 pN.

FIGS. 3A-3L depict SMADNE characterization of transient DNA-binding interactions of DNA repair proteins. FIG. 3A depicts the structure of cGFP-XPC (PDB codes 6CFI of Rad4 the yeast homolog to XPC and 4EUL). FIG. 3B depicts a schematic of the DNA substrate used for XPC binding characterization, with UV damage sites shown in yellow and XPC binding shown in blue. Also shown is an example kymograph of cGFP-XPC binding and diffusing along the DNA in yellow. FIG. 3C depicts the results of a CRTD analysis of XPC binding DNA with UV damage. FIG. 3D depicts the distribution of motile and nonmotile XPC events. FIG. 3E depicts an example MSD plot for analyzing XPC diffusion on DNA. FIG. 3F depicts the diffusion and alpha values for the diffusion of XPC on DNA. FIG. 3G depicts a structural model of APE1-tGFP from PDB code (5WNO and 4EUL). FIG. 3H depicts a schematic and example kymograph of APE1 binding to DNA with nicks. FIG. 3I depicts the results of a CRTD analysis of APE1 binding nicked DNA, with fit shown in blue. FIG. 3J depicts a structural model of pol β-tGFP, taken from PDB codes (4KLO and 4EUL) and the tGFP modeled in. FIG. 3K depicts an example schematic of pol β binding DNA containing nicks as well as a corresponding kymograph of an observation of pol β binding. FIG. 3L depicts the results of a CRTD analysis of pol β binding nicked DNA, with the fit shown in blue.

FIGS. 4A-4G depicts SMADNE characterization of dual-labeled UV-DDB binding UV damage. FIG. 4A depicts the structure of UV-DDB bound to DNA (PDB ID: 4E5Z, 4EUL, 5UY1) with modeled fluorescent tags. FIG. 4B depicts an example kymograph of cGFP-DDB1 (blue) and HaloTag-DDB2 (red) binding to 48.5 kb DNA with UV damage. When both colors bind together the color appears magenta. The white asterisk marks an event where DDB1 and DDB2 bound together followed by DDB1 dissociation. Also shown is a graph of the positions of events in the kymograph. FIGS. 4C and 4D depict cumulative residence time distribution (CRTD) for DDB1 (FIG. 4C) and DDB2 (FIG. 4D) binding UV-damaged DNA. FIG. 4E depicts the percentage of events that were DDB1 alone, DDB2 alone, or colocalized (middle). FIG. 4F depicts a diagram showing the 11 possible colocalization categories for two colors of molecules binding DNA. FIG. 4G depicts the distribution of the 11 categories for DDB1 and DDB2 binding UV-damaged DNA. Error bars represent the SEM of four experiments.

FIGS. 5A-5L depict the facilitated dissociation and movement behavior of DDB2 K244E. FIG. 5A depicts a diagram of dual-labeled UV-DDB (with cGFP and HaloTag-JF-635) and unlabeled purified UV-DDB included (PDB ID: 4E5Z, 4EUL, 5UY1). FIG. 5B depicts an example kymograph of labeled DDB1 and DDB2 binding transiently to UV-damaged DNA. FIG. 5C depicts a CRTD plot of DDB1 (blue) and FIG. 5D depicts a CRTD plot of DDB2 (red). Dotted lines indicate CRTD curves without added unlabeled UV-DDB. FIG. 5E, Distribution of events that were DDB1 alone, DDB2 alone, or colocalized. FIG. 5F depicts colocalization categories for DDB1 and DDB2 binding to damaged DNA with error bars as the SEM of three experiments. FIG. 5G depicts the structure of DDB2 bound to a 6-4 photoproduct, with the site of the K244E mutation marked in red. FIG. 5H depicts a kymograph of motile DDB2 K244E binding. The tracked position of the line is shown in orange. FIG. 5I depicts the CRTD plot for all K244E binding events, with motile shown in red and nonmotile events shown in gray. FIG. 5J depicts the distribution of motile and nonmotile events for WT and DDB2 K244E. FIG. 5K depicts the Mean Squared Displacement analysis of motile binding events shown in FIG. 5H. FIG. 5L depicts the diffusivity (D) and a values for K244E events.

FIGS. 6A-6F depict OGG1 and UV-DDB binding to DNA with oxidative damage. FIG. 6A depicts a structural model of mScarlet-tagged OGG1 bound to 8-oxoG containing DNA (PDB codes 1YQR and 5LK4). FIG. 6B depicts a schematic of DNA with 8-oxoG damage shown in blue. The accompanying kymograph shows many transient OGG1 binding events on the DNA in green. FIG. 6C depicts the kymograph of the catalytically dead variant K249Q indicating increased binding lifetimes (blue). FIG. 6D depict the CRTD analysis for WT and K249Q OGG1 at 10 pN. The weighted average lifetime for the mutant was 15.4 s (42.9 and 7.7 s, 78% fast), over tenfold longer than the 1.4 s single-exponential fit. FIG. 6E depicts the kymograph of mScarlet-OGG1 (green), cGFP-DDB1 (blue), and HaloTag-DDB2 (red) with binding positions shown on the right. FIG. 6F depicts the distribution of events that bound alone vs colocalizing for all three proteins.

FIG. 7 depicts the standard curves collected on purified HaloTag protein conjugated to JF-635 and a GFP standard with a linear fit. These measurements were collected by flowing the sample into the flow cell until the photon count stabilized, stopping the flow, and collecting the resultant intensities. These measurements were taken in channel 4 of the flow cell, in the same scan position and Z position used for SMADNE imaging (i.e., the focus was on diffusing fluorescent particles in the flow cell, not on the surface of the glass).

FIG. 8 depicts a representative western blot of overexpressed HaloTag-DDB2 and cGFP-DDB1 in nuclear extracts. Lanes 1-3: Three dilutions of purified UV-DDB, containing 4.8, 2.4, and 1.2 ng of DDB2 and 12.7, 6.4, and 3.2 ng of DDB1, respectively. Lanes 4-6: Various concentrations of nuclear extract loaded, including 3 μL, 1.5 μL, and 0.75 μL. Samples were also blotted for DDB1 and DDB2, but the bands containing the overexpressed proteins are shifted higher because the fluorescent fusion protein increases molecular weight compared to the endogenous protein.

FIG. 9 depicts a schematic showing the proteins identified in nuclear extracts. Nuclear extract was characterized via LC/MS/MS. Out of 1551 proteins identified with annotated Gene Ontology Cellular Component, the most common cellular location was that of nuclear proteins. Additionally, some mitochondrial proteins were also identified out of the nuclear extract.

FIGS. 10A-10B depict the lifetimes of YFP-PARP1 bound to nicked DNA at various tensions. FIG. 10A depicts the Cumulative Residence Time Distribution (CRTD) of YFP-PARP1 binding to nicked DNA at various tensions. Altering the tension only created modest impacts on the lifetime. FIG. 10B depicts the quantification of the weighted average lifetimes of YFP-PARP1 at four different tensions. Error bars represent the SEM of three experiments. Mean weighted average lifetimes were 1.6, 4.3, 4.6, and 3.5 seconds for 5, 10, 20, and 30 pN, respectively.

FIGS. 11A-11I depict SMADNE characterization of proteins at various levels of DNA damage. FIG. 11A depicts a kymograph of YFP-PARP1 on undamaged DNA. FIGS. 11B-11D depicts kymographs of cGFP-XPC, polβ-tGFP, and APE1-tGFP, respectively, on undamaged DNA. FIGS. 11E-G depict representative kymographs of HaloTag-JF635-DDB2 binding DNA treated with 0, 20, and 40 J of UV irradiation. FIG. 11H depicts the quantification of events per minute vs UV dose. Error bars represent SEM of three kymographs each. FIG. 11I depicts a kymograph of mScarlet-OGG1 binding events on undamaged DNA, with a few transient events apparent in green.

FIGS. 12A-12C depict positional accuracy and limitation of MSD analysis. FIG. 12A depicts a representative kymograph of a 705 nm Qdot linked to DNA and scanned at various tensions from 0.1-10 pN. Line tracking is shown in orange and tension over time shown in blue (bottom). Only segments of the lines without blinks were used to determine precision. FIG. 12B depicts the localization precision of a single Qdot at various tensions. FIG. 12C depicts fits of the mean square displacement plots of positions at various tensions. These values represent the minimum diffusivity that can be measured with the C-trap. Dimmer fluorophores like eGFP and HaloTag-JF-635 exhibited maximum positional accuracy of 53 and 40 nm at 10 pN, respectively, based on the line tracking from nonmotile DDB1 and DDB2 events.

FIGS. 13A-13F depict colocalization of HaloTag-JF635-DDB2 and HaloTag-JF503-DDB2. FIGS. 13A-13E depict examples of kymographs of HaloTag-DDB2 labeled with two color dyes (JF-503 in blue and JF-635 in red) on DNA treated with 40J of UV damage. Colocalization could occur if two DDB2 molecules bound to two sites of damage within the C-trap localization precision or if two UV-DDB molecules formed a dimer of heterodimers. FIG. 13F depicts a schematic showing colocalization statistics for the two colors of DDB2, with only 2% of events colocalizing.

FIGS. 14A-14C depict the lifetime analysis of UVDDB binding using the widefield C-trap system. FIG. 14A shows cGFP-DDB1 lifetimes were fit well to a single exponential with attached lifetimes of 8.4±1.3 s. FIG. 14B shows JF-DDB2 lifetimes fit to a double exponential with revealing two lifetimes of 2.76±0.36 s and 184.8±363.8 s. FIG. 14C shows colocalized events for DDB1 and DDB2 fitted to a single exponential with an attached lifetime of 38.8±31.9 s. Photobleaching corrections were based on measurements of surface associated bleached molecules with a rate constant for JF635 of 0.09±0.009 s⁻¹ and for cGFP of 0.19±0.02 s⁻¹.

FIGS. 15A-15H depict a single-molecule Forster resonance energy transfer (smFRET) approach to confirm SMADNE analysis of DDB1 and DDB2. To probe the structure of colocalized events at resolution beyond the limits of the C-trap, a single-molecule Förster resonance energy transfer (smFRET) approach was employed. FIG. 15A depicts a diagram of the excitation and emission spectrum of eGFP and mCherry. FIG. 15A depicts the emission of eGFP overlaps with the excitation of mCherry as necessary for FRET. FIG. 15B shows the structure of cGFP-DDB1 (donor) and mCherry-DDB2 (acceptor), with fluorophores modeled in at their respective termini. FIG. 15C depicts an example kymograph of four events to assay eGFP signal in the channel used for mCherry (green). FIG. 15D depicts a consistent ratio of 9.0% of the cGFP photon counts observed in the mCherry channel, which was used as a correction factor. FIG. 15E depicts an example FRET-positive event with quantification of photon counts shown in FIG. 15F. FIG. 15G depicts a known Förster radius of eGFP and mCherry, with distances calculated based on the ratiometric FRET efficiency. FIG. 15H depicts the FRET positive events, where the average distance between fluorophores was 51.0 Å, in agreement with the structural model shown in FIG. 15B.

FIGS. 16A-16H depict increasing amounts of added purified UV-DDB decreased binding lifetimes of labeled proteins. FIG. 16A depicts the binding lifetimes of eGFP-DDB1 shown in blue and HaloTag-DDB2 shown in red with various concentrations of unlabeled UV-DDB added. Lifetimes shown with an asterisk were measured with no purified UV-DDB added. FIG. 16B depicts a fit of the k_{off} vs. competitor concentration (from 0-3 nM) in the linear range (solid lines). Plateau range of DDB1 shown with dotted line. Rate constants for the fits are 0.76 nM⁻¹ s⁻¹ for DDB1 and 0.59 nM⁻¹ s⁻¹ for DDB2. FIGS. 16C-16H depicts example kymographs of cGFP-DDB1 (blue) and HaloTag-DDB2 (red) binding DNA with 40 J of UV damage upon increasing concentration of unlabeled purified protein.

FIGS. 17A-17D depict dual labeled UV-DDB bound to oxidative damaged DNA. FIG. 17A depicts a schematic of the DNA containing oxidative damage showing transient binding events from both HaloTag-JF635-DDB2 (red) and cGFP-DDB1 (blue) with colocalized events appearing purple. Also shown are the binding positions on the full-length kymograph shown (5 minutes). FIG. 17B depicts a CRTD plot of eGFP-DDB1 on oxidative damage. FIG. 17C depicts a CRTD plot of HaloTag-DDB2 on DNA with oxidative damage. FIG. 17D depicts colocalization patterns between the two proteins. Continuous scan at 33 msec per scan. A minimum of time difference of 3 pixel=100 msec were scored as a colocalization event.

FIGS. 18A-18C depict labeling nick DNA with F1-dUTP. FIG. 18A shows that A-DNA contains 10 Nt.BspQI cut sites (map shown) with the positions in nucleotide number labeled. FIG. 18B depicts after nick translation with pol I, fluorescent dUTP is incorporated at the sites of nicks (a representative DNA strand shown with F1-dUTP appearing as blue streaks). The positions of these nicks (black bars) agreed with the expected positions (red spheres). The nick (*) near 100 percent is too close to the fluorescence of the beads to resolve. The two nicks (**) are two close together to resolve separately. FIG. 18C depicts the nicks that agreed with anticipated sites (an average of 7.6 out of 8 observable) compared with rare off target F1-dUTP incorporation (0.3 off-target incorporations per DNA). Error bars represent SEM from 7 DNA strands. In this example the orientation is shown as in FIG. 18A, other DNAs were also observed in the opposite orientation.

FIGS. 19A-19C depict the outcome of SMADNE analysis of YFP-PARP2. FIG. 19A shows the structure of PARP2 generated with alphafold (yellow), along with the YFP tag positioned at the N-terminus of the protein. FIG. 19B shows the cumulative residence time distribution of PARP2 binding events, with a binding lifetime of 11.7 s. FIG. 19C shows a cartoon of the nicked DNA substrate used for the experiment as well as a representative kymograph, with the PARP2 binding event displayed in yellow.

FIGS. 20A-20D depict the SMADNE events of YFP-XRCC1 and Halotag-Lig3α. FIG. 20A shows a diagram of the DNA substrate used, with 10 nicks generated with a site-specific nickase. Representations of binding events including YFP-XRCC1 (blue), Halotag-Lig3α (red), or both together (purple) are shown, as well as a representative kymograph. FIG. 20B shows the cumulative residence time distribution analysis of both proteins, with the weighted averages and number of events displayed in their respective colors. FIG. 20C shows a key for the categories of assembly and disassembly with FIG. 20D showing which types were observed, including the most common colocalization consisting of XRCC1 and Lig3α binding together, followed by XRCC1 dissociating from the DNA first.

FIGS. 21A-21C depict the binding events from stable expression of mNeonGreen-DDB2. FIG. 21A shows a western blot of multiple concentrations of purified DDB2 and nuclear extracts from cells stably expressing mNeonGreen-DDB2. Overexpression is much lower than the transient overexpression, with the endogenous DDB2 at around 25% as concentrated as the fluorescently tagged version. FIG. 21B shows a cartoon and kymograph of mNeonGreen-DDB2 binding UV-damaged DNA and FIG. 21C shows the cumulative residence time distribution of mNeonGreen-DDB2 binding the UV-damaged DNA.

FIGS. 22A-22D depict the lifetime of TDG-HaloTag-JF635 bound to DNA containing 5-formyl-cytosine (5fC) and undamaged DNA. FIG. 22A depicts the incorporation of a dNTP mix containing a fluorescently labeled nucleotide as well as a damage nucleotide, as depicted in FIG. 18. Any nucleotide recognized by DNA polymerase I can be incorporated. FIG. 22B depicts an example kymograph of TDG-HaloTag (red) binding to 48.5 kb lambda DNA after nick translation to incorporate 5fC and F1-dUTP (blue). Nick sites are indicated with a blue star and specific binding events are indicated with a red arrow. FIG. 22C depicts an example kymograph of TDG-HaloTag binding to undamaged 48.5 kb lambda DNA. FIG. 22D depicts a CRTD plot of TDG-HaloTag binding to 5fC DNA and undamaged DNA.

FIGS. 23A-23D depict SMADNE characterization of GFP-AAG. FIG. 23A depicts the structure of alkyladenine glycosylase (AAG) with an N-terminal turbo GFP tag. Structures taken from PDB codes 1F4R and 4KW4. FIG. 23B shows nick translation allows for the simultaneous incorporation of Cy3-dUTP and dITP (inosine triphosphate). Cy3 incorporation positions were determined via fluorescence and are shown as orange stars and dotted lines. In the example kymograph, GFP-AAG binding events are shown in blue, with off-target events shown with a blue asterisk and on-target events with double green asterisks. FIG. 23C shows the distribution of off-target and on-target events. FIG. 23D shows the cumulative residence time distribution of all GFP-AAG events, fitting to a single-exponential with a lifetime of 2.5 s.

FIG. 24 shows a Western blot of DDB2-mNeonGreen. Cells stably expressing mNeonGreen-DDB2 were lysed and run on an SDS-PAGE to determine protein levels. There was an approximate 3-fold overexpression of mNeonGreen-DDB2 compared to endogenous DDB2.

FIGS. 25A-25E demonstrate the SMADNE approach to shows MCV LT specifically binds to the MCV replication origin. FIG. 25A shows MCV and SV40 LT helicase oncoprotein domains. MCV and SV40 LT are homologous helicases sharing DnaJ, retinoblastoma (Rb) protein-binding, DNA origin binding (OBD), multimerization Zn-finger and helicase domains. MCV LT contains an MCV unique region (MUR) that is not present in SV40 LT. FIG. 25B shows a microfluidic chip for DNA capture. (Left) Schematic flow cell. (Right) Details of the laminar flow channels. Initial DNA-polystyrene bead capture occurs in channel 1, followed by DNA tethering in channel 2, DNA quality check in PBS buffer in channel 3, and protein binding and imaging in channels 4 and 5. FIG. 25C shows the cloning of biotinylated Ori98 DNA. pMC.Ori98 Plasmids were digested at XmaI/EcoRI sites and self-ligated to form random origin multimers (1× to 7×), and CG ends were filled with biotinylated dCTP and dGTP using Klenow fragment. The number and location of Ori98 sequences were determined from DNA length in each assay. FIG. 25D shows a representative kymograph of mN-LT bound to multimeric pMC.Ori98 (3×) DNA showing both prolonged and transient binding events. Three origin (white arrows) and three non-origin-binding events (orange arrows) are shown. Transient binding events (<5 s duration, blue arrows) were not included in subsequent analyses. FIG. 25E shows Ori98 sequence has high binding specificity compared to non-Ori98 pMC.BESPX backbone DNA sequence. Binding frequency for Ori98 and non-Ori98-binding events were collected from 30 DNAs, 5 min exposure each. Statistical analysis was performed using an unpaired/test, P=0.0085.

FIGS. 26A-26D shows MCV LT multimerize on the MCV origin. FIG. 26A shows LT specifically bound to wild-type origin but was reduced for tumor-derived mutant, MCV Ori98.Rep-originDNA. Frequency plots from six DNAs each, with 61 and 22 binding events, respectively. Data collected from multimeric pMC-Ori98 and Ori98.Rep-(1× to 7×) were realigned as single copies. FIG. 26B shows the LT protein multimerized on the wild-type Ori98. Representative kymograph for mN-LTK331A (Top) shows that K331A mutation in the LT origin binding domain (OBD) eliminated specific binding to Ori98. Binding was restored (Bottom) when mN-LTK331A was flowed together in the same channel with nonfluorescent wild-type LT. FIG. 26C shows frequency plots for mN-LTK331A binding to Ori98 without and with nonfluorescent wild-type LT. Data collected from 6 DNA each with 2 and 17 binding events, respectively. FIG. 26D shows the coimmunoprecipitation of LT-FLAG and mN-LT expressed in 293 cells revealed LT multimerization in the absence of origin DNA. Retinoblastoma protein (Rb) detection was used as a positive control for LT pulldown. Representative blot of three repetitions.

FIGS. 27A-27C show the melting of origin DNA by MCV LT. FIG. 27A shows mN-LT bound and melted dsDNA to ssDNA to allow Cy5-RAD51 cobinding. (A, Top) Cy5-RAD51 (red) did not bind pMC-Ori98 dsDNA in the absence of LT protein. No binding events were observed for 5 DNAs examined for 5 min each. (A, Bottom) Cy5-RAD51 (red) colocalized with mN-LT (green) bound to pMC.Ori98 DNA. Representative image from 12 DNAs, 5 min each, 122 events. FIG. 27B shows single-strand S1 nuclease cleaved Ori98 DNA only after mN-LT binding. Top force diagram for Ori98 dsDNA without mN-LT (Top) exposed to S1 nuclease. The captured dsDNA was exposed to empty vector nuclear extract for 40 s and then moved into the S1 nuclease channel (200 units/mL). The DNA retained tension at 10 pN for 320s. When mN-LT was captured on Ori98 (Bottom), and then moved into the S1 nuclease channel, tension was lost after 4 s, indicating DNA cleavage. FIG. 27C shows mN-LT bound and melted dsDNA as measured by GFP-RPA70 cobinding. (C, Top) representative GFP-RPA70 (green) flowed on multimeric Ori98 dsDNA alone. No binding events were observed for 5 DNAs, 5 min each. (C, Bottom) cobinding of LT-mS (red) and GFP-RPA70 present for 6 DNAs, 5 min each, 38 events.

FIG. 28A-28D show the quantitation of assembly and mean lifetime of LT on the MCV origin. FIG. 28A shows the photobleaching of mN-LT. Representative mN-LT photobleaching (green) measured by photon counts per second. FIG. 28B shows the Hidden Markov Model simulation (HMM) to estimate mN-LT molecule numbers for each initial binding event based on photobleaching. Photon counts from initial binding events were recorded using LUMICKS Pylake software and the best model for equal steps of photon loss was determined for each captured DNA. Estimated photon levels are displayed by red dashed lines. Monomer and dimer assemblies were not reliably discriminated and were removed from the analysis. FIG. 28C shows mN-LT assembled to a dodecamer on wild-type Ori98 but not on Ori98.Rep-DNA. Frequency of mN-LT molecules initially bound to Ori98 as determined by HMMs (blue bars, Top) vs. Ori98.Rep-(yellow bars, Bottom). mN-LT dodecamer assembly was observed in 22% of Ori98 binding events, whereas no assemblies greater than nonamer were observed for Ori98.Rep-. Approximately 30% of assemblies were trimers for both Ori98 and Ori98.Rep-. Rare Ori98 assemblies >12 molecules (3.6%) may represent binding to nonreplication pentads in the MCV origin in addition to origin assemblies. Error bars represent SEM among DNAs. A two-sample Kolmogorov-Smirnov test was significantly different for Ori98 and Ori98.Rep-distributions with D=0.283, P<0.05. FIG. 28D shows the mean binding lifetime for dodecamer, hexamer, and trimer mN-LT on Ori98 DNA as determined from k_{off} rates corrected for photobleaching. The LT dodecamer has a 17-fold longer mean binding lifetime on origin DNA than the LT hexamer. The two-sample t test showed a significant difference of mean lifetime for 12-mers compared to 6-mers, with P<0.0001.

FIGS. 29A-29D show the partially assembled MCV or SV40 LT proteins melt MCV origin dsDNA. FIG. 29A shows ssDNA RAD51 binding occurs after LT assembly on Ori98.Rep. A representative kymograph from 26 colocalization events for mN-LT (green) and Cy5-RAD51 (red) bound to Ori98.Rep-using six DNAs. The white arrow marks initial mN-LT DNA binding, and the red arrow marks subsequent Cy5-RAD51 assembly. FIG. 29B shows the RAD51 cobinding was proportional to LT multimerization and lag time for RAD51 cobinding, after LT binding, decreased exponentially with size of the initially bound LT multimer. (Top) Maximum Cy5-RAD51 fluorescence versus mN-LT molecule assembly number on wild-type Ori98 for dual LT-RAD51 binding events (n, 94). Increased LT multimerization was associated with increased RAD51 ssDNA deposition, R2=0.8608 for a linear regression, and F=49.48 for the F-test with P=0.0001. six DNAs, 5 min exposure each. No RAD51 binding was seen for 52 origins that did not bind LT during the experiment. (Bottom) lag time between initial mN-LT and initial Cy5-RAD51 binding to the same origin for dual LT-RAD51 binding events (n, 94). Lag time was inversely related to initial LT multimerization. Dodecameric mN-LT recruited Cy5-RAD51 almost immediately, whereas trimeric mN-LT required 67 s (on average) to attract Cy5-RAD51 binding, R2=0.9509 for an exponential regression. FIG. 29C shows the nonreplicative SV40 LT melts MCV origin. (Top) GFP-SV40 LT (green) did not form hexamers on MCV Ori98 but was associated with DNA melting and subsequent Cy5-RAD51 (red) colocalization (white arrows). (Bottom) frequency of estimated SV40 LT-GFP multimers initially binding to MCV origin. Data collected from 12 DNAs, 5 min each. FIG. 29D shows SV40 LT melts the SV40 origin. GFP-SV40 LT (green) was associated with DNA melting and Cy5-RAD51 (red) colocalization (white arrows) on SV40 origin. (Bottom) frequency of estimated SV40 LT-GFP multimers initially binding to SV40 origin showed preferred hexamer and dodecamer assembly. Data were collected from six DNAs, 5 min each. Notably, sub-double-hexameric SV40 LT binding events were also observed to melt SV40 origin in a fashion similar to MCV LT on MCV origin.

FIGS. 30A-30D show MCV LT melts MCV origin dsDNA in the absence of helicase activity. FIG. 30A shows MCV LT domains with truncation and site-directed mutation sites denoted. FIG. 30B shows elections of the MCV LT helicase domain (LT700 and LT610), but not the zinc-finger multimerization domain (LT455), retained capacity to melt MCV origin DNA. Representative kymographs for full-length LT, LT700, LT610, and LT455 binding are shown with 5 pMC-Ori98 DNAs for 5 min each. FIG. 30C shows MCV origin DNA melting by MCV LT required ATP binding but not hydrolysis. mN-LT (green) and Cy5-RAD51 colocalization (Top) was lost when nuclear extracts were treated with apyrase to eliminate ATP. Both mN-LT and Cy5-RAD51 binding to Ori98 were restored after apyrase treatment by exposure to 1 mM nonhydrolyzable AMP-PNP. Representative kymographs from 5 pMC-Ori98 (4×) DNAs each, 5 min exposure. FIG. 30D shows MCV LT formed dodecameric assemblies on MCV origin DNA in the absence of hydrolyzable ATP. Frequency of estimated mN-LT multimers initially binding to MCV Ori with 1 mM AMP-PNP. Data were collected from six DNAs, 5 min each. Error bars represent SEM among DNAs.

FIGS. 31A and 31B show models for CMG and MCV LT helicase initiation of dsDNA melting for recruitment of replication machinery. FIG. 31A shows a model for eukaryotic CMG helicase initiation of DNA replication (4, 6). CMG double hexamer first assembles around dsDNA during the late M/G1 phase. On S phase entry, CMG melts origin DNA by ATP-driven DNA distortion and then hexamers remodel around ssDNA. The two hexamers bypass each other to initiate dsDNA unzipping and recruitment of the replisome. FIG. 31B shows a model for MCV and SV40 LT origin melting. After initial LT binding to viral DNA pentads using LT origin binding domains, LT multimerizes to pry apart the MCV origin sequence and melt dsDNA in the absence of ATP hydrolysis. Hexamers then directly assemble around ssDNA. Once assembled, the MCV LT initiates ATP-driven helicase processivity similarly to cellular CMG.

FIGS. 32A-32D show the validation and characterization for MCV LT binding to Ori98. (FIG. 32A shows an alignment of MCV and SV40 origin and pentad sequences (PS). Pentads required for in vitro replication are shown in red and non-essential pentads are shown in black for each virus. G (A/G) GGC repeats are colored in blue, and the inverse complement orientation GCC (C/T)) C pentads are shown in orange, with overlapping nucleotides are shown in green. Site I is not required for SV40 in vitro replication, but Site A is required for MCV replication. The AT-rich regions and the SV40 early palindrome (EP) are indicated. Figure was adapted from Harrison et al.²⁷. FIG. 32B shows the fabrication of biotinylated multimeric Ori98 DNA. pMC.Ori98 plasmids were digested at XmaI sites to produce 5′ CCGG overhangs and EcoRI to produce 5′ AATT overhangs, then self-ligated. Biotinylation was performed using biotin-dCTP and biotin-dGTP with Klenow fragment to fill into 5′ CCGG overhangs only. Only the DNAs with biotins at both ends were captured by two streptavidin coated beads. DNAs containing only one biotinylated end, or two blunt ends would not be tethered between the beads. Slash lines “//” indicate varying copies of the multimeric DNA. A replication. FIG. 32C shows the replication efficiency for fluorophore-tagged codon-optimized LT constructs was determined by a replicon replication assay. containing plasmid Ori350 (98) and untagged LT, mN-LT, or LT-mS were co-transfected into 293 cells and the replication efficiency of each construct was determined by qPCR. Mean from four repeats, SEM. Western blot showed the corresponding protein expressions. FIG. 32D shows the frequency plots for mN-LT binding events for pMC-Ori98 (3×) (12,540 bp; 6 DNAs, 40 events) and 1 phage genome (48,502 bp; 6 DNAs, 26 events).

FIGS. 33A and 33B show on-rate constant (kₒₙ) for MCV LT binding to Ori98 and non-Ori98 sequences. FIG. 33A shows the distribution of mN-LT binding event start time on the Ori98 site and the pMC.BESPX backbone. Data was collected from 6 DNAs, 5 min each, for a total of 90 events (39 events for Ori98, 51 events for vector backbone). FIG. 33B shows data fitted to an exponential equation to calculate the relative kₒₙ for Ori98 and pMC.BESPX backbone. Kₒₙ for mN-LT is 47.2-fold higher at Ori98 sites than on the non-Ori98 pMC.BESPX backbone sequence. To determine the relative kₒₙ for mN-LT binding to Ori98 and non-Ori98 (pMC. BESPX backbone) regions: Binding percentage=1−e^{-(konC(mN-LT)L}DNA)LDNA)t, where kₒₙ is the association constant, C(mN-LT) is the concentration of mN-LT in solution, LDNA is the DNA binding length, and t is the initial binding time of each event. By exponential fitting the binding percentage, the exponential constant equals to kₒₙ C(mN-LT) LDNA, with the effective resolution of LOri98=500 bp, LNon-Ori98=7860 bp and C(mN-LT) is the same for Ori98 and Non-ori98:kon(Ori98)/kon(non-Ori98)=47.2

FIG. 34 shows the colocalization of mN-LT and LT-mS on Ori98. Top: Representative kymograph image of mN-LT (green) and LT-mS (red) binding to Ori98 (3×). Bottom: Cross-sectional Gaussian distribution fitting at white dashed line for LT-mS and mN-LT signals. Repeated on 5 DNAs with 32 colocalization events and 14 non-colocalization events

FIGS. 35A-35C show nuclease and GFP-RPA70 binding specificity to ssDNA. FIG. 35A shows Cy5-RAD51 (red) does not bind I phage dsDNA but does bind ssDNA regions caused by stretching the dsDNA to 65 pN. Representative kymograph from 5 DNAs. FIG. 35B shows co-immunoprecipitation of mN-LT and T7-RAD51 expressed in 293 cells. No specific interaction was found. Rb, retinoblastoma protein, positive control for LT interaction. FIG. 35C shows stretching multimeric Ori98 DNA up to 65 pN caused local ssDNA formation and caused GFP-RPA70 binding (arrow). Data was collected from 3 DNAs, 100s each.

FIGS. 36A and 36B show mN-LT multimerization and photobleaching lifetime. FIG. 36A shows a size-exclusion chromatography on LT-expressing 293 nuclear extracts incubated with wild-type NCCR (464 bp) or with NCCR.Rep-DNAs (464 bp). Quantitative PCR (DDCt) for DNA bound to LT revealed maximum elution for wild-type NCCR in fractions 7-10 whereas NCCR. Rep-peaked in fractions 14-16. Corresponding molecular mass markers were Dextran Blue (2000 kDa), thyroglobulin (669 kDa). FIG. 36B shows a photobleaching mN-LT on a glass substrate fitted to an exponential decay function to determine photobleaching lifetime. Representative graph from five samples at excitation wavelength=488 nm, line scanning time 0.1 s, 30% laser power.

FIGS. 37A-37D show MCV and SV40 LT melts dsDNA in the absence of helicase activity. FIG. 37A shows replication assays for mN-LT, mN-LT455, mN-LT610, and mN-LT700 adjusted to equal amounts of LT expression. All C-terminal LT truncations eliminated replication activity. mN-LTK331A is a negative control. Error bar is SEM from three repeats. FIG. 37B shows a Walker A box mutant mN-LTK599R mutation eliminates replication activity. mN-LTK331A is a negative control. Single replication. FIG. 37C shows mN-LTK599R binds and melts MCV origin DNA. mN-LTK599R (green, white arrows) assembled on MCV Ori98 with notable diffusion along the DNA position axis and was associated with subsequent Cy5-RAD51 (red) colocalization. FIG. 37D shows SV40 LT melting MCV origin requires ATP binding but not hydrolysis. GFP-SV40 LT (green) and Cy5-RAD51 (red) colocalization was absent without ATP but was rescued by 1 mM AMP-PNP. Representative data from 6 DNAs, 5 min each, 32 events.

FIG. 38 illustrates the strategy for generating a substrate containing nucleosomes for use with SMADNE.

FIG. 39 shows YFP-PARP1 binding events at a nicked superhelical location (SHL). A representative example of kymograph of PARP1 binding DNA at 4 pN tension. Dwell times/off rates and apparent on rate are depicted in right panels.

FIG. 40 shows the binding of Halo-635-LIG3 and XRCC1-YFP to a nucleosome with a non-ligatable nicked SHL. A representative example of kymograph of PARP1 binding DNA at 5 pN tension.

FIG. 41 shows a representative example of kymograph of TDG binding to nucleosome containing DNA substrate. Deletion of the N-terminus of TDG demonstrates its important for interacting with nondamaged nucleosomes.

FIGS. 42A-42H show OGG1 binds to undamaged DNA with multiple modes.

FIG. 42A shows a structural model of GFP-tagged OGG1 bound to damaged DNA, from PDB codes 1YQR and 5LK4. FIG. 42B shows a representative kymograph of OGG1 binding undamaged DNA, with a cartoon on the left showing the positions of the beads and DNA. The times at which microfluidic flow was present are also indicated. FIG. 42C shows a representative motile OGG1 event with line tracking shown beneath the raw kymograph. FIG. 42D shows a cumulative residence time distribution (CRTD). The fit of double-exponential decay functions is shown in orange, and nonmotile dwell times shown in black, and motile dwell times shown in green. FIG. 42E shows the distribution of motile to nonmotile events. FIG. 42E shows the diffusivity and anomalous diffusion coefficient for motile OGG1 events. FIG. 42G shows a representative five-minute kymographs for purified OGG1, purified OGG1 plus non-transfected nuclear extract, and OGG1 generated in mammalian cells prior to nuclear extraction. FIG. 42F demonstrates the activity of OGG1-GFP. FIG. 42H demonstrates GFP-label does not interfere with OGG1 activity, as the purified protein was highly active.

FIGS. 43A-43C show the impact of proteins in nuclear extracts on OGG1 binding damaged DNA. FIG. 43A shows a representative kymograph of purified OGG1 binding DNA treated with methylene blue and light to form 8-oxoguanine. Schematic on left shows positions of the beads and DNA. CRTD plot for purified OGG1 on damaged DNA is also shown. FIG. 43B shows a representative kymograph of purified OGG1 spiked into nuclear extracts is shown in green, and the resultant CRTD plot is displayed below. FIG. 43C shows a kymograph obtained with the single-molecule analysis of DNA-binding proteins from nuclear extracts (SMADNE) approach, with the resultant CRTD plots and fits shown underneath.

FIGS. 44A-44D show the catalytically dead OGG1 engaged undamaged DNA. FIG. 44A shows undamaged DNA was incubated with purified OGG1-K249Q-GFP, and transient interactions were observed (shown in green). FIG. 44B shows a CRTD plot from the dwell times observed is displayed with a single-exponential decay fit. FIG. 44C shows a representative Kymographs depicting that there were no events observed when the purified protein was spiked into nuclear extracts. FIG. 44D shows a representative Kymographs depicting that there were no events observed when the sample was generated with SMADNE. Data was collected on similar time scales.

FIGS. 45A-45C show OGG1-K249Q binds 8-oxoG longer than WT as purified protein or with extract present. FIG. 45A shows a Kymograph of OGG1-K249Q-GFP with a cartoon of streptavidin beads and DNA position shown on the left. The CRTD plot determined from the dwell times is shown beneath the kymograph. FIG. 45B shows OGG1-K249Q-GFP (green kymograph) also engaged damage sites when in the presence of nuclear extracts. CRTD plot is displayed below. FIG. 45C shows representative binding events from OGG1-K249Q-GFP, with a corresponding CRTD plot below.

FIGS. 46A-46D show the roles of proteins in nuclear extracts on single-molecule analysis. FIG. 46A illustrates the nuclear extract approach allowing for variants (colored circles) and PTMs to be rapidly characterized. FIG. 46B illustrates that nuclear proteins (gray) increase data collection efficiency by stabilizing sample proteins (green) with chaperones and providing consistent functional protein concentrations. FIG. 46C illustrates that the low-affinity engagement of nuclear proteins on undamaged DNA competes for nonspecific interactions of target proteins, increasing binding specificity. FIG. 46D illustrates that nuclear extract proteins assist in protein turnover on damage sites through a facilitated dissociation mechanism.

### 5. DETAILED DESCRIPTION

The present disclosure relates to assays, methods and kits for characterizing protein-DNA binding dynamics. In certain embodiments, the DNA-binding proteins are heterologously expressed and are present within a nuclear extract, and DNA binding events are captured by single molecule fluorescence microscopy. The present disclosure also relates to in vitro high-throughput screening methods for characterizing DNA-binding protein variants.

For purposes of clarity of disclosure, but not by way of limitation, the detailed description of the presently disclosed subject matter is divided into the following subsections:
5.1. Definitions;
5.2. Assays;
5.3. Methods of Use;
5.4. Kits; and
5.5. Exemplary Non-Limiting Embodiments.

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

The use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s)” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and still up to 1% of a given value. Alternatively, particularly with respect to biological systems or processes, the term can mean within an order of magnitude, within 5-fold, and within 2-fold, of a value.

The term “culturing” refers to contacting a cell with a cell culture medium under conditions suitable to the survival, growth and/or proliferation of the cell.

The term “culture medium” refers to a nutrient solution used for growing cells, e.g., prokaryotic or eukaryotic cells, that typically provides at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

The term “cell” refers to any suitable cell for use in the present disclosure, e.g., eukaryotic cells. For example, but not by way of limitation, suitable eukaryotic cells include animal cells, e.g., mammalian cells. In certain embodiments, suitable cells are cultured cells. In certain embodiments, suitable cells are host cells, recombinant cells, and recombinant host cells. In certain embodiments, suitable cells are cell lines obtained or derived from mammalian tissues which are able to grow and survive when placed in media containing appropriate nutrients and/or growth factors.

The terms “host cell,” “host cell line” and “host cell culture” are used interchangeably and refer to cells and their progeny into which exogenous nucleic acid can be subsequently introduced to create recombinant cells. In certain embodiments, these host cells can also be modified (i.e., engineered) to alter or delete the expression of certain endogenous host cell proteins. Host cells can include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein. The introduction of exogenous nucleic acid (e.g., by transfection) to these host cells would create recombinant cells that are derived from the original “host cell,” “host cell line” or “host cell line”. The terms “host cell,” “host cell line” and “host cell culture” can also refer to such recombinant cells and their progeny.

The term “mammalian host cell” or “mammalian cell” refers to cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. In certain embodiments, the mammalian cell is a cell that can be transfected to express recombinant proteins and/or fluorescent proteins. In certain embodiments, the mammalian cell can be a human cell, hamster cell, mouse cell, rat cell, sheep cell, goat cell, monkey cell, dog cell, cat cell, horse cell, cow cell, pig cell or a combination thereof. Additional examples of suitable mammalian host cells within the context of the present disclosure can include, but are not limited to, U2OS cells, Sf9 cells, Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989); CHO-K1 (ATCC, CCL-61); monkey kidney CV1 line transformed by SV40 (COS-7, ATCC CRL 1651); fibroblasts, e.g., human fibroblasts; retinal pigment epithelium (RPE) cells, e.g., human RPE cells; human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). Additional cell types are described in Section 5.2 below.

The terms “expression” or “expresses,” as used herein, refer to transcription and translation occurring within a cell, e.g., mammalian cell. In certain embodiments, the level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “recombinant” when used with reference, e.g., to a cell, or nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. For example, recombinant cells express genes that are not found within the native (non-recombinant) form of the cell or express native genes that are otherwise abnormally expressed, under expressed, overexpressed or not expressed at all.

The terms “vector” or “plasmid”, which can be used interchangeably, as used herein, refer to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

As used herein, “polypeptide” refers generally to peptides and proteins having more than about ten amino acids. In certain embodiments, the polypeptides can be homologous to the host cell, or preferably, can be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used.

The term “protein” is meant to refer to a sequence of amino acids for which the chain length is sufficient to produce the higher levels of tertiary and/or quaternary structure. This is to distinguish from “peptides” or other small molecular weight polypeptides that do not have such structure. In certain embodiments, the protein herein will have a molecular weight of at least about 15-20 kDa, e.g., about 20 kDa or greater. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “protein variant” or “polypeptide variant”, refers to a protein or polypeptide that comprise modifications and/or truncations compared to a parent or wild type protein or polypeptide. In certain embodiments, a protein variant can differ from the parent protein or wild type protein by at least one amino acid modification, e.g., from about one to about ten amino acid modifications. In certain embodiments, the sequence of a protein variant sequence has at least about 80%, at least about 90%, at least about 95% or at least about at least about 99% identity to a parent or wild type protein sequence. In certain embodiments, a protein variant can differ from another variant of the protein by at least one amino acid modification, e.g., from about one to about ten amino acid modifications. In certain embodiments, the sequence of a protein variant sequence has at least about 80%, at least about 90%, at least about 95% or at least about at least about 99% identity to a different variant of the protein.

The term “functional fragment thereof” of a molecule, polypeptide or protein includes a fragment of the molecule or polypeptide or protein that retains at least about 80%, at least about 85%, at least about 90%, at least about 95%, or at least about 100% of the primary function of the molecule, polypeptide or protein.

As used herein the terms “amino acid” and “residue” refer to organic compounds composed of amine and carboxylic acid functional groups, along with a side-chain specific to each amino acid. In particular, alpha- or α-amino acid refers to organic compounds in which the amine (—NH₂) is separated from the carboxylic acid (—COOH) by a methylene group (—CH₂), and a side-chain specific to each amino acid connected to this methylene group (—CH₂) which is alpha to the carboxylic acid (—COOH). Different amino acids have different side chains and have distinctive characteristics, such as charge, polarity, aromaticity, reduction potential, hydrophobicity and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The term “nucleic acid,” “nucleic acid molecule” or “polynucleotide” as used herein, refers to any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby the bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a nucleic acid of the disclosure in vitro, e.g., in a mammalian cell. For example, but not by way of limitation, a nucleic acid of the present disclosure can encode a heterologous receptor for detecting an analyte. Such DNA (e.g., cDNA) or RNA (e.g., mRNA) vectors can be unmodified or modified.

The term “nucleotide analogue,” as used herein, refers to a nucleotide that has one or more modifications to the nucleoside, the nucleobase, pentose ring or phosphate group.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity.

The term “mutation” can refer to a deletion, an insertion of a heterologous nucleic acid, an inversion or a substitution, including an open reading frame ablating mutations as commonly understood in the art.

The term “gene” as used herein, can refer to a segment of nucleic acid that encodes an individual protein or RNA (also referred to as a “coding sequence” or “coding region”), optionally together with associated regulatory regions such as promoters, operators, terminators and the like, which can be located upstream or downstream of the coding sequence.

The term “vector” as used herein, refers to a nucleic acid molecule capable of transporting another nucleic acid to which it has been linked.

The term “binding” can refer to the connecting or uniting of two or more components by an interaction, bond, link, force or tie in order to keep two or more components together. In certain embodiments, the term “binding” encompasses either direct or indirect binding where, for example, a first component is directly bound to a second component, or one or more intermediate molecules are disposed between the first component and the second component. Exemplary bonds comprise covalent bonds, ionic bonds, van der Waals interactions and other bonds identifiable by a skilled person. The term “binding” can refer to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, nucleic acids, carbohydrates, lipids, and small organic molecules such as pharmaceutical compounds. Proteins that form stable complexes with other molecules are often referred to as receptors while their binding partners are called ligands. Nucleic acids can also form stable complex with themselves or others, for example, DNA-protein complex, DNA-DNA complex, DNA-RNA complex. In certain embodiments, the binding can be direct, such as a polypeptide or protein, e.g., DNA-binding protein, that directly binds to a protein-binding element of a DNA substrate. In certain embodiments, the binding can be indirect, such as the co-localization of multiple protein elements on one scaffold. In certain embodiments, binding of a component with another component can result in sequestering the component, thus providing a type of inhibition of the component. In certain embodiments, binding of a component with another component can change the activity or function of the component, as in the case of allosteric or other interactions between proteins that result in conformational change of a component, thus providing a type of activation of the bound component. Examples described herein include, without limitation, binding of a protein to DNA. In certain embodiments, binding of protein to a DNA substrate can be directly or indirectly

The terms “microfluidic”, “microfluid system”, “microfluidic cell” or “microfluidic flow cell,” as used herein, can generally refer to a device through which materials, particularly fluid borne materials, such as liquids, can be transported. In certain embodiments, the microfluidic devices described by the presently disclosed subject matter can comprise microscale features, nanoscale features, and combinations thereof. For example, but not by way of limitation, the microfluidic device can transport fluids at the microliter scale. In certain embodiments, a microfluidic device can exist alone or can be a part of a microfluidic system which, for example and without limitation, can include: pumps for introducing fluids, e.g., samples, reagents, buffers and the like, into the system and/or through the system; detection equipment or systems; data storage systems; and

control systems for controlling fluid transport and/or direction within the device, monitoring and controlling environmental conditions to which fluids in the device are subjected, e.g., temperature, current, and the like.

The terms “channel”, “microfluidic channel”, “fluidic channel”, “flow channel” are used interchangeably and can mean a recess or cavity formed in a material by imparting a pattern from a patterned substrate into a material or by any suitable material removing technique, or can mean a recess or cavity in combination with any suitable fluid-conducting structure mounted in the recess or cavity, such as a tube, capillary, or the like.

In the present disclosure, channel size means the cross-sectional area of the microfluidic channel.

The terms “detect” or “detection” as used herein, indicates the determination of the existence and/or presence of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “isolated” biological component (such as a cell, nucleosome, nucleic acid molecule, or protein) has been substantially separated, produced apart from, or purified away from other biological components in the tissue or cell of the organism in which the component naturally occurs, such as other chromosomal and extrachromosomal DNA and

RNA, and proteins. Cells which have been “isolated” thus include cells harvested or extracted from an organism, such as a human, by standard methods (e.g., blood draw, tissue biopsy). Nucleic acid molecules and proteins which have been “isolated” include nucleic acids and proteins purified by standard purification methods. The term also embraces nucleic acid molecules and proteins prepared by recombinant expression in a host cell as well as chemically synthesized nucleic acids. A purified or isolated cell, protein, nucleosome, or nucleic acid molecule can be at least 70%, at least 80%, at least 90%, at least 95%, at least 96%, at least 97%, at least 98%, or at least 99% pure.

The term “chromatin,” as used herein, refers to a complex of molecules including proteins and polynucleotides (e.g., DNA, RNA), as found in a nucleus of a eukaryotic cell. Chromatin is composed in part of histone proteins that form nucleosomes, genomic DNA, and other DNA binding proteins (e.g., transcription factors) that are generally bound to the genomic DNA. The nucleosome core particle is approximately 150 base pairs (bp) of DNA wrapped in 1.67 left-handed superhelical turns around a histone octamer consisting of 2 copies each of the core histones H2A, H2B, H3, and H4. Core particles are connected by stretches of linker DNA, which are up to about 90 bp long.

### 5.2 Assays

Current approaches for studying protein-nucleic acid binding dynamics at the single molecule level have proven technically challenging. Resolving individual proteins within live cells is difficult, while the use of purified protein samples provides limited information. The present disclosure provides assays for characterizing nucleic acid-binding proteins within the complex milieu of a nuclear extract. For example, but not by way of limitation, the assays disclosed herein can be used to characterize the binding of proteins to DNA or the binding of proteins to RNA, e.g., mRNA.

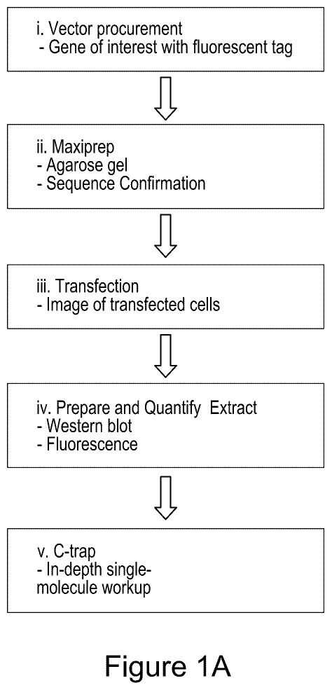

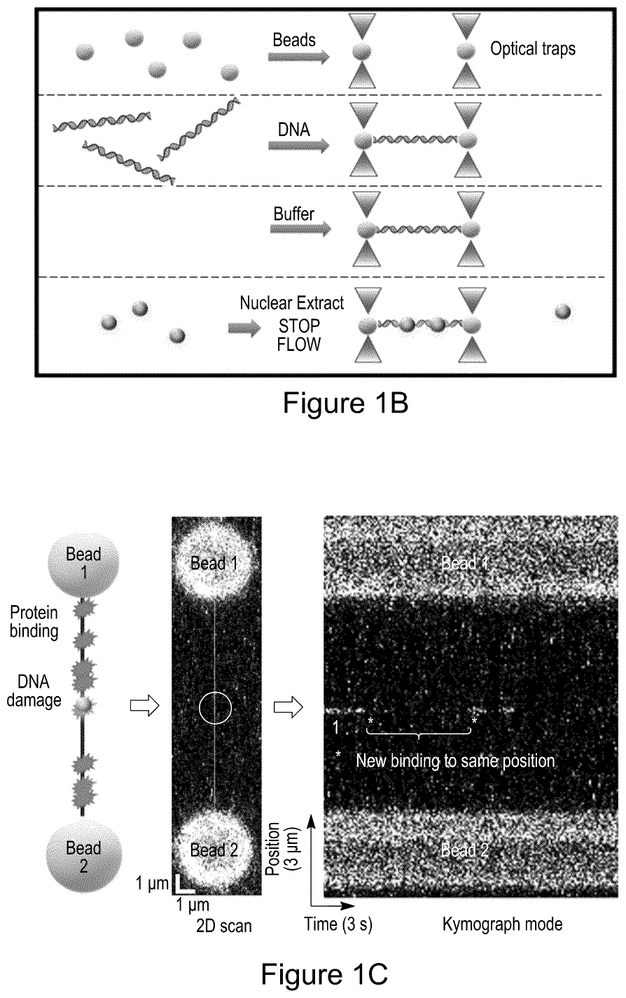

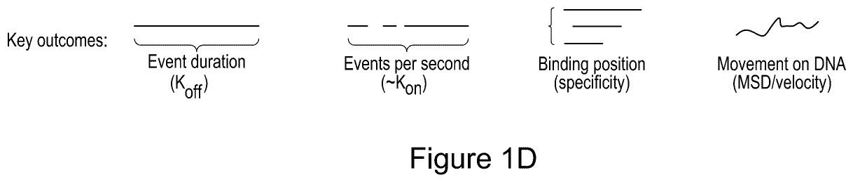

FIG. 1A provides an exemplary embodiment of the assays disclosed herein. In certain embodiments, assays of the present disclosure include expressing one or more recombinant proteins of interest, collecting nuclear extracts containing the one or more recombinant proteins interest, contacting the nuclear extract with a nucleic acid substrate and analyzing nucleic acid binding events, e.g., in real time. In certain embodiments, analyzing nucleic acid binding events includes acquiring images capturing nucleic acid binding events in real time, e.g., via fluorescent microscopy, and then performing single molecule imaging analysis to obtain binding stoichiometry, order of assembly and disassembly, and to understand how proteins diffuse to find their nucleic acid targets. The assays disclosed herein allow to a significant improvement of traditional single molecule approaches for assessing protein-nucleic acid binding dynamics.

In certain embodiments, the present disclosure includes expressing one or more recombinant proteins of interest a cell. In certain embodiments, one recombinant protein of interest is expressed in a cell. In certain embodiments, two or more, three or more, four or more or five or more recombinant proteins of interest are expressed in a cell. For example, but not by way of limitation, if the protein of interest is part of a protein complex in a cell, the cell can be genetically engineered to express more than one protein present in the complex, e.g., all the proteins that are part of the protein complex. In certain embodiments, the protein of interest can form a dimer or trimer, e.g., heterodimers, homodimers, heterotrimers or homotrimers.

In certain embodiments, the recombinant protein is a protein derived from a mammal (e.g., a human), a bacteria, a virus (e.g., a DNA or an RNA virus) and/or a fungus. In certain embodiments, the recombinant protein is a protein derived from a mammal (e.g., a human). In certain embodiments, the recombinant protein is a protein derived from a virus.

In certain embodiments, the recombinant protein can be a nucleic acid binding protein. In certain embodiments, the recombinant protein can be a DNA-binding protein. In certain embodiments, the recombinant protein can be an RNA-binding protein. In certain embodiments, the recombinant protein includes, but is not limited to, DNA repair proteins, DNA modifying proteins, transcription factors, chromatin remodeling factors, methylated DNA binding proteins, polymerases, e.g., DNA polymerases and/or RNA polymerases, nucleases, e.g., endonucleases and/or exonucleases, splicing factors, methylases, glycosylases, demethylases, acetylases, deacetylases, kinases, phosphatases, integrases, recombinases, ligases, topoisomerases, proteases, gyrases, and helicases. In certain embodiments, the recombinant protein is a DNA repair protein. In certain embodiments, the recombinant protein is a helicase. In certain embodiments, the recombinant protein is a polymerase.

In certain embodiments, the recombinant protein can be a natural protein, synthetic protein, modified protein, or other protein analogue. In certain embodiments, the recombinant protein is a variant, homolog, derivative, mutant, inactive or a functional fragment thereof of a wild type protein. In certain embodiments, the one or more recombinant proteins is post-translationally modified. In certain embodiments, the post-translational modification comprises a proteolytic cleavage, glycosylation, or the addition of modifying group, such as acetyl, phosphoryl, glycosyl or methyl, to one or more amino acids of the protein. In certain embodiments, the recombinant protein can be a variant, homolog, derivative, mutant, inactive or a functional fragment thereof of a protein disclosed herein. In certain embodiments, the recombinant protein can be a variant, homolog, derivative, mutant or a functional fragment thereof of a DNA-binding protein disclosed herein. For example, but not by way of limitation, the recombinant protein can be the protein variant or mutant disclosed in Table 5. In certain embodiments, the recombinant protein can be catalytically inactive form of a protein, e.g., by mutation.

In certain embodiments, a DNA-binding protein can be a “DNA repair protein”, which refers to an enzyme capable of repairing base mutagenic damage of DNA. Such DNA repair proteins are often classified according to the type of DNA damage they repair.

For example, but not by way of limitation, the DNA repair protein can be a BER (base excision repair) enzyme, a nucleotide excision repair (NER) enzyme and/or a mismatch repair (MMR) enzyme. For example, but not by way of limitation, mutations such as 8-oxo-7,8-dihydro-2′-deoxyguanosine are repaired by OGG1 (8-oxoguanine glycosylase). In certain embodiments, thymine dimers and/or 6-4 photoproducts are repaired by NER enzyme Photolyase. In certain embodiments, O⁶-methylguanine is repaired by O⁶-methylguanine-DNA methyltransferase. Additional non-limiting examples of DNA repair proteins are provided in Wood et al. Science 291:1284 (2001); Wood et al. Mutation Res. 577:275 (2005), DNA Repair and Mutagenesis, 2nd edition (ASM Press, Washington, DC) (2006); Lange et al. Nature Reviews Cancer 11:96 (2011); Ronen and Glickman, Environ. Mol. Mutagen. 37:241 (2001); Eisen and Hanawalt, Mutat. Res. DNA Repair 435:171 (1999); Aravind et al. Nucleic Acids Res. 27:1223 (1999) and Knijnenburg et al. Cell Rep., 23:239 (2018), the contents of each of which are incorporated herein by reference in their entireties, and listed below.

In certain embodiments, the DNA-binding protein includes HMGB2, DCLRE1B, POT1, CREBBP, EP300, DCLRE1A, AUNIP, RPS3, QOZNB5, MOR2N6, CRY2, E9PQ18, HMGB1, CUL4B, DCLRE1C, UNG, SMUG1, MBD4, TDG, OGG1, MUTYH (MYH), NTHL1 (NTH1), MPG, NEIL1, NEIL2, NEIL3, APEX1 (APE1), APEX2, LIG3, XRCC1, PNKP, APLF, HMCES, PARP1 (ADPRT), PARP2 (ADPRTL2), PARP3 (ADPRTL3), PARG, PARPBP, MGMT, ALKBH2 (ABH2), ALKBH3 (DEPC1), TDP1, TDP2 (TTRAP), SPRTN (Spartan), MSH2, MSH3, MSH6, MLH1, PMS2, MSH4, MSH5, MLH3, PMS1, PMS2P3 (PMS2L3), HFM1, XPC, RAD23B, CETN2, RAD23A, XPA, DDB1, DDB2 (XPE), RPA1, RPA2, RPA3, TFIIH, ERCC3 (XPB), ERCC2 (XPD), GTF2H1, GTF2H2, GTF2H3, GTF2H4, GTF2H5 (TTDA), GTF2E2, CDK7, CCNH, MNAT1, ERCC5 (XPG), ERCC1, ERCC4 (XPF), LIG1, ERCC8 (CSA), ERCC6 (CSB), UVSSA (KIAA1530), XAB2 (HCNP), MMS19, RAD51, RAD51B, RAD51D, HELQ (HEL308), SWI5, SWSAP1, ZSWIM7 (SWS1), SPIDR, PDS5B, DMC1, XRCC2, XRCC3, RAD52, RAD54L, RAD54B, BRCA1, BARD1, ABRAXAS1, PAXIP1 (PTIP), SMC5, SMC6, SHLD1, SHLD2 (FAM35A), SHLD3, SEM1 (SHFM1) (DSS1), RAD50, MRE11A, NBN (NBS1), RBBP8 (CtIP), MUS81, EME1 (MMS4L), EME2, SLX1A (GIYD1), SLX1B (GIYD2), GEN1, FANCA, FANCB, FANCC, BRCA2 (FANCD1), FANCD2, FANCE, FANCF, FANCG (XRCC9), FANCI (KIAA1794), BRIP1 (FANCJ), FANCL, FANCM, PALB2 (FANCN), RAD51C (FANCO), SLX4(FANCP), FAAP20 (Clorf86), FAAP24 (C19orf40), FAAP100, UBE2T (FANCT), XRCC6 (Ku70), XRCC5 (Ku80), PRKDC, LIG4, XRCC4, DCLRE1C (Artemis), NHEJ1 (XLF, Cernunnos), NUDT1 (MTH1), DUT, RRM2B (p53R2), PARK7 (DJ-1), DNPH1, NUDT15 (MTH2), NUDT18, (MTH3), POLA1, POLB, POLD1, POLD2, POLD3, POLD4, POLE (POLE1), POLE2, POLE3, POLE4, REV3L (POLZ), MAD2L2 (REV7), REV1 (REV1L), POLG, POLH, POLI (RAD30B), POLQ, POLK (DINB1), POLL, POLM, POLN (POL4P), PRIMPOL, DNTT, FEN1 (DNase IV), FAN1 (MTMR15), TREX1, TREX2, EXO1 (HEX1), APTX (aprataxin), SPO11, ENDOV, DNA2, DCLRE1A (SNM1A), DCLRE1B (SNM1B), EXO5, UBE2A (RAD6A), UBE2B (RAD6B), RAD18, SHPRH, HLTF (SMARCA3), RNF168, RNF8, RNF4, UBE2V2 (MMS2), UBE2N (UBC13), USP1, WDR48, HERC2, H2AX (H2AFX), CHAF1A (CAF1), SETMAR (METNASE), ATRX, BLM, RMI1, TOP3A, WRN, RECQL4, ATM, MPLKIP (TTDN1), RPA4, PRPF19 (PSO4), RECQL (RECQ1), RECQL5, RDM1 (RAD52B), NABP2 (SSB1), ATR, ATRIP, MDC1, PCNA, RAD1, RAD9A, HUS1, RAD17 (RAD24), CHEK1, CHEK2, TP53, TP53BP1 (53BP1), RIF1, TOPBP1, CLK2, PER1, Apolipoprotein B MRNA editing enzyme catalytic subunit 3A (APOBEC3A), Histone PARylation factor 1 (HPF1), DNA polymerase β (Pol-β), Merkel cell polyomavirus (MCV) large tumor (LT) (MCV-LT), SV40 large T antigen (LT) (SV40-LT) or a combination thereof.

In certain embodiments, the DNA-binding protein can be a gene-editing protein. For example, but not by way of limitation, the DNA-binding protein can be a CRISPR/Cas nickase, a meganuclease, a zinc finger protein, a transcription activator-like effector, a Zinc finger nuclease nickase, a TALEN nickase, or a meganuclease nickase.

In certain embodiments, the one or more recombinant protein of interest can be labeled to allow detection and/or monitoring. For example, but not by way of limitation, the recombinant protein of interest can be fluorescently labeled, e.g., to be resolved by microscopy. In certain embodiments, non-limiting examples of a fluorescent label includes the fluorescent proteins GFP, sfGFP, deGFP, cGFP, yEGFP, tGFP, Venus, ym Venus, ymTagBFP2, iFP1.4, YFP, Cerulean, Citrine, ymTurquoise2, ymNeonGreen, CFP, cYFP, cCFP, RFP, mRFP, ytdTomato, mCherry, mmCherry, NEON, Halo-tag, or SNAP-tag. In certain embodiments, the one or more recombinant proteins can be conjugated to a fluorophore, e.g., Janelia Fluor 635 dye. Proteins containing such labels can be distinguished from proteins not labeled with fluorescent tag, e.g., by the detection or absence, respectively, of the fluorescence emitted by the protein. In certain embodiments, the one or more recombinant proteins can be labeled with quantum dot (Qdot) nanocrystals. For example, but not by way of limitation, the recombinant protein can be biotinylated, which is then coupled to a streptavidin-coated Qdot (non-limiting examples of using Qdots for protein labeling can be found in Kad et al., Molecular Cell 37:702-713 (2010), the contents of which are incorporated by reference herein in their entirety). Additional non-limiting examples of fluorescent proteins are provided in Table 1.

In certain embodiments, a gene encoding a fluorescent protein can be integrated into a host cell genome via gene editing techniques. In certain non-limiting embodiments, a gene encoding a fluorescent protein is integrated into a host cell via CRISPR/Cas gene editing (e.g., CRISPR/Cas9 gene editing). In certain non-limiting embodiments, CRISPR/Cas mediated gene editing is performed to create a knock-in cell line that includes a gene that encodes for a fluorescent protein integrated into or coupled to the N- or C-terminus of the protein. For example, but not by way of limitation, a fluorescent protein such as Halo-tag or SNAP-tag is integrated into or coupled to the N- or C-terminus of a protein of interest by CRISPR/Cas mediated gene editing.

In certain embodiments, the expression construct encoding the polypeptide or protein of interest is integrated into one or more expression vectors. In certain embodiments, the expression vector is a nucleic acid and provides all required elements for the amplification of said vector in a mammalian cell. In certain embodiments, an expression vector is a vehicle for the introduction of an expression construct into a modified mammalian cell according to the subject matter of the present disclosure. In certain embodiments, a construct can be introduced as a single DNA molecule encoding multiple genes, or different DNA molecules having one or more genes. In certain embodiments, multiple constructs can be introduced simultaneously or consecutively, each with the same or different DNA molecule.

Constructs encoding DNA-binding proteins, or constructs encoding related protein variants, as described herein, can be introduced into cells as one or more DNA molecules or constructs, in many cases in association with one or more markers to allow for selection of host cells which contain the construct(s). The constructs can be prepared in conventional ways, where the coding sequences and regulatory regions can be isolated, as appropriate, ligated, cloned in an appropriate cloning host, analyzed by restriction or sequencing, or other convenient means. Particularly, using PCR, individual fragments including all or portions of a functional unit can be isolated, where one or more mutations can be introduced using “primer repair”, ligation, in vitro mutagenesis, etc. as appropriate. The construct(s) once completed and demonstrated to have the appropriate sequences can then be introduced into a host cell by any convenient means. The constructs can be integrated and packaged into non-replicating, defective viral genomes like Adenovirus, Adeno-associated virus (AAV), or Herpes simplex virus (HSV) or others, including retroviral vectors, for infection or transduction into cells. In certain embodiments, the constructs can include viral sequences for transfection, if desired. Alternatively, the construct can be introduced by fusion, electroporation, biolistics, transfection, lipofection, or the like. The host cells will in some cases be grown and expanded in culture before introduction of the construct(s), followed by the appropriate treatment for introduction of the construct(s) and integration of the construct(s). The cells will then be expanded and screened by virtue of a marker present in the construct.

In certain embodiments, expressing one or more recombinant proteins of interest in a host cell includes culturing a cell comprising one or more nucleic acid(s) encoding the polypeptide or protein of interest, under conditions suitable for expression of the polypeptide or protein. Non-limiting examples of such cells are disclosed herein, e.g., mammalian cells can be used to express the polypeptide or protein. In certain embodiments, a host cell, such as, e.g., a U2OS cell according to the subject matter of the present disclosure, is transfected with a vector containing the nucleic acid sequence suitable for expression of said polypeptide or protein of interest.

In certain embodiments, the assay can include preparing nuclear extracts of the cells expressing the one or more recombinant proteins. Techniques for preparing nuclear extracts are known in the art. For example, but not by way of limitation, nuclear extracts can be prepared by incubation in an extraction buffer followed by centrifugation. In certain embodiments, commercial kits can be used to prepare nuclear extracts, e.g., nuclear extract kits from Abcam, Active Motif or Rockland.

In certain embodiments, the method can include analyzing the expression and/or calculating the expression level of the recombinant protein in the cell and/or nuclear extract. In certain embodiments, western blotting can be used for detecting and quantitating expression levels of the recombinant protein. For example, but not by way of limitation, cells can be homogenized in lysis buffer to form a lysate or nuclear extracts can be subjected to SDS-PAGE and blotting to a membrane, such as a nitrocellulose filter. Antibodies (unlabeled) can then be brought into contact with the membrane and assayed by a secondary immunological reagent, such as labeled protein A or anti-immunoglobulin (suitable labels including ¹²⁵I, horseradish peroxidase and alkaline phosphatase). Chromatographic detection can also be used. In certain embodiments, immunodetection can be performed with antibody using an enhanced chemiluminescence system (e.g., from PerkinElmer Life Sciences, Boston, Mass.).

In certain embodiments, the assay can further include contacting the nuclear extract containing said protein(s) of interest with a nucleic acid substrate (e.g., a DNA substrate), e.g., to allow the formation of protein-nucleic acid complexes. In certain embodiments, the nuclear extract containing said protein(s) of interest can be contacted with a nucleic acid substrate (e.g., a DNA substrate) within a microfluidic device, e.g., a microfluidic cell. For example, but not by way of limitation, a nucleic acid binding proteins (e.g., a DNA binding protein) is flowed through the microfluidic cell, whereby the protein of interest come into contact with the nucleic acid substrate (e.g., DNA substrate) traversing the flow cell. In certain embodiments, the microfluidic system further comprises optical tweezers. In certain embodiments, the microfluidic system comprises a microfluidic cell having at least 4 channels separated by laminar flow. In certain embodiments, channel 1 contains beads, channel 2 contains the nucleic acid substrate, channel 3 contains the flow buffer and/or channel 4 contains the cell extract. In certain embodiments, the beads are trapped in channel 1. In certain embodiments, the nucleic acid substrate is suspended between the beads in channel 2. In certain embodiments, a buffer solution is flowed through channel 3. In certain embodiments, the nuclear extract containing the one or more proteins contacts the nucleic acid substrate in channel 4. In certain embodiments, the flow rate is kept constant. In certain embodiments, the flow rate is pulsed. In certain embodiments, the flow is between about 0.05 and 0.5 bar.

In certain embodiments, the nucleic acid substrate (e.g., DNA substrate) is between about 1 and 100 kb in length. In certain embodiments, the nucleic acid substrate (e.g., DNA substrate) is between about 10 and 100 kb in length. In certain embodiments, the nucleic acid substrate (e.g., DNA substrate) is between about 1 to about 70 kb or about 10 and 70 kb in length. For example, but not by way of limitation, the nucleic acid substrate (e.g., DNA substrate) is between about 20 to about 60 kb in length, about 30 to about 50 kb in length, about 40 to about 50 kb in length, about 10 to about 60 kb in length, about 10 to about 50 kb in length, about 10 to about 40 kb in length, about 10 to about 30 kb in length, about 20 to about 70 kb in length, about 30 to about 70 kb in length, about 40 to about 70 kb in length, about 50 to about 70 kb in length. In certain embodiments, the nucleic acid substrate (e.g., DNA substrate) is at least about 10 kb in length, at least about 20 kb in length, at least about 30 kb in length, at least about 40 kb in length, at least about 50 kb in length, at least about 60 kb in length, at least about 70 kb in length, at least about 80 kb in length, at least about 90 kb in length or at least about 100 kb in length. In certain embodiments, the nucleic acid substrate (e.g., DNA substrate) is at least about 10 kb in length. In certain embodiments, the nucleic acid substrate (e.g., DNA substrate) includes a motif for binding the recombinant protein present in the nuclear extracts.

In certain embodiments, the nucleic acid substrate (e.g., DNA substrate) can include one or more nucleotide analogues. In certain embodiments, the nucleic acid substrate (e.g., DNA substrate) can include one nucleotide analogue. Alternatively, the nucleic acid substrate (e.g., DNA substrate) can include two or more nucleotide analogues, three or more nucleotide analogues, four or more nucleotide analogues or five more nucleotide analogues. In certain embodiments, the nucleotide analogue is a nucleotide that is fluorescently labeled. In certain embodiments, a nucleic acid substrate (e.g., DNA substrate) can include two or more fluorescently labeled nucleotides, three or more fluorescently labeled nucleotides, four or more fluorescently labeled nucleotides or five more fluorescently labeled nucleotides. Non-limiting examples of nucleotide analogues include 5-formyl-dCTP (5fC), 5-hm-dUTP, 6-thio-dGTP, 5-fluoro-dUTP, ara-CTP, Cy3-dUTP, dITP or a combination thereof. Additional non-limiting examples of nucleotide analogues are provided below.

In certain embodiments, the sugar group of a nucleotide present in the nucleic acid substrate (e.g., DNA substrate) can be modified. For example, but not by way of limitation, a nucleotide of the nucleic acid substrate (e.g., DNA substrate) can include one or more modifications to its sugar group, e.g., ribose. In certain embodiments, a sugar group can be modified at the 2′ hydroxyl group (OH). In certain embodiments, the 2′ hydroxyl group can be replaced with a different substituent. Non-limiting examples of substituents include hydrogen (H), a halogen, an alkyl or an alkoxy (OR, where R can be an alkyl, a cycloalkyl or an alkoxy). In certain embodiments, the hydrogen (H) of the 2′ hydroxyl group is substituted with a methoxyethyl group. In certain embodiments, modification of the 2′ hydroxyl group can include “locked nucleic acids” (LNA) in which the 2′ hydroxyl group is connected to the 4′ carbon of the same ribose sugar.

In certain embodiments, the phosphate group of a nucleotide present in the nucleic acid substrate (e.g., DNA substrate) can be modified. For example, but not by way of limitation, the phosphate group of a nucleotide can be modified by replacing one or more of the oxygens, e.g., bridging or non-bringing oxygens, in a phosphodiester linkage with a different substituent. Non-limiting examples of substituents include sulfur(S), nitrogen (N), hydrogen (H) and carbon (C). In certain embodiments, one or more oxygens in a phosphodiester linkage are substituted with S. In certain embodiments, the nucleic acid substrate (e.g., DNA substrate) can be modified with one or more phosphorothioate (PS) linkages. In certain embodiments, the nucleic acid substrate (e.g., DNA substrate) can be modified with one or more phosphorodithioate (PS2) linkages.

In certain embodiments, the nucleic acid substrate (e.g., DNA substrate) is positioned using optical tweezers, e.g., positioned within the microfluidic device using optical tweezers. In certain embodiments, the nucleic acid substrate (e.g., DNA substrate) is positioned using dual-trap optical tweezers, whereby the nucleic acid substrate (e.g., DNA substrate) is suspended between two beads, e.g., polystyrene beads, and the beads are positioned between the two traps in the path of the flowing nuclear extract sample. In certain embodiments, the nucleic acid substrate (e.g., DNA substrate) can be held in a constant position. In certain embodiments, the optical tweezers can be used to control tension applied to the nucleic acid substrate (e.g., DNA substrate). In certain embodiments, tension is applied to the nucleic acid substrate (e.g., DNA substrate) between 5 and 40 pN. This range allows nucleic acid (e.g., DNA) to be prepared and/or studied at forces that facilitate protein interaction without overstretching the nucleic acid substrate (e.g., DNA substrate). In certain embodiments, tension is applied to the nucleic acid substrate (e.g., DNA substrate) between about 5 to about 35 pN, between about 5 to about 30 pN, between about 10 to about 40 pN, between about 15 to about 40 pN, between about 20 to about 40 pN, between about 25 to about 40 pN, between about 30 to about 40 pN, between about 10 to about 35 pN or between about 10 to about 30 pN. In certain embodiments, tension is applied to the nucleic acid substrate (e.g., DNA substrate) between 5 and 40 pN. In certain embodiments, tension is applied to the nucleic acid substrate (e.g., DNA substrate) between about 5 to about 70 pN, e.g., 10 pN to about 65 pN.

In certain embodiments, the nucleic acid substrate (e.g., DNA substrate) is tethered to polystyrene beads via biotin-streptavidin interaction. In certain embodiments, the polystyrene beads can have a diameter between about 1 and 10 μm. In certain embodiments, the polystyrene beads can have a diameter between about 4 to about 5 μm, e.g., about 4.38 μm. In certain embodiments, the beads are generated from a polymer, e.g., polystyrene. In certain embodiments, the beads are coated with a functional group to facilitate nucleic acid substrate (e.g., DNA substrate) attachment, e.g., streptavidin. In certain embodiments, the nucleic acid substrate (e.g., DNA substrate) contains a functional group to facilitate bead attachment, e.g., biotin. In certain embodiments, the nucleic acid substrate (e.g., DNA substrate) is tethered to the beads by a biotin-streptavidin interaction. In certain embodiments, the nucleic acid substrate (e.g., DNA substrate) is tethered to the beads by poly-lysine.

In certain embodiments, the nucleic acid substrate (e.g., DNA substrate) is damaged. In certain embodiments, the assay comprises contacting the nuclear extract with damaged DNA. In certain embodiments, DNA damage is induced by ultraviolet light, enzymatic digestion, or by oxidative stress. In certain embodiments, DNA damage of the nucleic acid substrate (e.g., DNA substrate) is induced by ultraviolet light. In certain embodiments, DNA damage of the nucleic acid substrate (e.g., DNA substrate) is induced by enzymatic digestion. In certain embodiments, DNA damage of the nucleic acid substrate (e.g., DNA substrate) is induced by oxidative stress. Non-limiting examples of DNA damage include deamination (e.g., deamination of cytosine and/or adenine (e.g., deamination of cytosine forms hypoxanthine)), depurination, abasic sites, pyrimidine dimers (e.g., thymine dimers), alkylation, additional of bulky chemical groups, and nicks in a single strand of the DNA. In certain embodiments, DNA damage of the nucleic acid substrate (e.g., DNA substrate) is induced by deliberate modification or alteration of nucleosides. In certain embodiments, DNA damage of the nucleic acid substrate (e.g., DNA substrate) is induced by the incorporation of nucleoside analogs. In certain embodiments, the nucleoside analog comprises a modification in its base structure or sugar backbone.

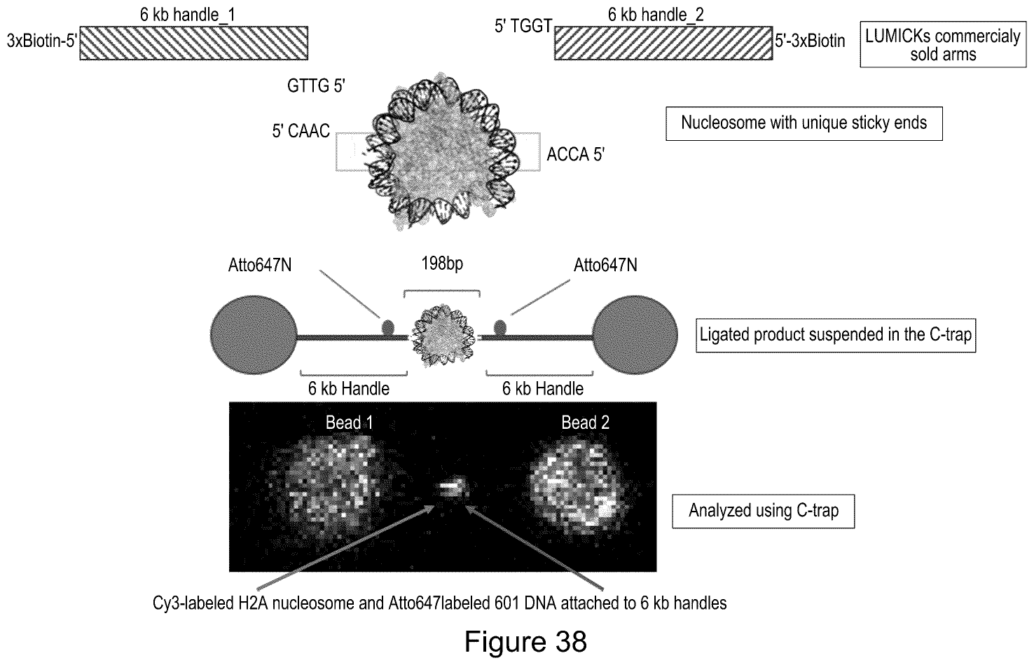

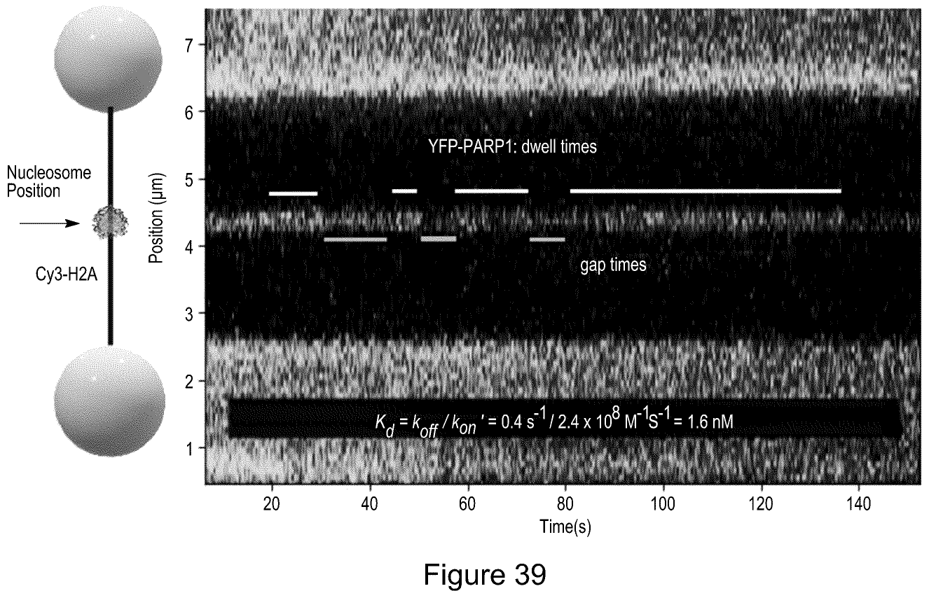

In certain embodiments, the nucleic acid substrate (e.g., DNA substrate) can include one or more nucleosomes. In certain embodiments, at least a portion of the nucleic acid substrate (e.g., DNA substrate) is wrapped around the core histone octamer (two copies of histone H2A, H2B, H3, and H4) to form a nucleosome. In certain embodiments, the nucleic acid substrate (e.g., DNA substrate) can include two or more nucleosomes, three or more nucleosomes, four or more nucleosomes or five more nucleosomes (e.g., to form a nucleosomal array). In certain embodiments, one or more histones of the nucleosome can be fluorescently labeled as described herein (e.g., H2A can be fluorescently labeled). Non-limiting examples of methods for preparing nucleosomal arrays are disclosed in Rogge et al., J. Vis. Exp. 79:50354 (2013), the contents of which are herein incorporated by reference herein in their entirety. In certain embodiments, a nucleic acid substrate (e.g., DNA substrate) comprising nucleosomes can be formed by contacting the nucleic acid substrate (e.g., DNA substrate) with purified histone proteins. In certain embodiments, the nucleosome-containing nucleic acid substrate (e.g., DNA substrate) can be generated as described in FIG. 38. For example, but not by way of limitation, a nucleosome that includes a DNA substrate with sticky ends can be ligated to nucleic acid arms coupled to beads to generate a nucleosome-containing nucleic acid substrate (e.g., DNA substrate) suspended between the beads. In certain embodiments, the nucleic acid arms can include one or more fluorescently labeled nucleotides.

In certain embodiments, the present disclosure utilizes fluorescent microscopy to acquire images over time that resolve individual proteins interacting with a nucleic acid substrate (e.g., a DNA substrate or an RNA substrate) at specific locations. In certain embodiments, fluorescent microscopy includes but not limited to confocal microscopy, TIRF microscopy or single molecule imaging systems. Methods of single molecule spectroscopy are well-known in the art. In certain embodiments of the present disclosure, the single molecule spectroscopy is cylindrical illumination confocal spectroscopy or microfluidic cylindrical illumination confocal spectroscopy. In certain embodiments, fluorescent imaging techniques can be used to measure the decay of fluorescence on a picosecond timescale. Accordingly, the levels and distribution of fluorescent tagged proteins can be assessed by fluorescence imaging methods.

In certain non-limiting embodiments, the present disclosure provides assay for determining key outcome to assess nucleic acid-binding proteins (e.g., DNA or RNA binding proteins), include binding event duration (K_{off}), binding events per second (related to the Kₒₙ), binding position (specificity), and protein movement on DNA or RNA (MSD/velocity). The event duration is obtained by measuring how long the proteins dwell on the nucleic acid substrate (e.g., DNA substrate) and fitting the resultant lifetimes to an exponential decay function. The events per second is measured by dividing the number of unique binding events observed within a certain period of time by the observation time. Binding position measurements are obtained by determining the location along the nucleic acid (e.g., DNA) that the proteins bind with respect to the edge of both beads. For mean squared displacement analysis and velocity measurements, each binding event is tracked over time and the way that it moves along the nucleic acid (e.g., DNA) quantified.

In certain embodiments, single molecule analysis is performed using the LUMICKS C-Trap system, which consists of a microfluidic-cell, dual-trap optical tweezers and three-color confocal fluorescence microscope. In certain embodiments, the LUMICKS C-Trap system comprises a microfluidic chip comprising at least 4 distinct flow channels separated by laminar flow that could be traversed by the two optical traps. In certain embodiments, the assay herein can incorporate fluorescence (single or multi-color) microscopy imaging in various configurations, which include but are not limited to bright-field, epi, confocal, trans, DIC (differential interference contrast), dark-field, Hoffman, or phase-contrast. In certain embodiments, the binding of proteins to a nucleic acid (e.g., DNA substrate) can be detected using fluorescence resonance energy transfer (FRET).

In certain embodiments, protein-nucleic acid interactions can be observed by oblique angle illumination (see Kong et al. Methods Enzymol. 592:213-257 (2017), the contents of which are incorporated by reference herein). In certain embodiments, oblique angle illumination is performed on a total internal reflection fluorescence (TIRF) microscope. Oblique angle illumination allows for the protein-nucleic acid interactions to occur above a surface, where a subcritical, oblique angle is used to maximize the signal-to-noise ratio. In certain embodiments, the oblique angle illumination can involve the use of Qdots to label proteins and provide sufficient fluorescence for visualization. In certain embodiments, the oblique angle illumination technique further comprises an atomic force microscope (AFM) for manipulating the nucleic acid substrate. In certain embodiments, the AFM system allows for analyzing properties such as homogeneity, stability, stoichiometry specificity, and DNA bend angles.

The disclosed subject matter can be readily adapted to a high throughput format, using automated (e.g., robotic) systems, which allow many measurements to be carried out simultaneously.

The order and numbering of the steps in the present disclosure herein are not meant to imply that the steps of any assay or method described herein must be performed in the order in which the steps are listed or in the order in which the steps are numbered. In certain embodiments, the steps of any method disclosed herein can be performed in any order which results in a functional assay or method. Furthermore, the assay or method can be performed with fewer than all of the steps, e.g., with just one step.

| Name | λex | λem | kDa |
|---|---|---|---|
| 10B | | | 26.87 |
| 11 | 502 | 512 | 26.93 |
| 22G | | | 25.46 |
| (3-F)Tyr-EGFP | 484 | 514 | 25.62 |
| 5B | | | 26.94 |
| 6C | | | 26.96 |
| A1a | | | 26.1 |
| A44-KR | 397 | 520 | 26.38 |
| aacuCP | | | 24.98 |
| aacuGFP1 | 478 | 502 | 25.7 |
| aacuGFP2 | 502 | 513 | 26.09 |
| AausFP1 | 504 | 510 | 25.72 |
| AausFP2 | 609 | | 25.56 |
| AausFP3 | 587 | | 25.52 |
| AausFP4 (On) | 477 | 513 | 25.61 |
| AausGFP | 398 | 503 | 27.45 |
| acanFP | | | |
| aceGFP | 480 | 505 | 26.77 |
| aceGFP-G222E-Y220L | 396 | 508 | 26.8 |
| aceGFP-h | 390 | 505 | 26.64 |
| AcGFP1 | 475 | 505 | 26.87 |
| Achilles | | | 26.77 |
| AdRed | 567 | 612 | 26.06 |
| AdRed-C148S | 560 | 599 | 26.05 |
| aeurGFP | 504 | 515 | 26.02 |
| afraGFP | 494 | 503 | 25.69 |
| ahyaCP | | | 24.96 |
| alajGFP1 | 509 | 517 | 25 |
| alajGFP2 | 509 | 517 | 24.67 |
| alajGFP3 | 494 | 504 | 25.15 |
| αGFP | 397 | 506 | 26.8 |
| amCyan1 | 453 | 486 | 25.27 |
| amFP486 | 458 | 486 | 25.32 |
| amFP495 | 467 | 495 | 25.32 |
| amFP506 | 483 | 506 | 25.32 |
| amFP515 | 500 | 515 | 25.28 |
| amilCP | | | 24.99 |
| amilCP580 | | | 25.01 |
| amilCP586 | | | 24.99 |
| amilCP604 | | | 25.02 |
| amilFP484 | 420 | 484 | 26.08 |
| amilFP490 | 424 | 490 | 25.95 |
| amilFP497 | 477 | 497 | 25.9 |
| amilFP504 | 488 | 504 | 25.56 |
| amilFP512 | 500 | 512 | 25.93 |
| amilFP513 | 504 | 513 | 25.98 |
| amilFP593 | 562 | 593 | 26.15 |
| amilFP597 | 558 | 597 | 26.09 |
| anm1GFP1 | 475 | 495 | 29.23 |
| anm1GFP2 | 490 | 504 | 25.02 |
| anm2CP | 572 | 597 | 25.89 |
| anobCFP1 | 462 | 490 | 25.9 |
| anobCFP2 | 477 | 495 | 25.69 |
| anobGFP | 502 | 511 | 25.88 |
| apulCP584 | | | 24.97 |
| apulFP483 | 420 | 483 | 25.92 |
| AQ14 | 590 | 663 | 25.76 |
| AQ143 | 595 | 655 | 25.79 |
| Aquamarine | 430 | 474 | 26.81 |
| asCP562 | 562 | 595 | 25.96 |
| asFP499 | 480 | 499 | 25.37 |
| AsRed2 | 576 | 592 | 25.7 |
| asulCP | 572 | 595 | 25.92 |
| atenFP | 504 | 515 | 24.64 |
| avGFP | 395 | 509 | 26.89 |
| avGFP454 | 410 | 454 | 26.9 |
| avGFP480 | 456 | 480 | |
| avGFP509 | 487 | 509 | |
| avGFP510 | 488 | 510 | |
| avGFP514 | 484 | 514 | |
| avGFP523 | 512 | 523 | |
| AvicFP1 | 481 | 503 | 26.8 |
| AvicFP2 (pre-conversion) | 480 | 515 | 25.61 |
| AvicFP3 (pre-conversion) | 480 | 520 | 25.54 |
| AvicFP4 | 500 | 512 | 25.5 |
| AzaleaB5 | 574 | 596 | 26.02 |
| AzamiGreen | 492 | 505 | 25.96 |
| Azurite | 383 | 447 | 26.88 |
| BDFP1.6 | 642 | 666 | 16.64 |
| BDFP2.0 (Fret) | 587 | 666 | 44.34 |
| bfloGFPa1 | 500 | 512 | 24.55 |
| bfloGFPc1 | 493 | 521 | 23.65 |
| BFP | 381 | 445 | 26.84 |
| BFP5 | 385 | 450 | 26.86 |
| BFP.A5 | 383 | 447 | 26.78 |
| BFPsol | | | 26.71 |
| Blue102 | | | 26.33 |
| BR1 (BR1) | 450 | 495 | 12.87 |
| BrUSLEE | 487 | 509 | 26.88 |
| bsDronpa (On) | 460 | 504 | 25.43 |
| CAR-GECO1 (calcium free) | 576 | 629 | |
| CAR-GECO1 (calcium | 562 | 609 | |
| saturated) | | | |
| ccalGFP1 | 504 | 517 | 24.78 |
| ccalGFP3 | 505 | 517 | 24.82 |
| ccalOFP1 | 508 | 561 | 25.47 |
| ccalRFP1 | 568 | 598 | 24.85 |
| ccalYFP1 | 514 | 523 | 25 |
| cEGFP | 489 | 507 | 27.29 |
| cerFP505 | 494 | 505 | 25.38 |
| Cerulean | 433 | 475 | 26.77 |
| CFP | 456 | 480 | 26.91 |
| CFP4 | | | 26.83 |
| cFP484 | 456 | 484 | 30.45 |
| cfSGFP2 | 493 | 517 | 26.91 |
| cgfmKate2 | 584 | 628 | 26.02 |
| CGFP | 463 | 506 | 26.89 |
| cgfTagRFP | 556 | 585 | 26.07 |
| cgigCP | | | 25.42 |
| cgigGFP | 399 | 496 | |
| cgreGFP | 485 | 500 | 26.38 |
| Channelrhodopsin2 (C1) | 485 | 1000 | 77.25 |
| CheGFP1 | 488 | 500 | 25.62 |
| CheGFP2 | 488 | 508 | 26.62 |
| CheGFP3 | | | 26.07 |
| CheGFP4 | 488 | 500 | 26.55 |
| Chronos | | | |
| Citrine | 516 | 529 | 26.99 |
| Citrine2 | 509 | 522 | 26.94 |
| cjFP510 | 500 | 510 | |
| Clomeleon | 485 | 535 | 26.95 |
| Clover | 505 | 515 | 26.79 |
| Clover1.5 | | | 26.84 |
| cpasCP | | | 25.45 |
| cpCitrine | 506 | 524 | 27.26 |
| cpEYFP(V68L/Q69K) | 506 | 520 | |
| cp-mKate | 588 | 620 | 26.24 |
| cpT-Sapphire 174-173 | 399 | 511 | |
| CpYGFP (Default) | 508 | 518 | 24.74 |
| Cy11.5 | | | |
| CyOFP1 | 497 | 589 | 26.42 |
| CyPet | 435 | 477 | 26.87 |
| CyRFP1 (CyRFP1) | 529 | 588 | 26.42 |
| D10 | 433 | 475 | 26.76 |
| d1EosFP (Green) | 505 | 516 | 25.79 |
| d1EosFP (Red) | 571 | 581 | 25.79 |
| d2EosFP (Green) | 506 | 516 | 25.82 |
| d2EosFP (Red) | 569 | 581 | 25.82 |
| dClavGR1.6 | | | 27.07 |
| dClover2 | | | 26.87 |
| dClover2 A206K | | | 26.93 |
| deGFP1 | 504 | 516 | 26.85 |
| deGFP2 | 496 | 517 | 26.85 |
| deGFP3 | 508 | 518 | 26.9 |
| deGFP4 | 509 | 518 | 26.85 |
| dendFP (Green) | 492 | 508 | 25.81 |
| dendFP (Red) | 557 | 575 | 25.81 |
| Dendra (Green) | 488 | 505 | 25.61 |
| Dendra (Red) | 556 | 575 | 25.61 |
| Dendra2 (Green) | 490 | 507 | 26.12 |
| Dendra2 (Red) | 553 | 573 | 26.12 |
| Dendra2-M159A (Green) | 471 | 504 | 26.06 |
| Dendra2-M159A (Orange) | 528 | 562 | 26.06 |
| Dendra2-T69A (Green) | 502 | 518 | 26.09 |
| Dendra2-T69A (Orange) | 563 | 578 | 26.09 |
| dfGFP | 505 | 524 | 25.83 |
| dhorGFP | | | 26.04 |
| dhorRFP | | | 24.71 |
| dimer1 | 551 | 579 | 25.92 |
| dimer2 | 552 | 579 | 25.89 |
| dis2RFP | 573 | 593 | 26.37 |
| dis3GFP | 503 | 512 | 26.02 |
| dKeima | 440 | 616 | 25.05 |
| dKeima570 | 440 | 570 | 25.01 |
| dLanYFP | 513 | 524 | 26.66 |
| dPapaya0.1 | | | 27.61 |
| DrCBD | 416 | 622 | 34.74 |
| Dreiklang (On) | 511 | 529 | 26.95 |
| d-RFP618 | 560 | 618 | 26.16 |
| Dronpa (On) | 503 | 518 | 25.48 |
| Dronpa-2 (On) | 490 | 515 | 25.45 |
| Dronpa-3 (On) | 490 | 515 | 25.43 |
| Dronpa-C62S | | | 25.46 |
| dsFP483 | 443 | 483 | 26.43 |
| DspR1 | 556 | 582 | 27.04 |
| DsRed | 558 | 583 | 25.93 |
| DsRed2 | 561 | 587 | 25.76 |
| DsRed-Express | 554 | 586 | 25.74 |
| DsRed-Express2 | 554 | 591 | 25.7 |
| DsRed.M1 | 557 | 592 | 25.43 |
| DsRed-Max | 560 | 589 | 25.74 |
| DsRed.T3 | 560 | 587 | 25.81 |
| DsRed.T4 | 555 | 586 | 25.78 |
| DsRed-Timer | | | 25.92 |
| DstC1 | 436 | 482 | 27.03 |
| dTFP0.1 | 456 | 485 | 27.42 |
| dTFP0.2 | 456 | 486 | 27.43 |
| dTomato | 554 | 581 | 26.96 |
| dVFP | 491 | 503 | 25.55 |
| E2-Crimson | 611 | 646 | 25.7 |
| E2-Orange | 540 | 561 | 25.66 |
| E2-Red/Green | 560 | 585 | 25.76 |
| EaGFP | 506 | 514 | 25.92 |
| EBFP | 380 | 440 | 26.9 |
| EBFP1.2 | 379 | 446 | 26.86 |
| EBFP1.5 | 381 | 449 | 26.77 |
| EBFP2 | 383 | 448 | 26.9 |
| ECFP | 434 | 477 | 26.91 |
| ECFP H148D | 433 | 475 | 26.88 |
| ECGFP | 463 | 506 | 26.97 |
| echFP | | | |
| echiFP | | | 25.88 |
| eechGFP1 | 497 | 510 | 26.11 |
| eechGFP2 | 506 | 520 | 25.5 |
| eechGFP3 | 512 | 524 | 25.91 |
| eechRFP | 574 | 582 | 25.6 |
| efasCFP | 466 | 490 | 25.86 |
| efasGFP | 496 | 507 | 24.42 |
| eforCP | 589 | 609 | 25.66 |
| EGFP | 488 | 507 | 26.94 |
| eGFP203C | 498 | 509 | 26.88 |
| eGFP205C | 489 | 509 | 26.88 |
| Emerald | 487 | 509 | 26.9 |
| emiRFP670 | 642 | 670 | 34.19 |
| emiRFP703 | 674 | 703 | 34.22 |
| Enhanced Cyan-Emitting GFP | 458 | 485 | 25.69 |
| EosFP (Green) | 506 | 516 | 25.79 |
| EosFP (Red) | 571 | 581 | 25.79 |
| eqFP578 | 552 | 578 | 26.14 |
| eqFP611 | 559 | 611 | 26.05 |
| eqFP611 V124T | 559 | 611 | 26.05 |
| eqFP650 | 592 | 650 | 26.18 |
| eqFP670 | 605 | 670 | 26.23 |
| EYFP | 513 | 527 | 26.99 |
| EYFP-F46L | | | 26.96 |
| EYFP-Q69K | 514 | 526 | 26.99 |
| eYGFP (Default) | 507 | 516 | 24.73 |
| eYGFPuv (Default) | 398 | 512 | 24.69 |
| eYGPdp (Default) | 501 | 513 | 24.77 |
| fabdGFP | 508 | 520 | 25.45 |
| fcFP | | | 28.56 |
| fcomFP | | | 29.66 |
| ffDronpa (On) | 503 | 517 | 25.45 |
| Flamindo2 | 504 | 523 | |
| Folding Reporter GFP | 490 | 530 | 26.78 |
| FP586 | 559 | 586 | 25.79 |
| Fpaagar | | | |
| Fpag_frag | | | 30.02 |
| Fpcondchrom | | | 25.38 |
| FPmann | | | |
| FPmcavgr7.7 | | | |
| FPrfl2.3 | 506 | 512 | |
| FR-1 | 569 | 594 | 25.59 |
| FusionRed | 580 | 608 | 25.58 |
| FusionRed-M | 571 | 594 | 25.6 |
| FusionRed-MQV | 566 | 585 | 25.59 |
| G1 | 487 | 503 | 26.9 |
| G2 | 487 | 503 | 26.9 |
| G3 | 498 | 515 | 26.87 |
| Gamillus (On) | 504 | 519 | 26.55 |
| Gamillus0.1 | 505 | 524 | 25.64 |
| Gamillus0.2 | 505 | 524 | 25.65 |
| Gamillus0.3 | 505 | 524 | 25.6 |
| Gamillus0.4 | 505 | 524 | 25.62 |
| Gamillus0.5 | | | 25.62 |
| GCaMP2 | 487 | 508 | 50.39 |
| GCaMP6f (In presence of Ca2+) | 496 | 513 | |
| GCaMP6f (In absence of Ca2+) | 496 | 513 | |
| gdjiCP | | | 24.84 |
| GdT | 476 | 500 | 27.02 |
| gfasCP | | | 24.95 |
| gfasGFP | 492 | 506 | 25.99 |
| GFP-151pyTyrCu | 375 | 510 | 26.41 |
| GFP(E222G) (Default) | 481 | 506 | 26.81 |
| GFPha1 | | | 26.73 |
| GFPmut2 | 485 | 508 | 26.87 |
| GFPmut3 | 500 | 513 | 26.84 |
| GFP (S65T) | 490 | 510 | 26.9 |
| GFP-Tyr151pyz | 397 | 508 | 26.71 |
| GFPxm16 | 485 | 504 | 26.98 |
| GFPxm161 | 500 | 510 | 27 |
| GFPxm162 | 514 | 525 | 27.03 |
| GFPxm163 | 512 | 523 | 27.05 |
| GFPxm18 | 472 | 502 | 27 |
| GFPxm181uv | 398 | 514 | 27.02 |
| GFPxm18uv | 400 | 513 | 27.02 |
| GFPxm19 | 475 | 502 | 27 |
| GFPxm191uv | 498 | 510 | 27 |
| GFPxm19uv | 393 | 505 | 27.01 |
| GGvT | 476 | 500 | 54.79 |
| GRvT (Red excitation) | 557 | 583 | 53.96 |
| GRvT (FRET excitation) | 477 | 583 | 53.96 |
| gtenCP | | | 24.92 |
| GZnP3 (GZnP3 (apo state)) | 488 | 512 | |
| GZnP3 (GZnP3 (Zn²⁺-bound)) | 488 | 512 | |
| h2-3 | 506 | 516 | 26.5 |
| H9 | 399 | 511 | 26.96 |
| HcRed | 592 | 645 | 25.64 |
| HcRed1-Blue | 408 | 455 | 25.62 |
| HcRed7 | 592 | 645 | 25.66 |
| HcRed-Tandem | 590 | 637 | 25.91 |
| hcriCP | | | 25.64 |
| hcriGFP | 405 | 500 | 25.34 |
| hmGFP | 490 | 510 | 25.9 |
| HriCFP | 450 | 495 | 15.62 |
| HriGFP | 507 | 527 | 15.72 |
| iFP1.4 | 684 | 708 | 34.78 |
| iFP2.0 | 690 | 711 | 34.85 |
| iLov | 447 | 497 | 14.27 |
| iq-EBFP2 | 386 | 446 | |
| iq-mApple | 568 | 593 | |
| iq-mCerulean3 | 433 | 474 | |
| iq-mEmerald | 482 | 509 | 27.06 |
| iq-mKate2 | 580 | 632 | |
| iq-mVenus | 516 | 529 | |
| iRFP670 | 643 | 670 | 34.48 |
| iRFP682 | 663 | 682 | 34.55 |
| iRFP702 | 673 | 702 | 34.48 |
| iRFP713 | 690 | 713 | 34.6 |
| iRFP713/V256C | 662 | 680 | 34.61 |
| iRFP720 | 702 | 720 | 34.64 |
| IrisFP (Green) | 488 | 516 | 25.69 |
| IrisFP (Orange) | 551 | 580 | 25.69 |
| IrisFP-M159A (Green) | 484 | 513 | 25.63 |
| jRCaMP1a (calcium free) | 573 | 595 | |
| jRCaMP1a (calcium saturated) | 572 | 594 | |
| Jred | 584 | 610 | 29.31 |
| jREX-GECO1 (calcium free) | 577 | 599 | |
| jREX-GECO1 (calcium | 474 | 585 | |
| saturated) | | | |
| jRGECO1a (calcium saturated) | 562 | 588 | |
| jRGECO1a (calcium free) | 450 | 595 | |
| Kaede (Red) | 572 | 580 | 25.67 |
| Kaede (Green) | 508 | 518 | 25.67 |
| Katushka | 588 | 635 | 26.01 |
| Katushka2S | 588 | 633 | 26.39 |
| Katushka-9-5 | 588 | 635 | 26.29 |
| KCY | 455 | 488 | 24.67 |
| KCY-G4219 | 453 | 486 | 24.84 |
| KCY-G4219-38L | 464 | 494 | 24.83 |
| KCY-R1 | 461 | 492 | 24.87 |
| KCY-R1-158A | 459 | 489 | 24.85 |
| KCY-R1-38H | 459 | 489 | 24.88 |
| KCY-R1-38L | 467 | 496 | 24.85 |
| KFP1 (On) | 580 | 600 | 25.82 |
| K-GECO1 (calcium free) | 572 | 597 | |
| K-GECO1 (calcium saturated) | 569 | 593 | |
| KikG | | | 25.88 |
| KikGR1 (Green) | 507 | 517 | 25.76 |
| KikGR1 (Red) | 583 | 593 | 25.76 |
| KillerOrange | 455 | 555 | 26.36 |
| KillerRed | 585 | 610 | 26.4 |
| KO | 548 | 561 | 24.28 |
| KOFP-7 (KOFP-7) | 450 | 496 | 16.67 |
| Kohinoor (On) | 495 | 514 | 25.35 |
| Kohinoor2.0 (On) | 500 | 516 | 25.22 |
| laesGFP | 491 | 506 | 24.93 |
| laGFP | 502 | 511 | 25.88 |
| LanFP1 | 500 | 510 | 24.58 |
| LanFP2 | 500 | 516 | 23.66 |
| lanRFP-ΔS831 | 521 | 592 | 24.72 |
| LanYFP | 513 | 524 | 24.72 |
| laRFP | 521 | 592 | 25.77 |
| LEA | | | 26.41 |
| LSSmCherry1 | 450 | 610 | 26.69 |
| LSS-mKate1 | 463 | 624 | 26.21 |
| LSS-mKate2 | 460 | 605 | 26.2 |
| LSSmOrange | 437 | 572 | 26.72 |
| LSSmScarlet | 470 | 598 | 26.27 |
| Lumazine binding protein | 420 | 470 | 20 |
| M355NA | 572 | 595 | 25.92 |
| mAmetrine | 406 | 526 | 26.8 |
| mApple | 568 | 592 | 26.96 |
| Maroon0.1 | 610 | 650 | 27.5 |
| mAvicFP1 | 480 | 503 | 26.82 |
| mAzamiGreen | 492 | 505 | 25.85 |
| mBanana | 540 | 553 | 26.57 |
| mBeRFP | 446 | 611 | 26.38 |
| mBlueberry1 | 398 | 452 | 26.72 |
| mBlueberry2 | 402 | 467 | 26.76 |
| mc1 | 508 | 582 | 25.82 |
| mc2 | 505 | 515 | 26.02 |
| mc3 | 505 | 515 | 26.81 |
| mc4 | 505 | 515 | 26.05 |
| mc5 | 435 | 495 | 25.84 |
| mc6 | 495 | 507 | 25.83 |
| McaG1 | 492 | 514 | 26.06 |
| McaG1ea | 492 | 514 | 25.96 |
| McaG2 | 492 | 502 | 25.91 |
| mCardinal | 604 | 659 | 27.55 |
| mCarmine | 603 | 675 | 27.65 |
| mcavFP | 440 | 510 | 25.85 |
| mcavGFP | 506 | 516 | 26.74 |
| mcavRFP | 508 | 580 | 25.87 |
| mcCFP | 432 | 477 | 25.78 |
| mCerulean | 433 | 475 | 26.83 |
| mCerulean2 | 432 | 474 | 26.68 |
| mCerulean2.D3 | 434 | 472 | 26.75 |
| mCerulean2.N | 440 | 484 | 26.69 |
| mCerulean2.N(T65S) | 439 | 481 | 26.67 |
| mCerulean3 | 433 | 475 | 26.66 |
| mCerulean.B | 434 | 473 | 26.74 |
| mCerulean.B2 | 432 | 471 | 26.67 |
| mCerulean.B24 | 433 | 473 | 26.75 |
| mcFP497 | | | 25.89 |
| mcFP503 | | | 25.71 |
| mcFP506 | | | 25.74 |
| mCherry | 587 | 610 | 26.72 |
| mCherry1.5 | | | 26.71 |
| mCherry2 | 589 | 610 | 26.74 |
| mCherry-XL (Fluorescent) | 558 | 589 | 26.69 |
| mCitrine | 516 | 529 | 27.05 |
| mClavGR1 | | | 26.99 |
| mClavGR1.1 | | | 27.1 |
| mClavGR1.8 | | | 27.1 |
| mClavGR2 (Green) | 488 | 504 | 27.08 |
| mClavGR2 (Red) | 566 | 583 | 27.08 |
| mClover1.5 | | | 26.9 |
| mClover3 | 506 | 518 | 26.95 |
| mcRFP | | | 25.88 |
| mCRISPRed | 460 | 592 | 26.59 |
| mCyRFP1 | 528 | 594 | 26.44 |
| mECFP | 433 | 475 | 26.96 |
| meffCFP | 467 | 492 | 25.99 |
| meffCP | | | 24.97 |
| meffGFP | 492 | 506 | 26.54 |
| meffRFP | 560 | 576 | 26.51 |
| mEGFP | 488 | 507 | 27 |
| meleCFP | 454 | 485 | 25.9 |
| meleRFP | 573 | 579 | 25.84 |
| mEmerald | 487 | 509 | 26.95 |
| mEos2 (Red) | 573 | 584 | 25.84 |
| mEos2 (Green) | 506 | 519 | 25.84 |
| mEos2-A69T (Green) | 495 | 509 | 25.87 |
| mEos2-A69T (Orange) | 565 | 580 | 25.87 |
| mEos2-NA | | | 25.75 |
| mEos3.1 (Green) | 505 | 513 | 25.73 |
| mEos3.1 (Red) | 570 | 580 | 25.73 |
| mEos3.2 (Green) | 507 | 516 | 25.74 |
| mEos3.2 (Red) | 572 | 580 | 25.74 |
| mEos4a (Green) | 505 | 515 | 26.05 |
| mEos4a (Red) | 571 | 580 | 26.05 |
| mEos4b (Green) | 505 | 516 | 25.9 |
| mEos4b (Red) | 570 | 580 | 25.9 |
| mEosEM (Green) | 503 | 511 | 25.87 |
| mEosEM (Red) | 569 | 579 | 25.87 |
| mEosFP (Green) | 505 | 516 | 25.83 |
| mEosFP (Red) | 569 | 581 | 25.83 |
| mEosFP-F173S (Green) | 486 | 514 | 25.76 |
| mEosFP-F173S (Red) | 550 | 581 | 25.76 |
| mEosFP-M159A (Green) | 487 | 514 | 25.76 |
| meruFP | | | 25.93 |
| mEYFP | 515 | 528 | 27.05 |
| MfaG1 | 492 | 508 | 25.98 |
| mGarnet | 598 | 670 | 25.19 |
| mGarnet2 | 598 | 671 | 25.26 |
| mGeos-C (On) | 505 | 516 | 25.81 |
| mGeos-E (On) | 501 | 513 | 25.84 |
| mGeos-F (On) | 504 | 515 | 25.85 |
| mGeos-L (On) | 501 | 513 | 25.82 |
| mGeos-M (On) | 503 | 514 | 25.84 |
| mGeos-S (On) | 501 | 512 | 25.79 |
| mGinger1 | 587 | 637 | 24.85 |
| mGinger2 | 578 | 631 | 24.84 |
| mGold | 515 | 531 | 26.91 |
| mGrape1 | 595 | 625 | 25.43 |
| mGrape2 | 605 | 636 | 26.8 |
| mGrape3 | 608 | 646 | 26.77 |
| mGreenLantern | 503 | 514 | 26.82 |
| mHoneydew | 487 | 562 | 25.36 |
| MiCy | 472 | 495 | 26.15 |
| mIFP | 683 | 704 | 35.11 |
| miniSOG | 447 | 501 | 12.33 |
| miniSOG2 | 429 | 507 | 12.44 |
| miniSOG Q103V | 440 | 487 | 12.3 |
| miRFP | 674 | 703 | 34.32 |
| miRFP2 | 676 | 706 | 34.3 |
| miRFP670 | 642 | 670 | 34.49 |
| miRFP670-2 | 643 | 670 | 34.52 |
| miRFP670nano | 645 | 670 | 17.1 |
| miRFP670v1 | 644 | 670 | 34.48 |
| miRFP680 | 661 | 680 | 34.62 |
| miRFP682 | 663 | 682 | 34.56 |
| miRFP702 | 673 | 702 | 34.51 |
| miRFP703 | 674 | 703 | 34.51 |
| miRFP709 | 683 | 709 | 34.56 |
| miRFP713 | 690 | 713 | 34.62 |
| miRFP720 | 702 | 720 | 34.68 |
| mIrisFP (Green) | 486 | 516 | 25.69 |
| mIrisFP (Red) | 546 | 578 | 25.69 |
| mKalama1 | 385 | 456 | 26.79 |
| mKate | 588 | 635 | 26.02 |
| mKate2 | 588 | 633 | 26.07 |
| mKate2.5 | | | 25.65 |
| mKate M41G S158C | 593 | 648 | 25.96 |
| mKate S158A | 585 | 630 | 26.01 |
| mKate S158C | 586 | 630 | 26.04 |
| mKeima | 440 | 620 | 25.05 |
| mKeima (pH 5) | 586 | 620 | 25.05 |
| mKeima (pH 6) | 440 | 620 | 25.05 |
| mKeima (pH 8) | 440 | 620 | 25.05 |
| mKeima (pH 4) | 586 | 620 | 25.05 |
| mKelly1 | 596 | 656 | 24.69 |
| mKelly2 | 598 | 649 | 24.73 |
| mKG | 494 | 507 | 24.42 |
| mK-GO (Late) | 548 | 561 | 24.46 |
| mK-GO (Early) | 500 | 509 | 24.46 |
| mKikGR (Red) | 580 | 591 | 26.45 |
| mKikGR (Green) | 505 | 515 | 26.45 |
| mKillerOrange | 458 | 560 | 26.37 |
| mKO | 548 | 559 | 24.45 |
| mKO2 | 551 | 565 | 24.46 |
| mKOκ | 551 | 563 | 24.47 |
| mMaple (Green) | 489 | 505 | 27.25 |
| mMaple (Red) | 566 | 583 | 27.25 |
| mMaple2 (Green) | 492 | 506 | 27.15 |
| mMaple2 (Red) | 570 | 582 | 27.15 |
| mMaple3 (Green) | 491 | 506 | 27.21 |
| mMaple3 (Red) | 568 | 583 | 27.21 |
| mMaroon1 | 609 | 657 | 27.64 |
| mmGFP | 398 | 505 | 23.72 |
| mMiCy | 470 | 496 | 26.04 |
| mmilCFP | 404 | 492 | 25.56 |
| mNectarine | 558 | 578 | 26.59 |
| mNeonGreen | 506 | 517 | 26.65 |
| mNeptune | 600 | 650 | 27.41 |
| mNeptune2 | 599 | 651 | 27.45 |
| mNeptune2.5 | 599 | 643 | 27.53 |
| mNeptune681 | 604 | 681 | 27.54 |
| mNeptune684 | 604 | 684 | 27.51 |
| mOFP.T.12 | | | 26.7 |
| mOFP.T.8 | | | 26.73 |
| montFP | | | 20.66 |
| Montipora sp. #20-9115 | 580 | 606 | 24.99 |
| mOrange | 548 | 562 | 26.74 |
| mOrange2 | 549 | 565 | 26.82 |
| moxBFP | 385 | 448 | 26.89 |
| moxCerulean3 | 434 | 474 | 26.62 |
| moxDendra2 (Green) | 490 | 504 | 26.09 |
| moxDendra2 (Red) | 551 | 571 | 26.09 |
| moxEos3.2 | | | 25.75 |
| moxGFP | 486 | 510 | 26.88 |
| moxMaple3 (Green) | 490 | 506 | 27.19 |
| moxMaple3 (Red) | 569 | 584 | 27.19 |
| moxNeonGreen | 505 | 520 | 26.63 |
| moxVenus | 514 | 526 | 26.85 |
| mPA-GFP | | | 27.03 |
| mPapaya | 530 | 541 | 26.91 |
| mPapaya0.3 | | | 26.93 |
| mPapaya0.6 | | | 26.96 |
| mPapaya0.7 | 529 | 541 | 26.97 |
| mPlum | 590 | 649 | 25.59 |
| mPlum-E16P | 590 | 630 | 25.56 |
| mRaspberry | 598 | 625 | 25.51 |
| mRed7 | 589 | 606 | 26.21 |
| mRed7Q1 | 560 | 591 | 26.28 |
| mRed7Q1S1 | 569 | 595 | 26.29 |
| mRed7Q1S1BM | 569 | 596 | 26.36 |
| mRFP1 | 584 | 607 | 25.42 |
| mRFP1.1 | 589 | 612 | 25.41 |
| mRFP1.2 | 590 | 612 | 25.55 |
| mRFP1.3 | | | 26.75 |
| mRFP1.4 | | | 26.76 |
| mRFP1.5 | | | 26.74 |
| mRFP1-Q66C | 559 | 580 | 25.4 |
| mRFP1-Q66S | 555 | 569 | 25.38 |
| mRFP1-Q66T | 549 | 570 | 25.4 |
| mRhubarb713 (Pr) | 690 | 713 | 34.5 |
| mRhubarb719 (Pr) | 700 | 719 | 34.5 |
| mRhubarb720 (Pr) | 701 | 720 | 34.54 |
| mRojoA | 597 | 633 | 26.74 |
| mRojoB | 598 | 631 | 26.76 |
| mRouge | 600 | 637 | 26.79 |
| mRtms5 | 588 | 633 | 24.96 |
| mRuby | 558 | 605 | 25.22 |
| mRuby2 | 559 | 600 | 26.49 |
| mRuby3 | 558 | 592 | 26.56 |
| mRubyFT (Blue-form) | 408 | 457 | 26.43 |
| mRubyFT (Red-form) | 582 | 624 | 26.43 |
| mScarlet | 569 | 594 | 26.35 |
| mScarlet-H | 551 | 592 | 26.36 |
| mScarlet-I | 569 | 593 | 26.36 |
| mStable | 597 | 633 | 26.09 |
| mStrawberry | 574 | 596 | 26.64 |
| mTagBFP2 | 399 | 454 | 26.65 |
| mTangerine | 568 | 585 | 25.37 |
| mTFP* | 468 | 495 | 25.31 |
| mTFP0.3 | 458 | 488 | 27.51 |
| mTFP0.4 | | | 27.48 |
| mTFP0.5 | | | 27.48 |
| mTFP0.6 | | | 27.04 |
| mTFP0.7 (On) | 453 | 488 | 26.96 |
| mTFP0.8 | | | 26.94 |
| mTFP0.9 | | | 26.88 |
| mTFP1 | 462 | 492 | 26.91 |
| mTFP1-Y67H | | | 26.88 |
| mTFP1-Y67W | 424 | 461 | 26.93 |
| mT-Sapphire | 399 | 511 | 26.94 |
| mTurquoise | 434 | 474 | 26.88 |
| mTurquoise-146G | | | 26.82 |
| mTurquoise-146S | | | 26.85 |
| mTurquoise2 | 434 | 474 | 26.91 |
| mTurquoise2-G | | | 26.86 |
| mTurquoise-DR | | | 26.94 |
| mTurquoise-GL | | | 26.84 |
| mTurquoise-GV | | | 26.82 |
| mTurquoise-RA | | | 26.89 |
| muGFP | 490 | 508 | 26.78 |
| mUkG | 483 | 499 | 25.61 |
| mVenus | 515 | 527 | 26.89 |
| mVenus-Q69M | 515 | 528 | 26.9 |
| mVermilion | | | |
| mVFP | 491 | 503 | 25.64 |
| mVFP1 | 491 | 503 | 25.6 |
| mWasabi | 493 | 509 | 26.94 |
| Neptune | 600 | 650 | 27.44 |
| NijiFP (Green) | 469 | 507 | 26.06 |
| NijiFP (Orange) | 526 | 569 | 26.06 |
| NowGFP | 494 | 502 | 26.69 |
| NpR3784g | | | 17.13 |
| obeCFP | 400 | 499 | 26.19 |
| obeGFP | 502 | 515 | 26.35 |
| obeYFP | 514 | 528 | 26.37 |
| OFP | 548 | 573 | 25.12 |
| OFPxm | 509 | 523 | 27.02 |
| O-GECO1 (calcium free) | 558 | 570 | |
| O-GECO1 (calcium saturated) | 545 | 563 | |
| oxBFP | 385 | 448 | 26.86 |
| oxCerulean | 435 | 477 | |
| oxGFP | 486 | 510 | 26.85 |
| oxVenus | 514 | 526 | 26.82 |
| P11 | 471 | 502 | 26.9 |
| P4 | 382 | 448 | 26.86 |
| P4-1 | 504 | 514 | 26.84 |
| P4-3E | 384 | 448 | 26.75 |
| P9 | 471 | 502 | 26.9 |
| Padron (On) | 503 | 522 | 25.46 |
| Padron0.9 (On) | 505 | 522 | 25.38 |
| Padron2 (On-state) | 492 | 516 | 25.16 |
| Padron(star) (On) | 503 | 522 | 25.48 |
| PA-GFP (On) | 504 | 517 | 26.97 |
| PAmCherry1 (On) | 564 | 595 | 26.77 |
| PAmCherry2 (On) | 570 | 596 | 26.74 |
| PAmCherry3 (On) | 570 | 596 | 26.75 |
| PAmKate (On) | 586 | 628 | 26.28 |
| PATagRFP (On) | 562 | 595 | 26.08 |
| PATagRFP1297 (On) | 563 | 595 | 26.01 |
| PATagRFP1314 (On) | 562 | 596 | 26.09 |
| pcDronpa (Red) | 569 | 581 | 25.39 |
| pcDronpa (Green) | 505 | 517 | 25.39 |
| pcDronpa2 (Red) | 569 | 583 | 25.34 |
| pcDronpa2 (Green) | 504 | 515 | 25.34 |
| pcStar (Green) | 505 | 515 | 25.72 |
| pcStar (Red) | 567 | 579 | 25.72 |
| PdaC1 | 480 | 492 | 25.09 |
| pdae1GFP | 491 | 511 | 24.92 |
| PDM1-4 | | | 25.47 |
| phiLOV2.1 | 451 | 501 | 12.74 |
| phiLOV3 | 452 | 502 | 12.67 |
| phiYFP | 525 | 537 | 26.05 |
| phiYFPv | 524 | 537 | 25.94 |
| pHluorin2 (acidic) | 475 | 509 | 26.93 |
| pHluorin2 (alkaline) | 395 | 509 | 26.93 |
| pHluorin, ecliptic (acidic) | 395 | 509 | 26.96 |
| pHluorin, ecliptic | 395 | 509 | 26.96 |
| pHluorin, ratiometric (acidic) | 475 | 509 | 26.89 |
| pHluorin, ratiometric (alkaline) | 395 | 509 | 26.89 |
| pH-tdGFP | 488 | 515 | 54.21 |
| pHuji | 566 | 598 | 26.99 |
| PlamGFP | 502 | 514 | 26.5 |
| plobRFP | 578 | 614 | 26.45 |
| pmeaGFP1 | 489 | 504 | 24.98 |
| pmeaGFP2 | 487 | 502 | 24.94 |
| pmimGFP1 | 491 | 505 | 25.1 |
| pmimGFP2 | 491 | 505 | 25.11 |
| Pp2 FbFP | 449 | 495 | 16.99 |
| Pp2FbFP L30M | 449 | 495 | 17.01 |
| ppluGFP1 | 480 | 500 | 24.56 |
| ppluGFP2 | 482 | 502 | 24.65 |
| pporGFP | 495 | 507 | 24.73 |
| pporRFP | 578 | 595 | 26.05 |
| psamCFP | 404 | 492 | 25.88 |
| PS-CFP (Cyan) | 402 | 468 | 27.27 |
| PS-CFP (Green) | 490 | 511 | 27.27 |
| PS-CFP2 (Cyan) | 400 | 468 | 26.68 |
| PS-CFP2 (Green) | 490 | 511 | 26.68 |
| PSmOrange (Far-red) | 634 | 662 | 26.79 |
| PSmOrange (Orange) | 548 | 565 | 26.79 |
| PSmOrange2 (Orange) | 546 | 561 | 26.73 |
| PSmOrange2 (Far-red) | 619 | 651 | 26.73 |
| psupFP | | | |
| ptilGFP | 500 | 508 | 27.05 |
| Q80R | | | 26.91 |
| R3-2 + PCB | 620 | 648 | 14.96 |
| RCaMP | 575 | 602 | 49.27 |
| RDSmCherry0.1 | 598 | 625 | 26.59 |
| RDSmCherry0.2 | 600 | 630 | 26.65 |
| RDSmCherry0.5 | 604 | 636 | 26.69 |
| RDSmCherry1 | 600 | 630 | 26.64 |
| REX-GECO1 (calcium free) | 578 | 599 | |
| REX-GECO1 (calcium | 484 | 586 | |
| saturated) | | | |
| R-FlincA | 560 | 585 | |
| rfloGFP | 508 | 518 | 26.01 |
| rfloGFP2 | | | |
| rfloRFP | 566 | 574 | |
| RFP611 | 559 | 611 | 26 |
| RFP618 | 560 | 618 | 26.1 |
| RFP630 | 583 | 630 | 26.03 |
| RFP637 | 587 | 637 | 26.09 |
| RFP639 | 588 | 639 | 26.12 |
| R-GECO1 (calcium free) | 576 | 599 | |
| R-GECO1 (calcium saturated) | 563 | 588 | |
| R-GECO1.2 (calcium free) | 573 | 597 | |
| R-GECO1.2 (calcium saturated) | 558 | 585 | |
| roGFP1 | 475 | 508 | 26.83 |
| roGFP1-R1 | 400 | 508 | 26.94 |
| roGFP1-R8 | 400 | 511 | 26.88 |
| roGFP2 | 490 | 511 | 26.87 |
| RpBphP1 | | | 34.5 |
| RpBphP2 | | | 34.56 |
| RpBphP6 | | | 34.53 |
| rrenGFP | 485 | 508 | 25.99 |
| rrGFP | | | 26.18 |
| RRvT | 556 | 583 | 54.55 |
| rsCherry (On) | 572 | 610 | 26.77 |
| rsCherryRev (On) | 572 | 608 | 26.73 |
| rsCherryRev1.4 (On) | 572 | 609 | 26.71 |
| rsEGFP (On) | 493 | 510 | 26.97 |
| rsEGFP2 (On) | 478 | 503 | 26.94 |
| rsFastLime (On) | 496 | 518 | 25.44 |
| rsFolder (Green) | 477 | 503 | 26.88 |
| rsFolder2 (Green) | 478 | 503 | 26.9 |
| rsFusionRed1 (On) | 577 | 605 | 25.59 |
| rsFusionRed2 (On) | 580 | 607 | 25.6 |
| rsFusionRed3 (On) | 580 | 607 | 25.57 |
| RSGFP1 | | | 26.79 |
| RSGFP2 | | | 26.85 |
| RSGFP3 | | | 26.83 |
| RSGFP4 | | | 26.83 |
| RSGFP6 | | | 26.84 |
| RSGFP7 | | | 26.84 |
| rsGreen1 (Bright) | 486 | 509 | 26.94 |
| rsKame (On) | 503 | 517 | 25.49 |
| rsTagRFP (ON) | 567 | 585 | 26.63 |
| Rtms5 | | | 24.81 |
| SAASoti (Green) | 510 | 519 | 25.15 |
| SAASoti (Red) | 510 | 589 | 25.15 |
| Sandercyanin | 375 | 675 | 18.62 |
| Sapphire | 399 | 511 | 26.84 |
| sarcGFP | 483 | 500 | 25.83 |
| SBFP1 | 380 | 446 | 26.85 |
| SBFP2 | 380 | 446 | 26.84 |
| SCFP1 | 434 | 477 | 26.93 |
| SCFP2 | 434 | 474 | 26.92 |
| SCFP3A | 433 | 474 | 26.89 |
| SCFP3B | 433 | 474 | 26.8 |
| scleFP1 | | | 16.71 |
| scleFP2 | | | 16.51 |
| scubGFP1 | 497 | 506 | |
| scubGFP2 | 497 | 506 | |
| scubRFP | 570 | 578 | 26.06 |
| secBFP2 | 399 | 456 | 26.31 |
| SEYFP | 515 | 528 | 26.87 |
| sfCherry | | | 25.48 |
| sfCherry2 | | | 25.26 |
| sfCherry3C | | | 25.48 |
| sg11 | 385 | 506 | 26.92 |
| sg12 | 396 | 508 | 26.95 |
| sg25 | 474 | 506 | 26.94 |
| sg42 | 384 | 450 | 26.93 |
| sg50 | 384 | 450 | 26.9 |
| SGFP1 | 495 | 512 | 26.91 |
| SGFP2 | 495 | 512 | 26.89 |
| SGFP2(206A) | 496 | 512 | 26.84 |
| SGFP2(E222Q) | 496 | 512 | 26.89 |
| SGFP2(T65G) | 501 | 512 | 26.85 |
| SH3 (SH3) | 450 | 495 | 12.94 |
| ShadowG | 486 | 510 | 26.98 |
| SHardonnay | 511 | 524 | 26.98 |
| shBFP | 401 | 458 | |
| shBFP-N158S/L173I | 375 | 458 | |
| ShG24 | 486 | 506 | 26.96 |
| ShyRFP (Yellow state) | 450 | 596 | 26.9 |
| Sirius | 355 | 424 | 26.69 |
| SiriusGFP | 504 | 516 | 26.97 |
| Skylan-NS (On) | 499 | 511 | 25.71 |
| Skylan-S (On) | 499 | 511 | 25.68 |
| smURFP | 642 | 670 | 14.96 |
| SNIFP | 697 | 720 | 34.72 |
| SOPP | 440 | 487 | 12.31 |
| SOPP2 | 439 | 491 | 12.23 |
| SOPP3 | 439 | 490 | 12.09 |
| spGFP 11 | | | 1.83 |
| spGFP1-10 | | | 24.01 |
| spisCP | | | 24.95 |
| SPOON (on) | 510 | 527 | 26.83 |
| sREACh | 517 | 531 | 26.95 |
| StayGold | 496 | 515 | 24.61 |
| stylCP | | | 24.95 |
| stylGFP | 485 | 500 | 24.99 |
| Superfolder BFP | | | 26.75 |
| Superfolder CFP | 430 | 479 | 26.8 |
| Superfolder GFP | 485 | 510 | 26.78 |
| Superfolder mTurquoise2 | 434 | 474 | 26.85 |
| Superfolder mTurquoise2 ox | 434 | 474 | 26.84 |
| Superfolder YFP | 513 | 527 | 26.84 |
| SuperNova2 (SuperNova2) | 579 | 610 | 26.56 |
| SuperNova Green | 440 | 510 | 26.34 |
| SuperNova Red | 579 | 610 | 26.35 |
| super-TagRFP | 555 | 579 | 27.57 |
| SYFP2 | 515 | 527 | 26.88 |
| sympFP | | | |
| TagBFP | 402 | 457 | 26.28 |
| TagCFP | 458 | 480 | 26.68 |
| TagGFP | 482 | 505 | 26.68 |
| TagGFP2 | 483 | 506 | 26.87 |
| TagRFP | 555 | 584 | 26.09 |
| TagRFP657 | 611 | 657 | 26.31 |
| TagRFP658 | 611 | 658 | 26.39 |
| TagRFP675 | 598 | 675 | 26.38 |
| TagRFP-T | 555 | 584 | 27.58 |
| TagYFP | 508 | 524 | 26.97 |
| tdimer2(12) | 552 | 579 | 52.66 |
| tdKatushka2 | 588 | 633 | 26.08 |
| td-RFP611 | 558 | 609 | 26.14 |
| td-RFP639 | 589 | 631 | 26.17 |
| TDsmURFP | 642 | 670 | 31.96 |
| tdTomato | 554 | 581 | 54.19 |
| TeAPCα | | | 14.89 |
| tKeima | 440 | 616 | 25.03 |
| Topaz | 514 | 527 | 26.93 |
| tPapaya0.01 | | | 27.53 |
| TripartiteGFP | 480 | 514 | 26.77 |
| Trp-less GFP | | | 25.6 |
| T-Sapphire | 399 | 511 | 26.88 |
| TurboGFP | 482 | 502 | 25.69 |
| TurboGFP-V197L | 482 | 502 | 24.4 |
| TurboRFP | 553 | 574 | 26.14 |
| Turquoise-GL | 434 | 474 | 26.78 |
| Ultramarine | 586 | 626 | 24.93 |
| UnaG | 498 | 527 | 15.58 |
| usGFP | 490 | 508 | 26.81 |
| V127T SAASoti (Green) | 509 | 519 | 25.15 |
| V127T SAASoti (Red) | 579 | 589 | 25.15 |
| Venus | 515 | 528 | 26.84 |
| VFP | 491 | 503 | 25.58 |
| vsfGFP-0 | 485 | 510 | 38.39 |
| vsfGFP-9 | 485 | 510 | 39.46 |
| vsGFP | | | 26.83 |
| W1C | 435 | 495 | 26.82 |
| W2 | 432 | 480 | 26.84 |
| W7 | 433 | 475 | 26.86 |
| WasCFP | 494 | 505 | 26.77 |
| Wi-Phy | 701 | 719 | 36.1 |
| Xpa | | | |
| yEGFP | | | 26.94 |
| YFP3 | | | 26.98 |
| YGFPdp (Default) | 502 | 513 | 24.7 |
| YGFPuv (Default) | 398 | 512 | 24.71 |
| YPet | 517 | 530 | 26.91 |
| zFP538 | 528 | 538 | 26.17 |
| zGFP | | | 26.04 |
| zoan2RFP | 552 | 576 | 26.4 |
| zRFP | | | 25.94 |
| ZsGreen | 496 | 506 | 26.11 |
| ZsYellow1 | 529 | 539 | 26.14 |

### 5.3 Methods of Use

The present disclosure further provides methods of using the assays of the present disclosure.

In certain embodiments, the present disclosure provides methods for characterizing the interaction of one or more proteins with a nucleic acid. For example, but not by way of limitation, the methods disclosed herein can be used to obtain information regarding how proteins interact with DNA and/or RNA.

In certain embodiments, the present disclosure provides methods for determining DNA repair and/or DNA damage response mechanisms using the methods of the assays of the present disclosure. The methods disclosed herein can provide information as to how proteins interact with damaged DNA, as well provide information as to how protein modifications influence protein-DNA binding dynamics.

In certain embodiments, DNA damage can refer to physical or chemical changes to DNA. In certain embodiments, DNA damage can occur from normal cellular processes or due to exposure of DNA damaging agents. In certain embodiments, DNA bases can be damaged by oxidative processes, alkylation of bases, base loss caused by the hydrolysis of bases, bulky adduct formation, DNA crosslinking, and DNA strand breaks, including single and double stranded breaks.

In certain embodiments, the present disclosure relates to post-translational modifications of proteins. In certain embodiments, post-translational modifications include covalent processing events that change the properties of a protein by proteolytic cleavage and adding a modifying group, such as acetyl, phosphoryl, glycosyl and methyl, to one or more amino acids. In certain embodiments, the assays described herein can be used to analyze the effect post-translational modifications have on the DNA damage response or the binding of the post-translationally modified protein to DNA.

In certain embodiments, the present disclosure relates to nucleic acid (e.g., DNA) structural alterations. In certain embodiments, DNA structural alterations can be associated with genome instability, e.g., mutations and chromosome rearrangements. Accordingly, such mutations and chromosome rearrangements can be associated with pathological disorders, and the assays of the present disclosure can be used to analyze the interaction of proteins with such nucleic acid (e.g., DNA) structural alterations.

The present disclosure can provide methods for characterizing disease-associated protein variants. For example, but not by way of limitation, the assays of the present disclosure can be used to analyze the interaction of protein variants with nucleic acid (e.g., DNA). In certain embodiments, the term “variant protein” or “protein variant”, or “variant” as used herein is meant to be a protein that differs from a parent protein by virtue of at least one amino acid modification. In certain embodiments, the protein variant has at least one amino acid modification compared to the parent protein, e.g., from about one to about ten amino acid modifications, and preferably from about one to about five amino acid modifications compared to the parent. The protein variant sequence herein will preferably possess at least about 80% homology with a parent protein sequence, and most preferably at least about 90% homology, more preferably at least about 95% homology. The protein variants of the present disclosure can be derived from parent proteins that are themselves from a wide range of sources. The parent protein can be substantially encoded by one or more genes from any organism, e.g., eukaryotic organism. For example, but not by way of limitation, the parent protein can be substantially encoded by one or more genes from humans, mice, rats, hamsters, rabbits, sheep, goats, camels, llamas, dromedaries, dogs, cats, cows, horses, pigs, monkeys, plants, fungi and protists.

### 5.4 Kits

The presently disclosed subject matter further provides kits containing materials useful for performing the assay and methods disclosed herein. For example, but not by way of limitation, any combination of the materials useful in the present disclosure can be packaged together as a kit for performing any of the disclosed assays or methods.

In certain embodiments, a kit of the present disclosure can contain a disposable microfluidic cell device preloaded with a specific buffer, tracer particles, and/or fluorescent dye. In certain embodiments, a kit of the present disclosure can include cells, nucleic acid that encodes a recombinant protein and/or the nucleic acid substrate, e.g., DNA substrate or RNA substrate. Alternatively, the cells can be cells that have been genetically engineered to express the recombinant protein. Non-limiting of examples of recombinant proteins and nucleic acid substrates are described herein in Section 5.2. In certain embodiments, the reagents can be packaged in single use form, suitable for carrying one set of analyses.

In certain embodiments, the kit further includes a package insert that provides instructions for using the components provided in the kit. For example, a kit of the present disclosure can include a package insert that provides instructions for using the microfluidic device provided in the kit.

Alternatively or additionally, the kit can include other materials desirable from a commercial and user standpoint, including other buffers, diluents and filters. In certain embodiments, the kit can include materials for preparing nuclear extracts. In certain embodiments, a kit of the present disclosure can include beads and/or fluorescent labels, e.g., Qdots. In certain embodiments, a kit of the present disclosure can include nucleic acid (e.g., DNA) linkers.

Kits can supply reagents in pre-measured amounts so as to simplify the performance of the subject assay or methods. Optionally, kits of the present disclosure comprise instructions for performing the assay or method. Other optional elements of a kit of the present disclosure include suitable buffers, labeling reagents, packaging materials, etc. The kits of the present disclosure can further comprise additional reagents that are necessary for performing the disclosed assays and methods. The reagents of the kit can be in containers in which they are stable, e.g., in lyophilized form or as stabilized liquids.

### 5.5 Exemplary Non-Limiting Embodiments

A. The present disclosure provides an assay for determining the binding kinetics of one or more proteins with a nucleic acid substrate comprising:
(a) expressing one or more recombinant proteins in a host cell;
(b) preparing a nuclear extract from the host cell expressing the one or more recombinant proteins;
(c) contacting the nuclear extract with a nucleic acid substrate;
(d) visualizing the one or more recombinant proteins binding to the nucleic acid substrate; and
(e) determining protein-nucleic acid association and dissociation kinetics.

A1. The assay of claim A, wherein the nucleic acid substrate is positioned within a microfluidic cell system, and wherein the nuclear extract is flowed through the microfluidic cell system to contact the nucleic acid substrate.

A2. The assay of A or A1, wherein the one or more recombinant proteins is a natural protein, synthetic protein, modified protein, or other protein analogue.

A3. The assay of any one of A-A2, wherein the one or more recombinant proteins is a variant, homolog, derivative, mutant or a functional fragment thereof of a wild type protein.

A4. The assay of any one of A-A3, wherein the one or more recombinant proteins is post-translationally modified.

A5. The assay of A4, wherein the post-translational modification comprises a proteolytic cleavage, glycosylation, or the addition of modifying group, such as acetyl, phosphoryl, glycosyl or methyl, to one or more amino acids of the protein.

A6. The assay of any one of A-A5, wherein the one or more recombinant proteins is labeled.

A7. The assay of any one of A-A6, wherein the one or more recombinant proteins is selected from the group consisting of DNA-binding proteins, RNA-binding proteins, DNA repair proteins, DNA damage response proteins, DNA modifying proteins, DNA polymerases, RNA polymerases, transcription factors, nucleases, chromatin remodeling factors, methylated DNA binding proteins, proteases, methylases, demethylases, acetylases, deacetylases, glycosylases, kinases, phosphatases, integrases, recombinases, ligases, topoisomerases, gyrases, helicases or a combination thereof.

A8. The assay of any one of A-A7, wherein the one or more recombinant proteins is selected from a group consisting of poly(ADP-ribose) polymerase 1 (PARP1), heterodimeric ultraviolet-damaged DNA-binding protein (UV-DDB), xeroderma pigmentosum complementation group C protein (XPC), 8-oxoguanine glycosylase 1 (OGG1), apurinic/apyrimidinic endonuclease 1 (APE1), DNA polymerase beta (Polbeta), Thymine DNA glycosylase (TDG), X-ray repair cross complementing 1 (XRCC1), DNA ligase 3 (Lig3α), poly(ADP-ribose) polymerase 2 (PARP2), alkyladenine glycosylase (AAG) or a combination thereof.

A9. The assay of any one of A-A8, wherein the one or more recombinant proteins is fluorescently labeled.

A10. The assay of A9, wherein the fluorescent label is a dye, fluorophore or fluorescent protein.

A11. The assay of any one of A-A10, wherein the host cell is a mammalian cell.

A12. The assay of A11, wherein the mammalian cell is selected from a group consisting of a human cell, hamster cell, mouse cell, rat cell, sheep cell, goat cell, monkey cell, dog cell, cat cell, horse cell, cow cell, pig cell or a combination thereof.

A13. The assay A11 or A12, wherein the host cell is selected from a group consisting of a U2OS cell, Sf9 cell, CHO cell, COS-7 cell, HEK293 cell, BHK cell, TM4 cell, CV1 cell, VERO-76 cell, HELA cell, MDCK cell, BRL cell, W138 cell, Hep G2 cell, MMT cell, TRI cell, MRC 5 cell, FS4 cell, RPE cell, hTERT-RPE cell, hTERT-BJ fibroblast or a combination thereof.

A14. The assay of any one of A-A13, wherein the assay further comprises analyzing the expression level of the one or more recombinant proteins in the nuclear extract.

A15. The assay of any one of A-A14, wherein the nucleic acid substrate is between about 10 and 100 kb in length.

A16. The assay of any one of A-A15, wherein the nucleic acid substrate is damaged.

A17. The assay of A16, wherein the damage is a physical or a chemical change.

A18. The assay of A15 or A16, wherein the nucleic acid damage is induced by UV exposure, enzymatic digestion, or oxidative damage.

A19. The assay of any one of A-A18, wherein the nucleic acid substrate comprises one or more nucleic acid analogues.

A20. The assay of A-A20, wherein the nucleic acid analogues are incorporated into the nucleic acid DNA by nick translation.

A21. The assay of A19 or A20, wherein the nucleic acid analogue is selected from a group consisting of 5-formyl-dCTP (5fC), 5-hm-dUTP, 6-thio-dGTP, 5-fluoro-dUTP, ara-CTP, Cy3-dUTP, dITP or a combination thereof.

A22. The assay of any one A1-A21, wherein the microfluidic system further comprises optical tweezers.

A23. The assay of any one of A1-A22, wherein the microfluidic system comprises a microfluidic cell having at least 4 channels separated by laminar flow.

A24. The assay of A23, wherein:
(a) channel 1 contains beads;
(b) channel 2 contains the nucleic acid substrate;
(c) channel 3 contains the flow buffer; and/or
(d) channel 4 contains the cell extract.

A25. The assay of A24, wherein the beads are trapped in channel 1.

A26. The assay of A24 or A25, wherein the nucleic acid substrate is suspended between the beads in channel 2.

A27. The assay of any one of A24-A26, wherein a buffer solution is flowed through channel 3.

A28. The assay of any one of A24-A27, wherein the nuclear extract containing the one or more proteins contacts the nucleic acid substrate in channel 4.

A29. The assay of any one of A24-A28, wherein the flow rate is kept constant.

A30. The assay of any one of A24-A29, wherein the flow rate is pulsed.

A31. The assay of any one of A24-A30, wherein the flow is between about 0.05 and 0.5 bar.

A32. The assay of any one of A24-A31, wherein protein-nucleic acid interactions were observed without flow.

A33. The assay of any one of A24-A32, wherein the beads have a diameter between about 1 and 10 μm.

A34. The assay of A33, wherein the beads are polystyrene.

A35. The assay of any one of A24-A34, wherein the surface of the beads is modified to facilitate nucleic acid substrate attachment.

A36. The assay of A35, wherein the surface of the bead is modified to have a functional group selected from streptavidin, biotin, or poly-lysine.

A37. The assay of any one of A24-A36, wherein the nucleic acid substrate contains a functional group to facilitate bead attachment.

A38. The assay of A37, wherein the functional group is selected from a group consisting of biotin or streptavidin.

A39. The assay of any one of A24-A38, wherein the nucleic acid substrate is tethered to the beads by a biotin-streptavidin interaction.

A40. The assay of any one of A24-A39, wherein the nucleic acid substrate is held at a tension of about 5 to 40 pN.

A41. The assay of any one of A1-A40, wherein the microfluidic cell system further comprises fluorescence microscopy.

A42. The assay of any one of claims A-A41, wherein the one or more recombinant proteins is detected by fluorescence microscopy.

A43. The assay of A42, wherein the fluorescence microscopy can resolve an individual one or more proteins binding to a specific location along the nucleic acid substrate.

A44. The assay of any one of A41-A43, wherein the fluorescence microscopy comprises single-molecule-FRET imaging.

A45. The assay of claims A41-A43, wherein the fluorescence microscopy comprises confocal imaging.

A46. The assay of any one of A-A45, wherein the association and dissociation kinetics of the one or more recombinant protein comprise:
(a) a binding event duration (k_{off});
(b) number of binding events per second (kₒₙ);
(c) a binding position; and/or
(d) a movement on nucleic acid (MSD/velocity).

A47. The assay of any one of A-A46, wherein the nucleic acid substrate is DNA.

A48. The assay of any one of A-A47, wherein the nucleic acid substrate is RNA.

A49. The assay of A48, wherein the RNA is mRNA.

A50. The assay of any one of A-A49, wherein the nucleic acid substrate comprises one or more nucleosomes.

B. The present disclosure further provides a method for determining nucleic acid binding kinetics of one or more proteins using the assay of any one of A-A50.

B1. A method for determining DNA damage recognition of one or more proteins using the assay of any one of A-A50.

B2. A method for determining DNA repair mechanisms using the assay of any one of A-A50.

B3. A method for determining single molecule analysis of nucleic acid-binding proteins from nuclear extract using the assay of any one of A-A50.

C. A kit for performing the assays or methods of any one A-B3, wherein the kit comprises:
(a) a microfluid cell;
(b) a buffer fluid;
(c) a set of beads; and/or
(d) a nucleic acid substrate.

C1. The kit of C, wherein the kit further comprises:
(a) instructions for performing single molecule analysis of nucleic acid-binding proteins from nuclear extracts;
(b) tracer dyes; and/or
(c) reagents for conjugating functional groups.

### 6. EXAMPLES

### 6.1 Example 1

The presently disclosed subject matter will be better understood by reference to the following Example, which is provided as exemplary of the presently disclosed subject matter, and not by way of limitation.

### 6.1.1 SMADNE Workflow and Characterization of DNA Binding Events

This Example discloses a method for single-molecule characterization of protein-DNA dynamics referred to herein as Single-Molecule Analysis of DNA-binding proteins from Nuclear Extracts (SMADNE). SMADNE applies similar principles of previous single-molecule work with cellular extracts while making several significant improvements, allowing application to human cells and scalability to numerous proteins that bind DNA. The LUMICKS C-trap combined with optical tweezers, microfluidics, and 3-color confocal microscope, allowed for precise defined positions of fluorescently-tagged DNA repair proteins along a DNA substrate and at specific sites of damage. As shown below, SMADNE provides binding specificity and diffusivity measurements including characterizing multiple proteins simultaneously binding DNA damage with over 4 orders of magnitude of duration (0.1 to >100 s) and a wide range of 1D diffusivity values (from 0.001 to 1 μm² s⁻¹), with similar precision as other single molecule techniques. At the same time, SMADNE bridges the complex milieu of the nuclear environment containing thousands of proteins to a system where fluorescently tagged single particles can be followed and characterized. Thus, SMADNE has broad applicability to provide detail mechanistic information about diverse protein-DNA and protein-protein interactions.

### 6.1.1 Results

### SMADNE Characterization of PARP1 Binding to Damaged DNA.

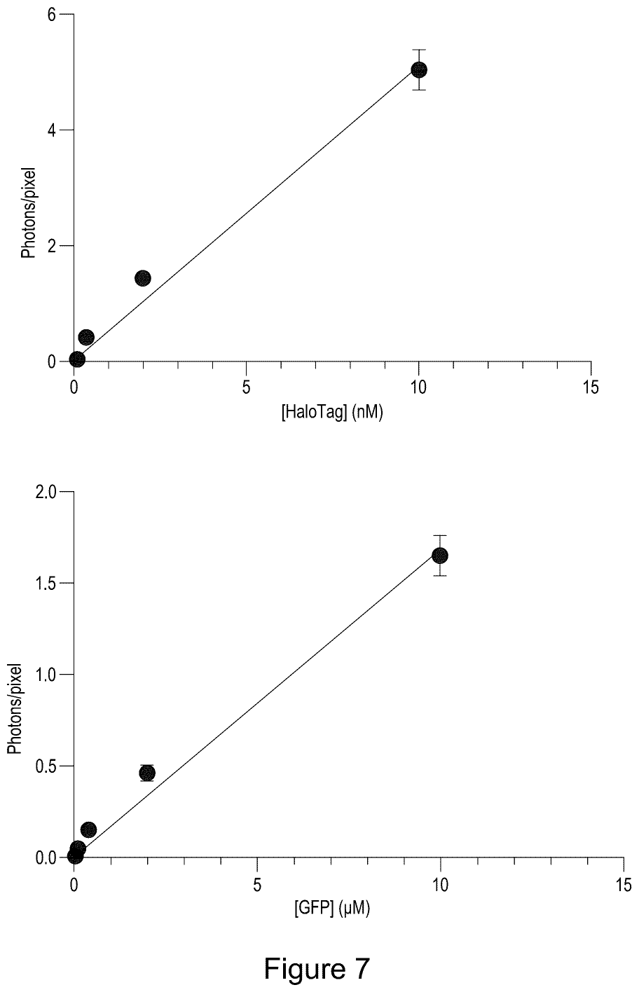

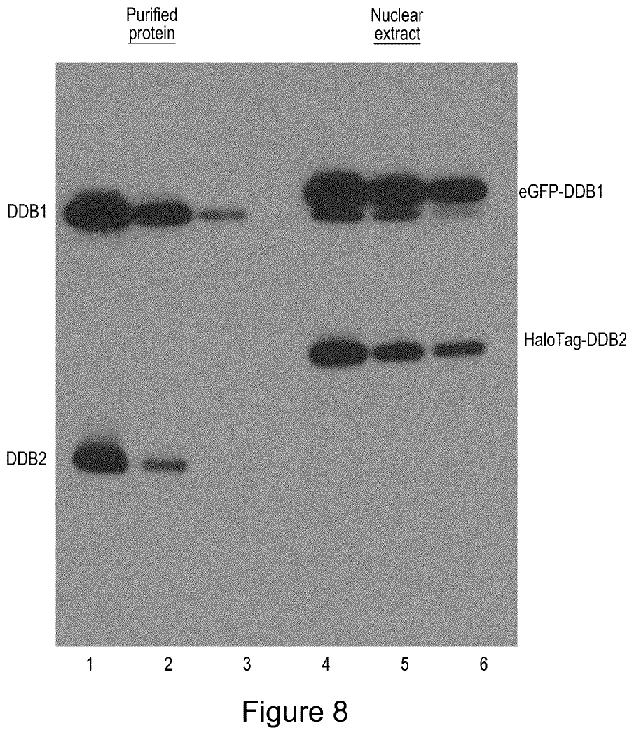

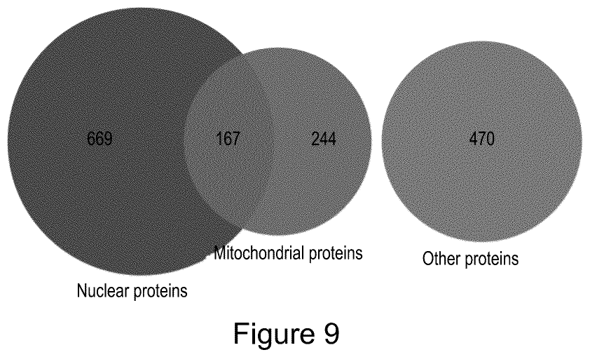

The present disclosure characterized fluorescently tagged DNA-binding proteins from nuclear extracts following the workflow shown in FIGS. 1A and 1B. Western blotting and fluorescence intensity of the tagged protein were utilized to provide estimates of the amount of target protein in the extract (FIGS. 7 and 8; Table 2), which are generally 50-100 times more prevalent than the endogenous protein under study. Endogenous proteins were considered too dilute to affect overall binding of the fluorescently labeled-proteins (Table 3)¹. Mass spectrometry confirmed that nuclear extraction protocol enriched for nuclear proteins (FIG. 9). Using the LUMICKS C-trap optical traps, streptavidin coated polystyrene beads were captured and biotinylated 48.5 kb DNA was suspended between the beads (FIG. 1C, left panel). After flowing in the nuclear extract containing the fluorescently labeled protein of interest, flow was stopped, and 2D confocal images were collected to verify binding of the protein to the DNA (FIG. 1C, middle panel). Then, the area being scanned was reduced to only the central DNA position. In 1-dimensional scanning mode, imaging rates as fast as 6 msec per scan were achieved. The data appeared as fluorescent time streaks (kymographs) and showed the fluorescently-tagged protein position over time, where the Y-axis represents the position on the DNA and the X-axis shows the scan time (FIG. 1C, right panel). In this mode, the Y-axis represents the position on the DNA where binding occurred, and the X-axis shows the scan time, which in this kymograph is 30 msec increments.

| | Average | Standard error | |
|---|---|---|---|
| | concentration | of the mean | Number of |
| Protein | (nM) | (nM) | measurements |
| HaloTag-JF635-DDB2 | 0.20 | 0.05 | 9 |
| eGFP-DDB1 | 0.41 | 0.15 | 9 |
| mNeon-DDB2 K244E | 0.31 | 0.04 | 6 |
| eGFP-OGG1(K249Q) | 0.79 | 0.09 | 6 |
| YFP-PARP1 | 0.76 | 0.31 | 6 |
| eGFP-XPC | 0.31 | 0.06 | 6 |
| tGFP-APE1 | 0.41 | 0.11 | 6 |
| tGFP-Polbeta | 0.29 | 0.09 | 6 |

| | | | | Emission |
|---|---|---|---|---|
| | | | Excitation | filters |
| Fluorophore | Lifetime (s) | SEM (s) | laser (nm) | (nm) |
| HaloTag-DDB2* | 95 | 20.5 | 488, 638 | 650-750 |
| eGFP-DDB1** | 56 | 13.1 | 488, 638 | 500-550 |
| mNeon-DDB2 | 24 | 3.6 | 488 | 500-550 |
| mNeon-DDB2 K244E | 28 | 4.1 | 488 | 500-550 |
| mScarlet-OGG1*** | 15 | 3.5 | 488, 561 | 575-625 |
| YFP-PARP1 | 22 | 0.8 | 488 | 500-550 |
| eGFP-XPC | 62 | 10.7 | 488 | 500-550 |
| tGFP-APE1 | 36 | 9.7 | 488 | 500-550 |
| tGFP-Polbeta | 19 | 0.5 | 488 | 500-550 |

*Collected with 488 nm laser on (0% absorbance)
**Collected with 636 nm laser on (0% absorbance)
***Collected with 488 nm laser on (15% absorbance)

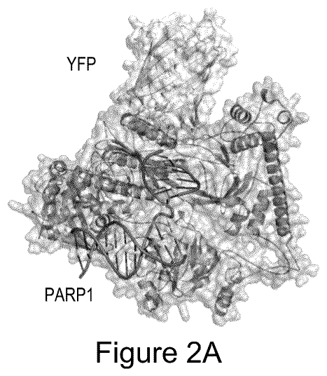

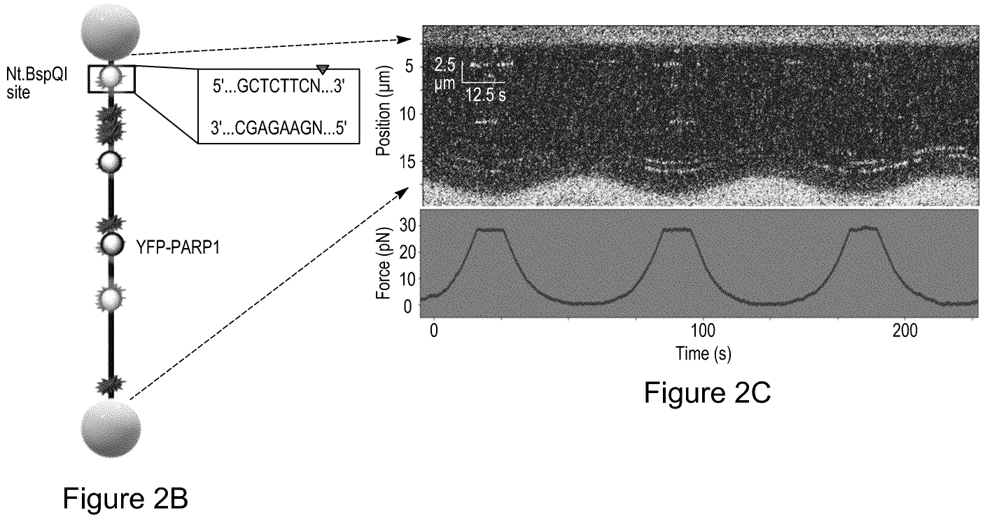

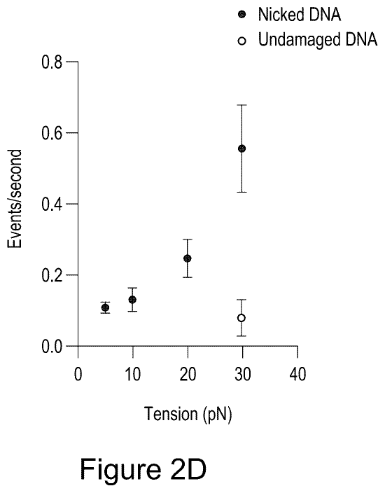

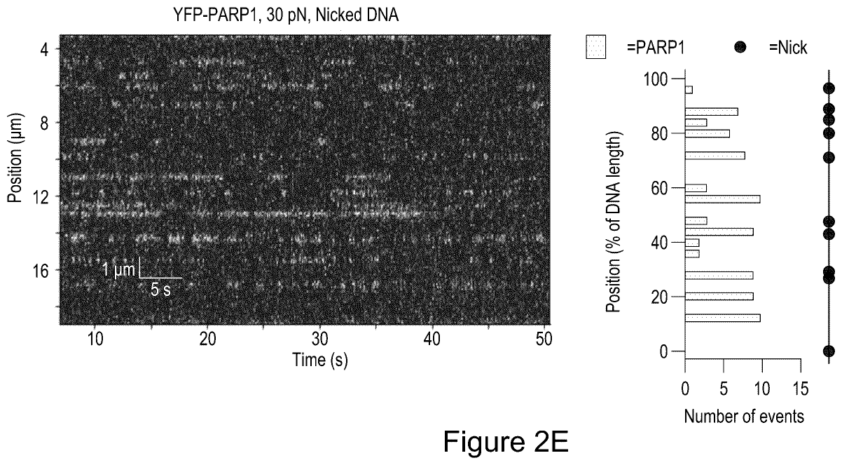

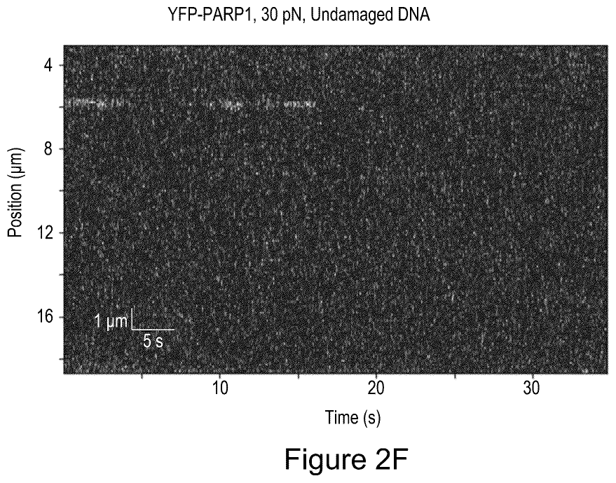

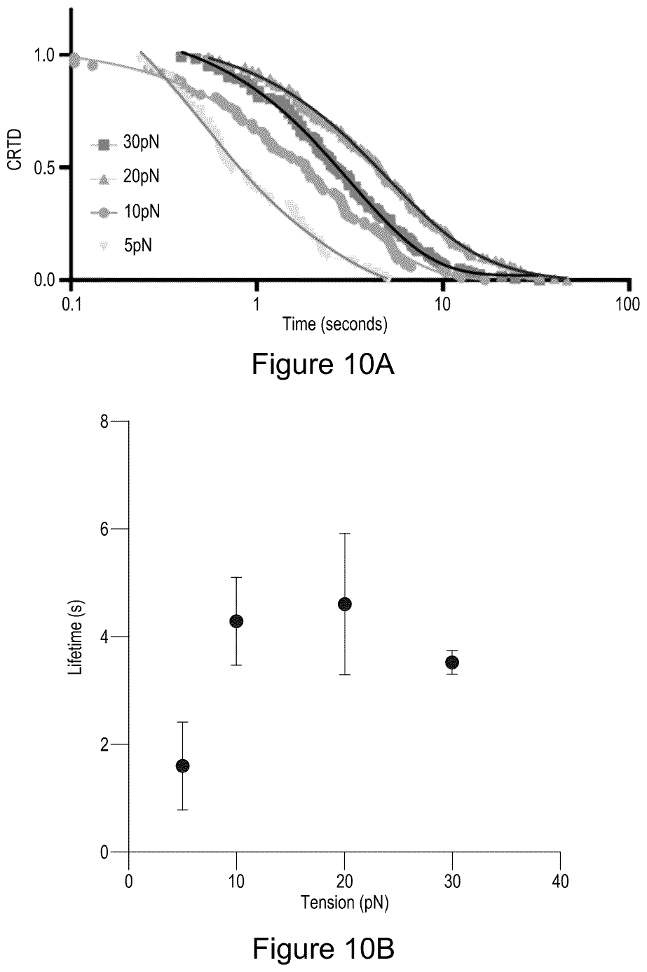

To validate the general utility of SMADNE, the present disclosure examined a series of fluorescently tagged-DNA repair proteins on various DNA substrates, namely poly(ADP-ribose) polymerase 1 (PARP1), poly(ADP-ribose) polymerase 2 (PARP2), xeroderma pigmentosum complementation group C protein (XPC), apurinic/apyrimidinic endonuclease 1 (APE1), DNA polymerase β (Pol B), DNA damage-binding protein 1 (DDB1), DNA damage-binding protein 2 (DDB2), DNA ligase 3 (Lig3α), X-ray repair cross-complementing protein 1 (XRCC1), thymine-DNA glycosylase (TDG), and alkyladenine glycosylase (AAG). In FIG. 1, YFP-PARP1 formed transient complexes on nicked DNA creating time streaks in the kymograph mode. Of note, multiple molecules revisited the same positions on the DNA (FIG. 1C, asterisks). These represented multiple events on the same damage site. The four key outcomes determined from SMADNE were: 1) how long a binding event lasted from start to finish (k_{off}); 2) how many binding events per second occurred (related to kₒₙ); 3) the position of binding events along the DNA; and 4) how bound proteins diffused along the DNA (FIG. 1D). For YFP-PARP1 at 10 pN of DNA tension, the average lifetime exhibited was 4.3 seconds, events occurred at 0.13 events per second, the positions agreed with the expected sites, and no diffusion along the DNA was observed (FIGS. 2 and 10).

### SMADNE Characterization of PARP Proteins Binding to Nicked DNA.

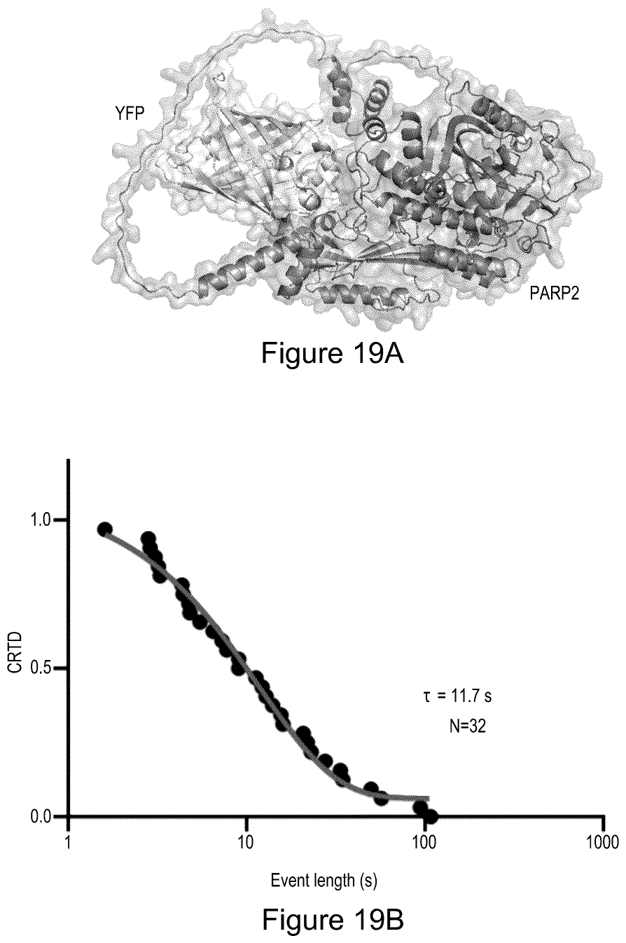

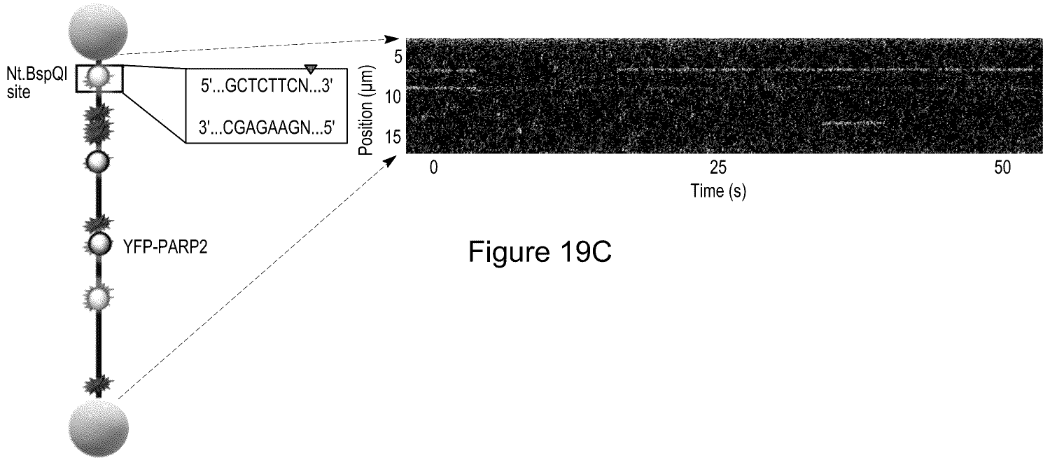

To demonstrate the broad applicability of SMADNE to various DNA repair proteins and different forms of DNA damage, the binding interactions were examined for YFP-tagged PARP1 from nuclear extracts on DNA containing ten nicks generated by a sequence-specific nickase (FIGS. 2A and 2B). Unexpectedly, increasing the tension on the DNA from 5 pN to 30 pN dramatically increased the number of YFP-PARP1 events per second. At 30 pN, new binding sites also appeared that were not observed at lower tension (FIG. 2C). It is possible that the higher tension makes previously existing nicks more identifiable by PARP1. Datasets were then collected at various constant DNA tensions. While binding lifetimes stayed relatively consistent as analyzed by fitting a cumulative residence time distribution (CRTD) to an exponential decay function (Table 4), events per second increased 4-fold at 30 pN of tension. In contrast, undamaged events per second remained low even at high tensions (FIG. 2D). YFP-PARP1 from nuclear extracts repeatedly bound at specific locations on the DNA, both on undamaged and damaged DNA (FIGS. 2E and 2F) indicating repeated specific binding events occurred at the nick sites. Datasets collected at 30 pN tension resulted in numerous binding events at 13 positions on the nicked DNA, indicating some off-target DNA damage present in the DNA sequence (FIG. 2E). While no previous studies have reported on PARP1 binding to nicked DNA at the single molecule level, single molecules of purified PARP1 labeled with Qdot binding to abasic sites, found PARP1 largely bound to its substrate via 3D diffusion, which agrees with the results observed with nicked DNA using SMADNE2. SMADNE was further used to explore the binding properties of PARP2, which is closely related to family member to PARP1 but the lacks N-terminal DNA binding domain. FIGS. 19A-19C demonstrates YFP-PARP2 binding events had a cumulative residence time distribution of 11.7 seconds.

| | | | | | | | | | | Binding | |
|---|---|---|---|---|---|---|---|---|---|---|---|
| | | | | | | Binding | Laser power | | Adjusted | lifetime: | |
| | | | | Lifetimes | | lifetime | at objective | Pulse | photo- | photo- | |
| | DNA | Tension | | (s) and | | (weighted | in μW | settings | bleaching | bleaching | |
| Protein | Substrate | (pN) | Buffer* | percentages | | average, s) | (488/561/638) | (on/total) | lifetime (s) | lifetime ratio | Notes |
| YFP- | Nicked | 10 | 3 | 4.2 ± 0.2 | s | 4.2 | (1.94/0/0) | Continuous | 22 | 5.1 | z |
| PARP1 | DNA | | | | | | | | | | |
| eGFP- | UV- | 10 | 1 | 0.9 ± 0.08 s | | 16.8 | (1.94/0/0) | Continuous | 62 | 3.7 | |
| XPC | damaged | | | (67 ± 4.9%) | | | | | | | |
| | DNA | | | 48.7 ± 26.3 s | | | | | | | |
| | (40 J) | | | (33 ± 4.9%) | | | | | | | |
| tGFP- | Nicked | 1 | 4 | 0.3 ± 0.02 | s | 0.3 | (1.94/0/0) | Continuous | 36 | 120 | |
| APE1 | DNA | | | | | | | | | | |
| tGFP- | Nicked | 10 | 1 | 1.8 ± 0.03 | s | 1.8 | (1.94/0/0) | Continuous | 19 | 10.6 | |
| Polbeta | DNA | | | | | | | | | | |
| HaloTag- | UV- | 10 | 1 | 3.9 ± 0.1 s | | 29 | (1.94/0/0.88) | 1/3 pulse | 285 | 9.8 | |
| JF635- | damaged | | | (43 ± 1.35%) | | | | | | | |
| DDB2 | DNA | | | 16.2 ± 1.1 s | | | | | | | |
| | (40 J) | | | (33 ± 1.79%) | | | | | | | |
| | | | | 90.3 ± 6.0 s | | | | | | | |
| | | | | (24 ± 1.17%) | | | | | | | |
| eGFP- | UV- | 10 | 1 | 1.8 ± 0.2 s | | 29 | (1.94/0/0.88) | 1/3 pulse | 168 | 5.8 | |
| DDB1 | damaged | | | (14 ± 1.2%) | | | | | | | |
| | DNA | | | 7.3 ± 0.2 s | | | | | | | |
| | (40 J) | | | (44 ± 1.28%) | | | | | | | |
| | | | | 60.9 ± 1.1 s | | | | | | | |
| | | | | (42 ± 0.43%) | | | | | | | |
| HaloTag- | 8-oxoG | 10 | 2 | 0.14 ± 0.0013 | s | 0.14 | (1.94/0/0.88) | Continuous | 95 | 679 | |
| JF635- | damaged | | | | | | | | | | |
| DDB2 | DNA | | | | | | | | | | |
| eGFP- | 8-oxoG | 10 | 2 | 0.25 ± 0.002 | s | 0.25 | (1.94/0/0.88) | Continuous | 56 | 224 | |
| DDB1 | damaged | | | | | | | | | | |
| | DNA | | | | | | | | | | |
| HaloTag- | UV- | 10 | 1 | 1.1 ± 0.01 | s | 0.56 | (1.94/0/0.88) | 1/3 pulse | 95 | 170 | Continuous |
| JF635- | damaged | | | | | | | and | | | and pulsed |
| DDB2 | DNA | | | | | | | continuous | | | scan data |
| | (40 J) + | | | | | | | | | | yielded |
| | 3 nM | | | | | | | | | | similar |
| | purified | | | | | | | | | | lifetimes |
| | UV-DDB | | | | | | | | | | and were |
| | | | | | | | | | | | combined |
| eGFP- | UV- | 10 | 1 | 0.6 ± 0.01 | s | 0.95 | (1.94/0/0.88) | 1/3 pulse | 56 | 58.9 | Continuous |
| DDB1 | damaged | | | | | | | and | | | and pulsed |
| | DNA | | | | | | | continuous | | | scan data |
| | (40 J) + | | | | | | | | | | yielded |
| | 3 nM | | | | | | | | | | similar |
| | purified | | | | | | | | | | lifetimes |
| | UV-DDB | | | | | | | | | | and were |
| | | | | | | | | | | | combined |
| mNeon- | UV- | 10 | 1 | 0.7 ± 0.06 s | | 8.5 | (1.94/0/0) | Continuous | 24 | 2.8 | |
| DDB2 | damaged | | | (53 ± 1.4%) | | | | | | | |
| K244E | DNA | | | 16.9 ± 1.8 s | | | | | | | |
| | (40 J) | | | (47 ± 1.4%) | | | | | | | |
| mScarlet- | 8-oxoG | 10 | 3 | 1.4 ± 0.01 | s | 1.4 | (1.94/2.80/0) | Continuous | 15 | 10.7 | |
| OGG1 | damaged | | | | | | | | | | |
| | DNA | | | | | | | | | | |

| | | | | | | | | | |
|---|---|---|---|---|---|---|---|---|---|
| eGFP- | 8-oxoG | 10 | 3 | 7.7 ± 0.27 s | 15.4 | (1.94/0/0) | Continuous | 56 | 3.6 |
| OGG1 | damaged | | | (78 ± 2.29%) | | | | | |
| (K249Q) | DNA | | | 42.9 ± 8.7 s | | | | | |
| | | | | (22 ± 2.29%) | | | | | |

*Buffer 1: 150 mM NaCl, 10% glycerol, 20 mM HEPES pH 7.5, 1 mM DTT, 5% glycerol, 0.5 mg/mL BSA, 1 mM Trolox; Buffer 2: 150 mM NaCl, 25 mM HEPES 7.5, 0.1 mg/mL BSA, 1 mM DTT; Buffer 3: 150 mM NaCl, 25 mM HEPES 7.5, 0.1 mg/mL BSA, 1 mM DTT, 1 mM Trolox; Buffer 4: 150 mM NaCl, 50 mM HEPES pH 7.4, 0.1 mg/mL BSA, 1 mM DTT, 1 mM Trolox

### Application of SMADNE to Study Transient DNA Interactions.

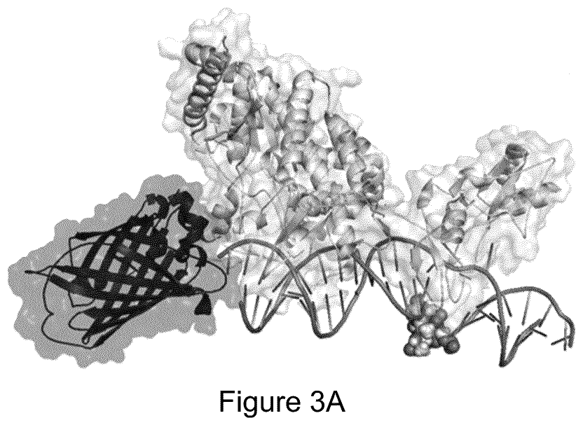

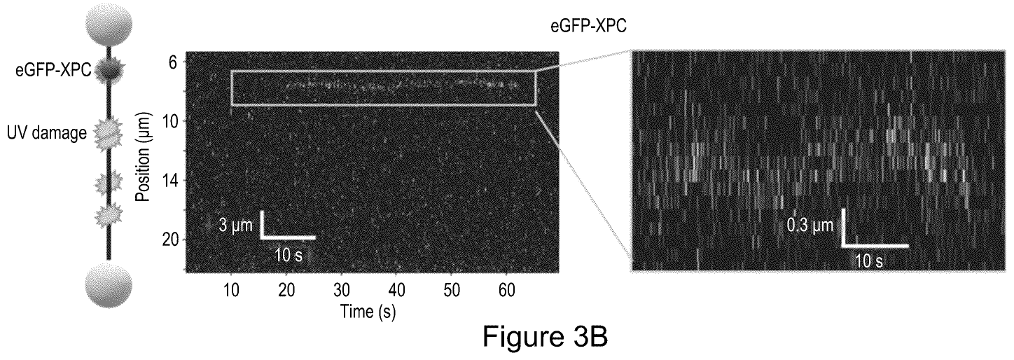

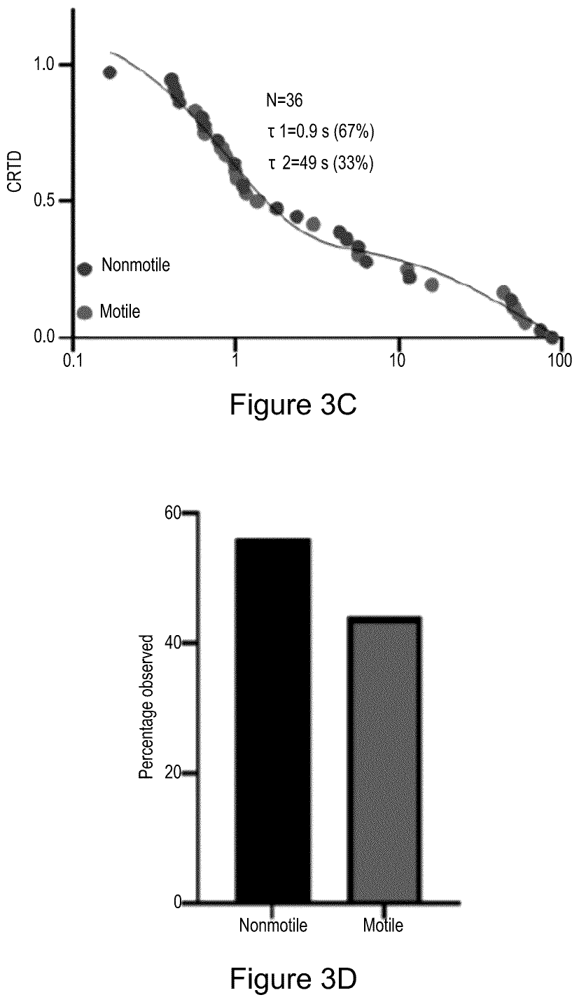

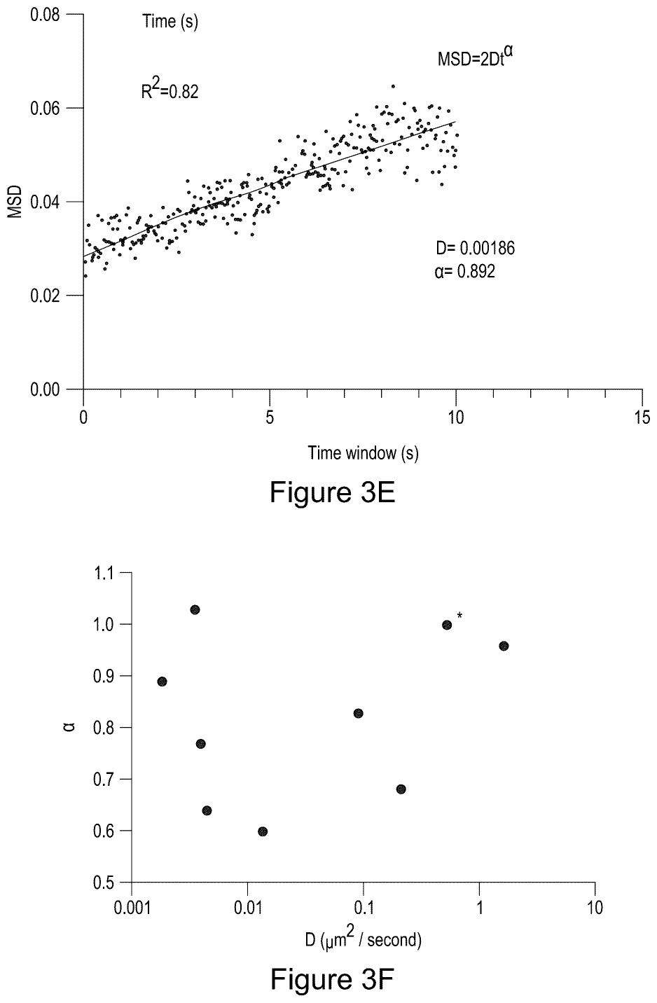

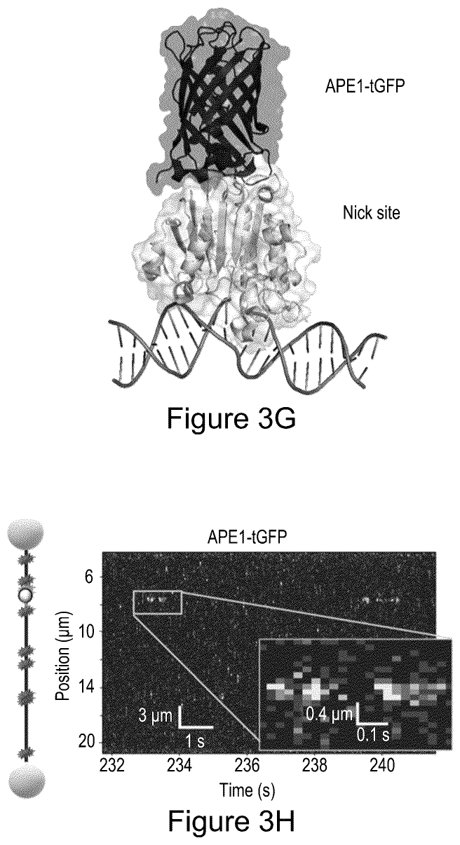

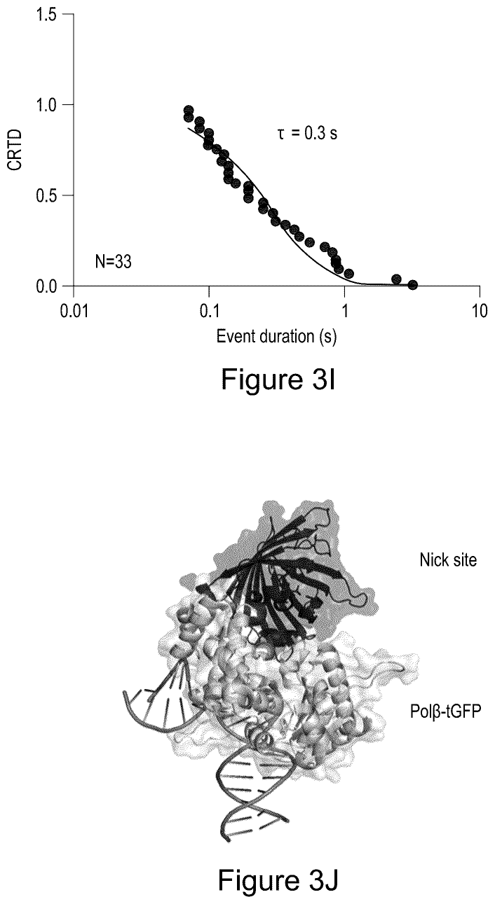

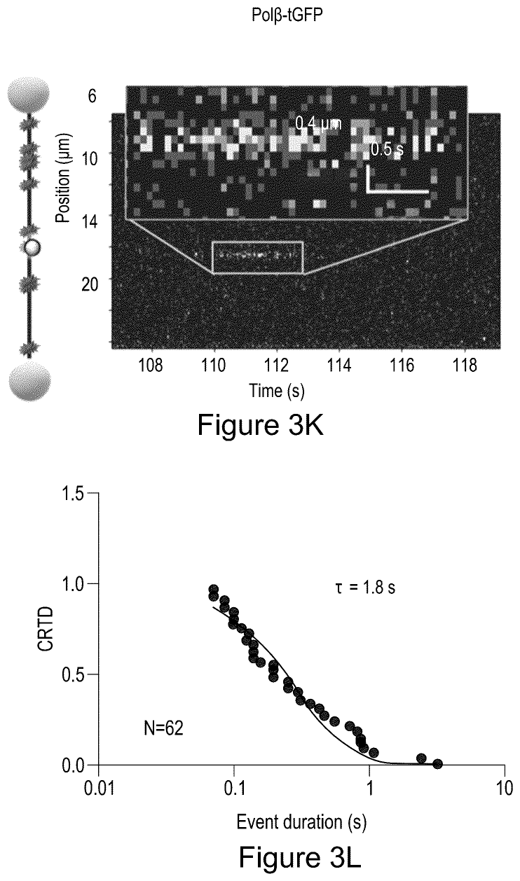

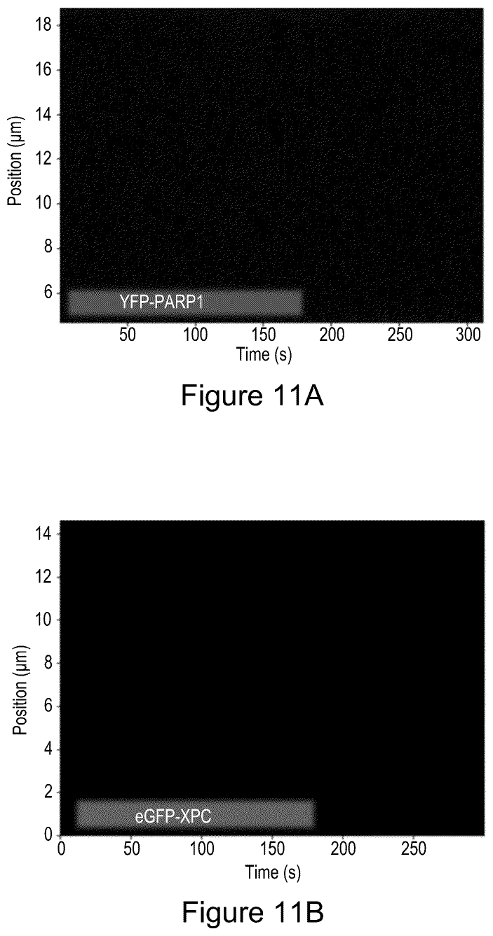

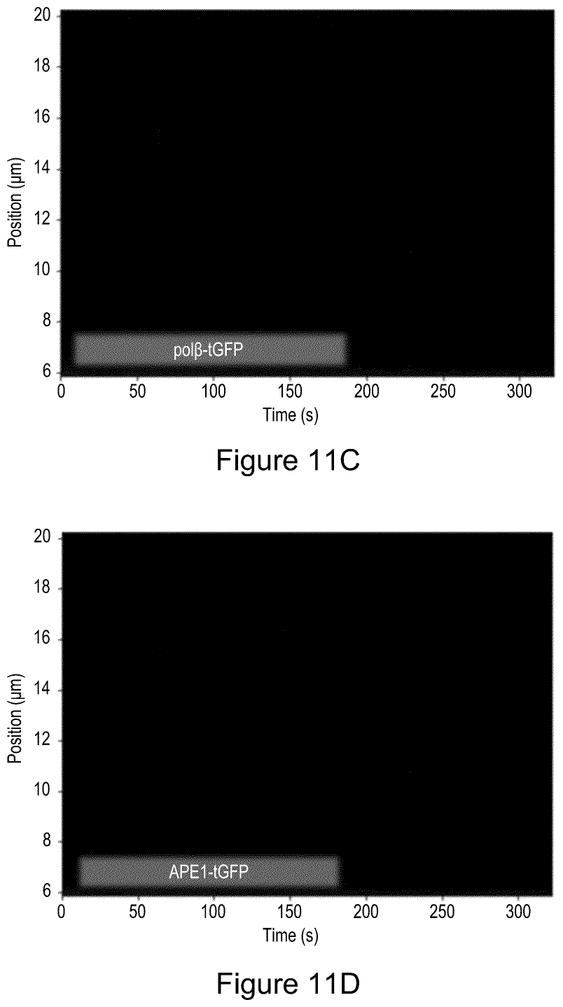

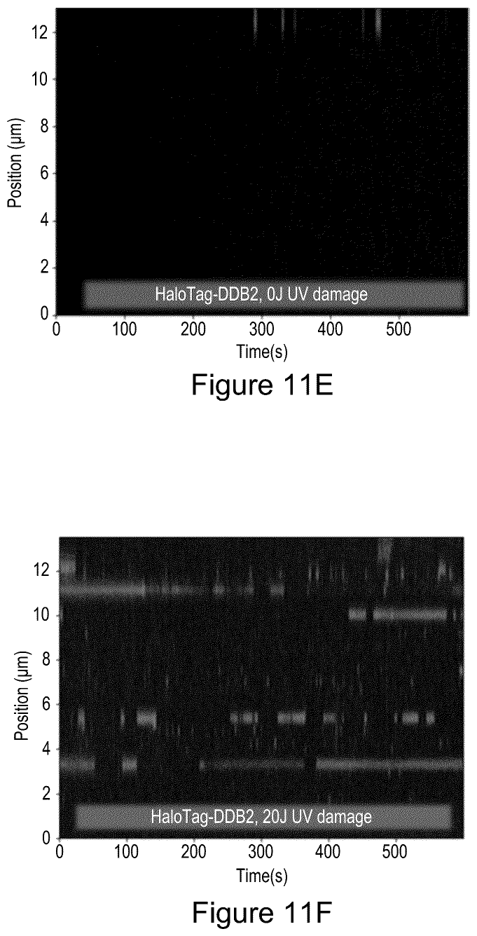

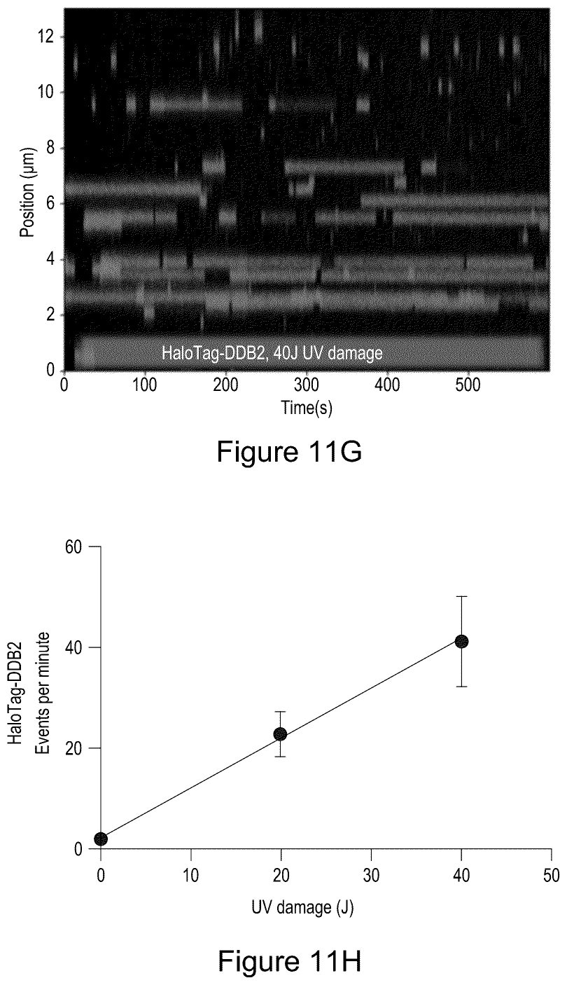

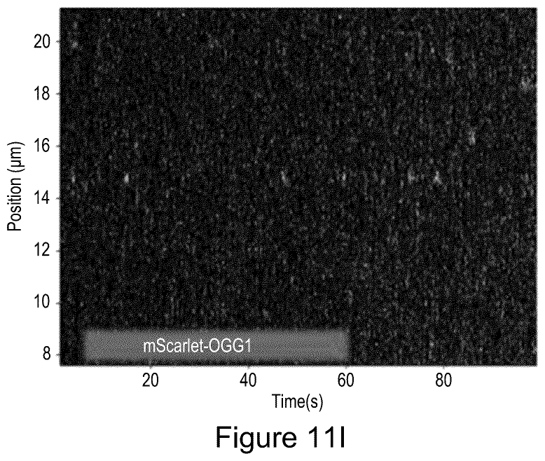

The SMADNE technique was applied to DNA binding proteins having transient interactions, such as XPC-RAD23B which diffuses along the DNA while detecting UV damage, as well as APE1 or Pol β which bind to nicks low affinity (FIG. 3)³⁻⁵. To study XPC-RAD23B, cGFP-tagged XPC and untagged RAD23B were co-transfected, and cGFP signal was observed on UV-damaged (40 J/m²) DNA (FIG. 3A). XPC bound to UV-damaged DNA and diffused along the DNA in 44% of the events observed (FIGS. 3B and 3D). Binding lifetimes for XPC in nuclear extracts were similar to those observed for purified XPC37, with the CRTD fitted to a double exponential to yield one lifetime at 48.6 seconds and a second lifetime at 0.89 seconds, while the fast component contributed to 67% (FIG. 3C). Mean squared dissociation (MSD) analysis performed on the motile XPC molecules (FIG. 3E) revealed a diffusion constant with a geometric mean of ˜ 0.03 μm²s⁻¹, which agreed with previously published work⁴ (FIG. 3F). Additionally, tGFP-tagged APE1 and Pol β binding were also characterized on DNA with 10 nicks as previously done with PARP1. Both proteins bound the nicked substrate with relatively lower affinity, with APE1 exhibiting a binding lifetime of 0.3 s (FIGS. 3G and 3I) and Pol B binding for 1.8 s (FIG. 3J-3L). No binding for these three proteins was observed for undamaged DNA (FIG. 11).

### SMADNE for Observing Protein Dynamics on DNA.

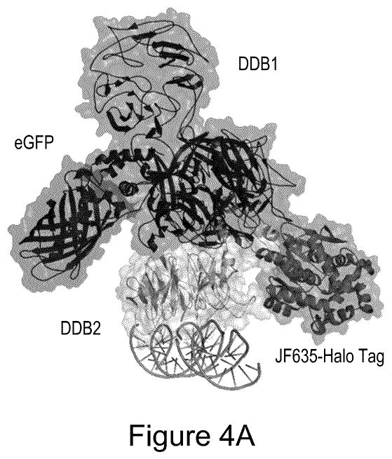

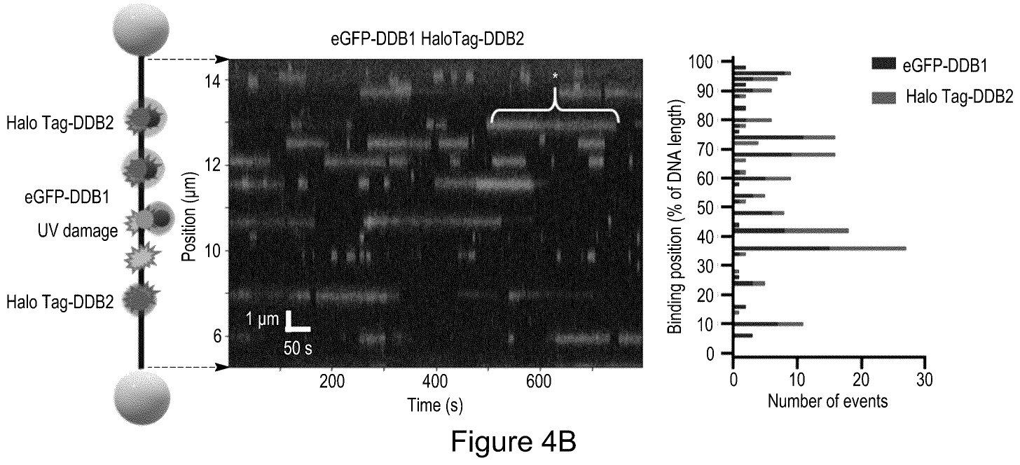

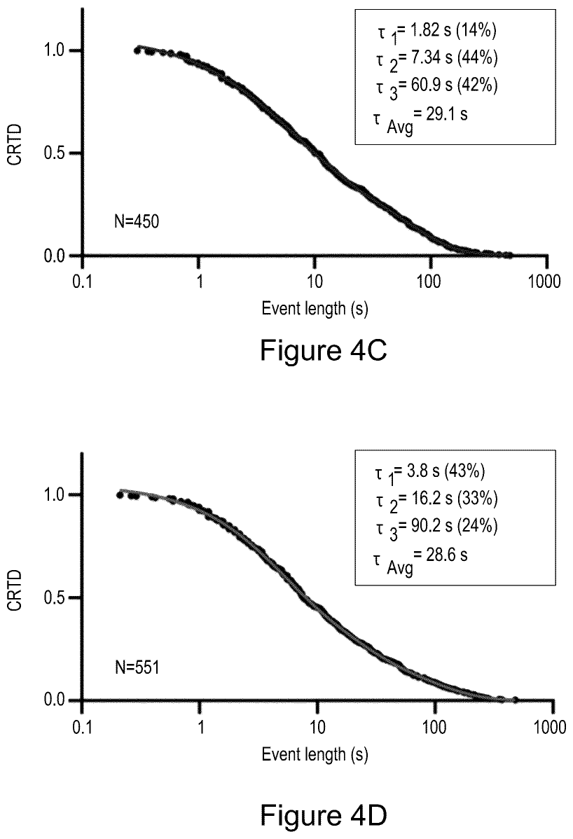

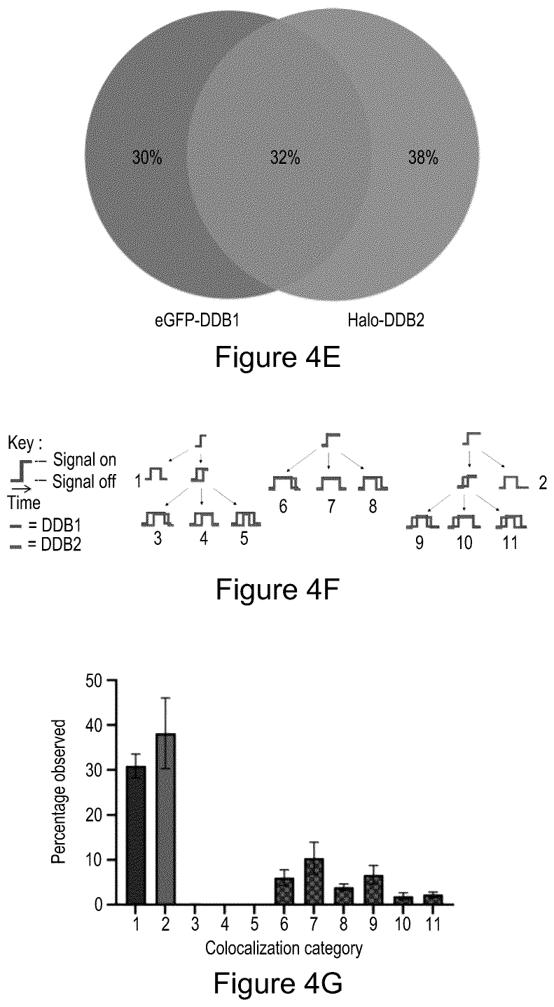

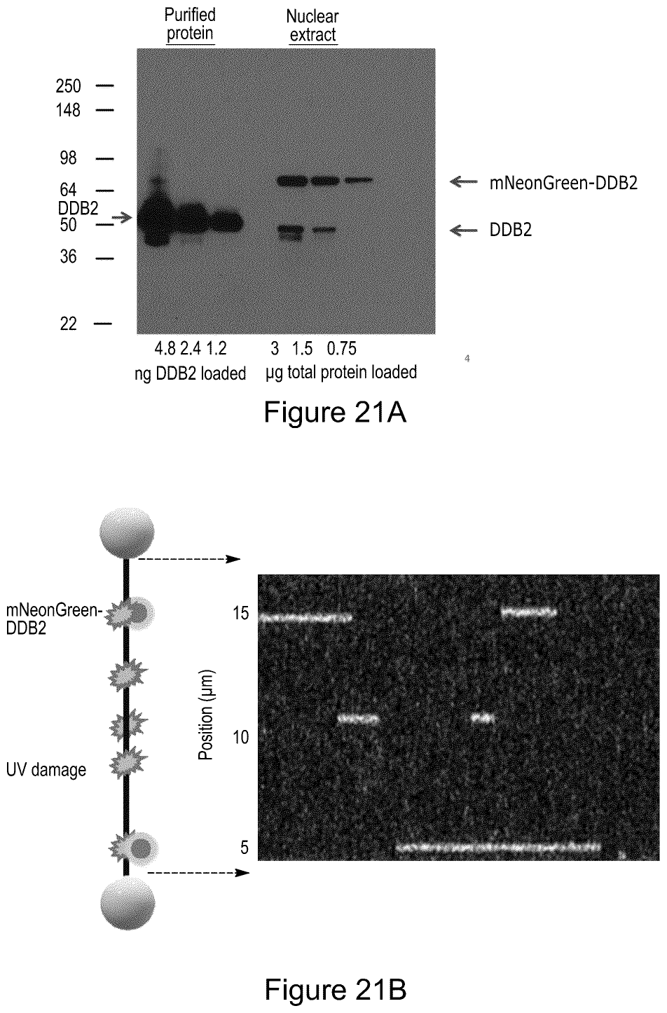

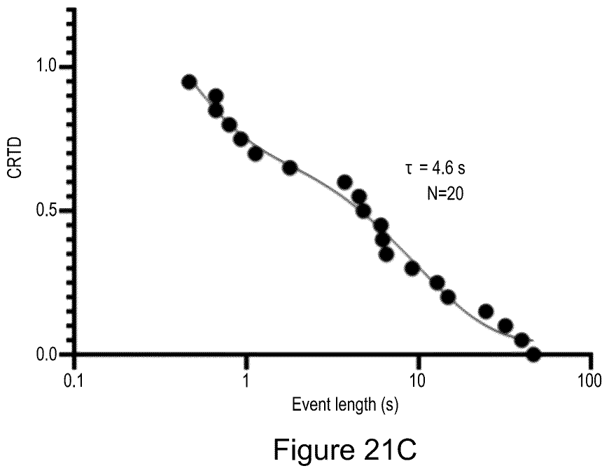

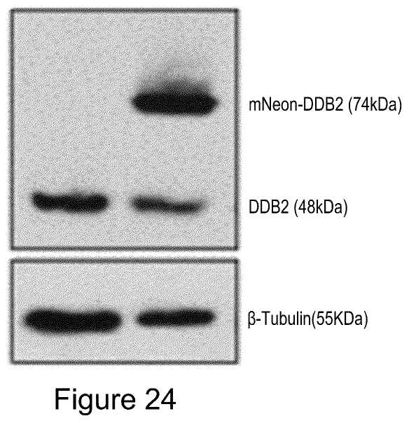

The SMADNE technique was used to study the DNA repair protein UV-DDB, which is composed of a heterodimer consisting of DNA damage-binding protein 1 (DDB1, 127 kDa) and DNA damage-binding protein 2 (DDB2, 48 kDa). The latter subunit engages DNA at the site of damage⁶. UV-DDB detects UV-induced photoproducts with high affinity⁷, and the purified protein has been extensively characterized at the single-molecule level for various DNA substrates^{6,8,9}. Thus, previous studies provided a benchmark to validate the behavior of UV-DDB by SMADNE. UV-DDB was orthogonally labeled, with DDB1 tagged with a N-terminal cGFP tag and DDB2 with an N-terminal HaloTag conjugated to JaneliaFluor 635 dye (FIG. 4A and FIG. 8)¹⁰. The two subunits were co-transfected into U2OS cells and the concentration of UV-DBB protein from nuclear extract was determined in the flow cell at ˜0.3 nM, which was 50-100-fold higher than that of the endogenous by western blot (Table 2). For SMADNE analysis, the transfection can be transient or can be performed with stable cell clones, as shown in FIG. 21, where mNeonGreen-DBB2 was stably transfected into in U2OS cells. U2OS cells stably expressing NeonGreen-DDB2 at about 3-fold higher expression than the endogenous DDB2 (FIG. 24),

The present disclosure confirmed UV-DDB did not exhibit 1D diffusion (sliding) on the DNA but rather found its damaged substrates via 3D diffusion⁸. Furthermore, DDB1 and DDB2 bound to specific positions on the DNA multiple times within a single viewing window (FIG. 4B). These long-lived binding positions (lifetimes >10 s) represented sites of UV photoproducts after UV treatment (40 J/m²). Non-damaged DNA supported significantly fewer and shorter binding events with short dwell-times (<10 s) (FIG. 11). With increasing UV dose, the number of binding events increased with emergence of long-lived UV-DDB complexes (FIG. 11E-11H). Within these damage sites, some positions had many short interactions over the course of a kymograph (consistent with a low-affinity substrate being weakly bound and released multiple times) and some positions only had a few long interactions (consistent with a high-affinity substrate strongly bound by UV-DDB). This pattern reflected binding to cyclobutane pyrimidine dimers and 6-4 photoproducts, respectively, both of which are products of UV irradiation¹¹.

The binding events of both DDB1 and DDB2 exhibited a wide distribution of binding durations (four orders of magnitude) in good agreement with studies performed on purified UV-DDB (FIGS. 4C and 4D). Binding event durations were fitted to CRTD to quantify the rate of dissociation (k_{off})⁶. The DDB1 and DDB2 plots were fitted to a triple-exponential decay function as was previously reported for purified UV-DDB, with one short lifetime (˜2 and 4 s respectively), one medium lifetime (7 and 16 s respectively), and one long lifetime (61 and 90 s respectively) (FIGS. 4C and 4D). The weighted average lifetime (all three lifetimes multiplied by their percentage contribution) for DDB1 was 29.1 s, relatively close to DDB2 at 28.6 s. These weighted average lifetimes were around 50% longer than the previous observations with purified UV-DDB on UV-damaged DNA (weighted average of 18.5 seconds)⁶. As the previous strategy relied on Qdot-conjugated UV-DDB, the previously reported shorter lifetime observed could be due to Qdot conjugation process causing a modest reduction in UV-DDB binding affinity and thus a decreased lifetime as compared to presently disclosed new fusion protein approach.

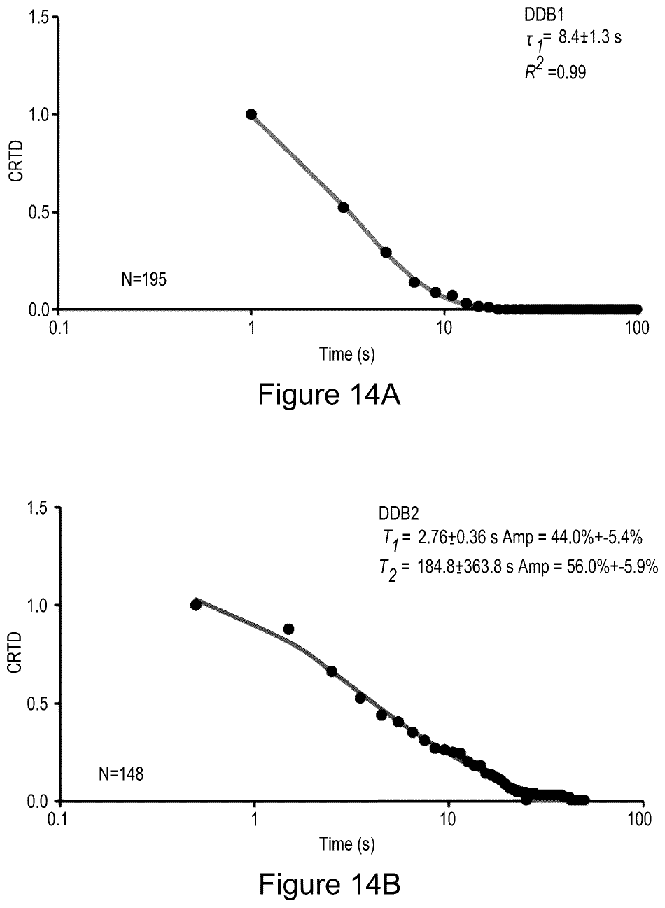

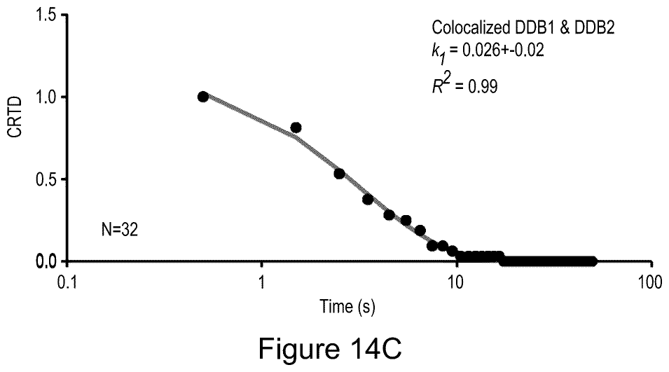

Alternatively, unlabeled interacting proteins in the nuclear extract, such as heat shock proteins (FIG. 9), could provide stability to UV-DDB12. The two-color results were also validated using a C-Trap instrument with total internal reflection fluorescence capabilities, and similar trends of colocalization and binding lifetimes were observed (FIG. 14).

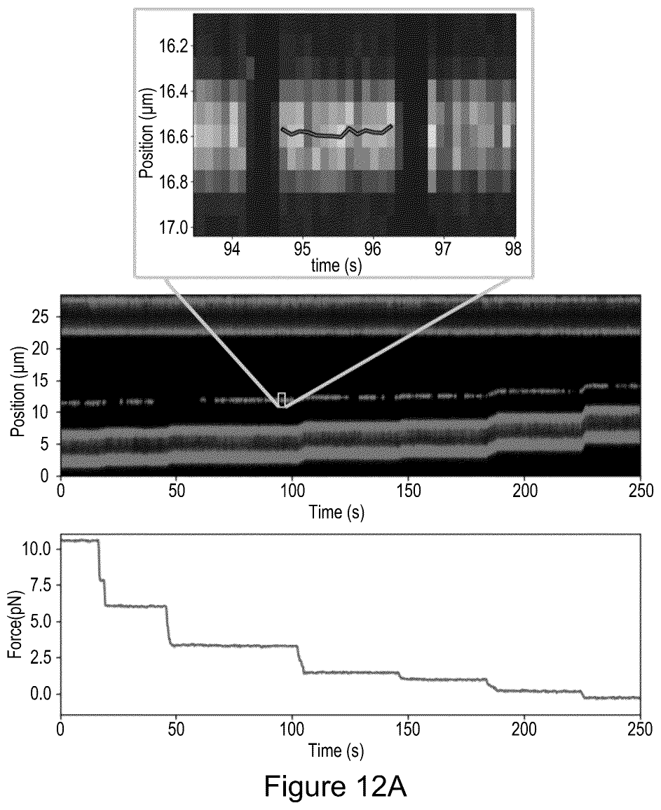

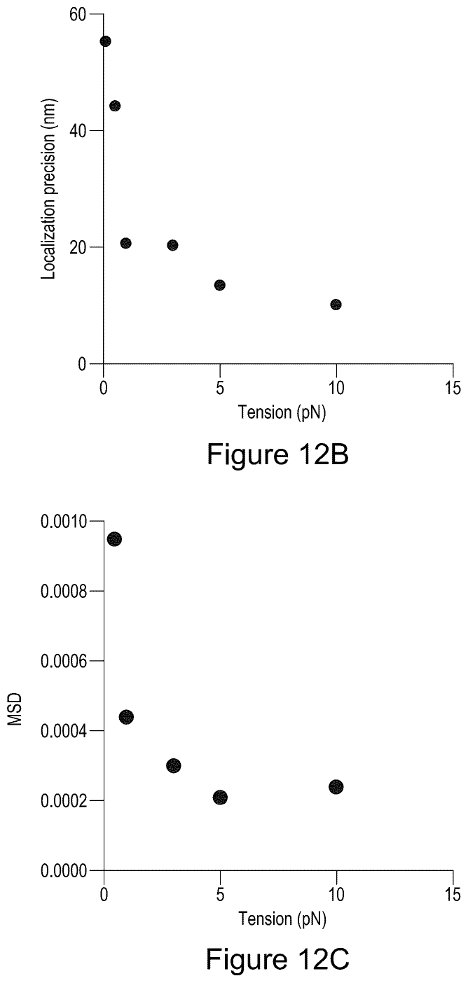

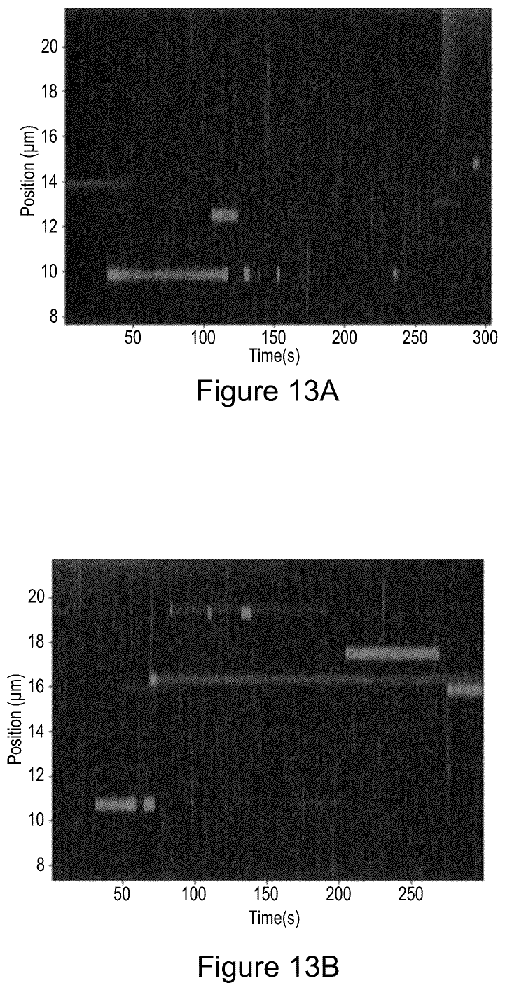

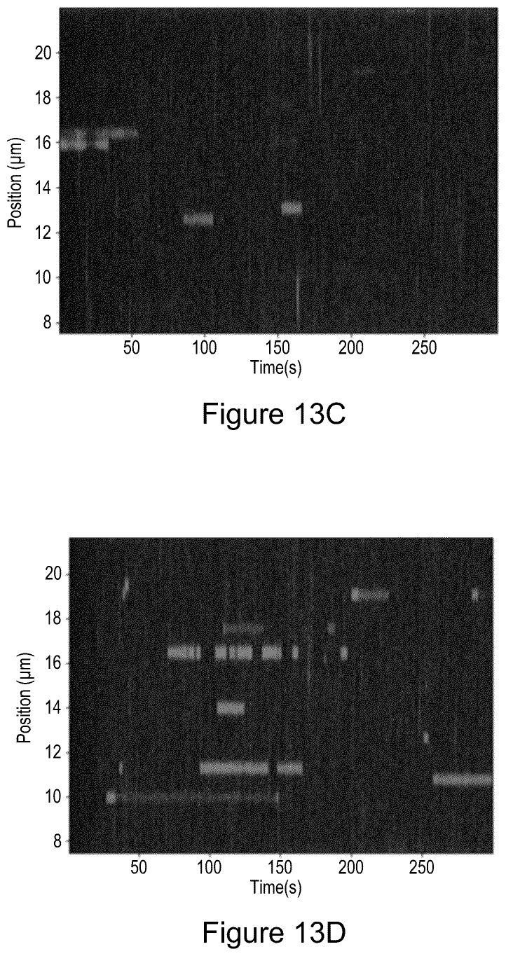

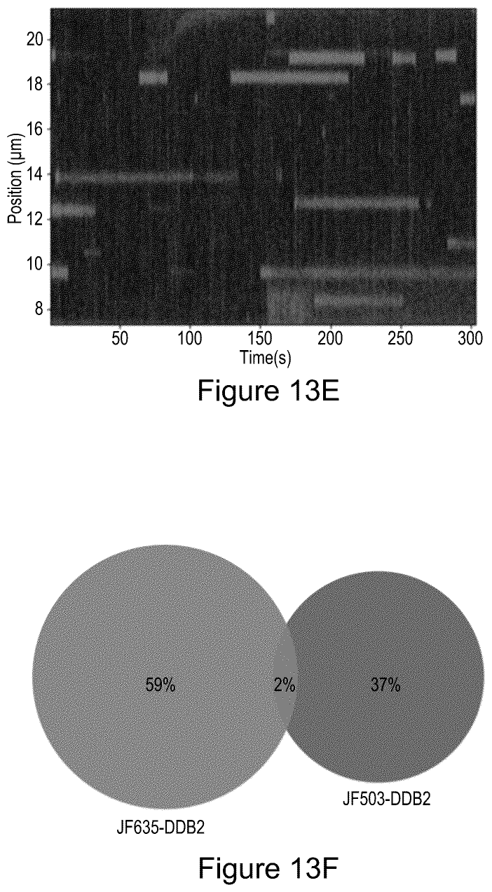

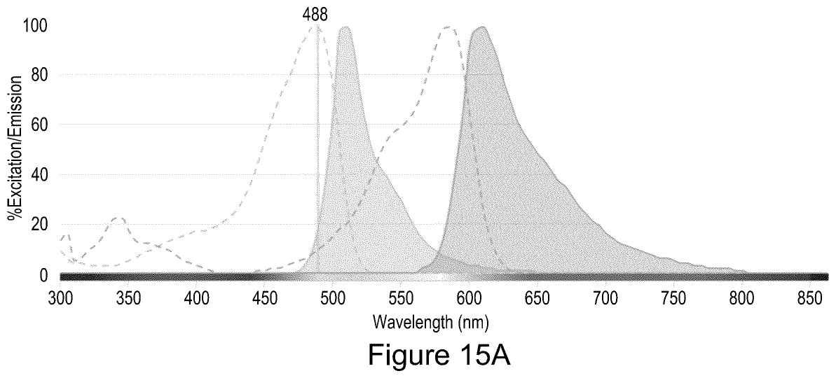

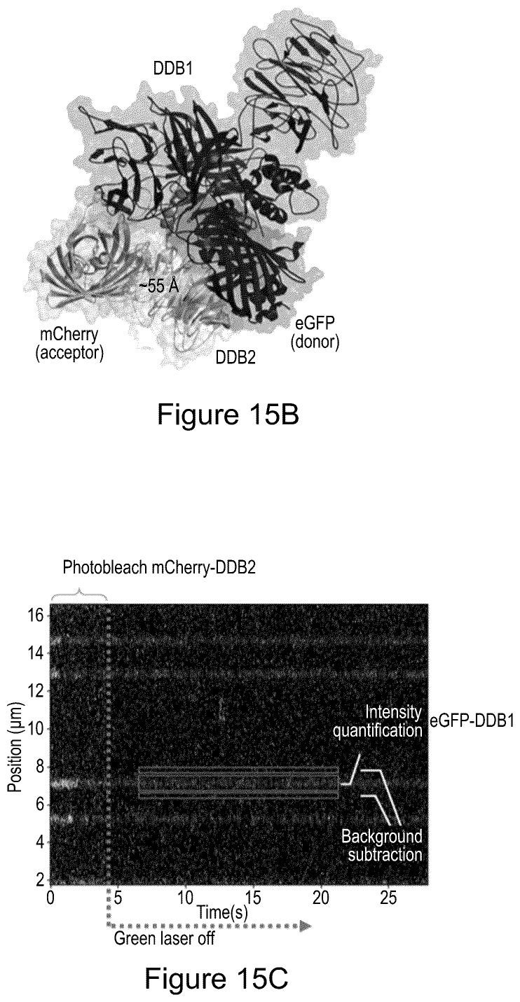

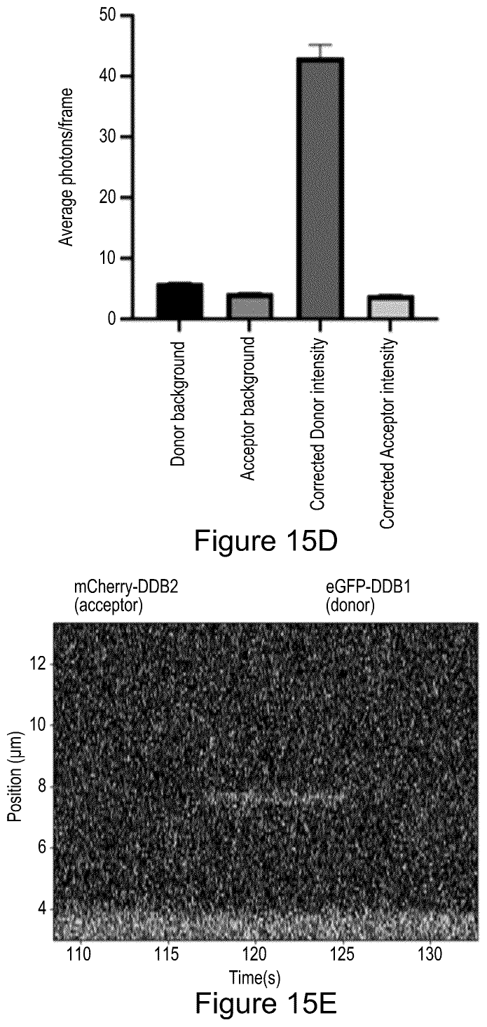

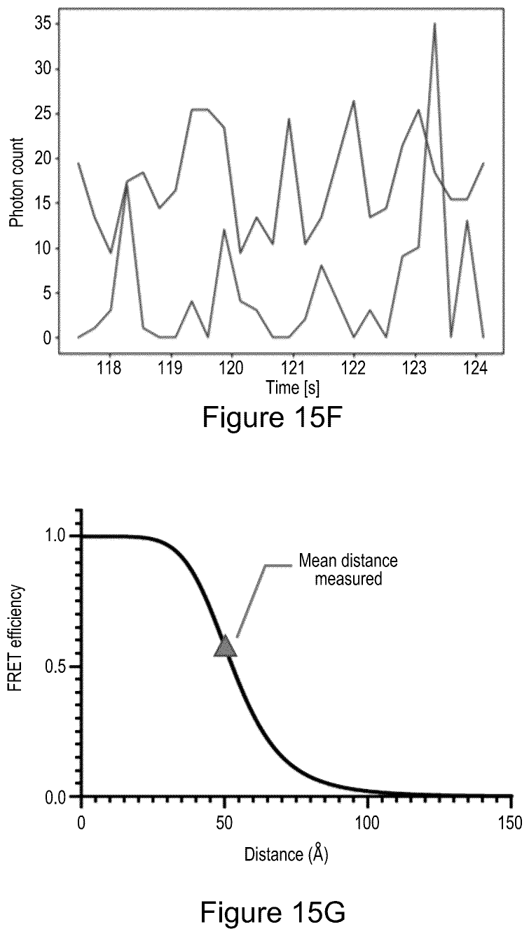

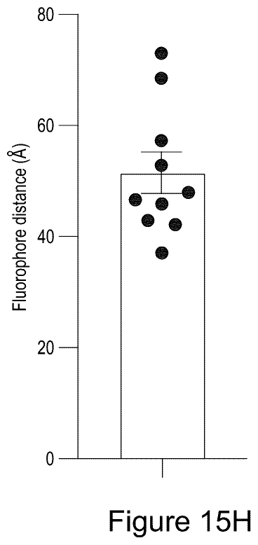

The presently disclosed dual-label approach allowed for the frequency of DDB1 and DDB2 co-localization within the localization precision of the instrument (˜150 bp with these fluorophores; FIG. 12), to be quantified. Consistent with UV-DDB acting as a stable heterodimer, many colocalize events were observed-32% of events had at least one colocalization with the second color, compared to 30% of events that were either one molecule of eGFP-DDB1 or 38% that were HaloTag-DDB2 (FIG. 4E). Colocalized binding events were confirmed to be from one heterodimer of UV-DDB rather than a dimer of heterodimers or two heterodimers bound closely together⁶, by examining a mix of two colors of HaloTag-DDB2 (JF503 and JF-635) which rarely colocalized (˜2%; FIG. 13). To further probe the structure of the colocalization events, a mCherry-DDB2 construct was utilized to act as the acceptor in a single-molecule Förster resonance energy (sm-FRET) approach. Clear FRET signal was observed for multiple events, confirming a direct interaction between the two subunits (FIG. 15).

SMADNE also allowed for the dynamics of multiprotein interactions on DNA to be analyzed. The present disclosure identified 11 possible event classes of molecular interactions on DNA (FIG. 4F), including single-color events (without colocalization). Nine event classes represented colocalization events with unique assembly and disassembly mechanisms. A script was developed to classify the 11 different types of events (publicly available on LUMICKS Harbor) and found that the most common event type was a category 7, in which DDB1 and DDB2 arrived and dissociated together.

Results are consistent with UV-DDB acting as a stable heterodimer. However, the next most common event was a category 9, where DDB2 bound first followed by DDB1 and then DDB1 dissociates before DDB2, suggesting that alternative modes of binding exist where the proteins sequentially assemble and disassemble from the damage. Of note, categories 3-5 appeared exceedingly rare (FIG. 4G).

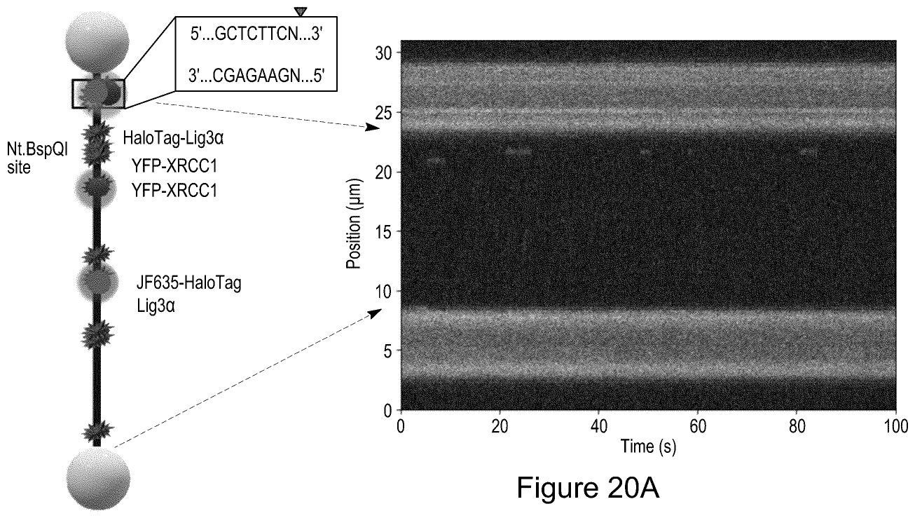

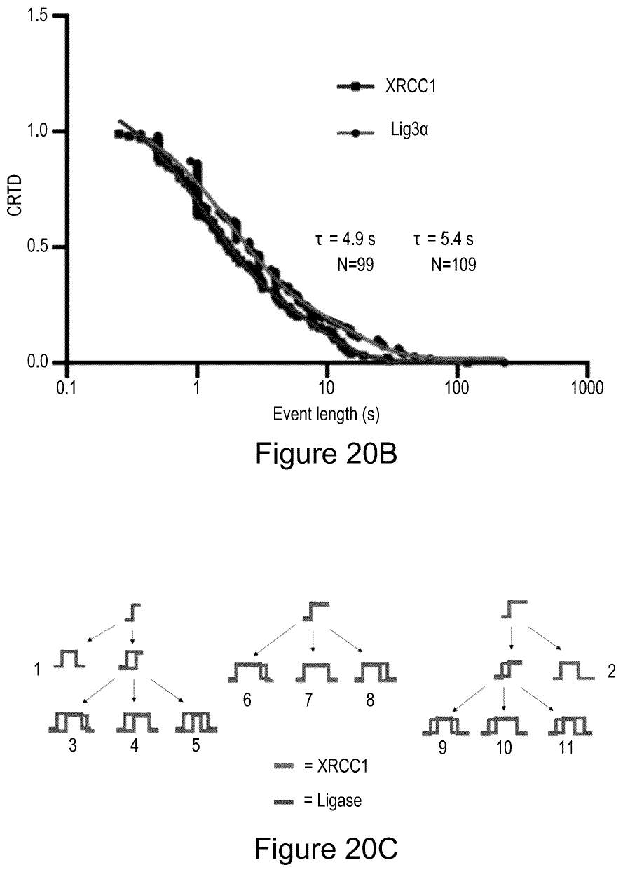

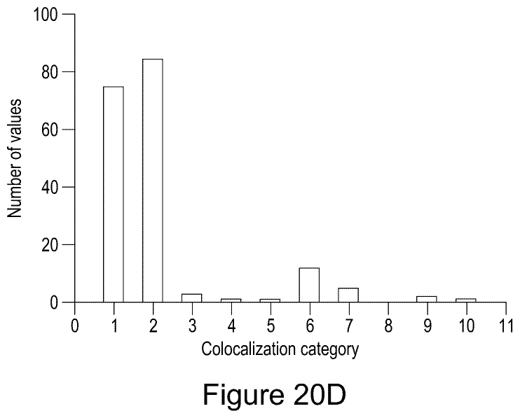

The present disclosure further demonstrated the multiprotein interaction approach with DNA repair proteins XRCC1 and Lig3α. As demonstrated in FIGS. 20A-20D, YFP-XRCC1 and Halotag-Lig3α most often colocalized when bound together, followed by the dissociation of XRCC1 first from the DNA substrate.

### Effects of Unlabeled Protein on Fluorescently Tagged Protein Behavior.

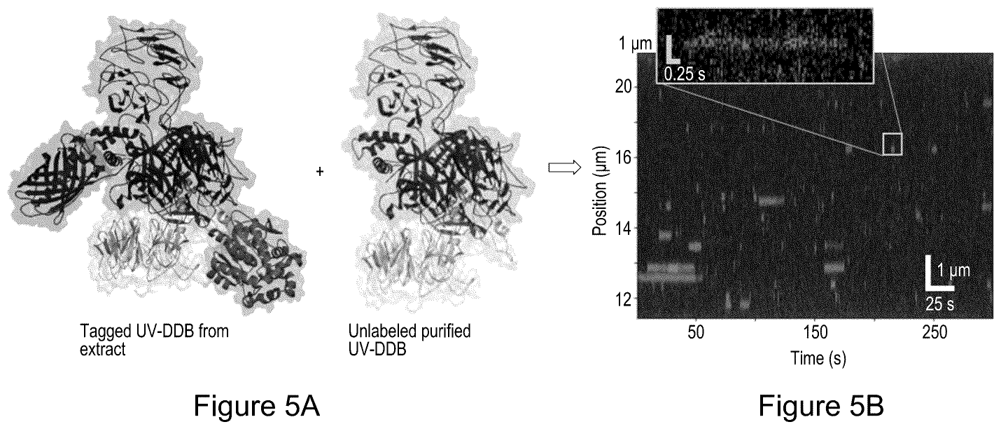

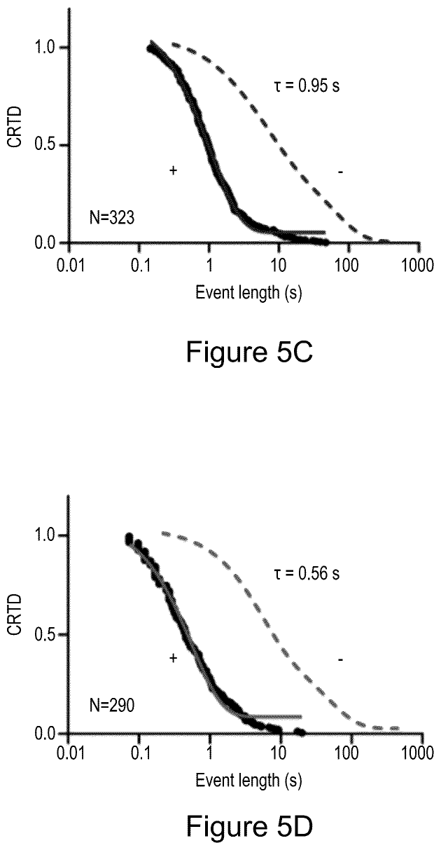

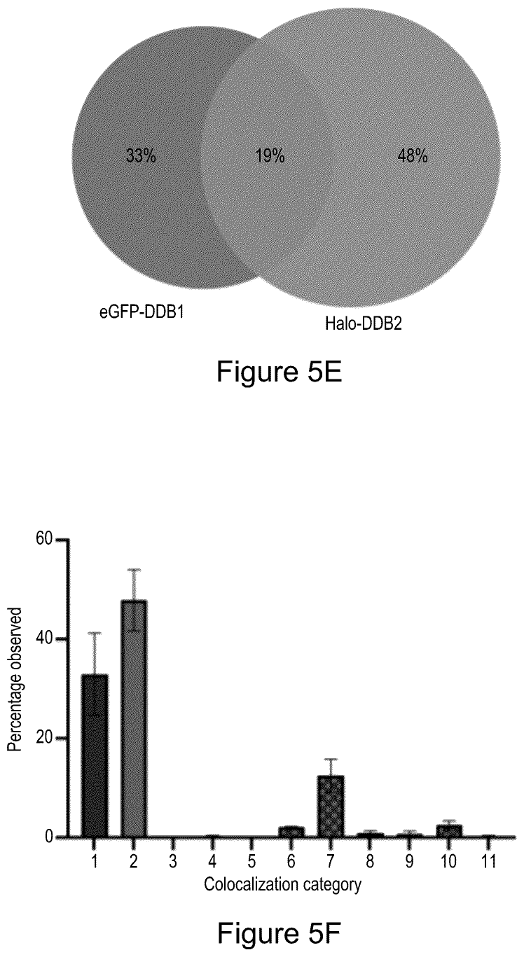

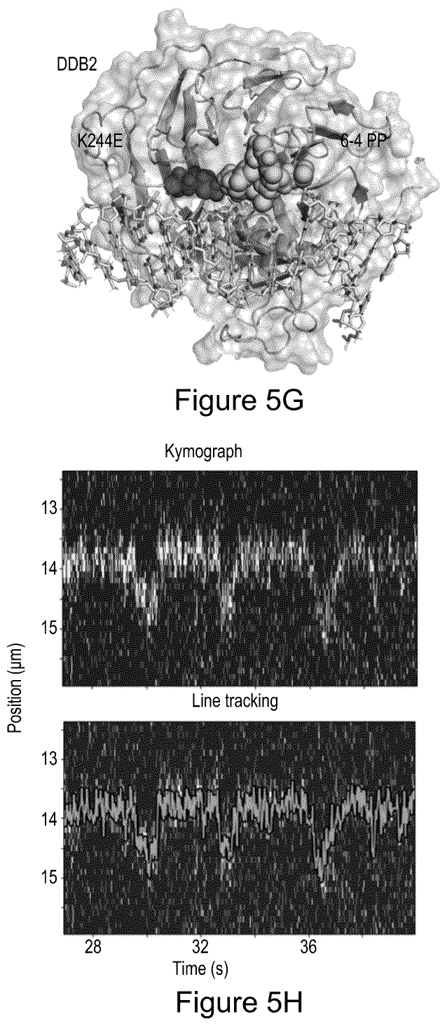

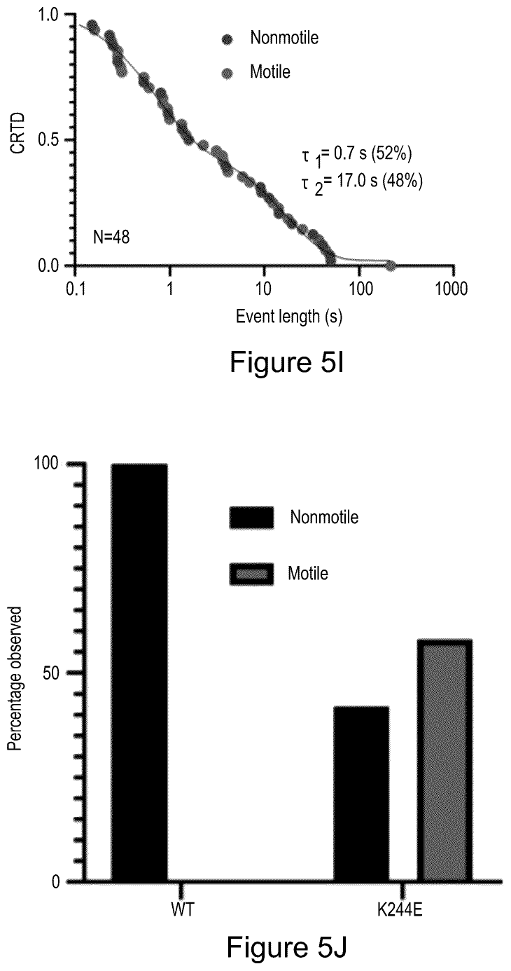

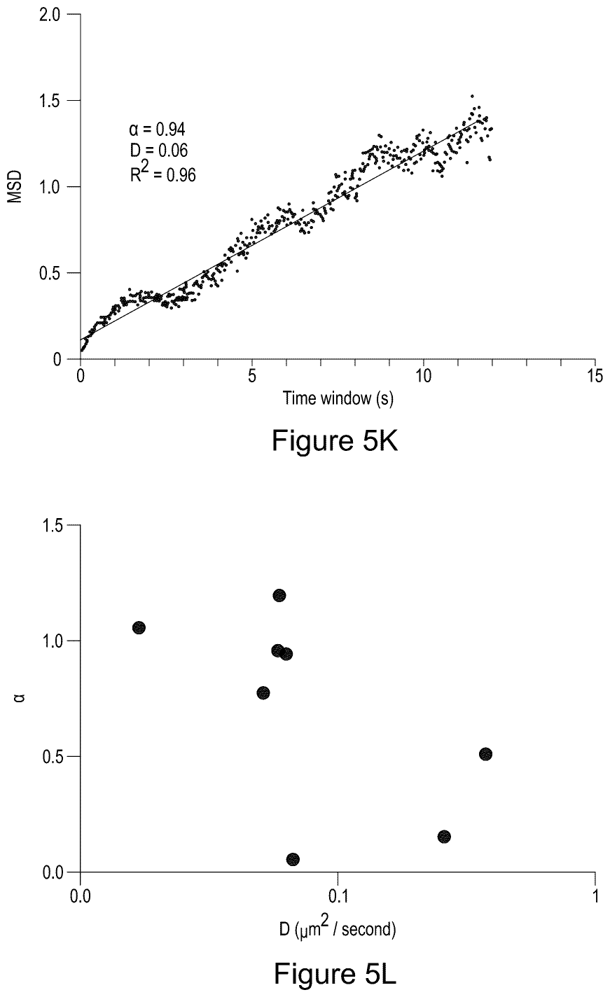

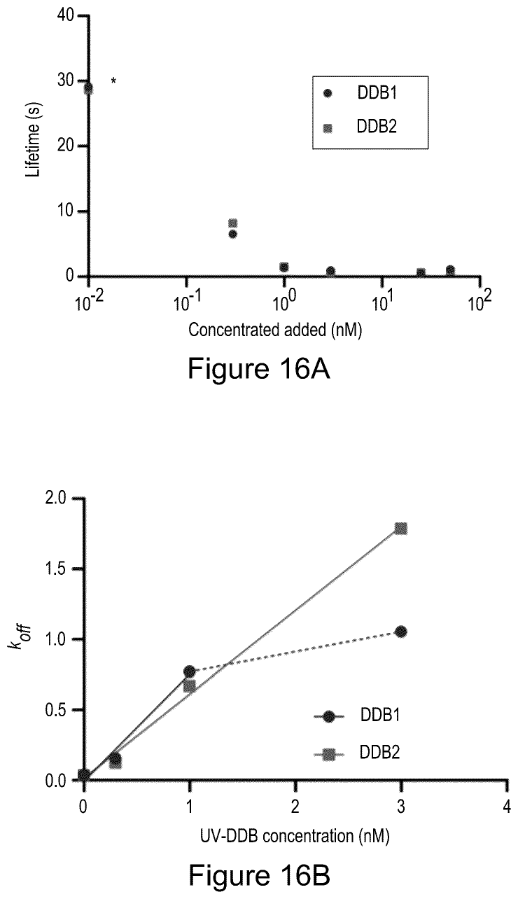

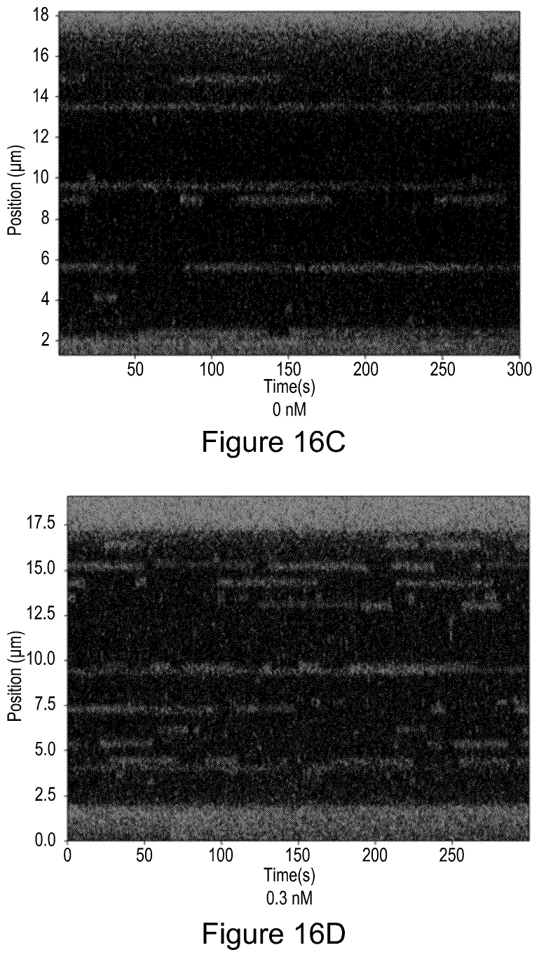

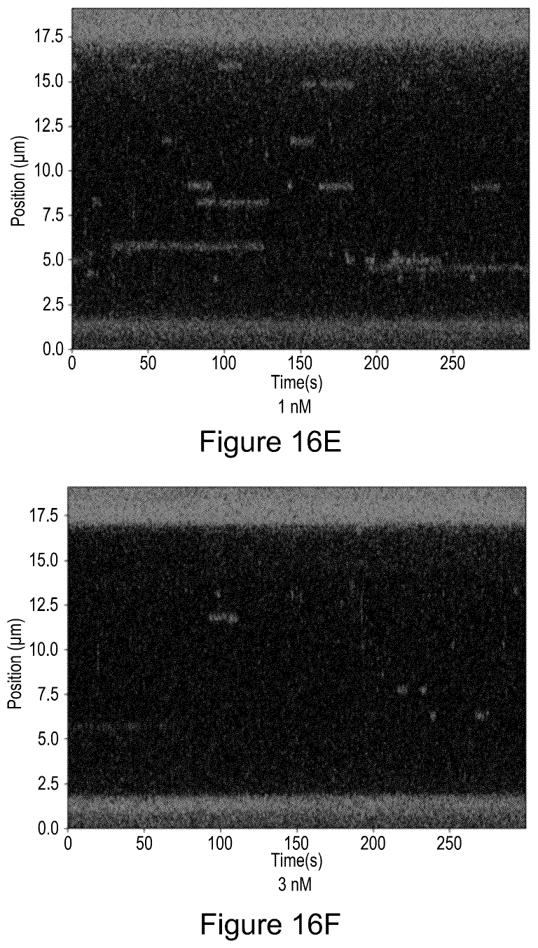

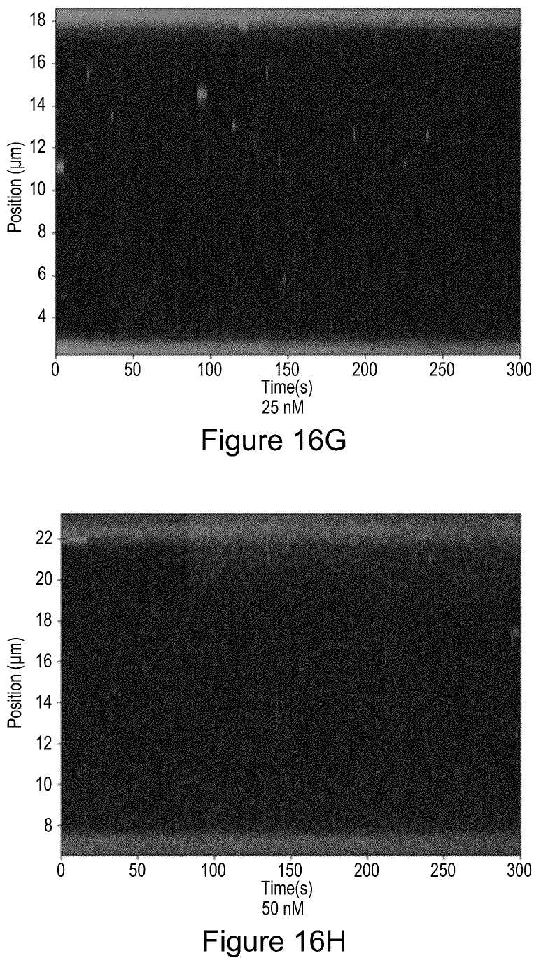

Although k_{off} values and thus binding lifetimes are traditionally thought to be concentration independent, a growing body of work has shown that the presence of competitor proteins can alter binding lifetimes¹³⁻¹⁵. This phenomenon would alter binding results observed by SMADNE if the endogenous unlabeled protein represented a significant fraction compared to the labeled protein of interest. To examine facilitated dissociation of the target labeled protein by the endogenous non-labeled protein, tenfold excess concentration of purified UV-DDB (3 nM) was included along with the cGFP-DDB1 and HaloTag-DDB2 tagged proteins in extracts (FIG. 5A). While a similar number of events were observed, the event lifetime was drastically reduced by ˜30-fold for DDB1 and ˜40-fold for DDB2 in the presence of purified protein (FIGS. 5B-5D). Additionally, various concentrations of unlabeled UV-DDB were added and a concentration-dependent response in binding lifetime was observed (FIG. 16). Interestingly, a decrease in colocalization frequency from 32% to 19% was observed, which suggested that the subunits from purified UV-DDB may exchange in solution; however, category 7 (binding together and dissociating together) was again the most common category (FIGS. 5E and 5F).

### SMADNE Allowed Rapid Characterization of DDB2 Variant (K244E).

SMADNE provided a rapid approach to determine the effects of naturally occurring mutations on function, without having to purify the protein and reduce yield and activity. SMADNE was used to study the K244E variant of DDB2, which is associated with the human syndrome xeroderma pigmentosum complementation group E (FIG. 5G). Previous single-molecule characterization of K244E variant demonstrated the substitution causes UV-DDB to lose specificity for damage sites by diffusing past UV-induced photoproducts⁶. Indeed, the mNeon-DDB2 K244E variant exhibited increased motility and decreased binding lifetimes (FIG. 5H), with 58% of the events observed exhibiting a detectable motion in contrast to 0% with WT DDB2 (FIGS. 51 and 5J). MSD analysis of the motile binding events indicated mNeonGreen-DDB2 K2444E behaved similarly to previously reported studies using a Qdot labeled variant (FIGS. 5K and 5L). The slower diffusivity observed with purified proteins is because the Qdot label increases the drag considerably compared to the smaller fusion tag in the SMADNE approach. In addition to the motion along the DNA, shorter binding lifetimes were observed with the mutant compared to the characterization of WT DDB2, with the slowest off rate disappearing and the data was best fitted to a double exponential instead. The average lifetime for DDB2 K244E was 8.5 s, which agreed with the hypothesis that the mutation prevents full engagement with the DNA (FIG. 5L).

Visualizing Oxidative Damage Repair Dynamics with SMADNE.

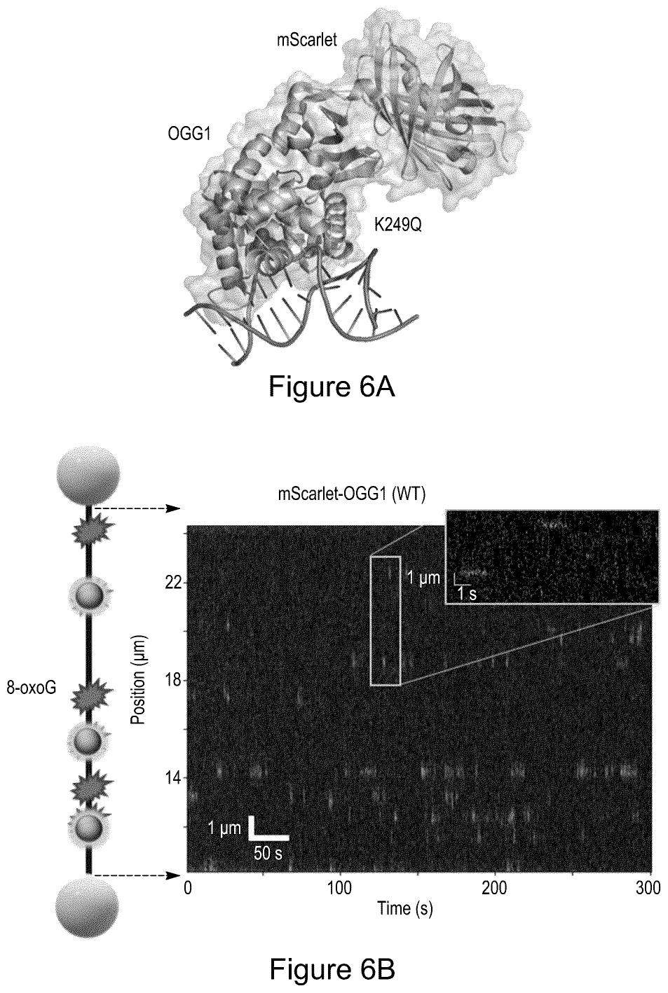

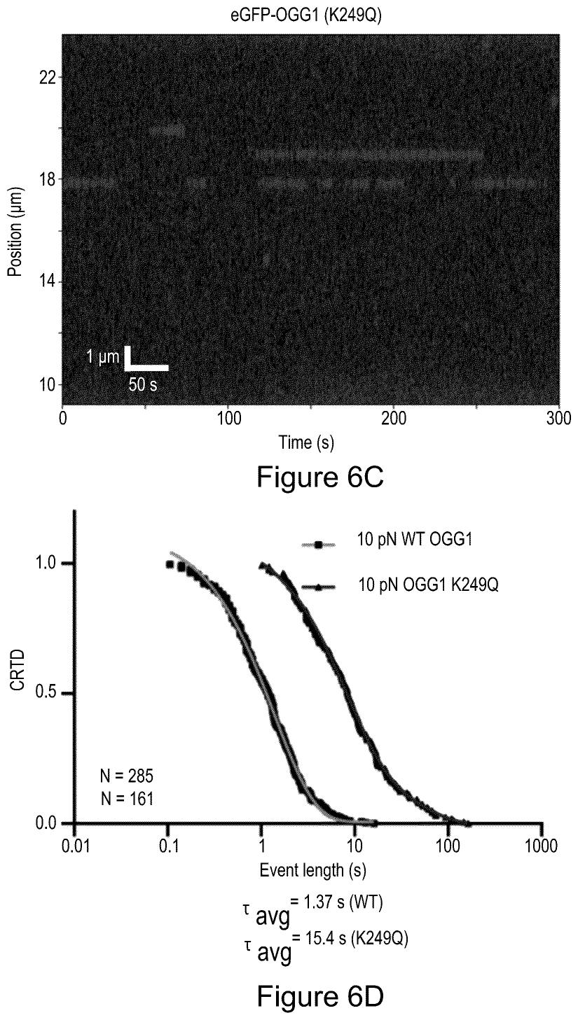

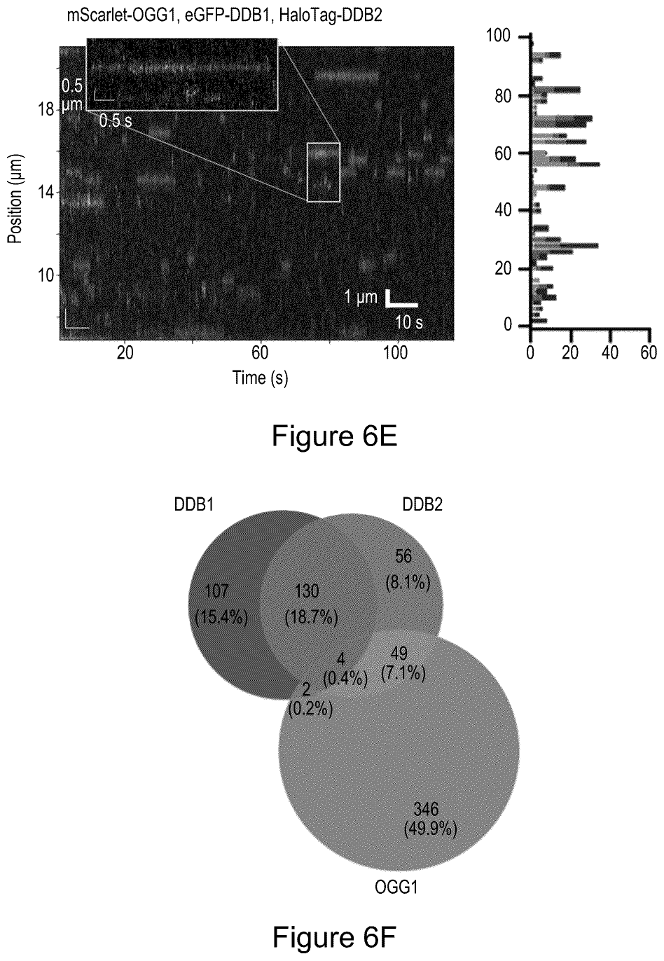

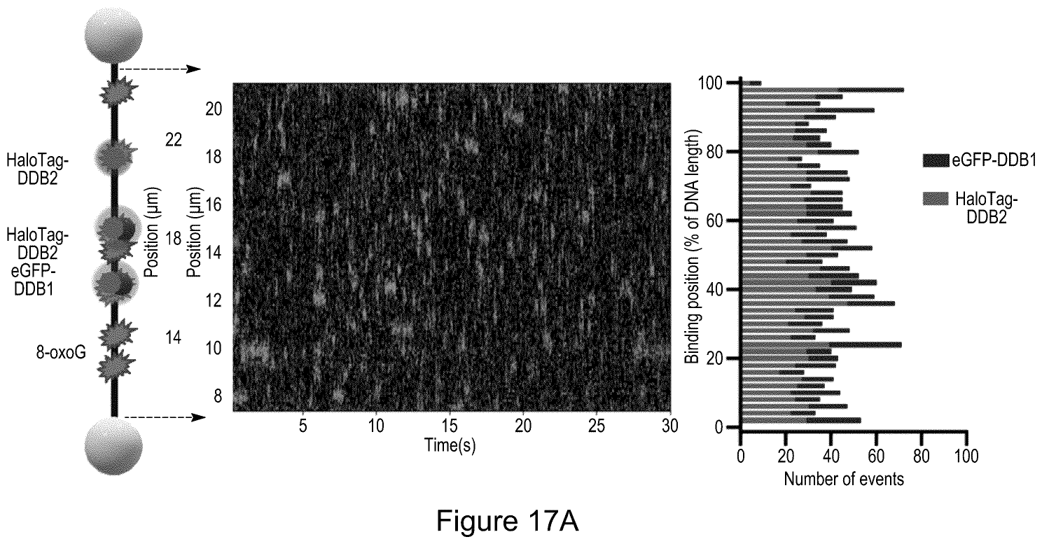

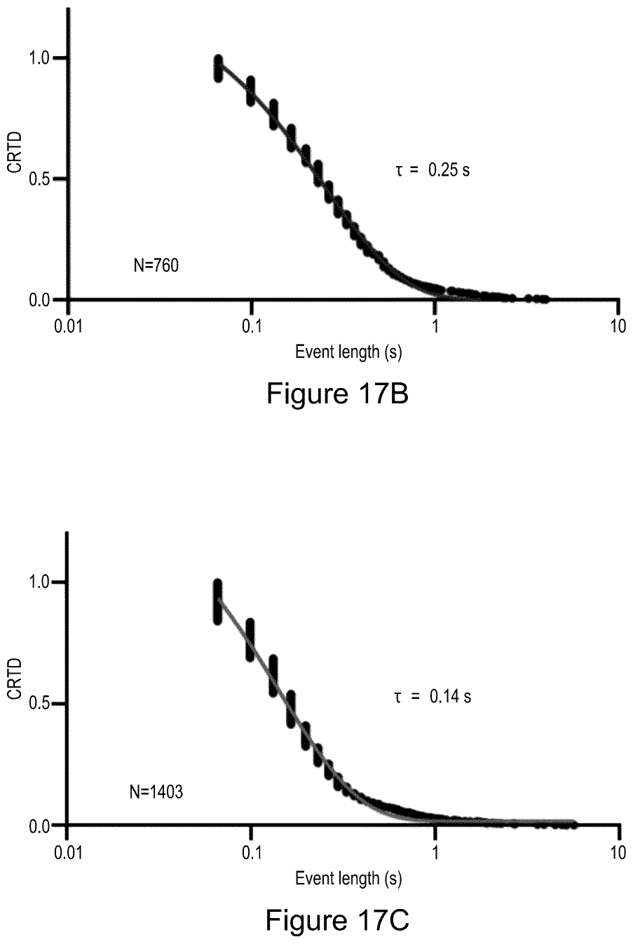

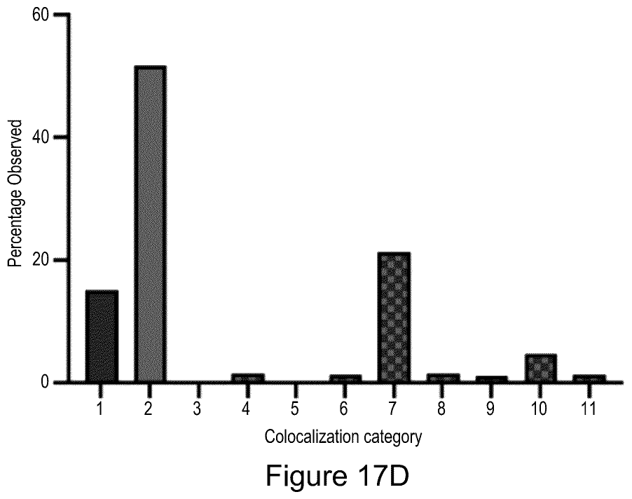

Single molecule and cellular studies demonstrated that UV-DDB interacts with OGG1 to process 8-oxoG lesions⁹. To this end, nuclear extracts from mScarlet-OGG1 expressing cells (FIG. 6A) were used to study OGG1 binding to DNA treated with oxidative damage (one 8-oxoG/440 bp)¹⁶. OGG1 bound to numerous positions along the length of the DNA, with many positions bound multiple times (presumably the sites of oxidative damage, FIG. 6B). Each bound position of OGG1 exhibited similar binding lifetimes: a CRTD plot revealed a best fit to a double-exponential function with a weighted average lifetime of 1.37 s (FIG. 6D). Also observed were short lifetimes of OGG1 bound to non-damaged DNA, although the frequency of binding was significantly less (FIG. 11E). These lifetimes agree with the ˜ 2 s lifetimes published by Wallace and coworkers for purified E. coli Fpg¹⁶, and Verdine and colleagues for OGG1 on non-damaged DNA¹⁷. The present disclosure tested the binding characteristics of a catalytically dead OGG1 variant containing a mutation in its active site, K249Q (FIG. 6C)¹⁸. The binding kinetics of eGFP-labeled OGG1 K249Q on a DNA substrate containing 8-oxoG revealed much longer binding lifetimes compared to WT OGG1 (binding lifetimes of 6.2 and 36 s, with the fast lifetime contributing 75%; FIG. 6D). It was previously found that UV-DDB interacts with OGG1 to process 8-oxoG lesions⁹, thus, the present disclosure sought to determine whether these interactions could be observed in nuclear extracts using SMADNE. To this end, mScarlet-OGG1, cGFP-DDB1, and HaloTag-JF635-DDB2 were recombinantly expressed and the interactions between all three proteins was observed (FIGS. 6F and 6G). UV-DDB bound to DNA with oxidative damage robustly, but the binding lifetimes of DDB2 (0.14 s) were reduced compared to their lifetime on UV damage, in agreement with its lower affinity to 8-oxoG compared to UV damage (FIG. 17)⁹. Furthermore, a moderate degree of transient colocalization between DDB2 and OGG1 was observed, but the majority of binding events were either OGG1 alone or DDB1 and DDB2 together at 49.9% and 15.4%, respectively (FIG. 6G).

Incorporating Base Analogues into the DNA Substrate.

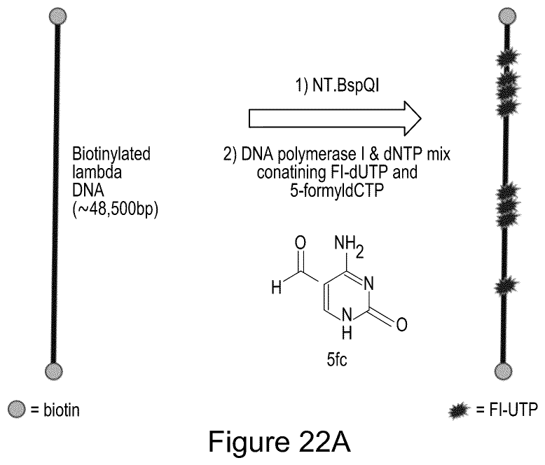

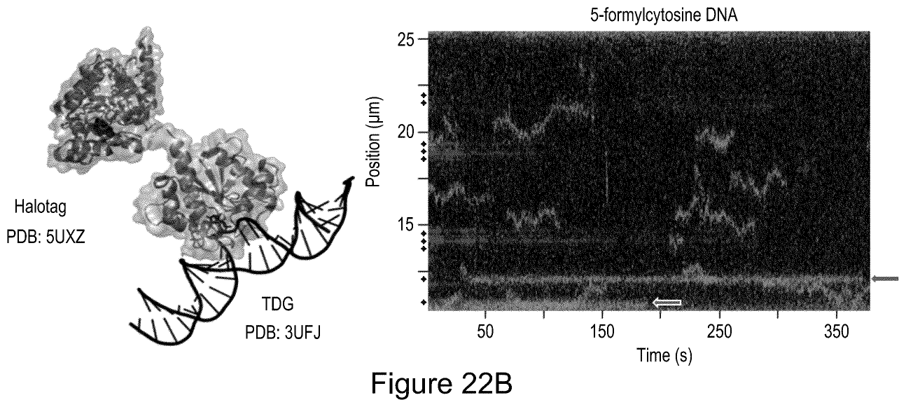

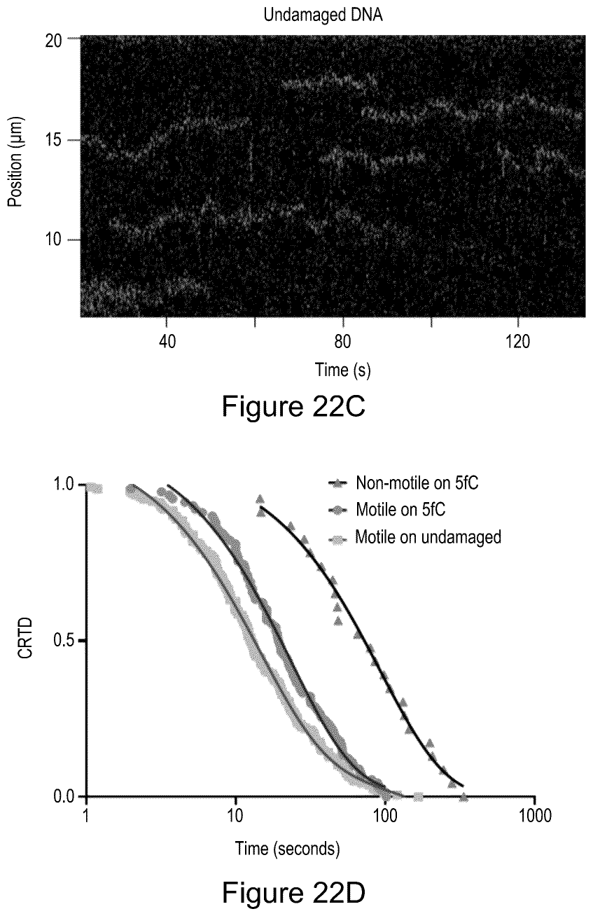

The present disclosure demonstrated the incorporation of base analogues into the DNA substrate during nick-translation mediated by DNA Polymerase I. As shown in FIGS. 22A-22D, the incorporated 5-formyl-cytosine (5fC) nucleotide analogues served as both a fiducial fluorescent marker and indicator of damaged DNA. The present disclosure shows TDG-HaloTag-JF635 bound to DNA after nick translation to incorporate the analogues, and to undamaged DNA (FIGS. 22B-22C).

### Following the Kinetics of AAG Interaction on Hypoxanthine Moieties

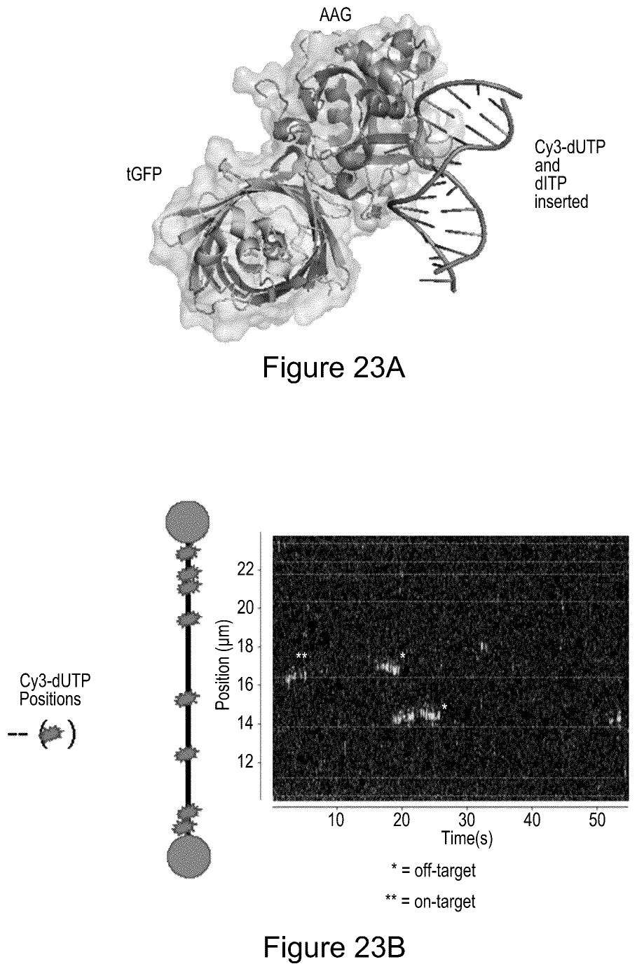

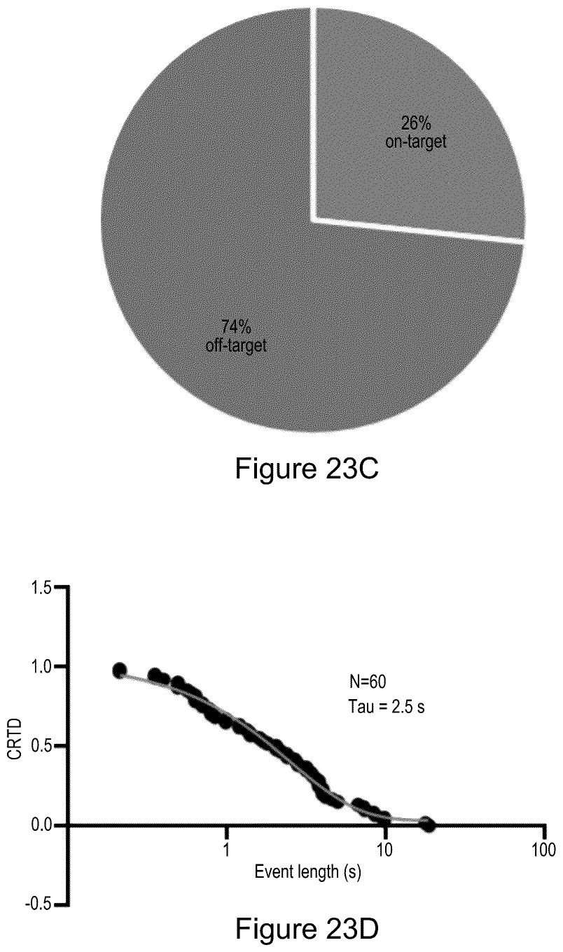

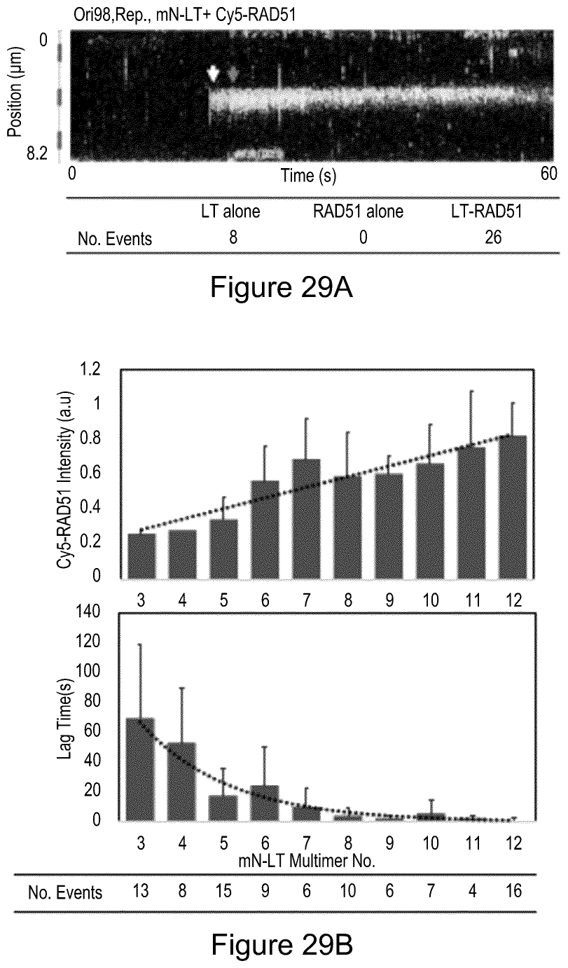

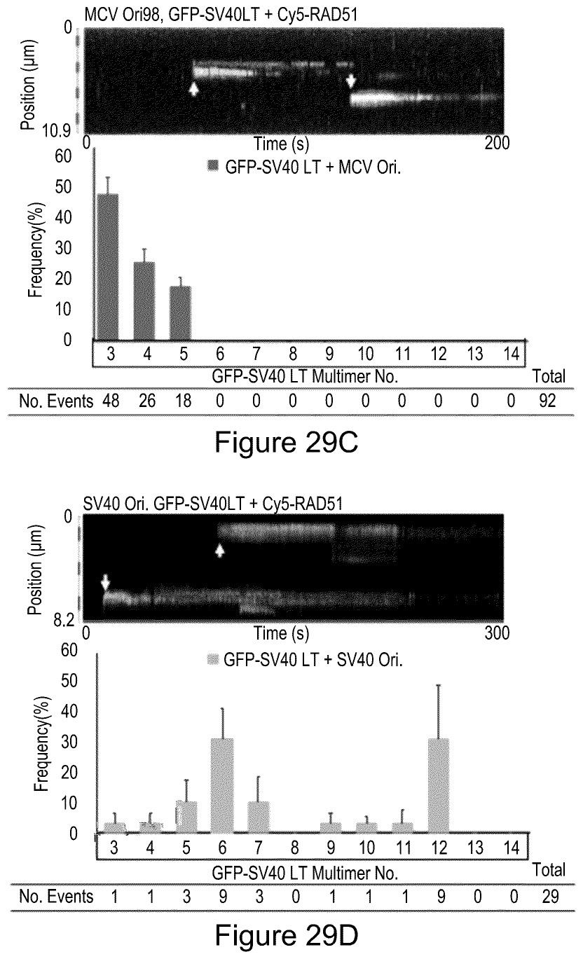

The present disclosure further investigated damage detection by AAG to substrates with hypoxanthine substrates. Current methods do not easily allow the analysis of transient (seconds) protein interactions with DNA, nor allow the positions of the abasic sites to be precisely known. Therefore, SMADNE followed AAG interacting with hypoxanthine moieties in lambda DNA. First, to create hypoxanthine sites within lambda DNA, dITP was incorporated at 10 nick sites created by the nickase Nt.BspQI via nick translation with Pol I. Cy3-labeled dUTP was also incorporated at the same time to provide fluorescent fiducial markers for the positions of hypoxanthine moieties. Cells transfected with a plasmid expressing GFP-tagged AAG (FIG. 23)²⁸. The fluorescent fiducial marker and hypoxanthine positions were measured by briefly toggling a 562 nm laser on and off, and events with GFP-AAG were collected by exciting with a 488 nm laser. Cumulative residence time distribution analysis of all events observed revealed a binding lifetime cumulative residence time distribution of all GFP-AAG events, fitting to a single-exponential with a lifetime of 2.5 (FIG. 23D). Of the binding events observed, a majority of them were brief sampling events that occurred on sites without the DNA damage (77%) but 23% of events did colocalize with the damage sites (FIG. 29C). The present disclosure showed that nick translation allowed for the incorporation of both Cy3-dUTP and dITP (inosine triphosphate). As shown in FIGS. 23A-23D, the incorporation of nucleotides into the DNA substrate allowed for characterization of GFP-AAG binding to on-target events, i.e., nicked labeled DNA sites, and off-target events.

### 6.1.2 Discussion

SMADNE offers several major advantages compared to traditional single-molecule studies in living cells or with purified proteins. First, nuclear extracts used in SMADNE rapidly generate similar mechanistic information in agreement with previous work using purified proteins (including binding lifetimes and other outcomes shown in FIG. 1). Second, since SMADNE utilized common fluorescence tags such as eGFP, nuclear extracts could be rapidly prepared from transfection of commercially available overexpression plasmids, including both transient and stable transfection (FIG. 21). Third, orthologous labeling allowed co-localization studies to be performed on heterodimeric complexes and interacting proteins. Fourth, SMADNE enables a wide range of interaction affinities to be studied, even transient interactions with K_{D} values of ˜1 μM. Because the k_{off} correlates with binding lifetime, a K_{D} value of ˜1 μM appears to be the limit of detection using SMADNE-binding events weaker than this would have a lifetime of <0.1 s and be challenging to detect. In all, the work on the UV-DDB and OGG1 variants indicated that SMADNE will provide mechanistic insights for proteins of interest via site-directed mutagenesis of specific residues.

Other methods exist that have been used to characterize proteins, RNA, and DNA at the single-molecule scale from extracts. These include Comparative Colocalization Single-Molecule Spectroscopy (CoSMoS) to study RNA-protein interactions out of yeast extracts 19.20, Xenopus laevis egg extracts to study DNA replication and repair²¹⁻²³ and single-molecule pulldown (SiMPull) to analyze protein complex stoichiometry and binding parameters from pulled-down proteins, among other techniques²⁴⁻²⁶. These single-molecule methods all represent major advances in bridging the gap between cellular and single-molecule studies by studying cell extracts at the single-molecule level. SMADNE for the first time, used human nuclear extracts to visualize protein binding on DNA strands in relation to defined genomic position and generated invaluable mechanistic information under the most physiological conditions possible. In this way post-translational modification of desired proteins after specific signaling events (e.g., DNA damage responses) can be monitored. Furthermore, performing SMADNE on the LUMICKS C-trap overcomes a disadvantage to single molecule approaches requiring TIRF microscopy that utilize DNA tethered to the bottom of the flow cells: nuclear debris can also stick to the bottom of flow chambers and obscure/overpower the fluorescence of single molecules. In contrast, with SMADNE the DNA strand remains in the center of the flow cell, circumventing debris accumulation in its focal plane. Also, the optical traps can additionally be used to keep the imaging zone clear from nuclear debris. SMADNE stands to lower the barrier of entry for research groups to understand DNA-binding proteins of interest at the single-molecule level without the burden of protein purification. While the applications shown in the present disclosure focused on DNA repair proteins, the method disclosed herein is applicable to many other types of DNA-binding proteins, including transcription factors, helicases, and DNA polymerases. Table 5 lists various proteins and variants that have been analyzed using the SMADNE approach. Furthermore, this new approach could be used to observe macromolecular interactions from extracts generated from a wide range of cells and tissues from animals expressing fluorescent proteins. With the rapid workflow of plasmid transfection to single-molecule data collection, SMADNE has created the possibility to screen numerous disease-associated protein variants in a high-throughput manner previously unattainable with purified proteins. Hence, SMADNE performed in conjunction with the LUMICKS C-trap represents a novel, scalable, and relatively high-throughput method to obtain single molecule mechanistic insights into key protein-DNA interactions in an environment resembling the nucleus of mammalian cells.

TABLE 5
Proteins, including variants and different conditions, successfully analyzed using
SMADNE

| | | fluorescent | | weighted |
|---|---|---|---|---|
| | | tag | | corrected |
| Protein | Function | N- or C- | Substrate | lifetime (S) |
| AAG | glycosylase | C-GFP | Hx | 2.8 |
| | BER | | | |
| APE1 | nicking BER | N-GFP | DNA nicks | 0.3 |
| APOBEC3A | deamination | N-GFP | SSDNA | 6.0 |
| DDB1 | recog. NER | N-eGFP | UV | 43.7 |
| DDB2 | recog. NER | N-Halo | UV | 39.0 |
| K244E | | N- | UV | 27.2 (motile) |
| | | mNeonGreen | | |
| HPF1 | modifying | C-GFP | Nicked | 27.5 |
| | PARP activity | | nucleosome + | |
| | | | PARP1 | |
| LIG3 | nick sealing | N-Halo | DNA nicks | 5.4 |
| | BER | | | |
| ATP + Mg | | N-Halo | DNA nicks | 1.7 |
| K421A | | N-Halo | DNA nicks | 1.7 |
| ZNF1-BRCT | | N-Halo | DNA nicks | 11 |
| DZNF1 | | N-Halo | DNA nicks | 11.5 |
| OGG1 | glycosylase | C-GFP | 8-oxoG | 2.0 |
| | BER | | | |
| K249Q | | C-GFP | 8-oxoG | 47.2 |
| SPRTN-E112A | DPC removal | N-Halo | DPC | 184.5 |
| PARP1 | nick recog. | N-Halo | DNA nicks | 4.3 |
| | BER | | | |
| F44A | | N-Halo | DNA nicks | 8.3 |
| ZNF1&2 | | N-Halo | DNA nicks | 2.3 |
| H862A/Y896A/E988A | | N-Halo | DNA nicks | 97.4 |
| PARP2 | BER | N-YFP | DNA nicks | 11.7 |
| Pol-β | gap filling BER | N-GFP | DNA nicks | 2.0 |
| TDG | | C-Halo | nondamaged | 7.5 (motile) |
| | | | DNA | |
| WT | | C-Halo | formyl-C | 72.1 |
| N140A | | C-Halo | formyl-C | 1.9 |
| R275A | | C-Halo | nondamaged | 2.8 |
| | | | DNA | |
| R275L | | C-Halo | nondamaged | 1.8 |
| | | | DNA | |
| XPC | recog. NER | N-eGFP | UV | 75.5 (motile) |
| ATP + Mg | | N-Halo | UV | 3.6 |
| XRCC1 | nick sealing | C-YFP | DNA nicks | 6.9 |
| | BER | | | |

Cellular DNA is prone to oxidation, deamination and alkylation from both endogenous and exogenous sources¹⁻³. The resulting DNA lesions are repaired through base excision repair (BER), which is initiated by one of eleven DNA damage specific mammalian glycosylases. Alkyladenine glycosylase (AAG), also known as N-methylpurine DNA glycosylase (MPG), is an interesting glycosylase that appears to recognize structurally diverse substrates. These include the alkylation products N7-methyl G and N3-methyl A, as well as 1,N6-ethenoadenine (EA), a product of lipid peroxidation from exposure to vinyl chloride, or chloroacetaldehyde as reviewed in 4 and finally, hypoxanthine (Hx), the deamination product of adenine. Hx has also been shown to increase during chronic inflammation and has been found to occur in animal tissue at a frequency of about 0.5 lesions/106 deoxynucleosides but can rise approximately 10-fold following a model of chronic colitis due to Helicobacter pylori infection in mice⁸. Since Hx can pair with cytidine, it is mutagenic and has been found to cause AT to GC transition mutations in human cell lines⁹. During one branch of BER, AAG efficiently recognizes the DNA damage by flipping out the modified nucleotide into a recognition pocket. Using its N-glycosylase activity, AAG excises these damaged bases leaving a potentially cytotoxic abasic site (AP-site)¹⁰. APE1 nicks the DNA at AP-sites leaving a 5-deoxyribose phosphate (dRP) moiety. This nick can activate PARP1, which produces poly-(ADP)-ribose chains and helps recruit the scaffold protein XRCC1, which further facilitates the recruitment of DNA polymerase β and DNA Ligase III. DNA polymerase β removes the deoxyribose moiety and fills in the nucleotide gap. Finally, a DNA ligase seals the nick and completes repair¹¹. Incomplete repair of alkylation damage has been shown to be toxic to cells¹²⁻¹⁴. Unlike other glycosylases that bind more tightly to their abasic site product, AAG would appear to have equal to or lower affinity for abasic sites than either εA or Hχ moieties^{15,16}.

Previous work using biochemical, single molecule and cellular studies have demonstrated a direct role of UV-DDB (Uv damaged DNA-binding protein) in processing 8-oxoG lesions stimulating OGG1, MUTYH and APE1 activities^{8,9}. UV-DDB has the ability to bind to abasic sites in reconstituted nucleosomes and change their register as much as 3 bp, thus making the lesion more accessible to repair¹⁹. UV-DDB is a heterodimeric protein consisting of DDB1 (127 kDa) and DDB2 (48 kDa). UV-DDB is part of a larger complex containing cullin-4A/4B and RBX1 that possess E3 ligase activity. UV-DDB ubiquitinates histones to destabilize the nucleosome, thereby allowing downstream repair proteins to access the lesion^{20,21}. Previous studies suggested that UV-DDB may play a damage sensor role during BER by interacting with specific types of base damage contained in nucleosomes and stimulating the activity of damage specific glycosylases. Glycosylases, such as AAG may be stimulated by UV-DDB.

While AAG shows less affinity for abasic sites than other glycosylases, the low rate of turnover of AAG is attributable to its ability to bind to abasic sites with equal affinity as εA or Hx^{15,16}. Previous studies have been designed to examine product release by AAG. The SMADNE approach allowed for AAG to detect hypoxanthine lesions within nuclear extracts. This method closely replicates nuclear conditions in contrast to investigations involving purified proteins. AAG remained stationary at sites of Hx incorporation, but has increased linear diffusion while binding non-specifically to DNA. While the diffusivity of events seemed relatively consistent between the approaches, the lifetime with the SMADNE approach was much reduced. This may be due to non-specific binding to DNA by AAG, which samples DNA briefly could also be detected on this new C-trap platform and were not readily observable with the tightrope assay which detects longer lived events. This shorter lifetime could also be due to other proteins in the nuclear extract such as UV-DDB or APE1 assisting with the dissociation of AAG.

### 6.1.3 Materials and Methods

Recombinant full-length UV-DDB (DDB1-DDB2 heterodimer) was expressed in Sf9 cells coinfected with recombinant baculovirus of His6-DDB1 and DDB2-Flag, as performed previously⁹. Briefly, a 5 ml His-Trap HP column pre-charged with Ni²⁺ (GE Healthcare) and anti-FLAG M2 affinity gel (Sigma) was used to purify DDB1-His6 and DDB2-Flag. The pooled anti-FLAG eluate containing UV-DDB (DDB1:DDB2 at a 1:1 ratio) was purified based on size with a HiLoad 16/60 Superdex 200 column (Amersham Pharmacia) in UV-DDB storage buffer (50 mM HEPES, pH 7.5, 200 mM KCl, 1 mM EDTA, 0.5 mM PMSF, 2 mM DTT, 10% glycerol and 0.02% sodium azide). Purified fractions of DDB1-DDB2 complex from the Superdex200 were aliquoted and flash-frozen with liquid nitrogen and stored at −80° C. AAG WT was purchased from NOVUS (Saint Charles, MO) and AAG 80 p.E125Q (EQ) was purified as previously described²².

U2OS cells were cultured in 5% oxygen in Dulbecco's Modified Eagle Medium (DMEM) supplemented with 4.5 g/l glucose, 10% fetal bovine serum (Gibco), 5% penicillin/streptavidin (Life Technologies). To obtain transient overexpression of the fluorescent-tagged proteins of interest, 4 μg of plasmid per 4 million cells was used to transfect using the lipofectamine 3000 reagent and protocol for 24 h (Thermo Fisher Cat #L3000008). Cells with overexpressed HaloTag fusions were treated with 100 nM (˜10-100 fold molar excess) of fluorescent HaloTag ligand for 30 minutes at 37° C. (Janelia Fluor® 635 or 503 HaloTag® Ligand from Dr. Luke Lavis Laboratory, Janelia Research Campus). In most cases, protein overexpression was performed one at a time, with the exception of the co-transfection of eGFP-DDB1 and HaloTag-DDB2 and a co-transfection of cGFP-XPC with unlabeled RAD23B. Protein overexpression was confirmed via western blot and by quantifying the fluorescence intensity in solution on the C-trap© correlative optical tweezers and fluorescent microscope (FIGS. 7 and 8; and Table 2). For the fluorescence intensity measurements, standard curves of the background photon counts apparent on the C-trap were created for purified GFP or purified HaloTag protein conjugated to the fluorescent dyes of interest. The intensities of the nuclear extracts were then interpolated into the standard curves to determine concentration (Table 2).

### Nuclear Extraction

Nuclear extraction was performed the day after transient transfection using a nuclear extraction kit from Abcam (ab113474). After extraction following the protocol from the Abcam kit, the tubes were aliquoted into single-use aliquots and flash-frozen in liquid nitrogen prior to storage at −80° C. Upon use for single-molecule experiments, nuclear extracts were immediately diluted after thawing in buffer for experiments at a ratio of 1:10. Table 4 provides a list of buffer conditions used in each experiment. Nucleic acid concentration was determined using a Quant-iT™ PicoGreen™ dsDNA Assay Kits (Invitrogen) and total protein concentration obtained using a Bradford assay (Bio-Rad) (Total protein was on average 1.2 mg/mL).

Western Blot of Overexpressed Proteins from Nuclear Extracts

Extracts and purified proteins (FIG. 2) were loaded onto 4-20% tris-glycine polyacrylamide gels (Invitrogen; XP04202BOX). Proteins were transferred onto a polyvinylidene difluoride membrane followed by blocking in 20% nonfat dry milk (diluted in PBST: phosphate-buffered saline containing 0.1% Tween 20) for 1 h at room temperature. Membranes were incubated with primary antibodies for 2 h at room temperature or overnight at 4° C., washed 3×10 min in PSBT, and incubated with peroxidase conjugated secondary antibodies for 1 h at room temperature. Membranes were washed again before developing using SuperSignal West Femto Maximum Sensitivity Substrate (Thermo Fisher Scientific; #34095). Primary antibodies used: DDB2 (1:1000; abcam #ab181136), DDB1 (1:1000; Invitrogen #37-6200). Secondary antibodies used: anti-rabbit IgG (1:50,000 Sigma #A0545), or anti-mouse IgG (1:50,000 Sigma #A4416). Blots were analyzed on ImageJ v1.53k.

### Mass Spectrometry of Nuclear Extracts

A 2 μg aliquot of each sample was analyzed by nano LC/MS/MS with a Waters M-class HPLC system interfaced to a ThermoFisher Fusion Lumos. Peptides were loaded on a trapping column and eluted over a 75 μm analytical column at 350 nL/min; both columns were packed with XSelect CSH C18 resin (Waters); the trapping column contained a 3.5 μm particle, the analytical column contained a 2.4 μm particle. The column was heated to 55° C. using a column heater (Sonation). A 2 h gradient was employed. The mass spectrometer was operated in data-dependent mode, with MS and MS/MS performed in the Orbitrap at 60,000 FWHM resolution and 15,000 FWHM resolution, respectively. APD was turned on. The instrument was run with a 3 s cycle for MS and MS/MS. Data were processed through the MaxQuant software v1.6.2.3 (www.maxquant.org) which served several functions: 1) recalibration of MS data, 2) filtering of database search results at the 1% protein and peptide false discovery rate (FDR), 3) calculation of peak areas for detected peptides and proteins, and 4) data normalization using the LFQ algorithm.

### DNA Substrate Generation

Lambda DNA for C-trap experiments was purchased from New England Biotechnologies. The ends were biotinylated by adding a mix of 6 μg lambda DNA, 50 μM nucleotide mix (with dATP, dGTP, dTTP, and biotinylated dCTP), 15 units of Klenow fragment polymerase (NEB) and 1× concentration of NEB Buffer 2. By filling in the overhangs on the cos sites of lambda DNA, the reaction labeled one side of the lambda DNA with four biotins and the other with six. The reaction was incubated for 30 minutes at 37° C. and then the free nucleotides were removed from solution via ethanol precipitation, with 1 μg/μl glycogen used as a co-precipitant to increase the yield. Biotinylation of the lambda DNA was confirmed by generating force-distance curves on the C-trap instrument and fractions were frozen down in aliquots of 20 ng/μL at −20° C. After thawing aliquots, they were stored at 4° C. for up to 2 weeks and then discarded.

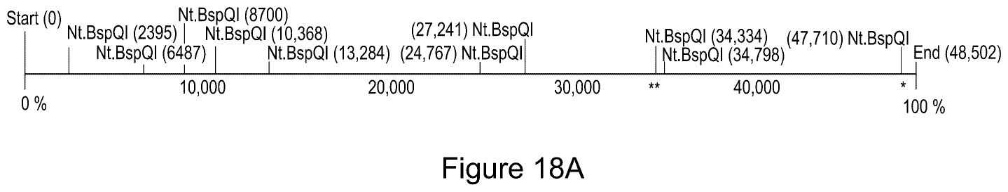

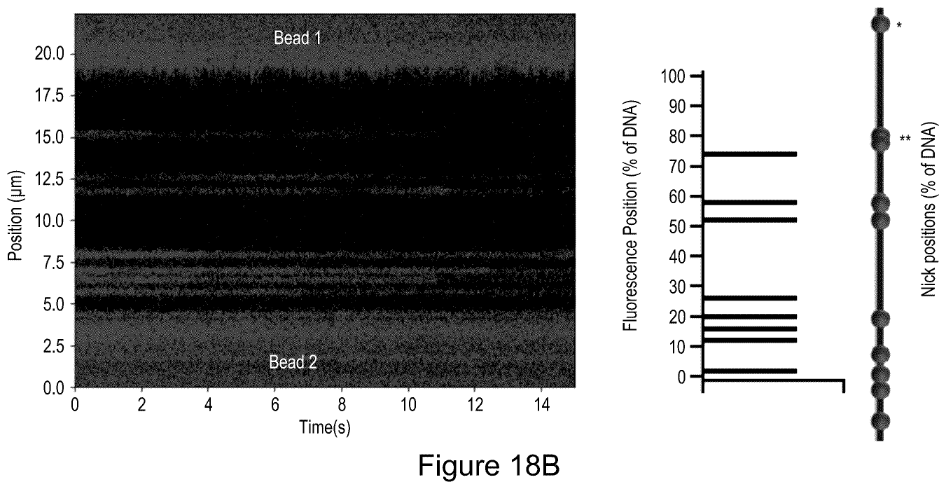

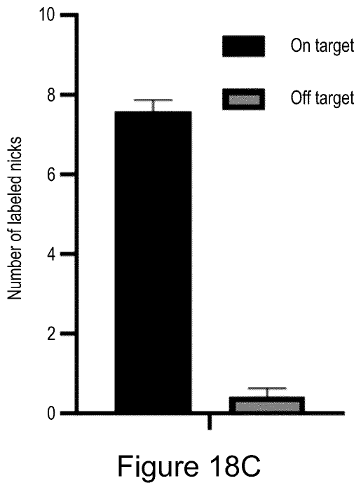

Biotinylated lambda DNA was then utilized to generate various forms of DNA damage for SMADNE characterization. To create UV-damage, biotinylated lambda DNA was irradiated with UV-C for 40 J/m². Similarly, to create oxidative damage on lambda DNA, a single use ali-quot was incubated with 0.2 μg/mL methylene blue16 and exposed to 660 nm light for 10 minutes. Lastly, DNA with single-stranded breaks (nicked DNA) was generated by digesting 1 μg of DNA with the nickase Nt.BspQI (NEB) following the manufacturer's instructions. This nickase recognized the 10 distinct sequences of 5′-GCTCTTCN-3′ along the lambda DNA to generate 10 nicks, cutting on the 3′ side of its recognition sequence (FIG. 18). After nicking the DNA, fluorescent nucleotides were incorporated at the sites using nick translation for identification of nick sites, using a 40 μM mix of dGTP, dCTP, dATP and fluorescein-tagged dUTP, as well as 10 units of pol I and 800 ng nicked lambda DNA. Results for this nick translation reaction agreed with the anticipated sites of DNA nicks with few off-target incorporations (FIG. 18). DNA containing Hx and Cy3 fiducial markers of the damage positions was generated by first treating 1 μg of DNA with the nickase Nt.BspQI (NEB) to generate 10 nicks in lambda DNA at specific sites. Two of the positions are close together and not resolved in the assay and another is too close to the bead to be observed so only 8 sites are observed. After nicking the DNA, fluorescent nucleotides were incorporated using nick translation for identification of nick sites, using a 40 μM mix of dGTP, dCTP, dITP (deoxyinosine triphosphate, the nucleotide form of hypoxanthine) and Cy3-labeled dUTP, in the presence of 10 units of pol I and 800 ng nicked lambda DNA.

### DNA Tether Formation and Positioning

Single-molecule experiments were performed on a LUMICKS C-Trap instrument, which consists of a three-color confocal fluorescence microscope and dual-trap optical tweezers²⁷. A microfluidic flow-cell from LUMICKS was used containing 5 distinct flow channels separated by laminar flow that could be traversed by the two optical traps. However, only 4 of the flow channels were utilized for these experiments (FIG. 1). To prepare the DNA substrates for single-molecule imaging, channels one, two, and three were filled with 4.38 μm polystyrene streptavidin beads (LUMICKS), biotinylated DNA, and buffer of interest, respectively. All three were flowed at a pressure of 0.3 bar to maintain laminar flow. While maintaining flow, single beads were caught in both optical traps in channel one. Then, the beads were moved to channel 2 for DNA capture. To suspend DNA between the two traps, the bead in trap 2 was held in a constant position while moving trap 1 downstream and upstream of the flow (keeping the two traps parallel in the flow but varying the distance). By measuring force-distance curve each time the traps were spread apart, an increase in the force with an increased distance indicated the binding of a DNA tether. The force-distance curves were then compared to the extensible wormlike chain model for DNA of 48,500 bp to verify that a single tether of dsDNA was caught²⁸.

After tether formation, the beads with the suspended DNA were moved to the buffer channel (channel three) and channel three and four were flowed at 0.3 bar for at least 10 seconds to introduce nuclear extracts into the flow cell. After flushing in the extract, the flow was stopped and the traps were moved to the position where channel four (the channel with nuclear extracts) joined the flow cell. Immediately after (unless otherwise indicated) the force-distance curve was re-zeroed and bead one was pulled to generate the tension desired for data collection (typically 10 pN). Of note, nuclear debris from the extract tended to get trapped in the optical traps and changed the apparent force measurement by positive or negative 6 pN over 5 minutes of collection. Therefore, after initial force curve was determined and the positions of the traps required to maintain the desired force were defined, the trap positions were not altered throughout the data collection to maintain a constant force on the DNA throughout the data collection.

### Confocal Imaging

Various fluorophores were utilized throughout this study, and each was excited with the laser closest to their maximum excitation wavelength. cGFP, tGFP, YFP, fluorescein, mNeonGreen and HaloTag-JF-503 were excited with a 488 nm laser and emission collected in a 500-550 nm band pass filter, mScarlet was excited at 561 nm and emission collected in a 575-625 nm band pass filter, and HaloTag-JF-635 was excited with a 638 nm laser and emission collected in a 650-750 nm band pass filter (Table 3). All data was collected with a 1.2 NA 60× water emersion objective and photons measured with single-photon avalanche photodiode detectors. With each fluorophore, the imaging settings were set with both the photostability and binding lifetimes in mind (Tables 3 and 4). Typically, each laser was set to 5% power and scanned continuously (0.1 msec of exposure for each pixel of size 100 nm; the frame rate depending on the length of the DNA but typically ˜34 ms per frame). However, for some binding events with long binding lifetimes and lower photostability (i.e., eGFP-tagged DDB1), a pulsed excitation was utilized. In this imaging scheme, the same exposure time and laser power was utilized, but brief pauses were included between each exposure. In the case of eGFP-DDB1, for instance, data was collected with a 34 ms exposure followed by 66 ms pause in exposure, thus increasing the fluorophore lifetime by threefold. Table 3 provides a list of laser powers, average binding lifetime, photobleaching lifetime with each fluorophore, and exposure settings.

### Single-Molecule Förster Resonance Energy Transfer Imaging

For the FRET approach in FIG. 15, data were collected at 50% power of the 488 nm laser at 34 ms per frame to excite the FRET donor eGFP-DDB1, and the intensity of mCherry-DDB2 was measured as the FRET acceptor. For quantification of the signal, lines that exhibited acceptor emission were tracked with pylake, and then downsampled by a factor of ten to increase the signal-noise of the fluorescence data. To subtract for background signal in the quantifications of the intensities, photon counts for each channel were taken for the region between 6-9 pixels on either side of the tracked line, resulting in zones that follow the path of the event in regions without fluorescent signal. Then bleedover was subtracted from the eGFP-DDB1 by collecting multiple events with both colors, photobleaching the mCherry-DDB2 signal, and then measuring the resultant intensities in the acceptor channel caused by eGFP emission. These intensities were consistently 9.0% of the intensity of eGFP in the FRET donor emission channel, so that ratio was used for subtracting the bleedover.

### TIRF C-Trap Experiments

Other single-molecule fluorescence experiments were performed on a commercial optical tweezers and microfluidics system using the TIRF objective (C-trap; LUMICKS). The system is equipped with 5 microfluidic channels, four were used as follows: channel 1 contained 3.7 μm diameter streptavidin-coated polystyrene beads (Spherotech), channel 2 contained biotinylated A-DNA (damaged beforehand with 40 J/m² UVC), channel 3 contained buffer and channel 4 contained nuclear extract with overexpressed eGFP-DDB1 and HaloTag-DDB2 conjugated to Janelia fluor 635.

Following bead capture in channel 1 the tethered DNA was held 10 μm above the surface in channel 2 using the laser tweezers at 30% power. Flow at 0.2±0.05 bar was used during DNA capture and a single strand of damaged biotinylated A-DNA was tethered between the beads. The DNA tensions used were 10 pN for experiments without flow and 30 pN with flow. The tether was then transferred to the nuclear extract in channel 4. Depending on the experiment, the flow was kept constant at 0.05±0.03 bar; pulsed at 0.05±0.03 bar for 3 seconds on then 10 seconds off; or the channel was flushed for ˜10 seconds at 0.1±0.05 bar to introduce fresh protein and binding was observed without flow. Fluorophores were excited with the 488 nm (80% power) and 638 nm (40% power) lasers for 200 ms with exposure synchronisation. Videos were taken over the region encompassing the tether and beads at a framerate of 4.3 Hz.

### Data Analysis

Images and force data collected from kymographs was exported and analyzed using custom software by LUMICKS (Pylake). For visualization of the kymographs and 2D scans after exporting, the utility C-Trap.h5 Visualization GUI was used²⁹. As data was collected with images containing both the DNA of interest and the polystyrene beads, the pixels on the edge of the beads were first defined to determine the start and the end positions of the DNA. Line tracking was performed using a custom script from LUMICKS based performing a Gaussian fit over the line intensity and connecting the time points to form a line using previous line tracking algorithms³⁰. Of note, fluorophores derived from GFP tended to blink for periods up to two seconds, which caused line tracking programs to identify a single event as two separate binding events. To address this issue, the tracked lines were curated to determine if any events occurred at the same position (<100 nm) with off times less than 2 seconds—the gaps in these lines were manually connected using a feature of the LUMICKS software. After tracking the lines, the position and time data for each line was used to determine each line's duration, the number of lines per minute, and the average position of each line.

For motile events, mean squared displacement (MSD) was calculated using a custom script provided by LUMICKS, with the equation:

$$MSD ( n Δ t ) = \frac{1}{N - n} {\sum }_{i = 1}^{N - n} {( {x}_{i + n} - {x}_{i} )}^{2}$$

### TIRF C-Trap Data Analysis

Videos were analyzed using ImageJ (imagej.nih.gov/ij/). In the case of DDB1+DDB2 images two channels were overlaid and aligned using Align RGB planes plugin (blog.bham.ac.uk/intellimic/g-landini-software/), using the laser tweezer captured beads as fiducial markers. Line traces along the position of the DNA tether were converted to kymographs, which provided continuous streaks corresponding to bound molecules. Lifetimes were determined by measuring the length of the streaks and converted to time, based on the known framerate. Bound lifetimes were analyzed using the CRTD approach³¹. CRTDs were then fitted to single (DDB1, DDB1+DDB2) or double (DDB2) exponentials based on fit quality and examination of residuals. Fitting was performed in Microsoft Excel using Solver. Fit errors are SEM. As the photobleaching rates were similar to the rates of dissociation in this data, corrections to the lifetimes were made as previously published³².

### Colocalization Analysis

For colocalization analysis, lines tracked from the trimmed data were compared against each other using a custom-made colocalization analysis script. Briefly, times and positions for each datapoint of each line were compared between the two sets of lines to determine if the distance and time agreed within an adjustable window (less than 200 nm and 400 ms apart). By calculating the data this way, even events that started without colocalization before diffusing a colocalized position would be counted-however no datasets with motile events were used for colocalization analysis. This script, named colocalization analyzer, is available at harbor.lumicks.com/scripts.

### Photobleaching Analysis

Photobleaching decay constants were determined for each fluorophore by collecting kymographs with continuous exposure on fluorophores immobilized at the bottom of the slide. To collect kymographs, the objective of the C-trap @ was lowered to the bottom of the flow chamber until defined single-molecule spots could be observed and photon counts per second reached a maximum. After focusing, a minimum of 3 kymographs were taken under the collection settings. Photon counts from the appropriate channel were binned into bins consisting of 1 second intervals and the resulting bins fit to a single-exponential decay function to determine photobleaching lifetimes (Table 3). This script, named photostability calculator, is publicly available at harbor.lumicks.com/scripts.

### Code Availability

Code for converting positional data to 2D movies is available on github at github.com/Kad-Lab/SMADNE.

### 6.1.4 References

1. Cho, N. H. et al. OpenCell: Endogenous tagging for the cartography of human cellular organization. Science 375, cabi6983, doi: 10.1126/science.abi6983 (2022).
2. Liu, L. et al. PARP1 changes from three-dimensional DNA damage searching to one-dimensional diffusion after auto-PARylation or in the presence of APE1. Nucleic Acids Res 45, 12834-12847, doi: 10.1093/nar/gkx1047 (2017).
3. Liu, T.-C. et al. APE1 distinguishes DNA substrates in exonucleolytic cleavage by induced space-filling. Nature Communications 12, 601, doi: 10.1038/s41467-020-20853-2 (2021).
4. Cheon, N. Y., Kim, H.-S., Yeo, J.-E., Schärer, O. D. & Lee, J. Y. Single-molecule visualization reveals the damage search mechanism for the human NER protein XPC-RAD23B. Nucleic Acids Research 47, 8337-8347, doi: 10.1093/nar/gkz629 (2019).
5. Freudenthal, B. D., Beard, W. A., Shock, D. D. & Wilson, S. H. Observing a DNA polymerase choose right from wrong. Cell 154, 157-168, doi: 10.1016/j.cell.2013.05.048 (2013).
6. Ghodke, H. et al. Single-molecule analysis reveals human UV-damaged DNA-binding protein (UV-DDB) dimerizes on DNA via multiple kinetic intermediates. Proceedings of the National Academy of Sciences 111, E1862, doi: 10.1073/pnas. 1323856111 (2014).
7. Fujiwara, Y. et al. Characterization of DNA recognition by the human UV-damaged DNA-binding protein. J Biol Chem 274, 20027-20033, doi: 10.1074/jbc.274.28.20027 (1999).
8. Jang, S. et al. Single molecule analysis indicates stimulation of MUTYH by UV-DDB through enzyme turnover. Nucleic Acids Res 49, 8177-8188, doi: 10.1093/nar/gkab591 (2021). 9. Jang, S. et al. Damage sensor role of UV-DDB during base excision repair. Nat Struct Mol Biol 26, 695-703, doi: 10.1038/s41594-019-0261-7 (2019).
10. Los, G. V. et al. HaloTag: a novel protein labeling technology for cell imaging and protein analysis. ACS Chem Biol 3, 373-382, doi: 10.1021/cb800025k (2008).
11. Lo, H.-L. et al. Differential biologic effects of CPD and 6-4PP UV-induced DNA damage on the induction of apoptosis and cell-cycle arrest. BMC Cancer 5, 135-135, doi: 10.1186/1471-2407-5-135 (2005).
12. Zou, Y., Crowley, D. J. & Van Houten, B. Involvement of molecular chaperonins in nucleotide excision repair. Dnak leads to increased thermal stability of UvrA, catalytic UvrB loading, enhanced repair, and increased UV resistance. J Biol Chem 273, 12887-12892, doi: 10.1074/jbc.273.21.12887 (1998).
13. Graham, J. S., Johnson, R. C. & Marko, J. F. Concentration-dependent exchange accelerates turnover of proteins bound to double-stranded DNA. Nucleic Acids Res 39, 2249-2259, doi: 10.1093/nar/gkq1140 (2011).
14. Ha, T. Single-molecule approaches embrace molecular cohorts. Cell 154, 723-726, doi: 10.1016/j.cell.2013.07.012 (2013).
15. Gibb, B. et al. Concentration-dependent exchange of replication protein A on single-stranded DNA revealed by single-molecule imaging. PLOS One 9, e87922, doi: 10.1371/journal.pone.0087922 (2014).
16. Nelson, S. R., Dunn, A. R., Kathe, S. D., Warshaw, D. M. & Wallace, S. S. Two glycosylase families diffusively scan DNA using a wedge residue to probe for and identify oxidatively damaged bases. Proceedings of the National Academy of Sciences 111, E2091, doi: 10.1073/pnas. 1400386111 (2014).
17. Blainey, P. C., van Oijen, A. M., Banerjee, A., Verdine, G. L. & Xie, X. S. A base-excision DNA-repair protein finds intrahelical lesion bases by fast sliding in contact with DNA. Proceedings of the National Academy of Sciences 103, 5752, doi: 10.1073/pnas.0509723103 (2006).
18. Nash, H. M., Lu, R., Lane, W. S. & Verdine, G. L. The critical active-site amine of the human 8-oxoguanine DNA glycosylase, hOgg1: direct identification, ablation and chemical reconstitution. Chemistry & biology 4, 693-702, doi: 10.1016/s1074-5521 (97) 90225-8 (1997).
19. Haraszti, R. A. & Braun, J. E. Comparative Colocalization Single-Molecule Spectroscopy (CoSMoS) with Multiple RNA Species. Methods Mol Biol 2113, 23-29, doi: 10.1007/978-1-0716-0278-2_3 (2020).
20. Hoskins, A. A. et al. Ordered and dynamic assembly of single spliceosomes. Science (New York, N.Y.) 331, 1289-1295, doi: 10.1126/science. 1198830 (2011).
21. Sparks, J. L. et al. The CMG Helicase Bypasses DNA-Protein Cross-Links to Facilitate Their Repair. Cell 176, 167-181.e121, doi: 10.1016/j.cell.2018.10.053 (2019).
22. Kanke, M., Tahara, E., Huis In't Veld, P. J. & Nishiyama, T. Cohesin acetylation and Wapl-Pds5 oppositely regulate translocation of cohesin along DNA. Embo j 35, 2686-2698, doi: 10.15252/embj.201695756 (2016).
23. Graham, T. G. W., Walter, J. C. & Loparo, J. J. Two-Stage Synapsis of DNA Ends during Non-homologous End Joining. Mol Cell 61, 850-858, doi: 10.1016/j.molcel.2016.02.010 (2016).
24. Aggarwal, V. & Ha, T. Single-molecule pull-down (SiMPull) for new-age biochemistry. BioEssays 36, 1109-1119, doi.org/10.1002/bies.201400090 (2014).
25. Jain, A., Liu, R., Xiang, Y. K. & Ha, T. Single-molecule pull-down for studying protein interactions. Nat Protoc 7, 445-452, doi: 10.1038/nprot.2011.452 (2012).
26. Jain, A. et al. Probing cellular protein complexes using single-molecule pull-down. Nature 473, 484-488, doi: 10.1038/nature 10016 (2011).
27. Hashemi Shabestari, M., Meijering, A. E. C., Roos, W. H., Wuite, G. J. L. & Peterman, E. J. G. in Methods in Enzymology Vol. 582 (eds Maria Spies & Yann R. Chemla) 85-119 (Academic Press, 2017).
28. Wang, M. D., Yin, H., Landick, R., Gelles, J. & Block, S. M. Stretching DNA with optical tweezers. Biophys J 72, 1335-1346, doi.org/10.1016/S0006-3495 (97) 78780-0 (1997).
29. Watters, J. W. C-Trap.h5 Visualization GUI. Retrieved from harbor.lumicks.com/(2020).
30. Mangeol, P., Prevo, B. & Peterman, E. J. KymographClear and KymographDirect: two tools for the automated quantitative analysis of molecular and cellular dynamics using kymographs. Mol Biol Cell 27, 1948-1957, doi: 10.1091/mbc.E15-06-0404 (2016).
31. Kastantin, M., Langdon, B. B., Chang, E. L. & Schwartz, D. K. Single-molecule resolution of interfacial fibrinogen behavior: effects of oligomer populations and surface chemistry. J Am Chem Soc 133, 4975-4983, doi: 10.1021/ja110663u (2011).
32. Suzuki, K. G. N., Kasai, R. S., Fujiwara, T. K. & Kusumi, A. in Methods in Cell Biology Vol. 117 (ed P. Michael Conn) 373-390 (Academic Press, 2013).
33. Aamodt, R. M., Falnes, P. O., Johansen, R. F., Seeberg, E., and Bjoras, M. (2004) The Bacillus subtilis counterpart of the mammalian 3-methyladenine DNA glycosylase has hypoxanthine and 1,N6-ethenoadenine as preferred substrates. J. Biol. Chem., 279, 13601-13606.
34. Mechetin, G. V., Endutkin, A. V., Diatlova, E. A., and Zharkov, D. O. (2020) Inhibitors of DNA glycosylases as prospective drugs. Int. J. Mol. Sci., 21, 3118.
35. Thelen, A. Z. and O'Brien, P. J. (2020) Recognition of 1,N(2)-ethenoguanine by alkyladenine DNA glycosylase is restricted by a conserved active-site residue. J. Biol. Chem., 295, 1685-1693.
36. Jelezcova, E., Trivedi, R. N., Wang, X. H., Tang, J. B., Brown, A. R., Goellner, E. M., Schamus, S., Fornsaglio, J. L., and Sobol, R. W. (2010) Parp1 activation in mouse embryonic fibroblasts promotes pol beta-dependent cellular hypersensitivity to alkylation damage. Mutat. Res., 686, 57-67.
37. Sobol, R. W., Watson, D. E., Nakamura, J., Yakes, F. M., Hou, E., Horton, J. K., Ladapo, J., Van Houten, B., Swenberg, J. A., and Tindall, K. R. et al. (2002) Mutations associated with base excision repair deficiency and methylation-induced genotoxic stress. Proc. Natl. Acad. Sci. USA, 99, 6860-6865.
38. Sobol, R. W. and Wilson, S. H. (2001) Mammalian DNA beta-polymerase in base excision repair of alkylation damage. Prog. Nucleic Acid Res. Mol. Biol., 68, 57-74.
39. Abner, C. W., Lau, A. Y., Ellenberger, T., and Bloom, L. B. (2001) Base excision and DNA binding activities of human alkyladenine DNA glycosylase are sensitive to the base paired with a lesion. J. Biol. Chem., 276, 13379-13387.
40. Admiraal, S. J. and O'Brien, P. J. (2015) Base excision repair enzymes protect abasic sites in duplex DNA from interstrand cross-links. Biochemistry, 54, 1849-1857.
41. Matsumoto, S., Cavadini, S., Bunker, R. D., Grand, R. S., Potenza, A., Rabl, J., Yamamoto, J., Schenk, A. D., Schubeler, D., Iwai, S. et al. (2019) DNA damage detection in nucleosomes involves DNA register shifting. Nature, 571, 79-84.
42. Fischer, E. S., Scrima, A., Böhm, K., Matsumoto, S., Lingaraju, G. M., Faty, M., Yasuda, T., Cavadini, S., Wakasugi, M., Hanaoka, F. et al. (2011) The molecular basis of CRL4DDB2/CSA ubiquitin ligase architecture, targeting, and activation. Cell, 147, 1024-1039.
43. Kapetanaki, M. G., Guerrero-Santoro, J., Bisi, D. C., Hsich, C. L., Rapic-Otrin, V., and Levine, A. S. (2006) The DDB1-CUL4ADDB2 ubiquitin ligase is deficient in xeroderma pigmentosum group e and targets histone H2A at UV-damaged DNA sites. Proc. Natl. Acad. Sci. U.S.A., 103, 2588-2593.

### 6.2 Example 2

### SMADNE: Real-Time Study of CMG and MCV LT Helicase Initiation

The primary control step regulating eukaryotic DNA replication involves helicase-mediated unwinding and melting of double-stranded DNA (dsDNA) by the minichromosome maintenance (MCM) protein complex. During late mitosis and early G1 phases, MCM is loaded on the origin by origin recognition complex (ORC) proteins as a dodecameric head-to-head double hexamer (1). MCM then associates with licensing factors Cdc45 and GINS to form Cdc45-MCM-GINS (CMG) during the late G1/early S phase (2). Crystallographic and cryoelectron microscopic studies show that CMG assembles as a fully formed dodecameric complex composed of two oppositely positioned hexamers (a “double hexamer”) surrounding the origin dsDNA. Once licensed to replicate during the S phase, each hexamer in the CMG complex hydrolyzes ATP to ratchet together the intervening dsDNA to achieve DNA melting³⁻⁵. The MCM hexamers then each remodel around a single-stranded (ss)DNA to generate a melted replication bubble that attracts assembly of the replisome machinery⁶. Generation of a melted bubble in this model requires the full assembly of the double hexamer and ATP hydrolysis for DNA melting.

Merkel cell virus (MCV) encodes its own replication helicase, the multifunctional large tumor (LT) oncoprotein, which is both necessary and sufficient to initiate viral DNA replication⁷. MCV is one of seven human cancer viruses and causes the clinically aggressive skin cancer, Merkel cell carcinoma (MCC)⁸. Nearly 3,000 people in the United States develop this cancer each year⁹, of which ˜80% are MCV infected. The remaining 20% of MCC cases have tumors negative for the virus but phenocopy viral infection through UV-driven somatic mutations¹⁰. MCV was identified by digital transcriptome subtraction and was the first human pathogen discovered by nondirected metagenomic cDNA sequencing¹¹.

Unlike the human CMG, MCV LT can initiate multiple rounds of viral genome replication within a single cell cycle (unlicensed replication)⁷. MCC oncogenesis generally occurs after viral replication¹² when fragmented viral genomes become integrated into the host cell genome^{7, 13}. Because LT can reinitiate DNA replication off of the integrated viral origin⁷, leading to replication fork collision and DNA fragmentation, the nascent cancer cell survives because another, independent mutation is present in the LT gene to truncate its C-terminal helicase domain preventing LT-dependent DNA replication^{7, 14}. It is unknown which mutation comes first, LT gene truncation⁷ or virus integration¹¹, but both are required, together with loss of effective cytotoxic T lymphocyte responses against early viral antigens^{15, 16}, for emergence of this virus-driven cancer⁸.

MCV LT binds to a 98 base pair (bp) viral origin (ori) located within the 464 bp noncoding control region (NCCR)¹⁷. MCV is related to the rhesus macaque SV40 polyomavirus that has been an extensively studied model for eukaryotic DNA replication for over 50 y. The first in vitro eukaryotic DNA replication studies were performed using the LT protein and DNA origin of SV40 (18, 19), leading to the discovery of critical cellular factors in eukaryotic replication^{20, 21}. SV40 LT helicase assembles as a head-to-head, double-hexameric homopolymer that is reported to unwind less than a single turn of DNA as it assembles through a mechanism requiring ATP binding but not hydrolysis²². Origin melting by SV40 LT, however, is also reported to occur through a dsDNA ratcheting mechanism similar to that of CMG helicase^{3, 23, 24}, while still other studies indicate that origin melting occurs in the absence of ATP hydrolysis and helicase activity^{25, 26}.

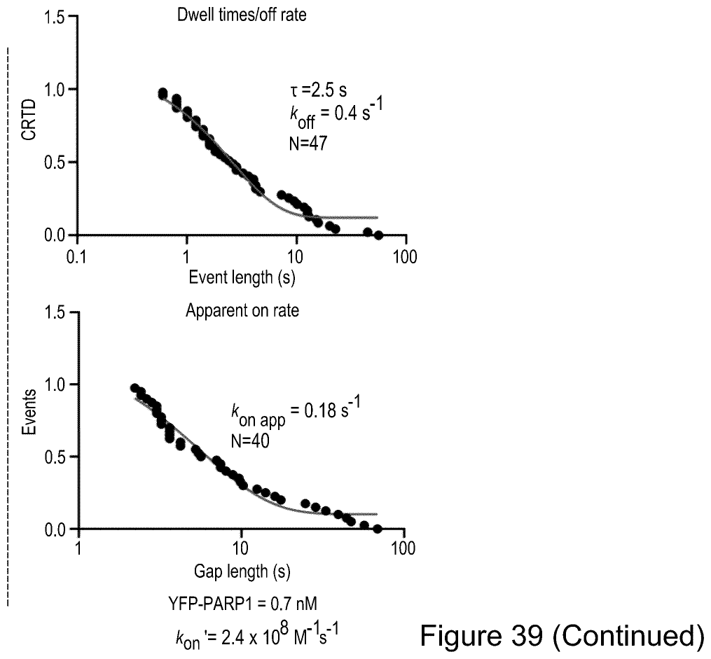

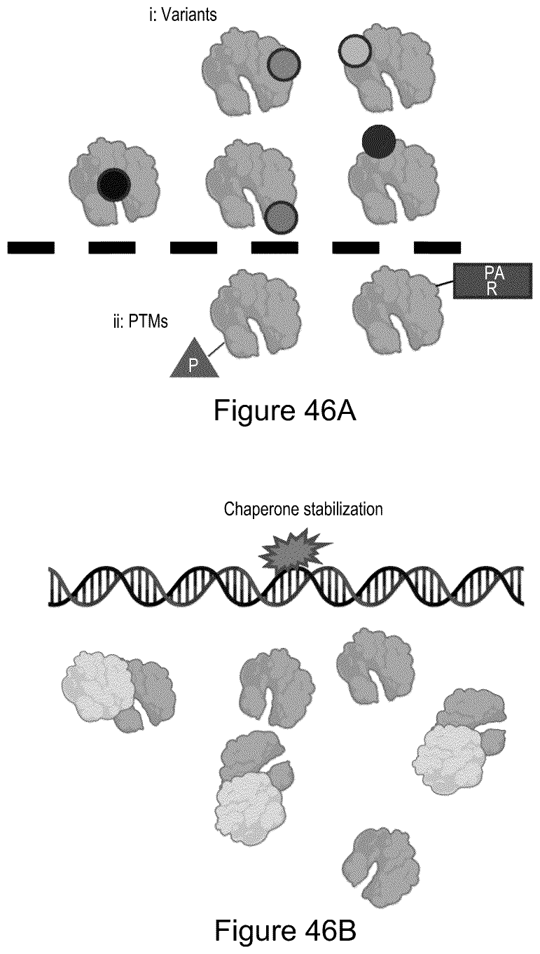

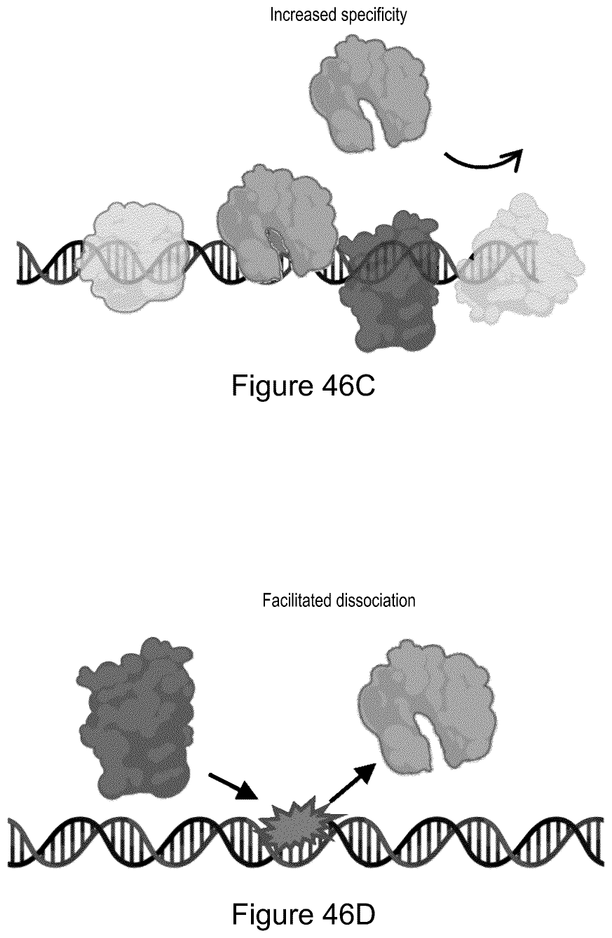

SV40 and MCV LT proteins are homologous, but not identical (FIG. 39A), and the extensive literature on SV40 LT can help guide experimentation on MCV LT. Both MCV and SV40 LT proteins have origin-binding domains that recognize canonical G(A/G) GGC pentanucleotide sequences (PS or pentads) in the origin¹⁷. Although pentad nucleotide sequences are identical for both viruses, their numbers and spacing differ at their respective origins (FIG. 46A) such that the two LT proteins cannot replicate each other's viral genomes^{(7, 27)}. Ten pentads are present in the MCV ori, but only four (PS1, 2, 4 and 7) are required for replication¹⁷. A single point mutation at one critical pentad (PS7) recovered from an MCC tumor genome (MCC350)¹¹ prevents LT-mediated DNA replication (here called Ori98.Rep-)^{17, 28}.

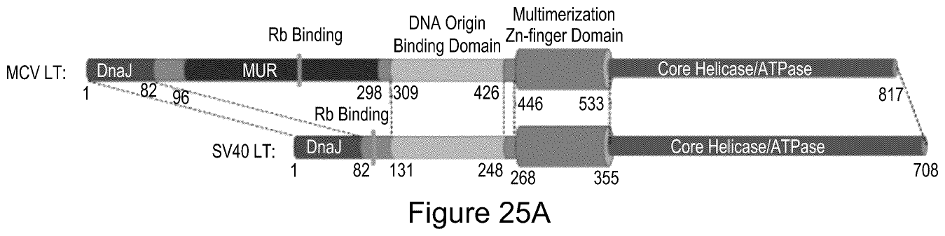

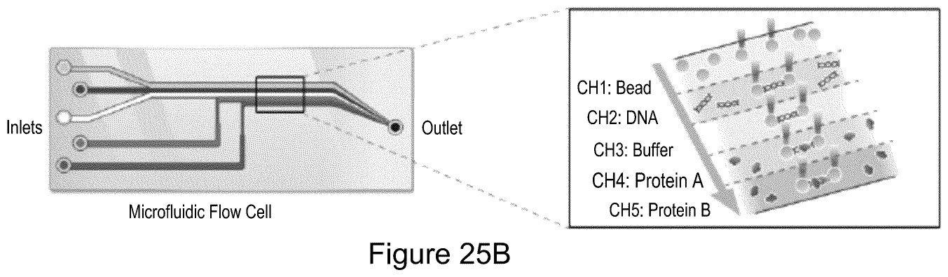

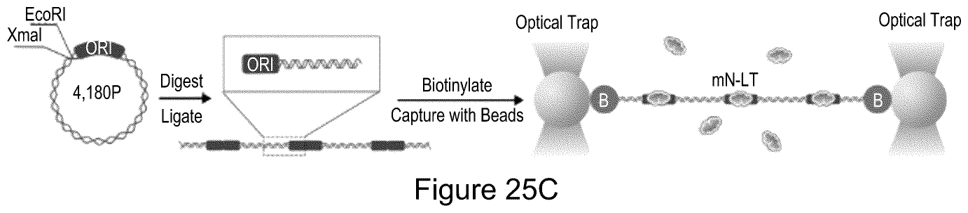

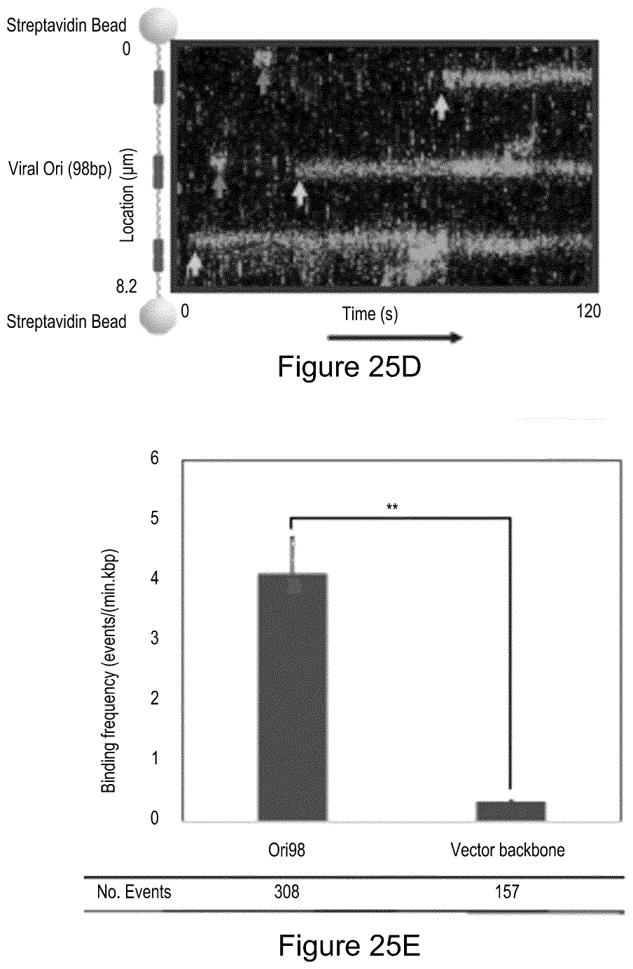

The present disclosure visualized the real-time assembly of MCV LT on single-molecule MCV DNA replication origins with an optical tweezers/fluorescence microscope (FIG. 25B), using a Hidden MarkovModel (HMM) simulation²⁹ to quantitate LT assembly. The present disclosure shows how MCV and SV40 helicases initiate dsDNA melting. The initial molecular steps in unlicensed MCV replication involve multimeric LT binding to the origin, which nonenzymatically pries apart the dsDNA. Unlike the reported DNA melting mechanism for cellular CMG, this initial viral DNA melting allows annular LT hexamers to directly form around single DNA strands to create a double-hexameric complex ready for subsequent helicase activation and DNA replication.

### 6.2.1 Results

### Single-Molecule MCV LT Binding to its Origin DNA

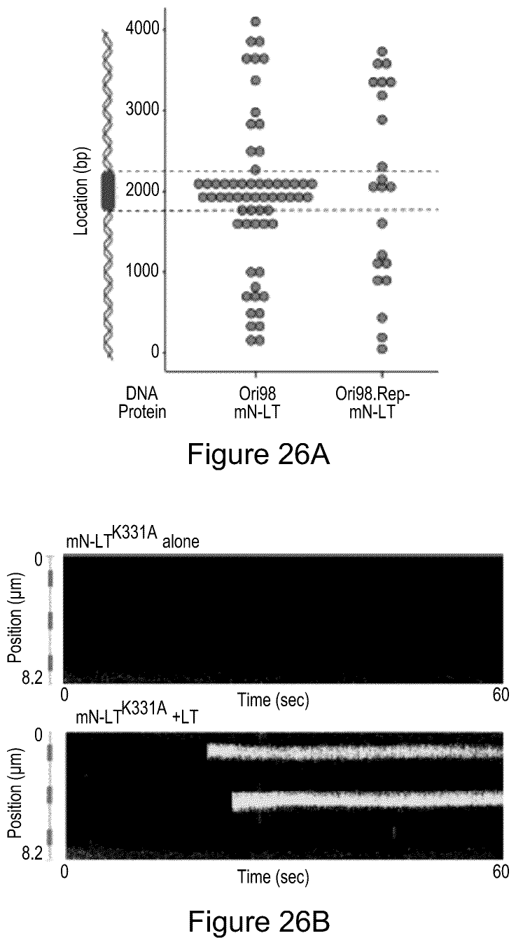

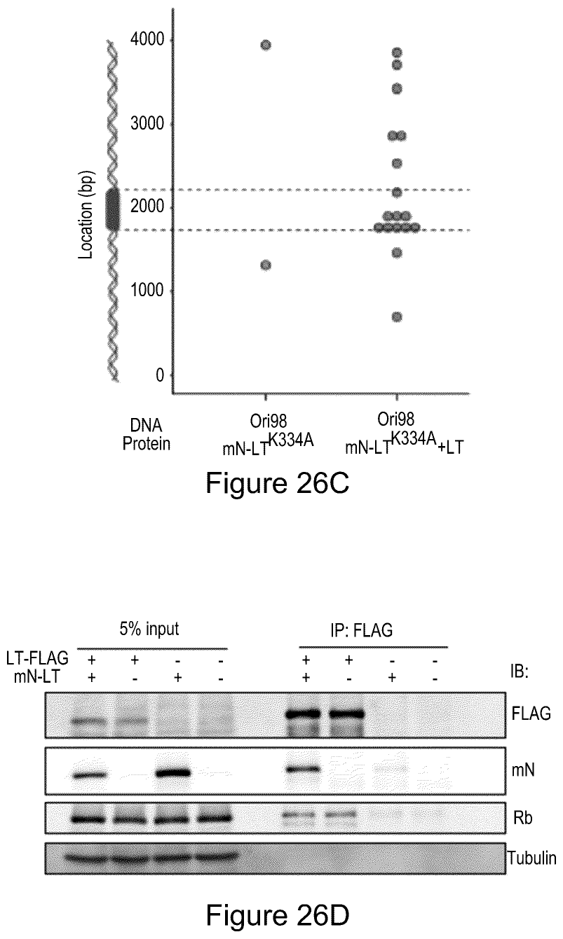

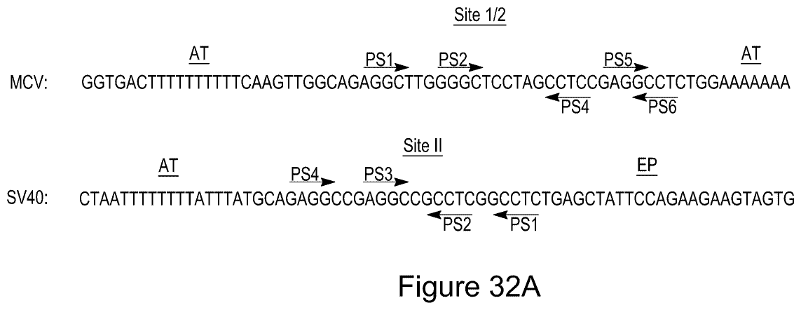

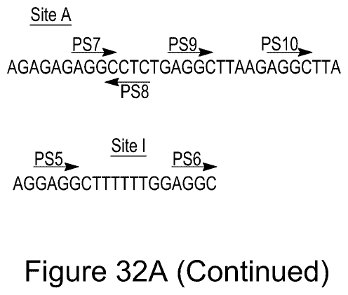

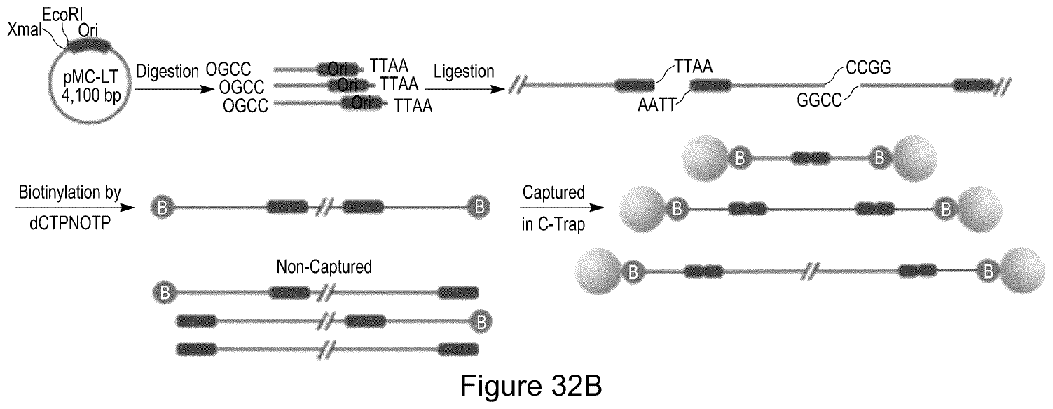

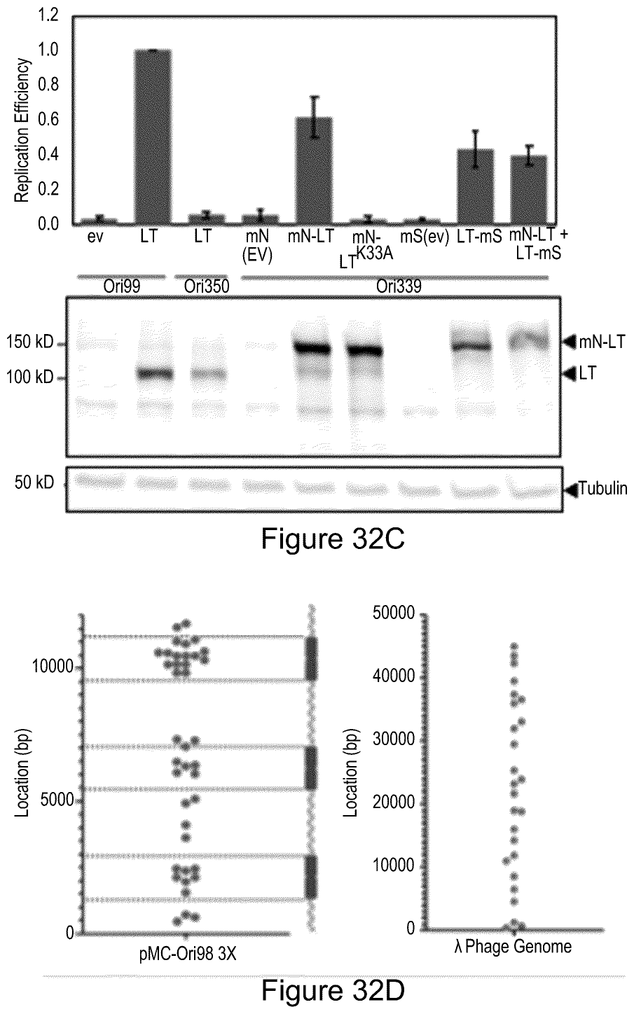

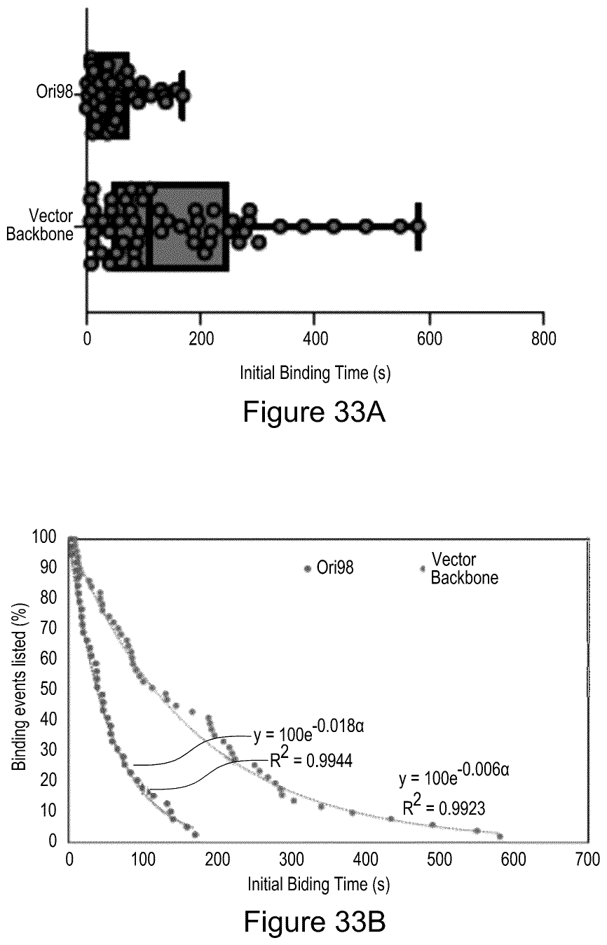

SMADNE was used to visualization in real-time viral origin assembly and DNA melting by MCV LT molecules. Ori98 was cloned into the pMC.BESPX vector, which was concatemerized (for observing multiple concurrent binding events in each experiment) and end biotinylated (FIG. 25C). A single DNA molecule was then captured between two streptavidin-coated beads and kept at 10 piconewton (pN) tension (FIG. 32B). Nuclear extracts from 293 cells³⁰ expressing fluorescent N-terminally tagged mNeonGreen LT (mN-LT) were flowed over the DNA in 1 mM ATP, 5 mM Mg²⁺ buffer at 25° C. [fluorescently tagged LT proteins were shown to retain replication competence in replicon assays (FIG. 32C)]. A representative example for specific mN-LT binding to Ori98 DNA is shown in FIG. 25D and Movie S1. The mN-LT on-rate constant (kon)³¹ and binding frequency were 47-fold and 15-fold higher, respectively, for Ori98 sites compared to the pMC.BESPX backbone sequence (FIG. 25E and FIGS. 33A and 33B). Similarly, mN-LT localized to wild-type MCV Ori98 sequences with ˜eightfold higher frequency per unit length of DNA than A phage genome DNA, which has 140 G (A/G) GGC pentad sites scattered across its genome (FIG. 32D). Capture of Ori98.Rep-DNA showed reduced mN-LT binding to levels not significantly greater than vector backbone sequence also confirming specificity of wild-type MCV origin recognition by LT in the C-Trap (FIG. 26A)

To demonstrate LT protein oligomerization, an alanine substitution mutation in the LT protein origin binding domain (OBD) at lysine 331 (mN-LTK331A) was introduced in the LT protein origin binding domain, which led to reduced LT-DNA binding (FIG. 26B). However, when untagged LT was flowed together with the mutated LT, the origin-specific DNA binding fluorescence was restored, indicating molecular multimerization on the origin (FIG. 26C). This multimerization was further confirmed by co-localization of fluorescently tagged LT proteins on Ori98. Importantly, LT protein multimerization could occur in solution and did not require MCV DNA, as shown in bulk immunoprecipitation and immunoblotting experiments in the absence of viral origin DNA (FIG. 26D).

### Origin DNA Melting by MCV LT at the Single Molecule Level

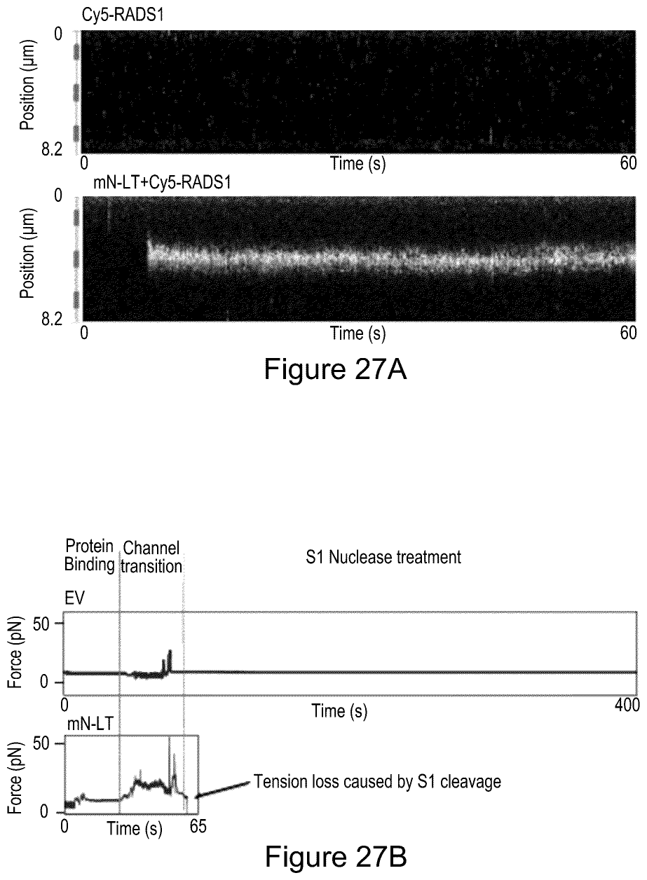

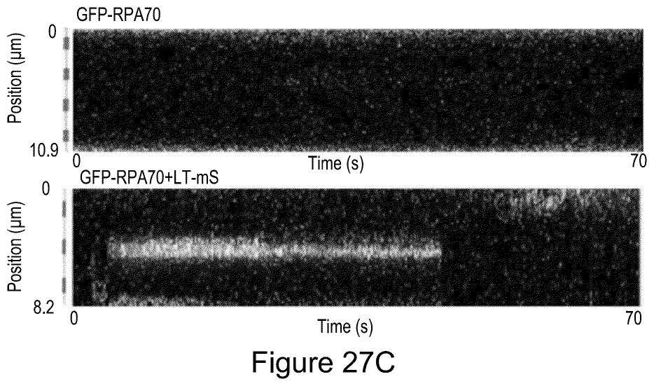

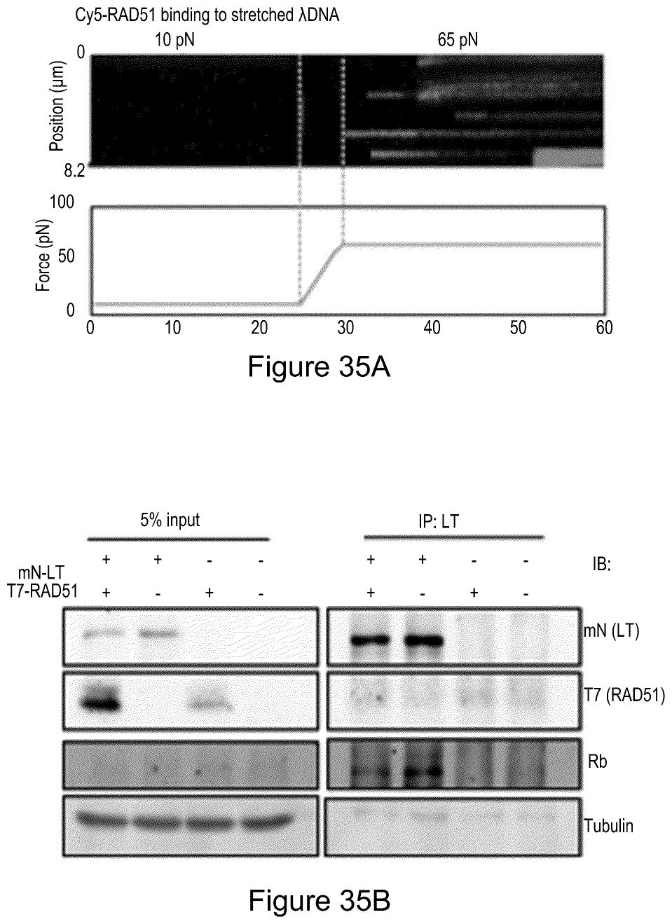

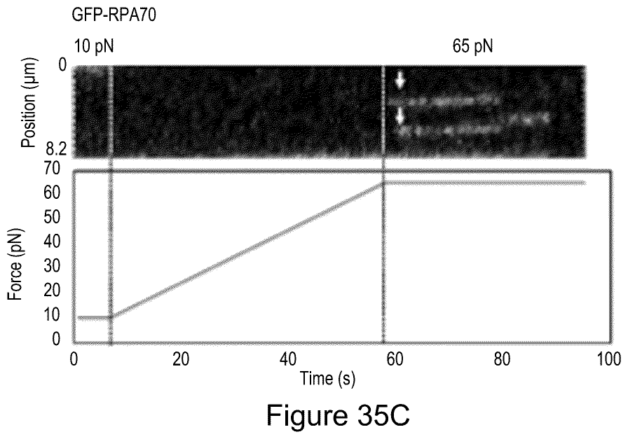

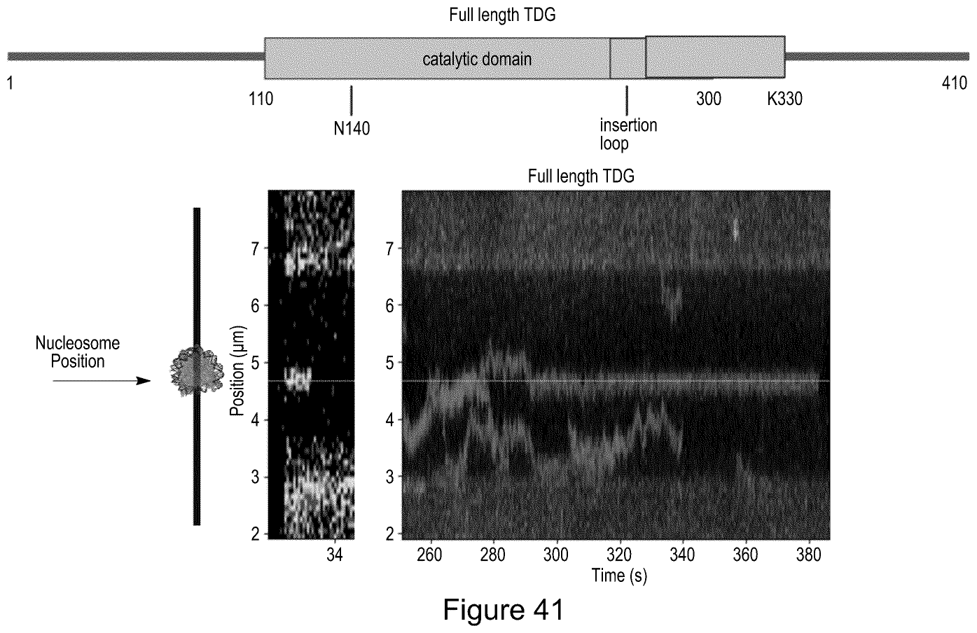

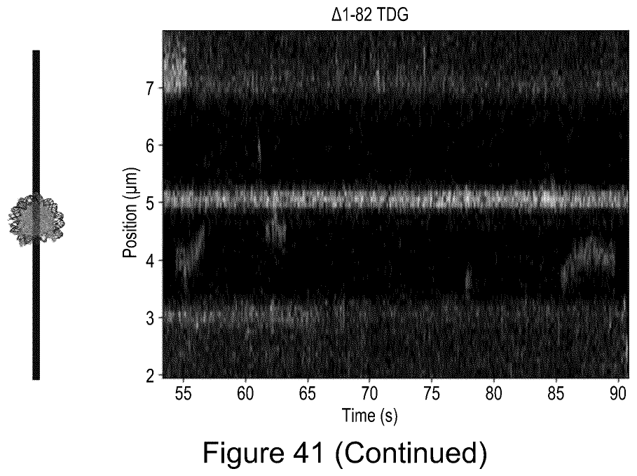

To determine the origin melting after LT binding, three independent approaches were tested. First, DNA cobinding by the ssDNA-binding protein RAD51^{33, 34} was examined in the presence or absence of mN-LT protein. To ensure that Cy5-RAD51 binding to ssDNA could be detected in the C-Trap, tethered dsDNA was stretched from 10 pN to 65 pN tension to generate local force-induced ssDNA regions³⁵, which then bound Cy5-RAD51 (FIG. 35A). Cy5-labeled RAD51 did not significantly interact with tethered Ori98 dsDNA alone (FIG. 41A, Top). When mN-LT was flowed in the same channel with Cy5-RAD51, Cy5-RAD51 bound to and colocalized with mN-LT (FIG. 27A, Bottom). mN-LT temporally assembled on DNA first, followed by Cy5-RAD51, in 72% (n, 81) of 112 dual-binding events. All remaining dual-binding events (n, 31) were concurrent. Cy5-RAD51 binding prior to mN-LT binding was not observed, and only rarely did Cy5-RAD51 bind DNA alone without mN-LT cobinding (twice during 30 min of monitoring for dual binding events). DNA tension (10 pN) did not appreciably affect DNA melting and mN-LT and Cy5-RAD51 cobinding similarly occurred in the absence of DNA tension. In contrast to LT-LT interaction, no direct protein-protein interaction between RAD51 and MCV LT was found by bulk coimmunoprecipitation (FIG. 35B).

Molecular DNA melting was assayed by cleavage of tethered DNA using the single strand-specific S1 nuclease. S1 cleaved mN-LT-bound DNA within 4 s after introduction into the flow cell whereas in the absence of LT, tethered dsDNA was not cleaved during 320 s of S1 exposure (FIG. 29B). Finally, GFP-labeled RPA7036, one of the three replication protein A (RPA) subunits^{37, 38}, colocalized with LT-mS on DNA but did not bind captured dsDNA in the absence of LT-mS (FIGS. 27C and 35C). Taken together, these experiments show that Cy5-RAD51 cobinding with mN-LT to DNA was a reliable marker for single-molecule dsDNA melting.

mN-LT Assembles as a Dodecamer on Ori98 DNA

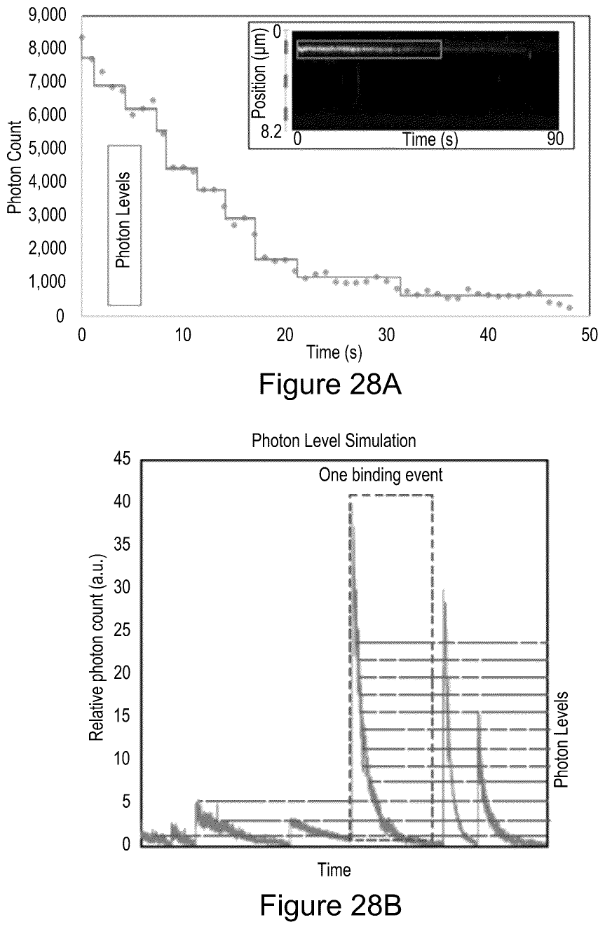

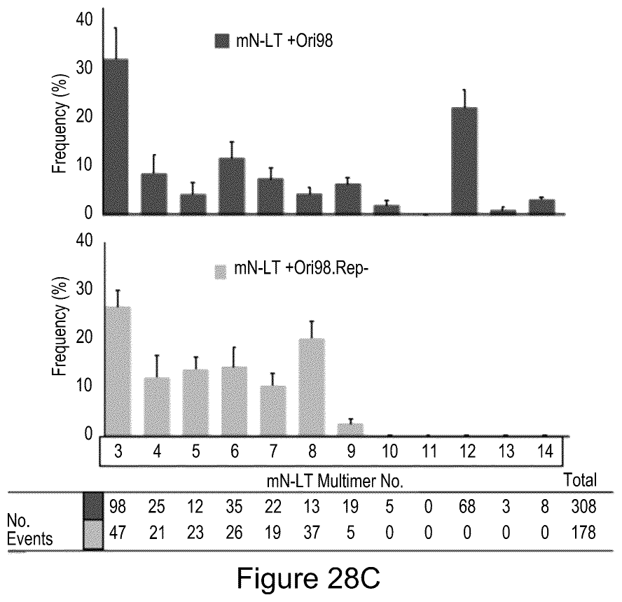

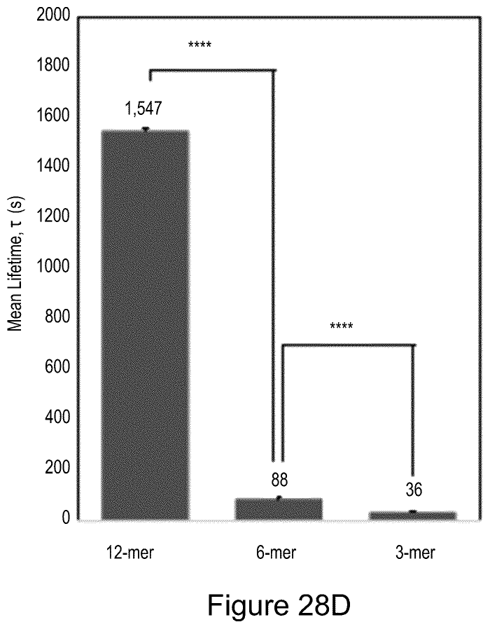

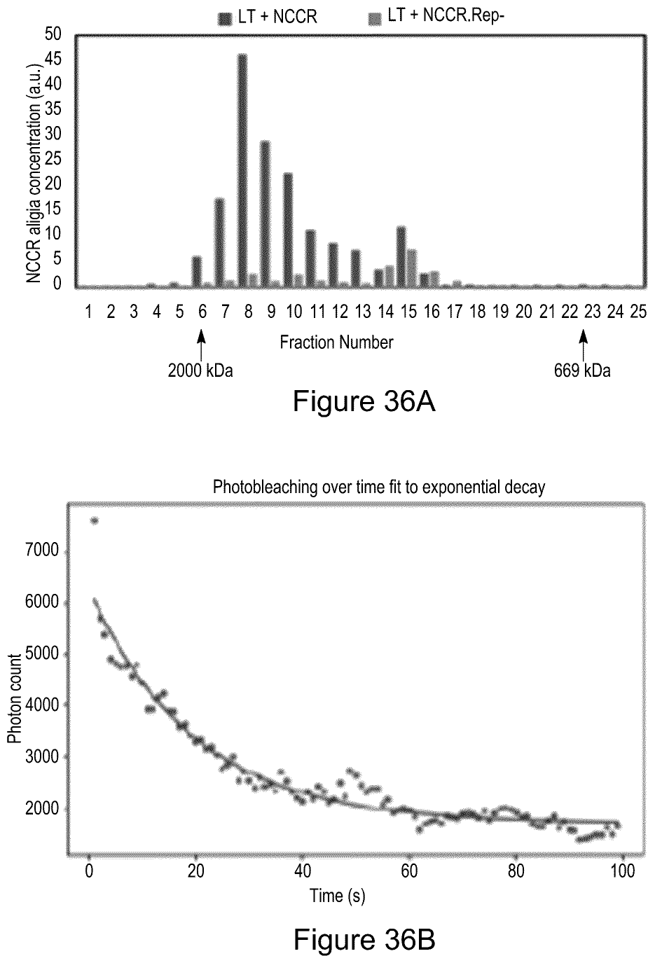

To quantitate molecular assembly of LT on DNA, a HMM simulation was used^{29, 39}. Based on the phenomenon that photobleaching causes equal, stepwise fluorescence decrements, fluorophore photo-oxidization was used to model the number of mN-LT molecules initially captured by DNA origins (FIGS. 28A and 28B). For technical reasons, the HHM could not reliably distinguish between monomer and dimer binding events. Therefore, these values were not included in the quantitative analysis. LT molecular assembly on Ori98 for 308 protein binding events, obtained from 30 captured DNAs, ranged from 3 to 14 mN-LT molecules, with notable maxima at 3-mer (32%) and 12-mer (22%) LT complexes (blue bars, FIG. 28C). Some configurations, such as 10 and 11-mer assemblies, were exceedingly rare, which may reflect rapid allosteric promotion to 12-mer complexes from these lower ordered assemblies. The dodecameric assembly most likely represented two separate hexamers (a double hexamer), and the term double hexamer is used below. Other 12-mer assemblies remain formally possible. Rare complexes greater than 12-mer may represent double-hexamer formation plus additional LT-origin binding at nonreplication site pentads, (e.g., PS5 or PS6)¹⁷. In contrast, when tumor-derived Ori98.Rep-DNA, having a single mutation in PS7, was substituted for Ori98, 12-mer assembly was not seen in 178 binding events (yellow bars, FIG. 28C). Maximum assembly on Ori98.Rep-reached only 6 to 8 mN-LT molecules, consistent with the origin having two separate hexamer nucleation sites, one at PS1, 2, and 4 and another at PS7. This is also supported by the reduced binding specificity seen in FIG. 26A, as well as bulk size-exclusion chromatography of nuclear lysates expressing untagged LT together with either 464 bp wild-type (WT) or Rep-NCCR DNA. Quantitative PCR revealed WT NCCR DNA eluted at higher molecular mass fractions than those eluting NCCR.Rep-DNA, consistent with higher order LT multimerization on the WT NCCR DNA (FIG. 36A).

### Double-Hexameric MCV LT Forms a Stable Complex on Origin DNA

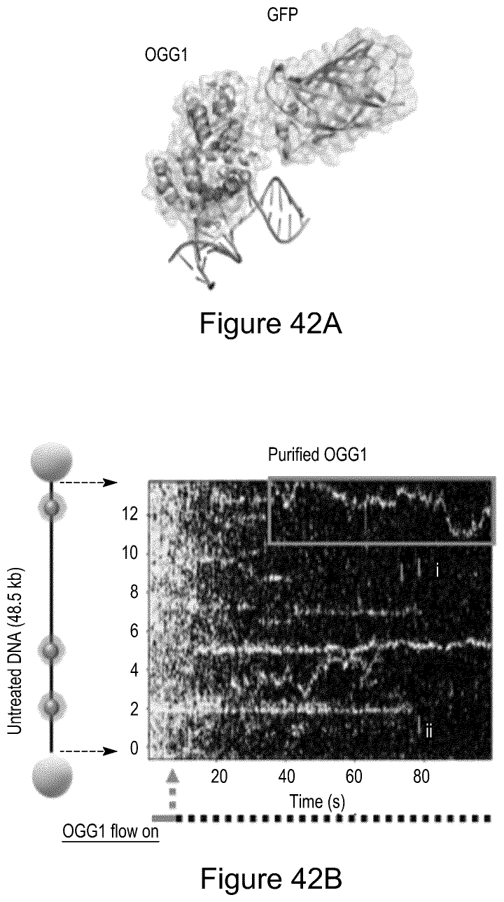

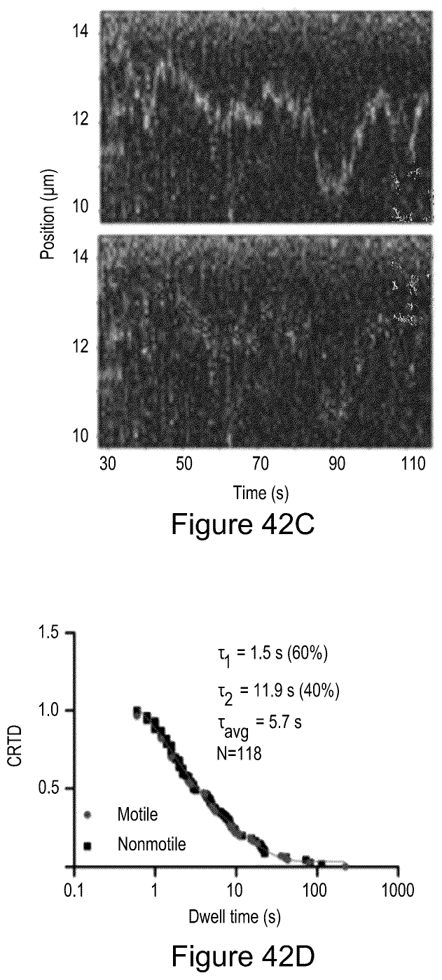

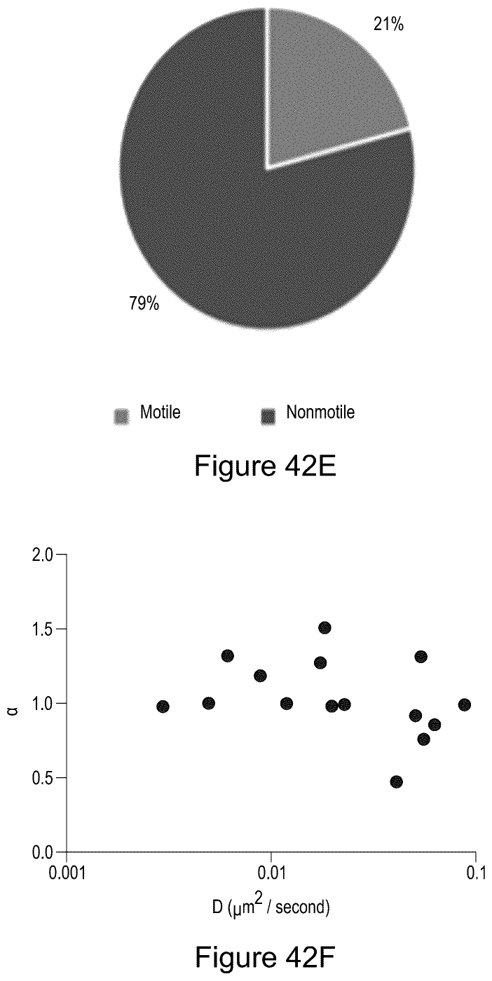

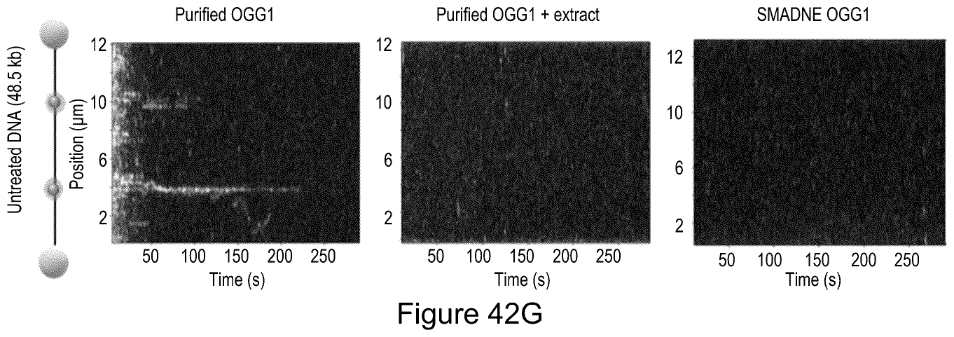

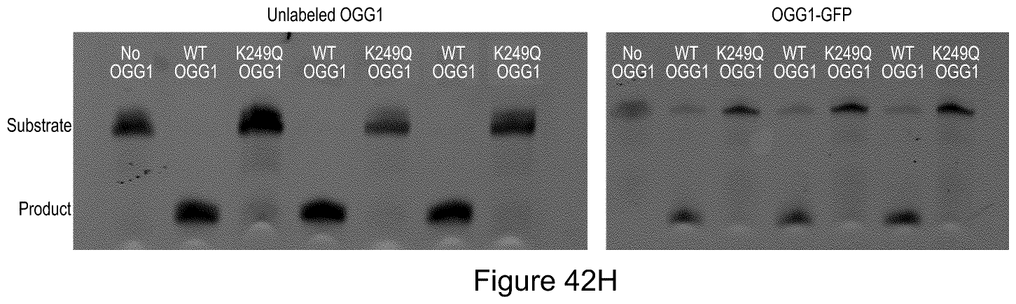

To determine the stability of mN-LT complexes on Ori98, the present disclosure estimated the mean lifetime (τ=l/koff) for mN-LT bound to DNA after correcting for photobleaching (t_{mN photobleaching}=33 s, FIG. 50B). The mean LT-DNA binding lifetime increased from 36 s to 88 s for 3-mer and 6-mer assemblies, respectively (FIG. 27D). Since this was performed under active-flow channel conditions, transient disassembly-reassembly was unlikely. In contrast, mN-LT 12-mer assemblies on origin DNA had calculated mean binding lifetimes >1,500 s or greater than 17 times the mean binding lifetime for a single hexamer (FIG. 42D).

MCV and SV40 LT Origin Melting does not Require an LT Hexamer

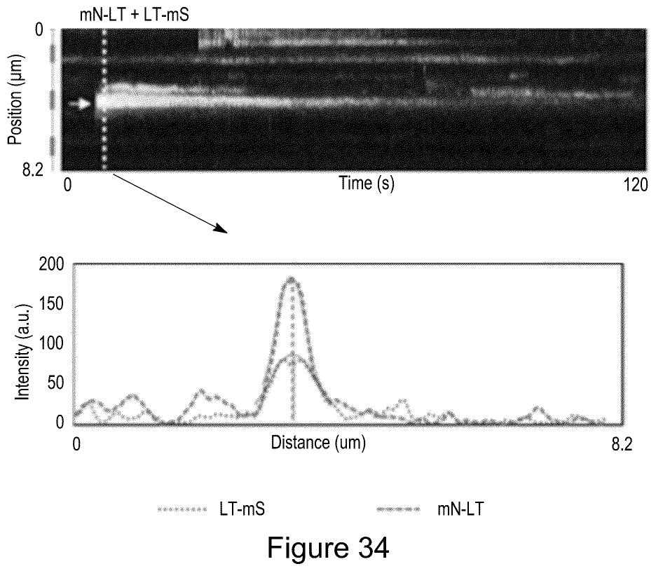

Ori98.Rep-did not form replication competent double-hexameric LT complexes, nevertheless, mN-LT recruited Cy5-RAD51 to Ori98.Rep-(FIG. 29A) as well as to Ori98 (FIG. 34A). Out of 34 mN-LT binding events on Ori98. Rep-, 26 (76%) were observed with Cy5-RAD51 cobinding. In the case of wild-type Ori98 DNA, Cy5-RAD51 binding and DNA melting was detected for subhexameric complexes (e.g., trimers). Bound Cy5-RAD51 fluorescence intensity increased linearly with the number of LT molecules coassembled on Ori98 (R2=0.86) with the shortest lag-time between initial mN-LT binding and subsequent Cy5-RAD51 binding (˜1 s) occurring for an LT double hexamer (FIG. 29B). LT-Cy5/RAD51 binding lag time was inversely related to the number of assembled LT molecules (e.g., ˜70 s for trimers; R2=0.95, FIG. 29B). Since RAD51 forms polymeric fibrils on ssDNA, Cy5-RAD51 fluorescence intensity is not a reliable measure of single-strand bubble size, but these data taken together are consistent with extensive DNA melting upon subdodecameric LT assembly.

When C-terminal GFP-tagged SV40 LT was flowed over MCV Ori98 DNA, only subhexameric SV40 LT binding was observed, and no SV40 LT hexamers or double hexamers were detected (FIG. 29C). All SV40 LT binding assemblies were trimeric (57%), tetrameric (27%), or pentameric (16%) in 92 binding events on 12 separate dsDNA molecules, consistent with the inability of SV40 LT to assemble a competent double-hexameric helicase on the MCV origin⁷. When the MCV origin was replaced by the SV40 LT origin in the pMC plasmid, however, SV40 LT was able to readily assemble as a dodecamer on its own origin (FIG. 29D). Despite being unable to replicate MCV DNA or even form a single hexamer on MCV origin, SV40 LT recruited Cy5-RAD51 to MCV Ori98 DNA (FIG. 29C), demonstrating that SV40 LT melts the MCV origin when binding alone. As with MCV LT on MCV origin, Cy5-RAD51 cobinding was observed for subdodecameric as well as dodecameric SV40 LT assemblies on the SV40 origin (FIG. 29D).

### MCV Origin DNA Melting Requires LT Multimerization but not the Viral Helicase Domain

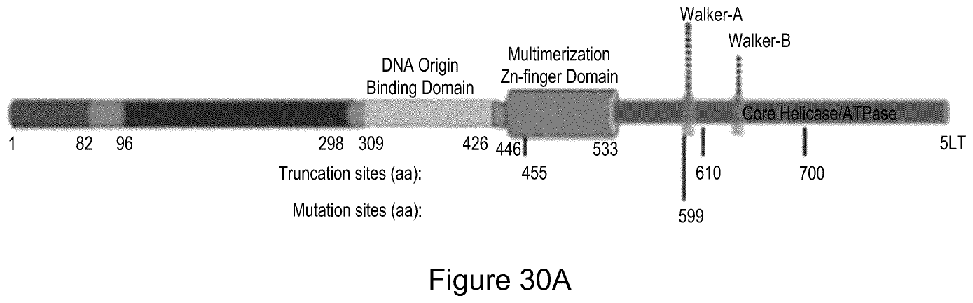

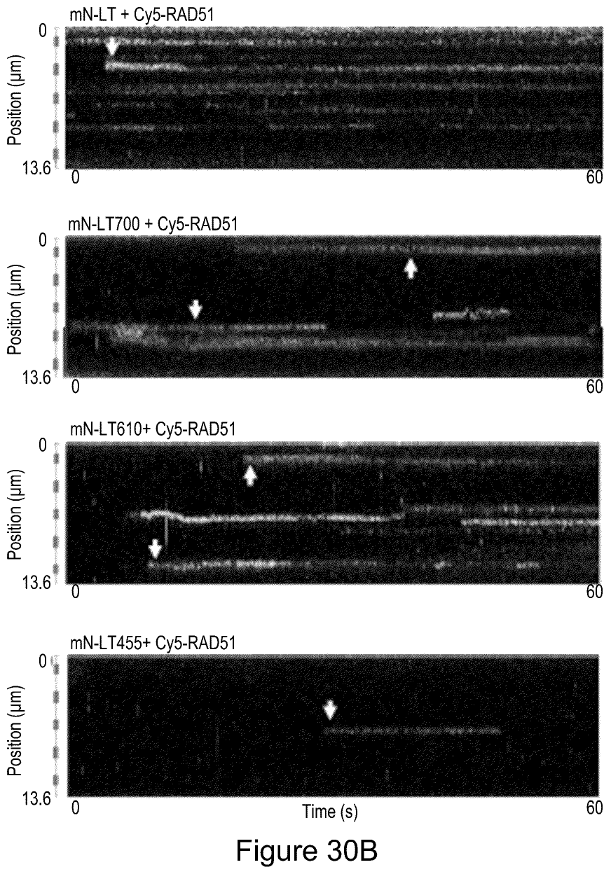

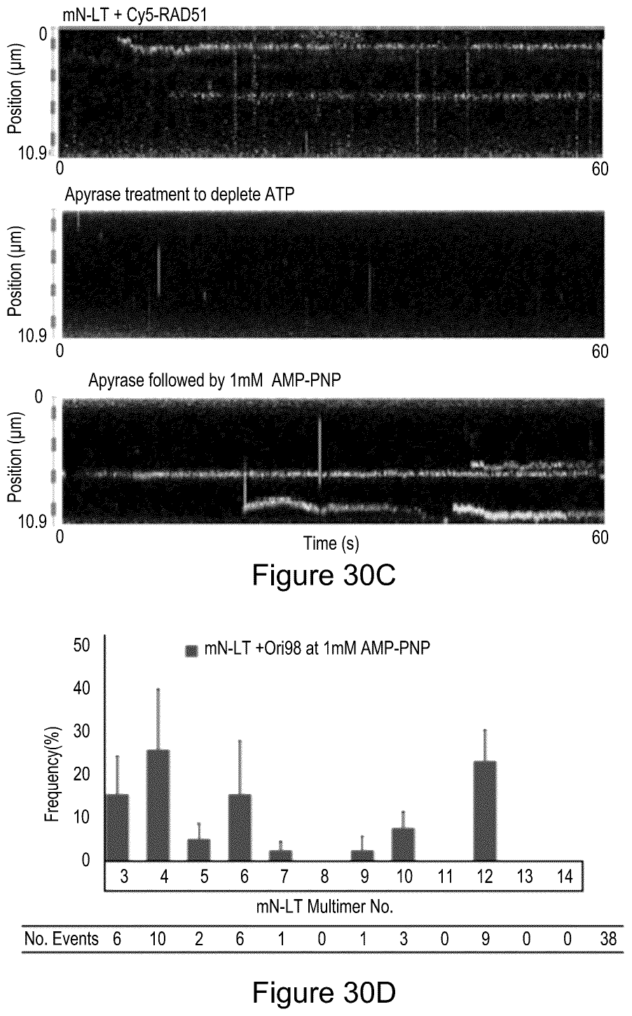

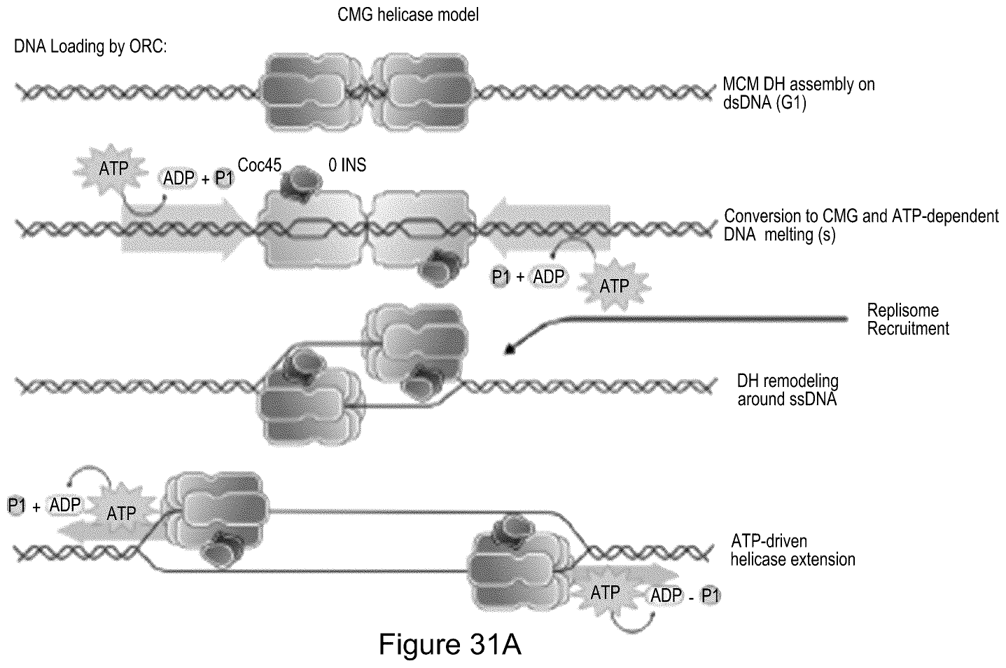

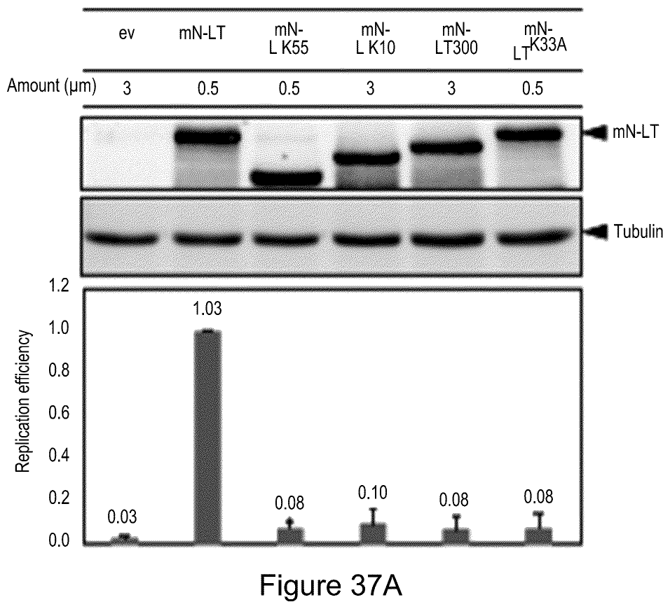

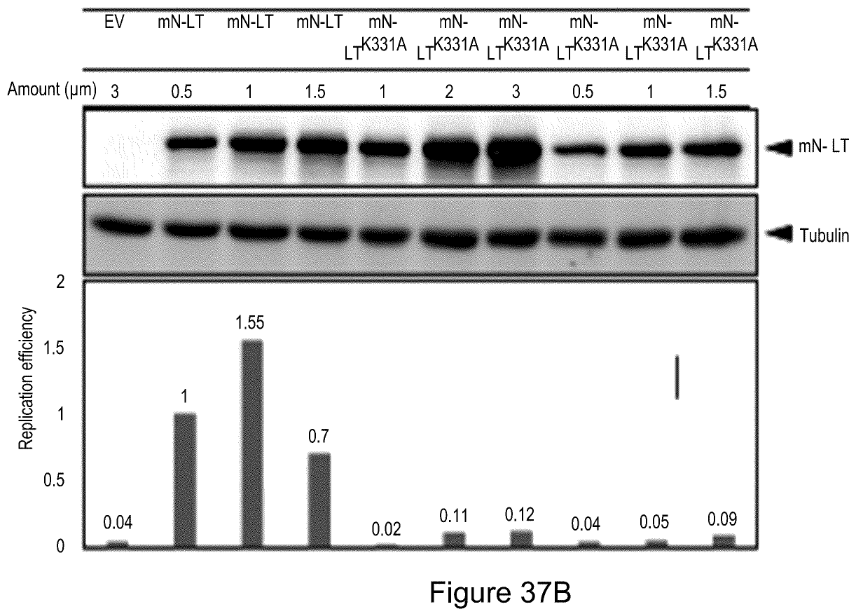

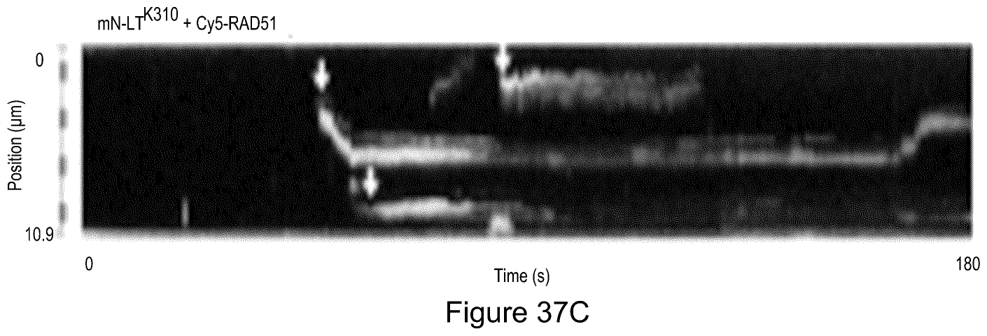

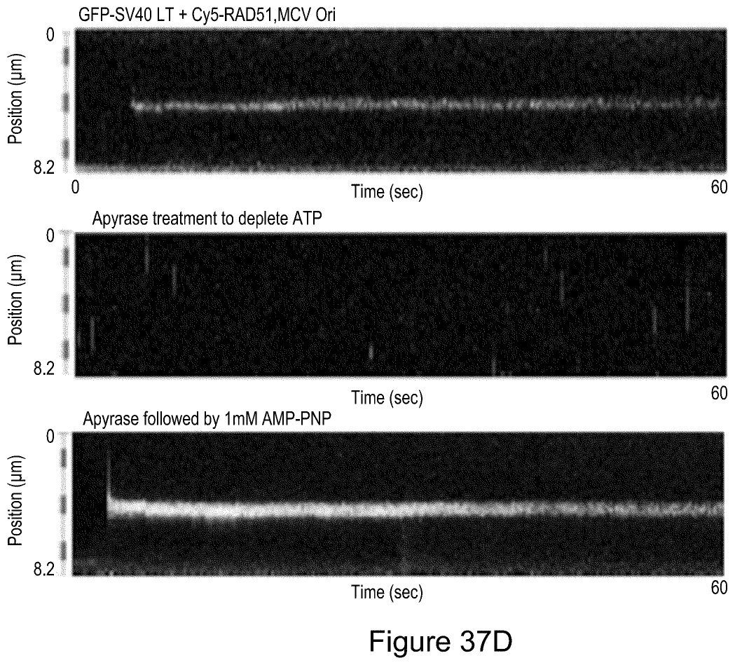

Dispensability of the MCV LT helicase function for initial DNA melting was demonstrated with successive C-terminal truncations of the 817 aa LT protein (FIG. 30A). These truncation mutants all abrogated replication when used in replicon assays (FIG. 37A). mN-LT700 lacks a critical cell growth-inhibitory domain⁴⁰ but retains canonical AAA+Walker A and B sites required for ATP binding and hydrolysis^{41, 42}. mN-LT610 retains the Walker A site but is deleted for the Walker B site. Both mN-LT700 and mN-LT610 bound origin MCV DNA and induced Cy5-RAD51 colocalization (FIG. 30B). A point mutation in the Walker A site (mN-LTK599R, FIG. 37B)^{9,41} also recruited Cy5-RAD51 (FIG. 37C). Unexpectedly, this mutant showed enhanced movement along the DNA (y axis of FIG. 37C), with the diffusion coefficient ranging from 0.05 to 0.4 μm²/s³⁰.

MCV LT C-terminally truncated at residue 455 (mN-LT455) corresponds to a tumor-derived (MCC339) mutant protein¹¹ that has an intact OBD but lacks the majority of the zinc-finger domain required for dimerization⁴³ as well as the helicase domain. This mutation only bound origin DNA as a monomer (FIG. 30B) and did not recruit Cy5-RAD51, consistent with LT multimerization being required for DNA melting and RAD51 Recruitment.

### MCV LT DNA Loading and Melting Requires ATP Binding but not ATP Hydrolysis

When mN-LT nuclear extracts were pretreated with apyrase, an ATP diphosphohydrolase, to deplete residual ATP from nuclear lysates, LT and Cy5-RAD51 binding to Ori98 DNA was eliminated (FIG. 30C). Binding and melting, however, was restored by addition of 1 mM nonhydrolyzable adenylyl-imidodiphosphate (AMP-PNP)⁴⁴. This is most consistent with ATP binding, but not enzymatic hydrolysis, being required for LT loading and initial origin melting. Notably, LT quantitation revealed that LT can assemble as a double hexamer (dodecamer) on origin DNA in the presence of AMP-PNP (FIG. 30D). Similar experiments using SV40 GFP-LT also revealed that SV40 LT/Cy5-RAD51 binding to the MCV origin was independent of ATP hydrolysis.

### 6.2.2 Discussion

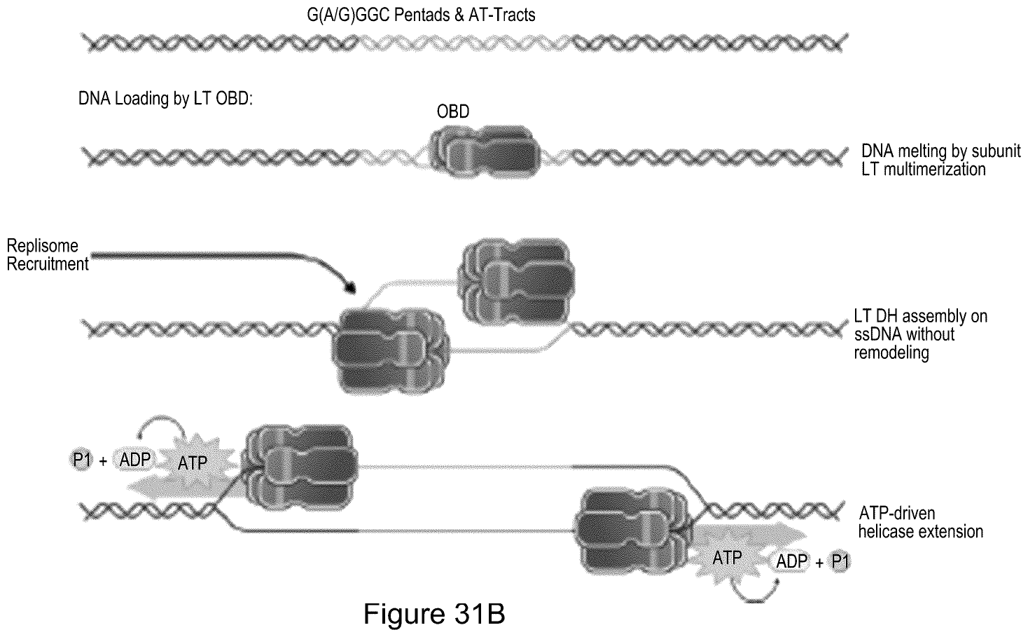

Results are most consistent with multimer MCV and SV40 LT, as small as a trimer, being able to nonenzymatically bind and pry open the dsDNA origin so that LT can directly form two hexamers (a double hexamer) around the ssDNA strands (FIG. 31A). This “strand invasion model” can occur if multimeric LT has a higher affinity for origin ssDNA than for dsDNA, as has been described for SV40 LT45, and LT's ssDNA affinity exceeds the local corresponding binding affinity of the complementary ssDNA strand. After the double hexamer is assembled onto complementary ssDNA strands, DNA unwinding and unzipping through helicase activity and ATP hydrolysis would be able to allow DNA polymerase processivity in replication. If MCV LT followed the same steps as the CMG origin melting model instead¹, MCV LT hexamers would have to first form as annuli around dsDNA, initiate helicase shearing even without two complete hexamers, and then remodel onto ssDNA without use of ATP hydrolysis, which is energetically unlikely to happen.

It is not surprising for viral helicases to have a molecular mechanism for origin melting that differs from cellular CMG since viruses initiate multiple rounds of replication during each cell cycle. CMG is preloaded by ORC onto dsDNA eukaryotic origins to assure complete replication of the genome, and thus, CMG double hexamers must wait until they are fully loaded and licensed before initiating origin melting. The LT strand invasion model may explain how these viruses can rapidly reinitiate origin melting on newly synthesized dsDNA strands to iteratively amplify viral genomes in a single cell cycle. While MCV and SV40 LT proteins have similarities, caution is needed to assume that both viral proteins have identical replication mechanisms. For example, initial SV40 LT origin melting is reported to occur at an early palindrome region that is not present in the MCV origin⁴⁶. Instead, MCV origin has an AT-rich tract (FIG. 32A) between PS6 and PS7 that may allow melting during LT assembly on the origin sequence. Despite having different origin sequences, these two viral LT proteins are similar enough to each other to initiate melting, but not replication, of the MCV origin (FIG. 29C)

There are several key pieces of data in the single-molecule experiments that support the MCV LT direct strand invasion mechanism rather than helicase-dependent compression of dsDNA between the two hexamers to initially melt DNA. Measurements of kon and koff rates allowed stability to be determined for different configurations of LT-DNA (FIG. 33 and FIG. 28D) and support the hypothesis that LT hexamers melt and directly surround ssDNA rather than first assembling around dsDNA. A free complementary ssDNA strand would compete to eject a single hexamer in the flow experiments making it more unstable than a double hexamer in which both strands are occupied. Further, the kinetics for initial RAD51 cobinding with partially multimerized (6-mer through 10-mer) MCV LT approached dodecameric LT rates, consistent with partial multimers and dodecamers of LT opening up similar-sized ssDNA bubbles (FIGS. 29B, Top). Additionally, double-hexamer loading and melting occurred in the absence of hydrolyzable ATP (FIGS. 30C and 30D), which is inconsistent with helicase activity being responsible for shearing dsDNA. Finally, MCV LT mutations eliminated functional helicase activity but retained dsDNA origin melting. The results for SV40 LT shown here (FIG. 29C and FIG. 37), in which no SV40 hexamers are formed on MCV origin, and studies using bulk KMnO4 oxidation assays on SV40 origin^{25,26}, suggest viral LT multimers pry apart origin dsDNA rather than using a helicase mediated shearing mechanism.

Single-molecule microscopy complements X-ray crystallography and cryo-EM studies in determining the functions for LT structural features. The requirement for ATP binding in LT assembly on dsDNA (FIG. 30C), for example, may be due to anchoring interactions of the AAA+domain on the dsDNA minor groove⁴⁷. A recent single molecule study for activated yeast CMG revealed that nucleotide binding anchors CMG to DNA to prevent bidirectional diffusion of the helicase along dsDNA⁴⁸. This can explain the diffusion along DNA for the MCV LT Walker A box mutant (mN-LTK599R, FIG. 37C). This mutant still generated Cy5-RAD51 cobinding, which tracked with the LT complex. The movement is most likely a result of physical flow conditions in the experiment, wherein LT hexamers are nudged along the DNA by the channel flow to unzip dsDNA. The minimum number of assembled LT subunits needed for MCV melting is not addressed by the study. Monomeric MCV mN-LT455 was incapable of melting origin DNA, in agreement with structure studies showing that monomer MCV OBD binding to the origin major groove at PS1 and PS2 causes a 5° bend in the DNA but not strand separation²⁷. For SV40, multimeric LT binding causes local distortion and melting of origin DNA^{25, 26}, but dimeric LT alone is not capable of melting origin DNA49. Structural studies of bovine papillomavirus E1 (a distantly related virus) suggest that E1 trimerization is sufficient to initiate viral strand separation 50 and is consistent with trimeric MCV LT (FIG. 29B) being able to initiate detectable melting in at least a fraction of bound DNAs.

In addition to binding to origin sequences, MCV LT and RAD51 can also bind to nonorigin DNA sequences, most likely at single G (A/G) GGC pentad sequences. This binding is not expected to allow adventitious replication but could promote single strand breaks if the bound LT persistently melts dsDNA. DNA damage responses due to LT expression—as well as expression of the replication accessory MCV small T protein inhibitor of anaphase-promoting complex/cyclosome⁵¹, might halt host cell DNA replication⁶, but not viral replication, thereby shifting cellular replication resources to the virus⁴⁶. It is not known whether MCV LT is inherently mutagenic, but both SV40 and MCV LT have been reported to induce cellular DNA damage responses independent of oncoprotein domains^{52,53}. Whether cellular DNA damage from MCV LT expression might contribute to clonal viral integration is unknown.

This study focused only on the initial steps in origin melting since directed LT movement in the DNA axis (expected for in situ DNA helicase processivity) was rarely seen on the kymographs in experiments performed at 25° C. Dynamic study complements static atomic resolution X-ray crystallography (54) and cryoelectron microscopy structural studies^{(23, 27)}, yet generates an unexpected model for viral DNA replication initiation. Use of nuclear extracts was particularly critical to these experiments, however, unmeasured, nonfluorescent cellular replication/repair proteins may also affect MCV DNA melting and should be considered. Extension of these single-molecule experiments to chromatinized DNA or by achieving in situ helicase activity will provide important additional information on events controlling replication of this human tumor virus.

### 6.2.3 Materials and Methods

### Cell Lines

293 cells (ATCC) were maintained in Dulbecco's modified Eagle medium (ThermoFisher) supplemented with 10% fetal bovine serum (FBS), in a 37° C. and 5% CO2 incubator.

### Plasmid Mutagenesis

mN-LT plasmid was constructed by inserting codon optimized MCPyV LT sequence to the C terminus of pmNeongreen-C1, using XhoI and BamHI cutting sites (a 6 a.a. GSTGSR nonspecific protein tag was appended to the C terminus of LT due to cloning strategy). To generate the pMC-Ori98 plasmid, a fragment of Ori98 sequence was produced through PCR from pMC-MCV and then inserted into the pMC.BESPX backbone using EcoRI and BamHI sites. All point mutations (mN-LTK331A, mN-LTK599R etc.) were produced using QuikChange Lightning Site-Directed Mutagenesis Kit (Agilent) following the manufacturer's protocol. All Chang-Moore (CM) laboratory plasmid numbers are listed in Table 6.

TABLE 6
List and description of plasmid constructs.

| | | | Chang- | |
|---|---|---|---|---|
| | | | Moore | Chang-Moore |
| Construct | Function | Parental Vector | Plasmid | Plasmid Name |
| pcDNA6 | Control Vector | pcDNA6.V5- | 2892 | pcDNA6 modified |
| pcDNA6-LT | Expresses MCV codon- | pcDNA6 | 2956 | pcDNA6.MCV LTco |
| | optimized LT in mammalian | | | |
| pmNeonGreen-C1 | Expresses mNeonGreen in | N/A | 4732 | pmNeonGreen-C1 |
| | mammalian cells | | | |
| pmNeonGreen-N1 | Expresses mNeonGreen in | N/A | 4733 | pmNeonGreen-N1 |
| | mammalian cells | | | |
| mN-LT | Expresses mNeonGreen- | pmNeonGreen-C1 | 4879 | mNeonGreen- |
| | fused codon-optimized LT in | | | MCV LTco |
| | mammalian cells | | | |
| mN-LTK331A | The origin binding domain | pmNeonGreen-C1 | 4757 | mNeonGreen- |
| | mutant for binding deficiency at | | | MCV LTco |
| pmScarlet-C1 | Expresses mScarlet in | pmScarlet-C1 | 4738 | pmScarlet-C1 |
| | mammalian cells | | | |
| LT-mS | Expresses mScarlet-fused codon- | pmScarlet-C1 | 4780 | MCV LTco-mScarlet |
| | optimized LT in mammalian cells | | | |
| mS-LT | Expresses mScarlet-fused codon- | pmScarlet-C1 | 4780 | mScarlet-MCV LTco |
| | optimized LT in mammalian cells | | | |
| pMC-Ori98 | Amplifies Ori97 sites in | pMC.BESPX | 4883 | pMC.BESPX- |
| | pMC.BESPX backbone in | | | MCV-97bp |
| | bacteria for self-ligation and | | | |
| | biotinylation to produce dsDNA | | | |
| pMC-Ori98.Rep- | Amplifies Ori97(rep-) sites in | pMC.BESPX | 4884 | pMC.BESPX- |
| | pMC.BESPX backbone in | | | MCV-97bp (rep- |
| | bacteria for self-ligation and | | | |
| | biotinylation to produce | | | |
| pMC-NCCR | Amplifies NCCR (464bp) in | pMC.BESPX | 4890 | pMC.BESPX-NCCR |
| | pMC.BESPX backbone in | | | |
| | bacteria for self-ligation and | | | |
| | biotinylation to produce | | | |
| pEGFP-N1 | Expresses EGFP in mammalian | N/A | 2437 | pEGFP-N1 |
| | cells | | | |
| LT-FLAG | Expresses LT with flag tags for | N/A | 4754 | MCV LT-FLAG |
| | immunoprecipitation | | | |
| pQCXIP-GFP- | Expresses GFP-fused RPA70 | N/A | 5039 | pQCXIP-GFP- |
| RPA70 | | | | RPA70 |
| mN-LT455 | Expresses mNeonGreen- | pmNeonGreen-C1 | 5001 | mNeonGreen-MCV |
| | fused truncated codon- | | | LT (339-trunc) |
| | optimized LT without | | | |
| mN-LT610 | Expresses mNeonGreen- | pmNeonGreen-C1 | 5002 | mNeonGreen-MCV |
| | fused codon-optimized LT in | | | LT (610-trunc) |
| | mammalian cells with | | | |
| | helicase domain partially | | | |
| mN-LT700 | Expresses mNeonGreen- | pmNeonGreen-C1 | 5003 | mNeonGreen-MCV |
| | fused codon-optimized LT in | | | LT (700-trunc) |
| | mammalian cells with | | | |
| | helicase domain partially | | | |
| mN-LTK599R | Expresses mNeonGreen- | pmNeonGreen-C1 | 4972 | mNeonGreen-MCV |
| | fused codon-optimized LT in | | | LT (K599R) |
| | mammalian cells with | | | |
| | mutation to the walker A site | | | |
| GFP-SV40 LT | Expresses GFP-fused SV40 LT in | N/A | 2823 | GFP-SV40 LT |
| | mammalian cells | | | |
| pCH1-RAD51 | Expresses T7 tagged RAD51 in | N/A | 5134 | pCH1-RAD51 |
| | mammalian cells | | | |

### Origin Replication Assay

293 cells were seeded in 6-well plates and transfected with appropriate sample plasmid combinations to equal 1 μg total plasmid using Lipofectamine 2000 (ThermoFisher). At 48 h post-transfection, cells were collected for DNA and protein extraction. Total genomic DNA was purified from cells using DNeasy Blood and Tissue Kit (Qiagen). To linearize the replicated Ori98 DNA and remove transfected bacterial DNA, 1.25 μg of total genomic DNA was digested overnight using BamHI and DpnI.

### Quantitation of Replication by Quantitative Real-Time PCR

After overnight digestion of DNA from harvested cells, qPCR was performed using PowerUp™ SYBR™ Green Master Mix (ThermoFisher) with 5 ng DNA and Ori98 primers Fw: 5′-GCCGCCAAGGATCTGATG-3′ and Rev: 5′-CTGCGCAAGGAACGCCCGTCG-3′, with GAPDH primers: Fw: 5′-TGTGTCCCTCAATATGGTCCTGT-C-3′ and Rev: 5′-ATGGTGGTGAAGACGCCAGT-3′ as the endogenous control. Using a QuantStudio™ three Real-Time PCR Machine (ThermoFisher) and the ΔΔCT comparative method, threshold cycle (CT) values were used to calculate relative DNA replication levels, normalized to GAPDH levels.

### Immunoblotting

Total protein was extracted from transfected cells using RIPA Lysis Buffer (150 mM NaCl, 1% NP-40, 0.5% DOX, 0.1% SDS, and 50 mM Tris-HCl, pH 7.4) and protease inhibitors (0.2 mM Vanadate, 0.3 mM PMSF, 1 mg/mL Leupeptin, 1 mg/mL Pepstatin A, and 1 mg/mL Aprotinin). Samples were then sonicated with Fisherbrand™ Model 505 Sonic Dismembrator (ThermoFisher) at 20% Amp 4× for 5 s each on ice. 2× Laemmli loading buffer (65.8 mM Tris-HCl pH 6.8, 26.3% glycerol, 2.1% SDS, and 0.01% bromophenol blue, 10% 2-mercaptoethanol were added to samples which were then separated by SDS-PAGE and transferred to a nitrocellulose membrane. Membranes were incubated with primary mouse monoclonal antibody to MCV LT (CM2B4) overnight at 4° C., followed by incubation with IRD800 conjugated goat anti-mouse secondary antibody (LI-COR Biotechnology) diluted 1:10,000 and Rhodamine conjugated anti-tubulin antibody (Bio-Rad) diluted 1:10,000 for 1 h at room temperature. A ChemiDoc™ MP Imaging system (Bio-Rad) was used to detect signals.

### Coimmunoprecipitation

293 cells were cotransfected with 1 ug each plasmid (LT-FLAG and mN-LT; mN-LT and T7-RAD51) with Lipofectamine 2000 (ThermoFisher) for 48 h. Lysates were precleared with Protein A/G PLUS-agarose beads (Santa Cruz) and incubated with antibody overnight at 4° C., then with protein A/G PLUS-agarose beads for 3 h at 4° C. The beads were then washed twice with IP buffer (50 mM Tris pH7.4, 150 mM NaCl) and twice with LiCl buffer (500 mM LiCl 50 mM Tris pH7.4). Beads were boiled in 50 μL SDS loading dye. 15 μL of sample was run on 10% acrylamide gel, transferred to nitrocellulose, blocked in 5% milk, incubated with antibody at 4° C. overnight, washed, and incubated with secondary antibody at room temperature for 1 h. Blots were imaged on a ChemiDoc™ MP Imaging system (Bio-Rad). Antibodies: for LT-FLAG and mN-LT, IP: Mouse anti-FLAG (Sigma) 1 μg; IB: Rabbit anti-FLAG (Sigma) 1:1,000, Mouse anti-mNeon (Chromotek) 1:1,000, Mouse anti-Rb (Cell Signaling) 1:1,000; for mN-LT and T7-RAD51, IP: CM2B4 anti-LT 1 μg; IB: Mouse anti-mNeon (Chromotek) 1:1,000, Mouse anti-T7 (Novagen) 1:3,000, Mouse anti-Rb (Cell Signaling) 1:1,000.

### Size-Exclusion Chromatography

293 cells were transfected with pcDNA6-LT, and nuclear extracts were prepared 48 h after transfection as described in SMADNE method below. 150 μL of nuclear extracts were added to an equal volume of 2× reaction buffer (50 mM Tris-acetate, 20 mM magnesium acetate, 100 mM potassium acetate, 0.2 mM EDTA, 4 mM TCEP, 2 mM ATP, and 6 mM DTT) and incubated at 37° C. for 1 h. Diluted nuclear extracts were loaded onto a Superose 6 10/300 GL column and eluted with BC150 buffer (20 mM HEPES pH 7.9, 150 mM KCl, 0.2 mM EDTA, 10% glycerol, 1 mM DTT, and 0.5 mM PMSF), and 250 μL fractions were collected. 100 μL of each fraction was trichloroacetic acid (TCA)-precipitated and boiled in 25 μL of 2× Laemmli loading buffer. 20 μL of each sample was loaded on a 10% SDS-gel and transferred to a nitrocellulose membrane at 30V overnight at 4° C. Membranes were treated with SuperSignal western blot enhancer (Thermo) according to the manufacturer's protocol and then incubated with primary antibody (CM2B4, 1:1,000 dilution) overnight followed by incubation with secondary antibody (goat anti-mouse-IR800, 1:10,000 dilution) for 1 h at room temperature. Images were taken with ChemiDoc™ MP Imaging system (Bio-Rad). Quantitative PCR was applied using SYBR Green Master buffer (ThermoFisher) and Primers: FW: 5′-ATCGGGATCCGGTGACTTTTTTTTTTCAAGTTG-3′ and Rev: 5′-ATCGGAATTCTAAGCCTCTTAAGCCTCAGAG-3′ to quantify NCCR oligo DNA copies in each sample. Thermal cycling was performed on a QuantStudio™ three Real-Time PCR machine. Threshold cycle (CT) values were used to calculate relative NCCR oligo DNA abundance.

### Smadne

Following the SMADNE protocol³⁰, 293 cells at 70% confluency were transfected with 2 μg of plasmid (e.g. mN-LT, LT-mS, or sT-GFP) and 2 μL of Lipofectamine 2,000 (Thermo Fisher) in six-well plates. Cells were collected for nuclear extract preparation 48 h after transfection using the NE-PER™ Nuclear and Cytoplasmic Extraction Reagents kit (ThermoFisher) to prepare 50 μL of nuclear extract per well. Immediately prior to single-molecular experiments, nuclear extracts were diluted in reaction buffer (25 mN Tris-acetate, 10 mM magnesium acetate, 50 mM potassium acetate, 0.1 mM EDTA, 2 mM TCEP, 1 mM ATP, and 3 mM DTT) at 1:100 ratio (denoted as 1×).

### Linear Biotinylated DNA Substrate Preparation

FIGS. 25B and 32A, show the schematic for multimeric Ori98 biotinylated DNA preparation. First, 2 μg of pMC.Ori98 plasmid was digested with XmaI and EcoRI-HF (NEB) overnight and then column purified using the NucleoSpin Gel and PCR Clean-up mini Kit (Macherey-Nagel). The resulting linear DNA was self-ligated using T4 ligase (NEB) for 48 h and column purified again, creating randomly multimerized pMC-Ori98 with 5′-GGCC and/or 5′-AATT overhangs. Then, the 5′-GGCC overhangs were filled-in with 10 mM biotin-14-dCTP (and 10 mM biotin-11-dGTP (AAT Bioquest) using 10U DNA Polymerase I Klenow Fragment (NEB) for 1 h at 37° C. After a final column purification, DNA was stored in 0.1×TE buffer and diluted 1:250 in 1× phosphate buffered saline (PBS) for use.

### Optical Tweezer-Fluorescence Microscope

Optical Tweezer-Fluorescence Microscope (C-Trap, LUMICKS) with triple-color confocal fluorescence microscope and dual-trap laser optical tweezers was used in single-molecule experiments and has successfully been used to characterize nuclear extracts³⁰. The instrument contains five microfluidic channels combined into one chamber (FIG. 25A). Polystyrene beads (Spherotech, IL) coated with streptavidin at a diameter of 4.5 to 4.9 μm were flowed into channel 1 and captured by two optical tweezers with a stiffness of 0.3 pN/nm. The beads were then moved by optical traps to channel 2 to capture biotin-conjugated linear dsDNA. The length of tethered DNAs was quantified by the force-distance curve and fit into a worm-like chain (WLC) model to verify presence of a single DNA tether in channel 3. All channels were flowed at 0.2 bar to maintain laminar flow. Channels 4 and 5 were loaded with nuclear extracts diluted in reaction buffers. For the mN-LT binding assay, channel 4 was loaded with nuclear extract of mN-LT transfected 293 cells. Cy5-RAD51/GFP-RPA70 was mixed with mN-LT immediately before loading into channel 4. For S1 nuclease assays (see details below), mN-LT was loaded to channel 4, and S1 nuclease was loaded to channel 5. 2D scanning images and kymographs were taken in these two channels. When DNA-tethered beads were moved to these channels, protein-DNA binding events were recorded at a DNA tension of 10 pN, unless otherwise specified. For high protein binding efficiency, the flow pressure was adjusted to 0.03 bar in channels 3 and 4 while images were taken. mNeongreen and GFP fluorophores were excited by laser at 488 nm and emission was collected in a 500-550-nm band-pass filter. mScarlet fluorophore was excited at 532 nm, and emission was collected in a 575 to 625 nm band-pass filter. All data were collected with a 1.2 numerical aperture 60× water immersion. Kymographs were generated via a ID scan through the center of the two beads, at pixel size=50 nm, pixel scanning time=0.1 ms, and line scanning time=0.1 s. 2D scanning was performed at a focal plane that passes the center of the two beads, with frame rate=2.0 s/frame.

### Data Extraction

Kymographs of protein-DNA binding were taken and then analyzed by LUMICKS custom codes, and the line tracking of each fluorophore over time was performed based on a Gaussian fit over the signal intensity and connected over time. Visual aids were performed to ensure that each tracking result was continuous and clear. Instantaneous events (<5 s) were discarded since they might represent unstable protein attaching temporarily to DNA. The graphical user interface (GUI) allowed for quantitation and extraction of each event start/end time, event location tracking, photon count of the event over time, and tension applied to the DNA. Kymographs were generated from LUMICKS Lakeview software and exported as PNG files. Since the software showed the 500 to 550-nm channel in blue, all kymographs containing this channel were further imported to ImageJ to pseudocolor the 500-550-nm channel to green.

Simulation for Fluorophore Levels with HMM Simulations

The LUMICKS C-Trap optical tweezer-fluorescence microscope records raw data of binding events including original photon counts over time. By defining each protein binding events with pylake, photon count distribution of each event was extracted (FIGS. 28A and 28B). Then a HMM was applied using Matlab to analyze the dataset to estimate each fluorophore level. The code was adapted from Sgouralis et al²⁹. Original code is available at https://github.com/JamesLiWan/MultimerizationCode. After each dataset was analyzed and the maximum multimer number was obtained, the fluorophore level of each binding event was recognized and recorded. A complete statistical analysis to count the frequency of each multimer was then applied across different DNA datasets. Monomers and dimers were excluded because the photon count distribution dataset does not clearly distinguish between adjacent monomer/dimer events, causing potential inaccuracy

### Localization Analysis

Colocalization analysis was performed using the “Colocalization Analyzer” script available at harbor.lumicks.com. This script functions by performing a Gaussian fit to determine the positions of each event and then comparing each time and position of the binding events in one color with the times and positions of all binding events in a second color to determine the frequency and nature of interactions.

### Photobleaching Analysis

Photobleaching decay constants for each fluorophore was experimentally determined by testing the fluorescently labeled proteins immobilized at the bottom of the flow cell on the glass slides. The objective of the confocal microscope in C-Trap was lowered to the glass surface with identical laser power settings. At least 5 kymographs were obtained using the same data collection setup to observe photobleaching decay of these fluorophores. The images were processed through event data extraction and the photon counts of all events were fit into a single-exponential decay function to determine photobleaching lifetimes. Then, the binding mean lifetime of all events on DNA was corrected for photobleaching effect with the following equation:

$$\frac{1}{τ ( binding )} = \frac{1}{τ ( visual )} - \frac{1}{τ ( photobleaching )} .$$

### Nuclease Cleavage Experiment

Channels 1, 2, and 3 were flowed with polystyrene beads, biotinylated Ori98 DNAs, and 1×PBS, respectively. Channel 4 was flowed with nuclear extracts of mN-LT or pcDNA6 empty vector (EV) diluted 1:100 in reaction buffer. Channel 5 was flowed with S1 nuclease (NEB) at 1 μL (100 Units) in 500 μL of reaction buffer (40 mM sodium acetate pH 4.5, 0.3 M NaCl, and 2 mM ZnSO₄.) DNA tension was monitored until breakage (0 pN) or for >300 s.

### Protein Purification and Native Page Complex Formation for Cy5-RAD51

Human RAD51 was purified from Escherichia coli (AB1157ARecA) as described⁵⁵. To label RAD51 N-terminally with Cy5, recombinant RAD51 was dialyzed in buffer containing 250 mM NaPi (pH 7.0), 150 mM NaCl, 1 mM DTT, and 10% glycerol and labeled with Cy5-Mono-Reactive Dye (VWR). Cy5-RAD51 was further purified as described (55). Labeling efficiency was determined by measuring the absorbance of RAD51 at 280 nm and of Cy5 at 650 nm using their extinction coefficients (ε280=14,900 M-1 cm-1 for RAD51 and ε650=250,000 M⁻¹ cm⁻¹ for Cy5). Labeling efficiency was determined to be 39.7% for Cy5-RAD51.

### ATP Hydrolysis Assay

5 μL of 293 cell nuclear extract transfected with mN-LT was mixed with 2 μL apyrase (NEB) and 1× apyrase reaction buffer in a total reaction volume of 20 μL for 20 min at 30° C. for ATP hydrolysis. The reaction mixture was immediately diluted in 500 μL of reaction buffer (25 mM Tris-acetate pH 7.5, 10 mM magnesium acetate, 50 mM potassium acetate, 0.1 mM EDTA, 2 mM TCEP, 1 mM ATP, and 3 mM DTT) for single-molecule DNA binding experiments. For recovery with nonhydrolyzable ATP, adenylyl-imidodiphosphate (AMP-PNP) (Sigma) was added to the solution after ATP hydrolysis to a 1 mM final concentration.

### Data, Materials, and Software Availability

All study data are included in the article and/or supporting information. Software: Code used for the simulation of fluorophore levels has been deposited in GitHub at https://github.com/JamesLiWan/MultimerizationCode⁵⁶.

### 6.2.4 References

1. A. Costa, J. F. X. Diffley, The initiation of eukaryotic DNA replication. Annu. Rev. Biochem. 91, 107-131 (2022).
2. I. T. Todorov et al., A human nuclear protein with sequence homology to a family of early S phase proteins is required for entry into S phase and for cell division. J. Cell Sci. 107, 253-265 (1994).
3. L. D. Langston, M. E. O'Donnell, An explanation for origin unwinding in eukaryotes. Elife 8, e46515 (2019).
4. J. S. Lewis et al., Mechanism of replication origin melting nucleated by CMG helicase assembly. Nature 606, 1007-1014 (2022).
5. F. Abid Ali et al., Cryo-EM structure of a licensed DNA replication origin. Nat. Commun. 8, 1-10 (2017).
6. M. E. Douglas, F. A. Ali, A. Costa, J. F. Diffley, The mechanism of eukaryotic CMG helicase activation. Nature 555, 265-268 (2018).
7. M. Shuda et al., T antigen mutations are a human tumor-specific signature for Merkel cell polyomavirus. Proc. Natl. Acad. Sci. U.S.A. 105, 16272-16277 (2008).
8. P. S. Moore, Y. Chang, Why do viruses cause cancer? Highlights of the first century of human tumour virology. Nat. Rev. Cancer 10, 878-889 (2010).
9. K. G. Paulson et al., Merkel cell carcinoma: Current US incidence and projected increases based on changing demographics. J. Am. Acad Dermatol. 78, 457-463.e452 (2018).
10. M. M. Ahmed, C. H. Cushman, J. A. DeCaprio, Merkel cell polyomavirus: Oncogenesis in a stable genome. Viruses 14, 58 (2021).
11. H. Feng, M. Shuda, Y. Chang, P. S. Moore, Clonal integration of a polyomavirus in human merkel cell carcinoma. Science 319, 1096-1100 (2008).
12. D. V. Pastrana et al., Quantitation of human seroresponsiveness to merkel cell polyomavirus. PLOS Pathog. 5, e1000578 (2009).
13. Y. Chang, P. S. Moore, Merkel cell carcinoma: A virus-induced human cancer. Annu. Rev. Pathol. 7, 123-144 (2012).
14. M. E. Spurgeon et al., Merkel cell polyomavirus large T antigen binding to pRb promotes skin hyperplasia and tumor development. PLOS Pathog. 18, e1010551 (2022).
15. M. Dowlatshahi et al., Tumor-specific T cells in human Merkel cell carcinomas: A possible role for Tregs and T-cell exhaustion in reducing T-cell responses. J. Invest. Dermatol. 133, 1879-1889 (2013).
16. O. K. Afanasiev et al., Merkel polyomavirus-specific T cells fluctuate with merkel cell carcinoma burden and express therapeutically targetable PD-1 and Tim-3 exhaustion markers. Clin. Cancer Res. 19, 5351-5360 (2013).
17. H. J. Kwun et al., The minimum replication origin of merkel cell polyomavirus has a unique large T-antigen loading architecture and requires small T-antigen expression for optimal replication. J. Virol. 83, 12118-12128 (2009).
18. S. Waga, G. Bauer, B. Stillman, Reconstitution of complete SV40 DNA replication with purified replication factors. J. Biol. Chem. 269, 10923-10934 (1994).
19. T. J. Kelly et al., Replication of adenovirus and SV40 chromosomes in vitro. Philos. Trans. R Soc. Lond. B Biol. Sci. 317, 429-438 (1987).
20. T. Melendy, B. Stillman, An interaction between replication protein A and SV40 T antigen appears essential for primosome assembly during SV40 DNA replication. J. Biol. Chem. 268, 3389-3395 (1993).
21. T. Tsurimoto, T. Melendy, B. Stillman, Sequential initiation of lagging and leading strand synthesis by two different polymerase complexes at the SV40 DNA replication origin. Nature 346, 534-539 (1990).
22. F. B. Dean, J. Hurwitz, Simian virus 40 large T antigen untwists DNA at the origin of DNA replication. J. Biol. Chem. 266, 5062-5071 (1991).
23. L. D. Langston, Z. Yuan, R. Georgescu, H. Li, M. E. O'Donnell, SV40 T-antigen uses a DNA shearing mechanism to initiate origin unwinding. Proc. Natl. Acad. Sci. U.S.A. 119, e2216240119 (2022).
24. D. Li et al., Structure of the replicative helicase of the oncoprotein SV40 large tumour antigen. Nature 423, 512-518 (2003).
25. J. A. Borowiec, J. Hurwitz, ATP stimulates the binding of simian virus 40 (SV40) large tumor antigen to the SV40 origin of replication. Proc. Natl. Acad. Sci. U.S.A. 85, 64-68 (1988).
26. A. Kumar et al., Model for T-antigen-dependent melting of the simian virus 40 core origin based on studies of the interaction of the beta-hairpin with DNA. J. Virol. 81, 4808-4818 (2007).
27. C. J. Harrison et al., Asymmetric assembly of merkel cell polyomavirus large T-antigen origin binding domains at the viral origin. J. Mol. Biol. 409, 529-542 (2011).
28. B. Abere et al., Replication kinetics for a reporter merkel cell polyomavirus. Viruses 14, 473 (2022).
29. I. Sgouralis, S. Pressé, Icon: An adaptation of infinite hmms for time traces with drift. Biophys. J. 112, 2117-2126 (2017).
30. M. A. Schaich et al., Single-molecule analysis of DNA-binding proteins from nuclear extracts (SMADNE). Nucleic Acids Res. 51, e39 (2023), 10.1093/nar/gkad095.
31. G. Vauquelin, Effects of target binding kinetics on in vivo drug efficacy: Koff, kon and rebinding. Br. J. Pharmacol. 173, 2319-2334 (2016).
32. S. Siebels et al., Merkel cell polyomavirus DNA replication induces senescence in human dermal fibroblasts in a Kap1/Trim28-dependent manner. mBio 11, e00142-00120 (2020).
33. F. E. Benson, A. Stasiak, S. C. West, Purification and characterization of the human Rad51 protein, an analogue of E. coli RecA. EMBO J. 13, 5764-5771 (1994).
34. T. van der Heijden et al., Real-time assembly and disassembly of human RAD51 filaments on individual DNA molecules. Nucleic Acids Res. 35, 5646-5657 (2007).
35. M. R. Wasserman, G. D. Schauer, M. E. O'Donnell, S. Liu, Replication fork activation is enabled by a single-stranded DNA gate in CMG helicase. Cell 178, 600-611.e616 (2019).
36. L. Mohr et al., ER-directed TREX1 limits cGAS activation at micronuclei. Mol. Cell 81, 724-738.e729 (2021).
37. M. S. Wold, D. H. Weinberg, D. M. Virshup, J. J. Li, T. J. Kelly, Identification of cellular proteins required for simian virus 40 DNA replication. J. Biol. Chem. 264, 2801-2809 (1989).
38. D. Coverley et al., Requirement for the replication protein SSB in human DNA excision repair. Nature 349, 538-541 (1991).
39. T. C. Messina, H. Kim, J. T. Giurleo, D. S. Talaga, Hidden Markov model analysis of multichromophore photobleaching. J. Phys. Chem. B 110, 16366-16376 (2006).
40. J. Cheng, O. Rozenblatt-Rosen, K. G. Paulson, P. Nghiem, J. A. DeCaprio, Merkel cell polyomavirus large T antigen has growth-promoting and inhibitory activities. J. Virol. 87, 6118-6126 (2013).
41. P. I. Hanson, S. W. Whiteheart, AAA+ proteins: Have engine, will work. Nat. Rev. Mol. Cell Biol. 6, 519-529 (2005).
42. E. V. Koonin, A common set of conserved motifs in a vast variety of putative nucleic acid-dependent ATPases including MCM proteins involved in the initiation of eukaryotic DNA replication. Nucleic Acids Res. 21, 2541-2547 (1993).
43. J. A. Wendzicki, P. S. Moore, Y. Chang, Large T and small T antigens of merkel cell polyomavirus. Curr. Opin. Virol. 11, 38-43 (2015).
44. N. Y. Yao, D. Zhang, O. Yurieva, M. E. O'Donnell, CMG helicase can use ATPγS to unwind DNA: Implications for the rate-limiting step in the reaction mechanism. Proc. Natl. Acad. Sci. U.S.A. 119, e2119580119 (2022).
45. N. O. Onwubiko et al., SV40 T antigen interactions with ssDNA and replication protein A: A regulatory role of T antigen monomers in lagging strand DNA replication. Nucleic Acids Res. 48, 3657-3677 (2020).
46. E. Fanning, K. Zhao, SV40 DNA replication: From the A gene to a nanomachine. Virology 384, 352-359 (2009).
47. D. Gai, D. Wang, S.-X. Li, X. S. Chen, The structure of SV40 large T hexameric helicase in complex with AT-rich origin DNA. ELife 5, e18129 (2016).
48. D. Ramírez Montero, Nucleotide binding halts diffusion of the eukaryotic replicative helicase during activation. Nat. Commun. 14, 2082 (2023).
49. Y. P. Chang et al., Mechanism of origin DNA recognition and assembly of an initiator-helicase complex by SV40 large tumor antigen. Cell Rep. 3, 1117-1127 (2013).
50. X. Liu, S. Schuck, A. Stenlund, Adjacent residues in the E1 initiator β-hairpin define different roles of the β-hairpin in Ori melting, helicase loading, and helicase activity. Mol. Cell 25, 825-837 (2007).
51. M. Shuda et al., Merkel cell polyomavirus small T antigen induces cancer and embryonic merkel cell proliferation in a transgenic mouse model. PLOS One 10, e0142329 (2015).
52. S. Boichuk, L. Hu, J. Hein, O. V. Gjoerup, Multiple DNA damage signaling and repair pathways deregulated by simian virus 40 large T antigen. J. Virol. 84, 8007-8020 (2010).
53. J. Li et al., Merkel cell polyomavirus large T antigen disrupts host genomic integrity and inhibits cellular proliferation. J. Virol. 87, 9173-9188 (2013).
54. G. Meinke et al., The crystal structure of the SV40 T-antigen origin binding domain in complex with DNA. PLOS Biol. 5, e23 (2007).
55. S. Subramanyam, C. D. Kinz-Thompson, R. L. Gonzalez Jr., M. Spies, Observation and analysis of RAD51 nucleation dynamics at single-monomer resolution. Methods Enzymol. 600, 201-232 (2018).
56.L. Wan, MultimerizationCode. Github. https://github.com/JamesLiWan/MultimerizationCode. Deposited 22 Feb. 2023.

### 6.3 Example 3

### Characterization of DNA Binding Proteins to a Nucleosome-Containing DNA Substrate.

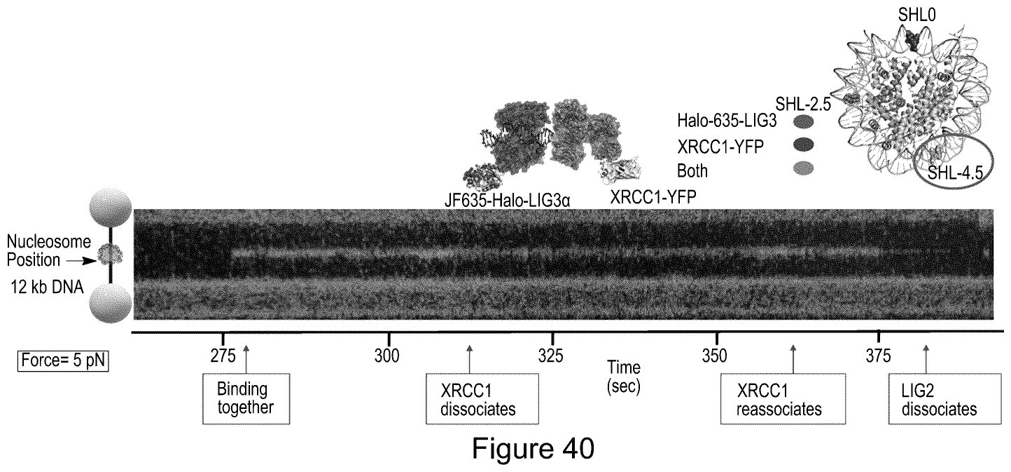

To observe protein-DNA interactions within the context of DNA packaging and chromatin-relevant structures, SMADNE was performed with a nucleosome-containing DNA substrate (FIG. 38). SMADNE analysis was performed on YFP-PARP1 (in nuclear extract) interacting with nicked DNA embedded in a nucleosome. Binding events were observed at a specific nicked superhelical location (SHL 0) at 4 pN of DNA tension. A K_{d} value of 1.6 nM (k_{off}/kₒₙ′=0.4 s⁻¹/2.4×10⁸ M⁻¹s⁻¹) had been observed for YFP-PARP1 (FIG. 39). Using this same approach, the binding of DNA single-strand break repair components, DNA Ligase III (LIG3) and XRCC1 were evaluated. As shown in FIG. 40, a LIG3-XRCC1 interaction was observed at nick site embedded in a nucleosome. Specifically, dwell times at SHL-4.5 were found to be the longest (10-14 s) and almost 54% of the complexes were heterodimers. Additionally, dwell times on “naked” non-ligatable nick were approximately 10 times shorter (1-2 s) and co-localization was approximately 4-5 fold less.

SMADNE analysis was further used to elucidate the importance of a specific domain in thymine-DNA glycosylase (TDG) interaction with non-damaged nucleosomes (FIG. 41). A 82 amino acid N-terminus was selectively removed from TDG and the protein was subjected to binding assays using SMADNE. The present disclosure shows that specific amino acids are essential for interaction, indicating a critical role for the N-terminal unfolded domain in TDG's binding with the nucleosome. Interestingly, this study indicates that the presence of an N-terminal unfolded domain, like in TDG, may be a general principle observed in many glycosylases. Furthermore, such protein-nucleosome interaction were previously unknown, highlighting the novel insights gained from SMADNE analysis.

### 6.4 Example 4

SMADNE approach compared to single-molecule analysis of a purified protein.

When proteins are properly purified, experimental results hold the distinct advantage of directly observing protein behavior without concern that unknown factors influence the results. Furthermore, protein purification has previously been an obligate requirement for numerous types of biophysical analyses, ranging from enzyme kinetics and structural studies to experiments where protein behavior is monitored at the single-molecule level. The present disclosure eliminates the need for protein purification in order to study proteins at the single-molecule level. By utilizing nuclear extracts directly expressed from mammalian cells, post-translational modifications (PTMs) can be preserved and fusion proteins expressed are highly active and can be frozen down within minutes of lysing cells, as opposed to the hours if not days of time necessary to fully purify a protein. SMADNE presents a unique opportunity as it encompasses many of the thousands of proteins found in a nucleus, allowing for a more comprehensive investigation of biomolecular interactions at the single-molecule level. As such, SMADNE results are more indicative of behavior in a biological context compared to a protein studied in isolation.

To better understand how unknown “dark” proteins in nuclear extracts impact single-molecule dynamics, the behavior of a purified protein was compared to that of the same protein expressed in a nuclear extract. The present example utilized 8-oxoguanine glycosylase 1 (OGG1) as a model system to determine how nuclear proteins present in extracts may alter single-molecule binding kinetics. OGG1 is a key protein in the repair of oxidative damage, and performs the first catalytic step of base excision repair by identifying 8-oxoguanine across from a cytidine and cleaving its glycosidic bond to leave behind an abasic site⁷. OGG1 faces the same challenge as many other glycosylases: billions of undamaged DNA base pairs must be rapidly sifted through to identify rare damage sites that would cause disastrous cellular consequences if left unrepaired⁸. Thus, it has been proposed and observed that OGG1 diffuses along the DNA helix to aid in its search for damage^{9,10}. The most direct way to understand the damage search process by OGG1 is fluorescent labeling of the protein and observing its search in real time. Thus, OGG1 has so far been characterized at the single-molecule level in many contexts, including on undamaged DNA with and without microfluidic flow^{1,9}, DNA containing abasic sites¹⁰, and DNA containing oxidative damage¹. Additionally, OGG1 tolerates numerous fluorescent labeling strategies, including Cy3 maleimide labeling, Qdot conjugation with an antibody, and fusing a fluorescent tag to the protein^{1,9,10}.

GFP-tagged OGG1 and a catalytically dead variant OGG1-K249Q were studied as a purified protein from a bacterial expression system, a hybrid approach where the purified protein was spiked into nuclear extracts, and finally with nuclear extracts with OGG1 overexpressed expressed in human cells. OGG1 binding dynamics were relatively similar on DNA substrates containing oxidative damage in all three conditions, with the weighted average binding lifetimes varying from 2.2 s in nuclear extracts to 7.8 s with purified OGG1 in isolation. In all three conditions, the binding lifetime greatly increased for the catalytically dead variant, with the weighted average lifetime for OGG1-249Q in nuclear extracts at 15.4 s vs 10.7 s for the purified protein. The presence of nuclear extracts also caused key differences in binding dynamics. In the presence of nuclear extracts, binding events on the undamaged DNA were not observed, compared to the purified protein results where OGG1 engaged undamaged DNA for an average lifetime of 5.7 s and 21% events diffused along the DNA after binding. The present disclosure indicates that proteins in the nuclear extracts compete for nonspecific interactions while still allowing for robust damage engagement by OGG1. Overall, the present disclosure showed that single-molecule studies performed in nuclear extracts complement studies performed with purified proteins and give a biological contextualization to proteins studied in isolation.

### 6.4.1 Results

### Purified OGG1 Scans Undamaged DNA for Damage

To test the mechanisms by which OGG1 searches for DNA damage, a purified a GFP-tagged OGG1 generated with bacterial overexpression was utilized. Notably, the GFP-label did not interfere with OGG1 activity, as the purified protein was highly active (FIG. 42A and FIG. 42H). The DNA substrate, a 48.5 kb of dsDNA was suspended in a flow chamber with a precise force measurement and control (FIG. 42B)¹. DNA tethering was performed before moving the DNA substrate into a new channel containing the protein of interest. The DNA was positioned in the middle of the flow channel away from the surface of the glass, which prevented imaging artifacts caused by debris nonspecifically adhering to the glass of the flow cell. Upon moving the tethered DNA into the channel of the flow cell containing purified GFP-labeled OGG1 obtained from bacterial cells, a variety of single-molecule binding events across the length of the DNA were observed, including 21% binding events that appeared to diffuse on the DNA and some that appeared to bind at a one position on the DNA before releasing (FIGS. 42B and 42C). Presented as a kymograph (with each pixel in the x-axis representing 100 ms and each pixel in the y axis representing 100 nm), stationary binding events appear as straight green lines on the DNA, whereas moving events appear as jagged lines from the diffusion on the DNA. Surprisingly, there was a rapid reduction in the of background fluorescence within 15-20 seconds which had been generated from OGG1-GFP molecules diffusing in solution and not bound to the DNA. This wave of fluorescence reduced relatively quickly after flowing in fresh protein—as the valves were sealed shut to the flow cell, this reduction in the available protein is likely caused by molecules sticking to the glass outside of the imaging plane, reducing the amount of protein available for binding. Because of this fading phenomenon, the majority of binding events occurred within the first few seconds of a kymograph, and once the background levels depleted binding events were much rarer.

Tracking the duration of binding events revealed dwell times occurred over a wide range, from transient events that occurred less than one second to long-lived events that lasted over 100 seconds (FIG. 42D). These events were sorted by duration and fit to a cumulative residence time distribution (CRTD) plot 1. Upon fitting to a double-exponential decay function, the events exhibited two lifetimes, one at 1.5 s (60% contributing) and one at 11.9 s (40% contributing) (Table 7). These two different binding lifetimes can result because of conformational proofreading by OGG1, where one protein conformation acts as a brief DNA sampling and where a second conformation resides longer on the DNA12. Alternatively, the fast phase can be a non-specific binding, while the longer lived binding events represent cryptic lesions that were introduced into the lambda DNA during purification and processing prior to stringing up in the C-trap. Of the events observed, 21% exhibited motile behavior (FIG. 42E) and diffused on the DNA before dissociating. The diffusivity of the motile events was determined using mean square displacement (MSD) analysis, and was on average 0.035 μm²/s (FIG. 42F). This average diffusivity value was much slower than the 0.58 μm²/s reported for Cy3 labeled OGG1, which could be explained in part by the 100 μm/s flow velocity of the previous collection, compared to data collected without flow⁹. In contrast to the events observed with purified OGG1 on undamaged DNA, when purified OGG1 was spiked into nuclear extract or nuclear extracts from human cells in which expressed OGG1 was expressed off a CMV promoter and used directly there was no observed binding events on undamaged DNA (FIG. 42G) and thus also did not observe any 1D diffusion by OGG1 on the DNA.

TABLE 7
Summary of single-molecule binding kinetics (* = 21% motile events).

| | | | Purified + | |
|---|---|---|---|---|
| DNA tether | OGG1 | Purified protein | extract lifetime | SMADNE |
| type | variant | lifetime (s) | (s) | lifetime (s) |
| Undamaged | WT | τ₁ = 1.5 s (60%) | No events | No events |
| | | τ₂ = 11.9 s (40%) | | |
| | | τ_{avg} = 5.7s * | | |
| | K249Q | τ = 0.47 s | No events | No events |
| 8-oxoguanine | WT | τ₁= 4.4 s (46%) | τ₁ = 2.0 s (88%) | τ₁ = 0.8 s (51%) |
| | | τ₂ = 10.6 s (54%) | τ₂ = 45.1 s (12%) | τ₂ = 3.2 s (49%) |
| | | τ_{avg} = 7.8 s | τ_{avg} = 7.1s | τ_{avg} = 2.0 s |
| | K249Q | τ₁= 4.7 s (46%) | τ₁ = 2.9 s (52%) | τ₁ = 7.7 s (78%) |
| | | τ₂ = 15.8 s (54%) | τ₂ = 24.8 s (54%) | τ₂ = 42.9 s (22%) |
| | | τ_{avg} = 10.7 s | τ_{avg} = 13.4 s | τ_{avg} = 15.4 s |

OGG1 Robustly Binds 8-oxoG as a Purified Protein and in the Presence of Nuclear Extract

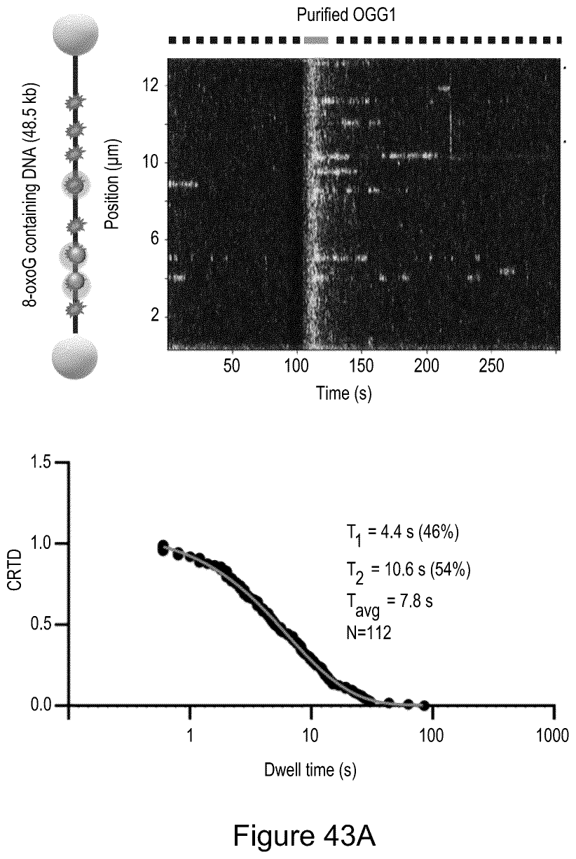

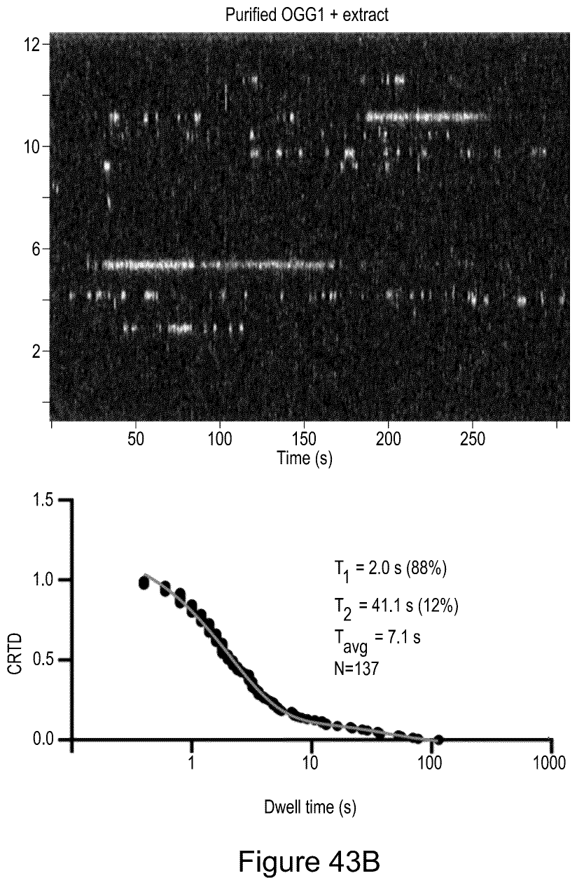

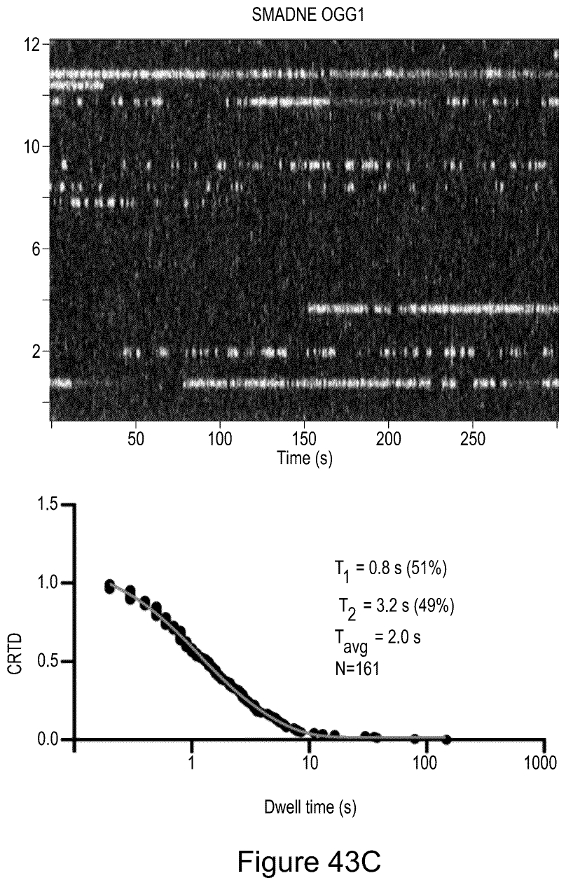

To assess the ability of OGG1 to bind 8-oxoG, the lambda DNA substrate had been exposed to methylene blue and light to generate oxidative damage. The generated oxidative damage, primarily 8-oxoG, was distributed approximately every 440 base pairs along the DNA sequence¹¹. With this damage load, motile binding events were no longer observed with purified OGG1. This can be attributed to higher affinities for 8-oxoG over non-damaged DNA, which allowed 3D diffusion to be sufficient for a binding event, or OGG1 did not need to scan very far before encountering a damage site since 440 bp fell below the resolution of the C-trap (FIG. 43A). All three tested conditions exhibited a wide range of dwell times, and the purified OGG1 bound with a lifetime of 4.4 s (46%) and 10.6 s (54%), for a weighted average lifetime of 7.8 s. When the purified protein was incubated in the presence of nuclear extract, events occurred at a similar rate over the course of five minutes of collection because the OGG1-GFP did not fade away in this context as it did in a purified protein setting (FIG. 43B). However, the dwell times for OGG1-GFP with extract present were similar to the behavior with purified protein, with one lifetime at 2.0 s (88%) and another at 45.1 s (12%), for a weighted average lifetime of 7.1 s. Lastly, it was found that OGG1-GFP generated from human cell nuclear extracts exhibited exclusively nonmotile events on the damaged DNA. While the range of dwell times were less than a second to over 100 seconds, many short binding events caused the CRTD plot to exhibit two shorter lifetimes, one at 0.8 s (51%) and one at 3.2 s (49%), for a weighted average lifetime of 2.0 s (FIG. 43C). These relatively short dwell times for OGG1 prepared in human cells indicated that post-translation modification of OGG1 may have been a factor in the changed off rate.

### Catalytically Dead OGG1 Transiently Engages Undamaged DNA

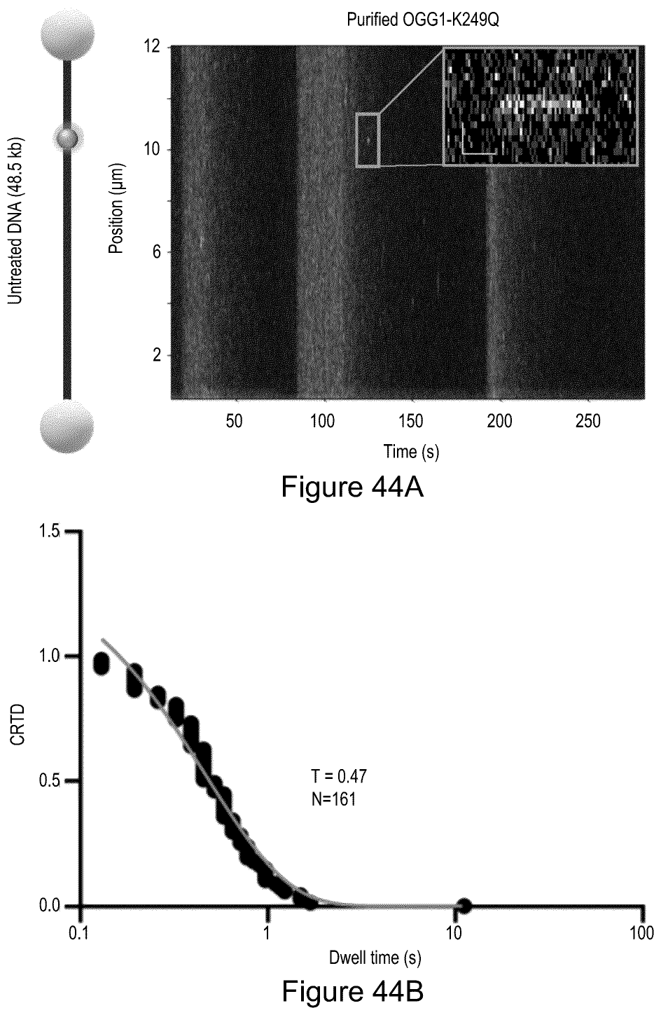

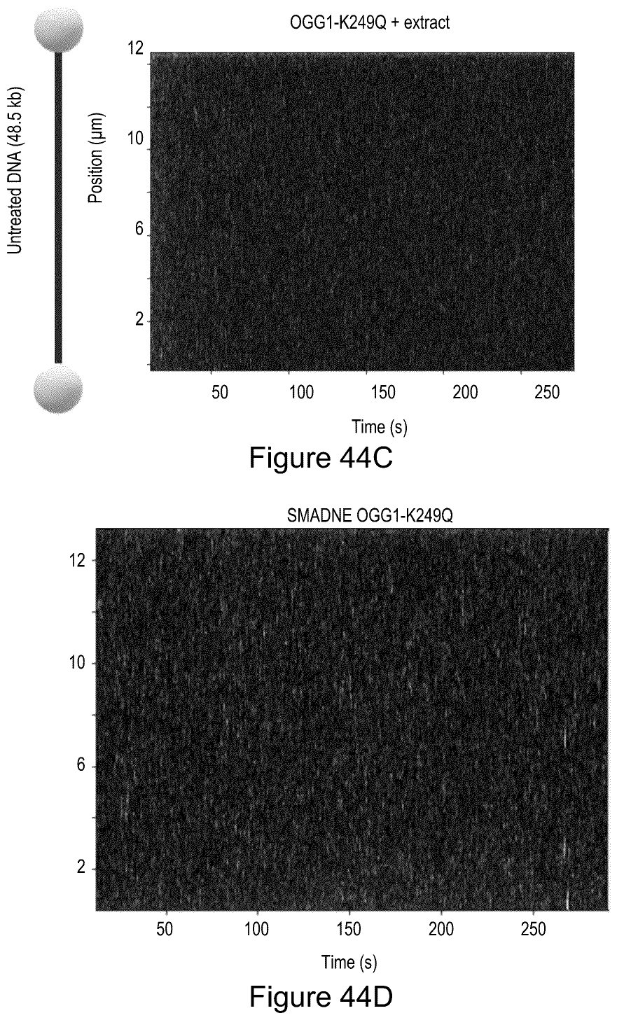

To investigate the impact of nuclear extracts on protein binding lifetimes, the present disclosure examined a catalytically dead variant K249Q, where the positively charged lysine that initiates the catalytic mechanism of breakage of the glycosidic bond between the 8-oxoG base and the sugar was replaced by a glutamine residue (K249Q)¹³. With the variant being catalytically dead, an unambiguous determination was made that the nature of binding events did not involve abasic sites created by the glycosylase activity of OGG1 removing 8-oxoG. The catalytic variant was tested on undamaged DNA, and similar trends were observed between the purified protein and the protein in a nuclear extract, as compared to the WT protein. Specifically, binding events were observed on the undamaged DNA with purified protein (FIGS. 44A and 44B) but that there was no “off-target” events when the purified OGG1 was spiked into nuclear extract or expressed via the SMADNE approach (FIGS. 44C and 44D). While binding events were evident with purified OGG1-K249Q-GFP, the binding lifetime of the variant was much shorter than WT OGG1-GFP, fitting to a single-exponential decay function with a lifetime of 0.47 s. Furthermore, no visibly motile events were observed with this catalytic mutant. This could be because the sampling events on the DNA were too transient to establish a search mode, or that the catalytic reside K249 itself is an essential residue for DNA scanning by OGG1.

OGG1-K249Q-GFP Engages Damage Sites with Longer Lifetimes than WT OGG1

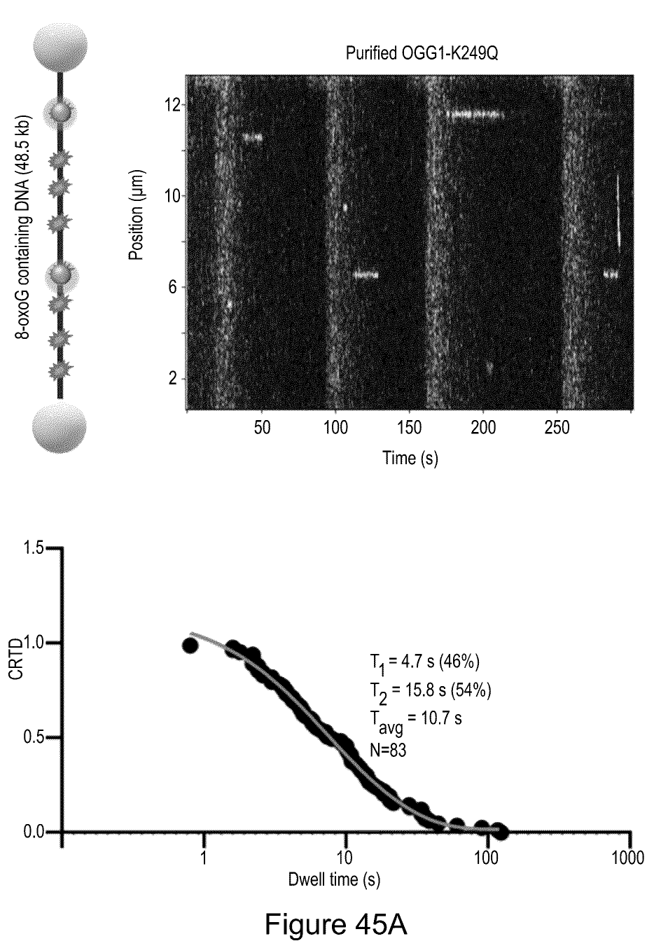

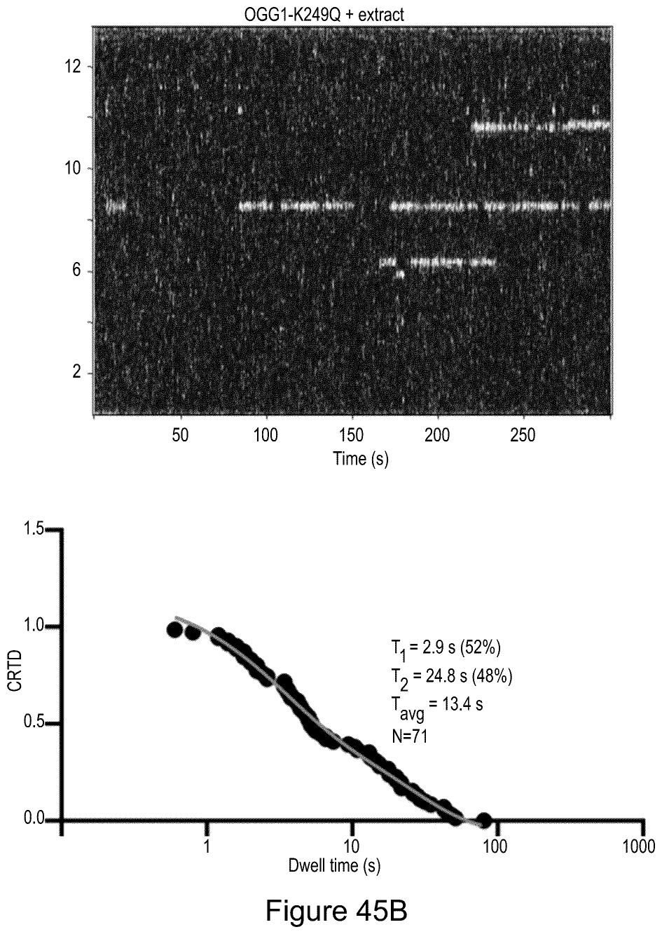

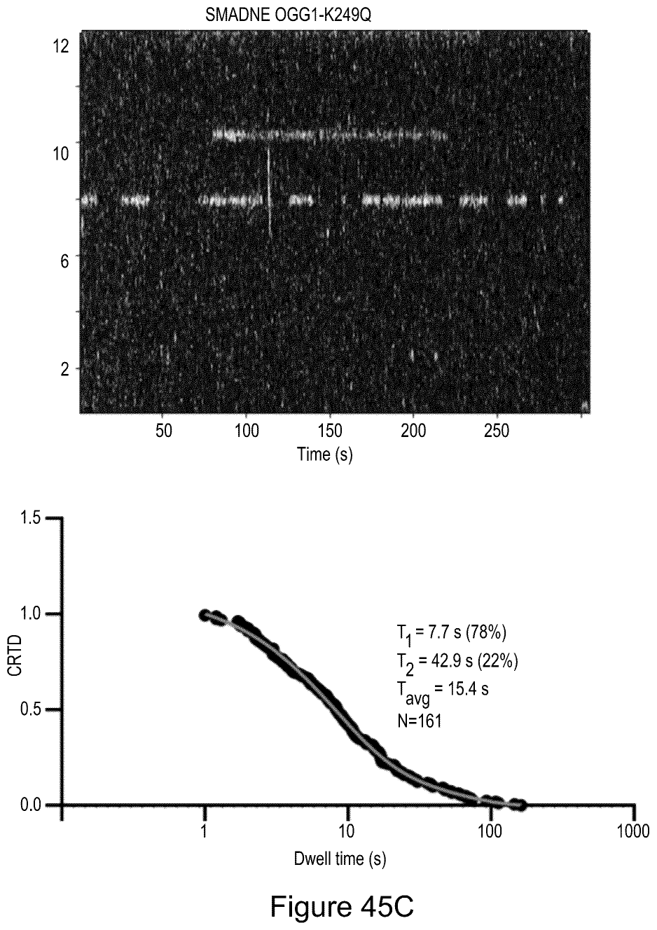

The catalytically-dead OGG1 produced longer-lived binding events on damaged DNA than WT OGG1 in all three experimental conditions tested (i.e., purified protein, purified protein plus nuclear extract, and SMADNE, FIGS. 45A-45C). This trend confirmed the behavior of WT OGG1, where the presence of nuclear extracts reduced non-specific binding events but still allowed for successful engagement of DNA damage. In the case of OGG1 purified from bacterial cells, exclusively nonmotile events were observed for this substrate, similar to the WT OGG1 on DNA containing 8-oxoG (FIG. 45A). These events exhibited dwell times that fit to a double-exponential decay function, with one lifetime at 4.7 s (46%) and the other at 15.8 s (54%), for a weighted average lifetime of 10.7 s. Thus, there was a 20-fold increase in the binding lifetime of OGG1 K249Q between undamaged DNA and DNA containing 8-oxoG. For the purified OGG1 that was spiked into nuclear extracts, a similar binding lifetime and behavior was observed. Dwell times fit a double-exponential decay function with one lifetime at 2.9 s and one lifetime at 24.8 s, with the short lifetime contributing 52% and a weighted average lifetime of 13.4 s (FIG. 45B). Lastly, in the case of the OGG1-K249Q-GFP expressed in mammalian cells for the SMADNE approach, the binding events exhibited two off-rates, with one lifetime at 7.7 s and the second at 42.9 s, where the fast lifetime contributed 78% (FIG. 45C). While the rates themselves were longer than the other two conditions, when the smaller contribution of the slow rate is taken into account, the weighted average lifetime for the SMADNE OGG-K249Q-GFP was 15.4 s, which was similar to the lifetimes of the other two conditions.

### 6.4.2 Discussion

While the SMADNE approach¹ promises to provide a large group of scientist access to the single molecule regime it is essential to understand how the “dark” proteins in the extract influence protein binding to DNA. The behavior of OGG1 was used as a test case and allowed for a direct comparison of single-molecule analysis of a purified protein from bacterial cells as compared to purified OGG1 added to nuclear extracts versus nuclear extracts containing OGG1 overexpressed in human cells during transient transfection. These latter conditions helped assess the effects of dilute nuclear proteins on the DNA binding behavior of a target protein. While the measured lifetimes varied in value, in all three experimental conditions increased the binding lifetime for the K249Q variant compared to the WT protein. There are several considerations to keep in mind when studying proteins overexpressed in nuclear extracts at the single-molecule level, single-molecule analysis of nuclear extracts (the SMADNE method) offers a rapid characterization of variant proteins, the presence of chaperones to stabilize the protein of interest, an increase in specificity by reducing nonspecific binding, and facilitated dissociation that allows for the efficient release of proteins from their substrates (FIG. 46A-46D).

### The Presence of Nuclear Proteins Allowed for Efficient and Rapid Data Collection

Because the SMADNE workflow is rapid (from plasmid to extracts to C-trap data analysis within a week), the ability to quickly analyze variant proteins at the single-molecule level acts as a major advantage of working in extracts (FIG. 46A). These variants could be rationally designed to better understand the protein function, as in this present work, or even chosen from online databases to better understand how variants found in a clinical context contribute to function and thus disease. Many genes present in the Catalog of Somatic Mutations in Cancer (COSMIC, https://cancer.sanger.ac.uk/cosmic) have thousands of variants reported. Even with an optimistic estimation of 2 weeks to express, purify, and analyze each variant protein, it would take around a year just to screen through ˜25 variants. In comparison, with the SMADNE approach, it takes around two days to transfect and perform a nuclear extract for each sample, so in principle this would cut down the time needed to analyze 25 variants to 1-2 months. Furthermore, by eliminating the necessity of protein purification and fluorescent labeling, SMADNE democratizes single-molecule biophysical studies for a broad scientific community¹⁴.

Aside from workflow considerations, the other nuclear proteins present in the experimental conditions also offer other key advantages. The present disclosure found that the concentrations of bacterially purified OGG1-GFP decreased over time, which caused difficulties in collection and analysis. Most notably, on rates cannot be reliably determined with such variability in concentration over time and setting a threshold level for line tracking becomes challenging with variable background signal. The present disclosure found that nuclear extracts with purified OGG1-GFP resolved the issue with purified protein. Secondly, chaperone proteins present in the nuclear extracts can increase the stability of proteins in the nuclear extract. Proteomic analysis of nuclear extracts made using the approach described here, indicated that two out of the top 20 most abundant proteins in the extract were identified as heat shock proteins (Heat shock protein HSP 90-beta and Heat shock cognate 71 kDa protein, see Table 8, FIG. 46B). Thus, the level of chaperone proteins were on par with highly abundant nuclear proteins involved in nuclear structure, such as actin or nuclear pore complex protein Nup160. Thus, these and other chaperones can stabilize proteins in solution during data collection. The present disclosure determined that nuclear extracts can be utilized for hours of collection without apparent loss of activity. Furthermore, chaperones increased protein stability can explain why there was an approximate 3 second increase in weighted average binding lifetime for OGG1-K249Q present in nuclear extracts compared to the purified protein alone. This stabilization phenomenon may be of even greater importance when studying variants that disrupt protein stability.

TABLE 8
The 20 most abundant proteins present in nuclear extracts. Proteins that
assist with protein folding are shown in bold text. Adapted from mass spectrometry
experiment in ¹.

| | | Mol. |
|---|---|---|
| | | weight |
| Protein names | Gene names | [kDa] |
| Actin | ACTG1; ACTB | 41.792 |
| Annexin A2; Putative annexin A2-like protein | ANXA2; ANXA2P2 | 38.604 |
| Vimentin | VIM | 53.651 |
| Plectin | PLEC | 531.78 |
| Nuclear pore complex protein Nup160 | NUP160 | 162.12 |
| Filamin-A | FLNA | 280.74 |
| Annexin A1 | ANXA1 | 38.714 |
| Neuroblast differentiation-associated protein AHNAK | AHNAK | 629.09 |
| Annexin A5 | ANXA5 | 35.936 |
| Myosin-9 | MYH9 | 226.53 |
| ATP synthase subunit alpha, mitochondrial | ATP5A1 | 59.75 |
| Putative elongation factor 1-alpha-like 3; Elongation factor 1- | EEF1A1P5; EEF1A1 | 50.184 |
| alpha 1 | | |
| ADP/ATP translocase 2; ADP/ATP translocase 2, N- | SLC25A5 | 32.852 |
| terminally processed | | |
| ATP synthase subunit beta, mitochondrial | ATP5B | 56.559 |
| Heat shock protein HSP 90-beta | HSP90AB1 | 83.263 |
| Heat shock cognate 71 kDa protein | HSPA8 | 70.897 |
| Glyceraldehyde-3-phosphate dehydrogenase | GAPDH | 36.053 |
| Moesin | MSN | 67.819 |
| Kinesin-like protein KIF20B | KIF20B | 210.63 |
| Ezrin | EZR | 69.412 |

### Nuclear Proteins in Extract Compete for Undamaged DNA Binding

One of the most striking differences between the purified OGG1 and OGG1 with nuclear extracts present was its behavior on undamaged DNA: numerous binding events on undamaged DNA were observed with purified OGG1, including some motile events that could scan along the DNA. However, when the nuclear extracts were present these “nonspecific” events did not occur. Thus, unknown and unlabeled “dark” DNA binding proteins in the nuclear extract bound to the undamaged DNA and interfered with OGG1 binding (FIG. 46C). However, the dark proteins did not interfere with the ability of OGG1 to engage damage present on the DNA. Other proteins blocking OGG1 from binding to undamaged DNA can increase OGG1 damage-binding specificity. This finding raised the question whether OGG1 utilized 1D diffusion in the nucleus for damage detection (where these dark proteins arc presumably at much higher concentrations). Of note, ID diffusion has been observed with the SMADNE approach for several other DNA repair proteins, including 3-alkyladenine DNA glycosylase (AAG)¹⁵, Xeroderma pigmentosum complementation group C protein (XPC), and a variant of damaged-DNA binding protein 2 (DDB2)¹. Studies conducted on AAG show that both the fraction of events that diffused and the rate of diffusion largely agreed between the data collected with nuclear extracts and the quantum dot-conjugated purified protein. The search process of AAG has not been shown to be altered by dark proteins, as observed in the present disclosure of OGG1.

### Proteins Present in Nuclear Extracts May Contribute to Efficient Repair Mechanisms Via Facilitated Dissociation.

With purified proteins, the off-rate is independent of protein concentration¹⁶. However, the presence of unlabeled competitors can cause the off rate to increase due to the concept of facilitated dissociation¹⁷⁻¹⁹. In this phenomenon, the unlabeled proteins compete for sites on the DNA where their target has partially dissociated, and thus shift the equilibrium towards dissociation of the target. An advantage of utilizing GFP-fusion proteins is that protein samples do not need to be conjugated to Qdots or adding dyes, which involves malcimide or N-hydroxysuccinimide reactions. Instead, fusion proteins are quantitatively labeled, i.e., there is one fluorophore per protein and 100% of the purified proteins are labeled. In the purified context, this minimizes the possibility that unlabeled OGG1 can remove labeled protein once it has engaged the DNA. With the nuclear extracts, an OGG1 knockout cell line was not used, so some endogenous OGG1 is present. However, with the overexpression of of the fusion protein using a CMV promoter, expression levels 30-50 times higher than the endogenous protein were observed, which translates to 97-98% labeled protein¹. The endogenous protein had no discernible impact until it reached approximately 25% unlabeled¹.

In nuclear extracts, however, several other proteins present in the extract could be assisting in OGG1 dissociation. This phenomenon was observed with UV-damaged DNA binding protein (UV-DDB), which stimulates the release of multiple DNA glycosylases from abasic sites, including OGG110.20, AAG15, MUTYH21, and SMUG122. Furthermore, endogenous apurinic/apyrimidinic endonuclease 1 (APE1) was also detected in nuclear extracts, which also has been shown to contribute to the efficient turnover of OGG 1.2.3. The present disclosure demonstrated that nuclear proteins shortened the binding lifetime on DNA damage. In experiments with WT OGG1 on DNA with 8-oxoG, both purified OGG1 resided longer on the DNA damage compared to purified protein spiked into nuclear extracts and OGG1 generated by SMADNE. The mechanism by which the lifetimes are being shortened can caused by facilitated dissociation (FIG. 46D).

The present disclosure showed that WT OGG1 expressed in mammalian cells exhibited an approximate threefold shorter lifetime than the purified protein, indicating that other factors may also be altering the binding lifetime. A potential factor could be the post-translational modification state of OGG1 when expressed in mammalian cells versus bacterial cells. OGG1 can be modified in numerous ways, including phosphorylated on a serine residue by protein kinase C²⁴, PARylated by PARP1²⁵, acetylated by p30026, or even O-GlcNAcylated^{27,28}. These modifications are likely not made to the purified protein when added to the extract because all of the cofactors needed for modification (NAD, ATP, and others) are greatly diluted during the nuclear extraction. Measurements of NAD and ATP in undiluted nuclear extracts were approximately in the high nanomolar to 1 μM range. Another possibility is that the OGG1 protein could be at a different oxidation state when made in extracts vs purified from bacteria. A recent study found that OGG1 contains a nitrogen-oxygen-sulfur redox switch, and that the nitrogen from K249 contributes the nitrogen to the bridge²⁹. The K249Q variant cannot form this bridge, which can explain why the purified variant protein spiked into extract condition exhibited a more similar lifetime to the SMADNE experiment compared to the WT protein where the switch was active. However, fresh DTT (1 mM) was used in all experimental conditions, which can reduce any redox bridges present.

### 6.4.3 Conclusion

The nucleus of a cell is “dirty” by definition, with thousands of factors that could potentially impact the function of a single protein. Removing a protein from the milieu of a nucleus unlocks many potential techniques that are unattainable without purification, including structural studies and countless enzymological experiments. However, removing the “dirt” from a protein comes at a cost, in terms of time, experience, and reagents consumed for the purification scheme but also at a cost of purifying out relevant factors to biological factors. In biology, no protein works in isolation, and growing literature on pathway interplay implies that unexpected or even unknown proteins may assist in functions that are lost by purification. Directly analyzing proteins expressed in nuclear extracts at the single-molecule level represents an intermediate approach, through which new information can be gained that complements traditional biophysical experiments with purified proteins and cellular experiments. SMADNE provides a new window of observation into the behavior of nucleic acid binding proteins heretofore only accessible by biophysicists trained in protein purification and protein labeling. Furthermore, SMADNE provides an opportunity for those who routinely study fluorescently tagged proteins in cell experiments to work within the single molecule regime.

### 6.4.3 Materials and Methods

### Protein Expression and Purification of Recombinant OGG1 Cell Lines

Transfection and nuclear extraction were performed as described above (SMADNE methodolog¹). Briefly, U2OS cells were cultured in Dulbecco's Modified Eagle Medium (DMEM) supplemented with 4.5 g/l glucose, 10% fetal bovine serum (Gibco), 5% penicillin/streptavidin (Life Technologies) with 5% oxygen. Four μg of plasmid per four million cells as a transfection with lipofectamine 2000. To prepare the nuclear extract control samples, the same lipofectamine protocol was followed but no plasmid was added. At 24 h after the transfection nuclear extracts were generated. Resultant nuclear extracts were aliquoted into single-use tubes and flash frozen in liquid nitrogen prior to storing them at −80 C.

### DNA Substrate Generation

Lambda DNA for C-trap experiments was purchased from New England Biolabs and its overhangs were biotinylated with biotinylated dCTP¹. Oxidative damage was introduced by incubating with 0.2 μg/mL methylene blue (as performed here 11) and exposed to 660 nm light for 10 minutes. The protocol introduced 1 damaged base per ˜440 bp throughout the length of the lambda DNA.

### Single-Molecule Experiments

Equipment: A LUMICKS C-trap consisting of three channel confocal microscope, five chamber flow cell and two optical traps were used in all experiments. Single photon detectors were used during kymograph acquisition at 10 frames per second and 100 nm pixels in the Y-axis.

### DNA Tether Formation and Positioning

All single-molecule experiments were performed on a Lumicks C-trap instrument, a platform that combines optical tweezers, confocal fluorescence microcopy, and a microfluidic flow cell, as described above. Utilizing four channels of the microfluidic flow cell, experimental design consisted of four major steps prior to imaging. First, after opening the valves of the flow cell and pressurizing to 0.3 bar to maintain laminar flow, streptavidin-coated polystyrene beads (4.4-4.8 micron) were immobilized in two separate optical traps. Then the beads were moved to the second channel of the flow cell where the biotinylated lambda DNA was flowing. DNA substrate generation method is described above.

By varying the distance between the beads between 10 microns to 15 microns and monitoring the force compared to an extensible worm-like chain model, a single DNA tether was obtained between the two beads. Then the tethered DNA was moved to a channel containing buffer that consisted of 150 mM NaCl, 20 mM HEPES pH 7.5, 5% glycerol, 0.1 mg/mL BSA, 1 mM freshly thawed DTT, and 1 mM Trolox. The DNA was washed for ten seconds before moving to the channel with the fluorescent OGG1 (either as purified proteins at 20 nM concentration, 10 nM purified protein spiked into nuclear extracts without overexpression diluted 1:10 in imaging buffer, or nuclear extracts diluted 1:10 in imaging buffer), pulling the tension to 10 pN, and collecting binding events along the DNA. For the experiments containing nuclear extracts, buffer and nuclear extracts were flowed in fresh every five minutes. For experiments with purified proteins, the sample was refreshed more frequently to account for the decay in fluorescent intensity, typically every 1-2 minutes and when binding events were no longer occurring.

### Confocal Imaging

GFP signals were collected by exciting with a 488 nm laser at 5% power (˜2 μW at the objective) and emission was collected through a 500-550 nm band pass filter. Imaging was performed with a 1.2 NA 60× water objective and intensities measured with single-photon avalanche photodiode detectors. Kymograph scans were collected along the length of the DNA and 10 frames per second with a pixel size of 100 nm and exposure time of 0.1 msec per pixel. In the case of WT OGG1-249Q on undamaged DNA, this time resolution made line tracking difficult given the short binding lifetime, so framerate was increased to 33 frames per second.

### Data Analysis

Kymographs were analyzed with custom software from Lumicks (Pylake). Images for publication were generated with the .h5 Visualization GUI (2020) by John Watters, accessed through harbor.lumicks.com. As GFP has been previously observed to blink up to two seconds, any events occurred at the same position with less than two seconds of non-fluorescent time between them were connected and counted as a single binding event.

Motile events were analyzed using by extracting the mean square displacement utility of Pylake, where the plots for each lag time were exported for custom fitting. The equation utilized is shown below:

$$MSD ( n Δ t ) = \frac{1}{N - n} {\sum }_{i = 1}^{N - n} {( {x}_{i + n} - {x}_{i} )}^{2} ,$$

where N is total number of frames in the phase, n is the number of frames at a given time step, Δt is the time increment of one frame, and xi is the particle position in the ith frame. The diffusion coefficient (D) was determined by fitting a model of one-dimensional diffusion to the linear portion of the MSD plots:

$$MSD ( n Δ t ) = \frac{1}{N - n} {\sum }_{i = 1}^{N - n} {( {x}_{i + n} - {x}_{i} )}^{2} ,$$

where α is the anomalous diffusion coefficient and y is a constant (y-intercept). Each plot was analyzed using Graphpad Prism, and the maximum time window adjusted to include as much of the linear portion of the graph as possible. Fittings resulting in R2 less than 0.8 or using less than 10% of the MSD plot were excluded.

### 6.4.5 References

1 Schaich, M. A. et al. Single-molecule analysis of DNA-binding proteins from nuclear extracts (SMADNE). Nucleic Acids Res 51, e39, doi: 10.1093/nar/gkad095 (2023).
2 Haraszti, R. A. & Braun, J. E. Comparative Colocalization Single-Molecule Spectroscopy (COSMOS) with Multiple RNA Species. Methods Mol Biol 2113, 23-29, doi: 10.1007/978-1-0716-0278-2_3 (2020).
3 Hoskins, A. A. et al. Ordered and dynamic assembly of single spliceosomes. Science (New York, N.Y.) 331, 1289-1295, doi: 10.1126/science.1198830 (2011).
4 Sparks, J. L. et al. The CMG Helicase Bypasses DNA-Protein Cross-Links to Facilitate Their Repair. Cell 176, 167-181.e121, doi: 10.1016/j.cell.2018.10.053 (2019).
5 Kanke, M., Tahara, E., Huis In't Veld, P. J. & Nishiyama, T. Cohesin acetylation and Wapl-Pds5 oppositely regulate translocation of cohesin along DNA. Embo j 35, 2686-2698, doi: 10.15252/embj.201695756 (2016).
6 Graham, T. G. W., Walter, J. C. & Loparo, J. J. Two-Stage Synapsis of DNA Ends during Non-homologous End Joining. Molecular cell 61, 850-858, doi: 10.1016/j.molcel.2016.02.010 (2016).
7 Whitaker, A. M., Schaich, M. A., Smith, M. R., Flynn, T. S. & Freudenthal, B. D. Base excision repair of oxidative DNA damage: from mechanism to disease. Front Biosci (Landmark Ed) 22, 1493-1522, doi: 10.2741/4555 (2017).
8 van der Kemp, P. A., Thomas, D., Barbey, R., de Oliveira, R. & Boiteux, S. Cloning and expression in Escherichia coli of the OGG1 gene of Saccharomyces cerevisiae, which codes for a DNA glycosylase that excises 7,8-dihydro-8-oxoguanine and 2,6-diamino-4-hydroxy-5-N-methylformamidopyrimidine. Proceedings of the National Academy of Sciences 93, 5197-5202, doi: 10.1073/pnas.93.11.5197 (1996).
9 Blainey, P. C., van Oijen, A. M., Banerjee, A., Verdine, G. L. & Xie, X. S. A base-excision DNA-repair protein finds intrahelical lesion bases by fast sliding in contact with DNA. Proc Natl Acad Sci USA 103, 5752-5757, doi: 10.1073/pnas.0509723103 (2006).
10 Jang, S. et al. Damage sensor role of UV-DDB during base excision repair. Nat Struct Mol Biol 26, 695-703, doi: 10.1038/s41594-019-0261-7 (2019).
11 Nelson, S. R., Dunn, A. R., Kathe, S. D., Warshaw, D. M. & Wallace, S. S. Two glycosylase families diffusively scan DNA using a wedge residue to probe for and identify oxidatively damaged bases. Proceedings of the National Academy of Sciences 111, E2091, doi: 10.1073/pnas. 1400386111 (2014).
12 Ghodke, H. et al. Single-molecule analysis reveals human UV-damaged DNA-binding protein (UV-DDB) dimerizes on DNA via multiple kinetic intermediates. Proceedings of the National Academy of Sciences of the United States of America 111, E1862-1871, doi: 10.1073/pnas. 1323856111 (2014).
13 Bruner, S. D., Norman, D. P. & Verdine, G. L. Structural basis for recognition and repair of the endogenous mutagen 8-oxoguanine in DNA. Nature 403, 859-866, doi: 10.1038/35002510 (2000).
14 Wan, L. et al. Unlicensed origin DNA melting by MCV and SV40 polyomavirus LT proteins is independent of ATP-dependent helicase activity. 120, e2308010120, doi: doi: 10.1073/pnas.2308010120 (2023).
15 Jang, S. et al. Cooperative interaction between AAG and UV-DDB in the removal of modified bases. Nucleic Acids Research 50, 12856-12871, doi: 10.1093/nar/gkac1145 (2022).
16 Jarmoskaite, I., AlSadhan, I., Vaidyanathan, P. P. & Herschlag, D. How to measure and evaluate binding affinities. eLife 9, e57264, doi: 10.7554/eLife.57264 (2020).
17 Kamar, R. I. et al. Facilitated dissociation of transcription factors from single DNA binding sites. Proc Natl Acad Sci USA 114, E3251-e3257, doi: 10.1073/pnas. 1701884114 (2017).
18 Hadizadeh, N., Johnson Reid, C. & Marko John, F. Facilitated Dissociation of a Nucleoid Protein from the Bacterial Chromosome. Journal of Bacteriology 198, 1735-1742, doi: 10.1128/jb.00225-16 (2016).
19 Gibb, B. et al. Protein dynamics during presynaptic-complex assembly on individual single-stranded DNA molecules. Nat Struct Mol Biol 21, 893-900, doi: 10.1038/nsmb.2886 (2014).
20 Kumar, N. et al. Global and transcription-coupled repair of 8-oxoG is initiated by nucleotide excision repair proteins. Nature Communications 13, 974, doi: 10.1038/s41467-022-28642-9 (2022).
21 Jang, S. et al. Single molecule analysis indicates stimulation of MUTYH by UV-DDB through enzyme turnover. Nucleic Acids Research 49, 8177-8188, doi: 10.1093/nar/gkab591 (2021).
22 Jang, S. et al. UV-DDB stimulates the activity of SMUG1 during base excision repair of 5-hydroxymethyl-2′-deoxyuridine moieties. Nucleic Acids Research 51, 4881-4898, doi: 10.1093/nar/gkad206 (2023).
23 Hill, J. W., Hazra, T. K., Izumi, T. & Mitra, S. Stimulation of human 8-oxoguanine-DNA glycosylase by AP-endonuclease: potential coordination of the initial steps in base excision repair. Nucleic Acids Res 29, 430-438, doi: 10.1093/nar/29.2.430 (2001).
24 Dantzer, F., Luna, L., Bjørås, M. & Seeberg, E. Human OGG1 undergoes serine phosphorylation and associates with the nuclear matrix and mitotic chromatin in vivo. Nucleic Acids Res 30, 2349-2357, doi: 10.1093/nar/30.11.2349 (2002).
25 Noren Hooten, N., Kompaniez, K., Barnes, J., Lohani, A. & Evans, M. K.

Poly(ADP-ribose) Polymerase 1 (PARP-1) Binds to 8-Oxoguanine-DNA Glycosylase (OGG1)*. Journal of Chemistry Biological 286, 44679-44690, doi: https://doi.org/10.1074/jbc.M111.255869 (2011).
26 Bhakat, K. K., Mokkapati, S. K., Boldogh, I., Hazra, T. K. & Mitra, S. Acetylation of human 8-oxoguanine-DNA glycosylase by p300 and its role in 8-oxoguanine repair in vivo. Mol Cell Biol 26, 1654-1665, doi: 10.1128/mcb.26.5.1654-1665.2006 (2006).
27 Cividini, F. et al. O-GlcNAcylation of 8-Oxoguanine DNA Glycosylase (Ogg1) Impairs Oxidative Mitochondrial DNA Lesion Repair in Diabetic Hearts*. Journal of Biological Chemistry 291, 26515-26528, doi: https://doi.org/10.1074/jbc.M116.754481 (2016).
28 Ba, X. & Boldogh, I. 8-Oxoguanine DNA glycosylase 1: Beyond repair of the oxidatively modified base lesions. Redox Biology 14, 669-678, doi: https://doi.org/10.1016/j.redox.2017.11.008 (2018).
29 Rabe von Pappenheim, F. et al. Widespread occurrence of covalent lysine-cysteine redox switches in proteins. Nature Chemical Biology 18, 368-375, doi: 10.1038/s41589-021-00966-5 (2022).

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. An assay for determining the binding kinetics of one or more proteins with a nucleic acid substrate comprising:
(a) expressing one or more recombinant proteins in a host cell;
(b) preparing a nuclear extract from the host cell expressing the one or more recombinant proteins;
(c) contacting the nuclear extract with a nucleic acid substrate;
(d) visualizing the one or more recombinant proteins binding to the nucleic acid substrate; and
(e) determining protein-nucleic acid association and dissociation kinetics.

2. The assay of claim 1, wherein the nucleic acid substrate is positioned within a microfluidic cell system, and wherein the nuclear extract is flowed through the microfluidic cell system to contact the nucleic acid substrate.

3. The assay of claim 1, wherein the one or more recombinant proteins is selected from a group consisting of poly(ADP-ribose) polymerase 1 (PARP1), heterodimeric ultraviolet-damaged DNA-binding protein (UV-DDB), xeroderma pigmentosum complementation group C protein (XPC), 8-oxoguanine glycosylase 1 (OGG1), apurinic/apyrimidinic endonuclease 1 (APE1), DNA polymerase beta (Polbeta), Thymine DNA glycosylase (TDG), X-ray repair cross complementing 1 (XRCC1), DNA ligase 3 (Lig3α), poly(ADP-ribose) polymerase 2 (PARP2), alkyladenine glycosylase (AAG) or a combination thereof.

4. The assay of claim 3, wherein the one or more recombinant proteins is fluorescently labeled.

5. The assay of claim 1, wherein the host cell is a mammalian cell.

6. The assay of claim 2, wherein the nucleic acid substrate is DNA between about 10 and 100 kb in length.

7. The assay of claim 6, wherein the nucleic acid substrate is damaged.

8. The assay of claim 7, wherein the damage is a physical or a chemical change.

9. The assay of claim 8, wherein the nucleic acid damage is induced by UV exposure, enzymatic digestion, or oxidative damage.

10. The assay of claim 9, wherein the nucleic acid substrate comprises one or more nucleic acid analogues incorporated into the nucleic acid DNA by nick translation; wherein the nucleic acid analogue is selected from a group consisting of 5-formyl-dCTP (5fC), 5-hm-dUTP, 6-thio-dGTP, 5-fluoro-dUTP, ara-CTP, Cy3-dUTP, dITP or a combination thereof.

11. The assay of claim 2, wherein the microfluidic cell system further comprises optical tweezers and a microfluidic cell having at least 4 channels separated by laminar flow;
wherein the at least 4 channels further comprise:
a) channel 1 comprising beads trapped by the optical tweezers;
b) channel 2 comprising the nucleic acid substrate suspended between beads trapped by the optical tweezers;
c) channel 3 comprising the flow of a buffer solution; and/or
d) channel 4 comprising the nuclear extract, wherein the nuclear extract containing one or more recombinant proteins contacts the nucleic acid substrate suspended between beads trapped by the optical tweezers;
wherein the flow rate is kept constant or pulsed;
wherein the flow is between about 0.05 psi and about 5.0 psi;
wherein protein-nucleic acid interactions are observed without flow.

12. The assay of claim 11, wherein the surface of the bead is modified to have a functional group selected from streptavidin, biotin, or poly-lysine.

13. The assay of claim 11, wherein the nucleic acid substrate contains a functional group to facilitate bead attachment;
wherein the functional group is selected from a group consisting of biotin or streptavidin.

14. The assay of claim 11, wherein the nucleic acid substrate is tethered to the beads by a biotin-streptavidin interaction;
wherein the nucleic acid substrate is held at a tension from about 5 to about 40 pN.

15. The assay of claim 11, wherein the microfluidic cell system further comprises fluorescence microscopy comprising single-molecule-FRET imaging;
wherein the fluorescence microscopy detects individual one or more recombinant proteins binding to specific locations along the nucleic acid substrate.

16. The assay of claim 1, wherein the association and dissociation kinetics of the one or more recombinant protein interacting with the nucleic acid substrate comprise:
(a) a binding event duration (k_{off});
(b) number of binding events per second (kₒₙ);
(c) a binding position; and/or
(d) a movement on the nucleic acid substrate (MSD/velocity).

17. A method for determining DNA damage recognition of one or more recombinant proteins using the assay of claim 1.

18. A kit for performing the assays of claim 1, wherein the kit comprises:
(a) a microfluid cell;
(b) a buffer fluid;
(c) a set of beads; and/or
(d) a nucleic acid substrate.

19. The kit of claim 18, wherein the kit further comprises:
(a) instructions for performing single molecule analysis of nucleic acid-binding proteins from nuclear extracts;
(b) tracer dyes; and/or
(c) reagents for conjugating functional groups.


SEQUENCE LISTING (9 sequences)

SEQ ID NO: 1 - DNA, 98 bp, unidentified
  misc_feature 1..98: note = Merkel cell polyomavirus
  source 1..98: mol_type = unassigned DNA
ggtgacttttttttttcaagttggcagaggcttggggctcctagcctccgaggcctctggaaaaaaaagagagaggcctctgaggcttaagaggctta

SEQ ID NO: 2 - DNA, 84 bp, Macaca mulatta
  source 1..84: mol_type = genomic DNA
ctaattttttttatttatgcagaggccgaggccgcctcggcctctgagctattccagaagtagtgaggaggcttttttggaggc

SEQ ID NO: 3 - protein, 6 aa, synthetic construct
  REGION 1..6: note = Synthetic
GSTGSR

SEQ ID NO: 4 - DNA, 18 bp, synthetic construct
  misc_feature 1..18: note = Synthetic
  source 1..18: mol_type = other DNA
gccgccaaggatctgatg

SEQ ID NO: 5 - DNA, 21 bp, synthetic construct
  misc_feature 1..21: note = Synthetic
  source 1..21: mol_type = other DNA
ctgcgcaaggaacgcccgtcg

SEQ ID NO: 6 - DNA, 23 bp, synthetic construct
  misc_feature 1..23: note = Synthetic
  source 1..23: mol_type = other DNA
tgtgtccctcaatatggtcctgt

SEQ ID NO: 7 - DNA, 20 bp, synthetic construct
  misc_feature 1..20: note = Synthetic
  source 1..20: mol_type = other DNA
atggtggtgaagacgccagt

SEQ ID NO: 8 - DNA, 33 bp, synthetic construct
  misc_feature 1..33: note = Synthetic
  source 1..33: mol_type = other DNA
atcgggatccggtgacttttttttttcaagttg

SEQ ID NO: 9 - DNA, 31 bp, synthetic construct
  misc_feature 1..31: note = Synthetic
  source 1..31: mol_type = other DNA
atcggaattctaagcctcttaagcctcagag